(19) United States
(12) Patent Application Publication
(10) Pub. No.: US 2012/0122924 A1
(43) Pub. Date: May 17, 2012

(54) NAMPT INHIBITORS

(75) Inventors: Michael L. Curtin, Pleasant Prairie, WI (US); Bryan K. Sorensen, Antioch, IL (US); Howard R. Heyman, Deerfield, IL (US); Rick F. Clark, Gurnee, IL (US); Michael Michaelides, Libertyville, IL (US); Chris Tse, Libertyville, IL (US)

(73) Assignee: ABBOTT LABORATORIES, Abbott Park, IL (US)

(21) Appl. No.: 13/294,318

(22) Filed: Nov. 11, 2011

Related U.S. Application Data
(60) Provisional application No. 61/413,646, filed on Nov. 15, 2010.

(51) Int. Cl. A61K 31/4709; A61K 31/47; C07D 487/00; A61P 25/28; A61P 1/00; A61P 3/10; A61P 35/00; A61P 9/10; A61P 37/02; A61P 11/06; A61P 11/00; C07D 217/16; A61P 19/10

(52) U.S. Cl. 514/314; 546/146; 514/311; 546/144

(57) ABSTRACT

Disclosed are compounds which inhibit the activity of NAMPT, compositions containing the compounds and methods of treating diseases during which NAMPT is expressed.

### RELATED APPLICATION INFORMATION

This application claims priority to U.S. Provisional Application Ser. No. 61/413,646, filed Nov. 15, 2010, which is incorporated by reference in its entirety.

### FIELD OF THE INVENTION

This invention pertains to compounds which inhibit the activity of NAMPT, compositions containing the compounds, and methods of treating diseases during which NAMPT is expressed.

### BACKGROUND OF THE INVENTION

NAD+ (nicotinamide adenine dinucleotide) is a coenzyme that plays a critical role in many physiologically essential processes (Ziegkel, M. Eur. J. Biochem. 267, 1550-1564, 2000). NAD is necessary for several signaling pathways including among others poly ADP-ribosylation in DNA repair, mono-ADP-ribosylation in both the immune system and G-protein-coupled signaling, and NAD is also required by sirtuins for their deacetylase activity (Garten, A. et al Trends in Endocrinology and Metabolism, 20, 130-138, 2008).

NAMPT (also known as pre-B-cell-colony-enhancing factor (PBEF) and visfatin) is an enzyme that catalyzes the phosphoribosylation of nicotinamide and is the rate-limiting enzyme in one of two pathways that salvage NAD.

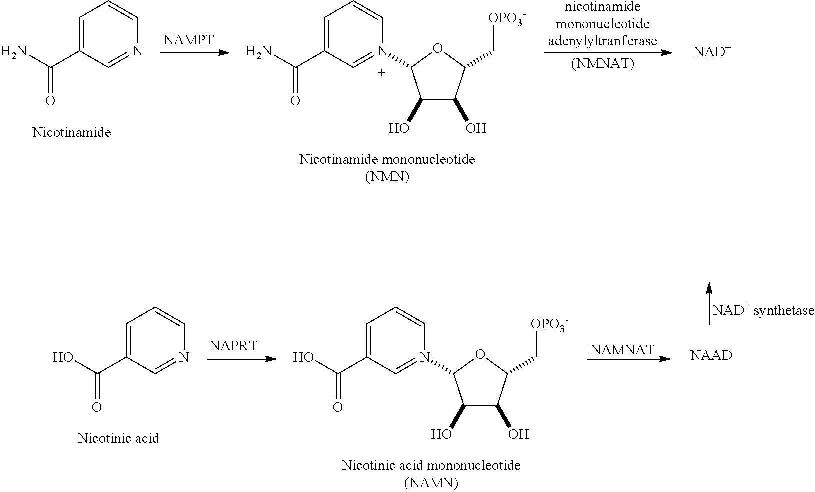

Increasing evidence suggests that NAMPT inhibitors have potential as anticancer agents. Cancer cells have a higher basal turnover of NAD and also display higher energy requirements compared with normal cells. Additionally, increased NAMPT expression has been reported in colorectal cancer (Van Beijnum, J. R. et al Int. J. Cancer 101, 118-127, 2002) and NAMPT is involved in angiogenesis (Kim, S. R. et al. Biochem. Biophys. Res. Commun. 357, 150-156, 2007). Small-molecule inhibitors of NAMPT have been shown to cause depletion of intracellular NAD+ levels and ultimately induce tumor cell death (Hansen, C M et al. Anticancer Res. 20, 42111-4220, 2000) as well as inhibit tumor growth in xenograft models (Olese, U. H. et al. Mol Cancer Ther. 9, 1609-1617, 2010).

NAMPT inhibitors also have potential as therapeutic agents in inflammatory and metabolic disorders (Galli, M. et al Cancer Res. 70, 8-11, 2010). For example, NAMPT is the predominant enzyme in T and B lymphocytes. Selective inhibition of NAMPT leads to NAD+ depletion in lymphocytes blocking the expansion that accompanies autoimmune disease progression whereas cell types expressing the other NAD+ generating pathways might be spared. A small molecule NAMPT inhibitor (FK866) has been shown to selectively block proliferation and induce apoptosis of activated T cells and was efficacious in animal models of arthritis (collagen-induced arthritis) (Busso, N. et al. Plos One 3, e2267, 2008). FK866 ameliorated the manifestations of experimental autoimmune encephalomyelitis (EAE), a model of T-cell mediated autoimmune disorders. (Bruzzone, S et al. Plos One 4, e7897, 2009). NaMPT activity increases NF-kB transcriptional activity in human vascular endothelial cell, resulting in MMP-2 and MMP-9 activation, suggesting a role for NAMPT inhibitors in the prevention of inflammatory mediated complications of obesity and type 2 diabetes (Adya, R. et. Al. Diabetes Care, 31, 758-760, 2008).

### SUMMARY OF THE INVENTION

One embodiment of this invention, therefore, pertains to compounds or pharmaceutically acceptable salts, which are useful as inhibitors of NAMPT, the compounds having Formula (I)

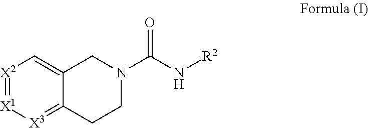

wherein

X¹ and X² and X³ are CH; or

X¹ and X³ are CH; and X² is N; or

X² and X³ are H; and X¹ is N; or

X¹ is CR¹; and X² and X³ are CH; or

X² is CR¹; and X¹ and X³ are CH; or

X³ is CR¹; and X¹ and X² are CH;

R¹ is R³, OR³, SR³, S(O)R³, SO₂R³, C(O)R³, C(O)OR³, OC(O)R³, NHR³, N(R³)₂, C(O)NH₂, C(O)NHR³, C(O)N(R³)₂, NHC(O)R³, NR³C(O)R³, NHC(O)OR³, NR³C(O)OR³, SO₂NH₂, SO₂NHR³, SO₂N(R³)₂, NHSO₂R³, NR³SO₂R³, NHSO₂NHR³, NHSO₂N(R³)₂, NR³SO₂NHR³, NR³SO₂N(R³)₂, C(O)NHSO₂R³, NHSO₂NHR³, F, Cl, Br, I, CN, NH₂, NO₂, N₃, OH, C(O)H, CF₃, C(O)OH, or C(O)NH₂;

R² is alkyl, alkenyl, alkynyl, phenyl, heterocyclyl, cycloalkyl, or cycloalkenyl; wherein each alkyl, alkenyl, and alkynyl is optionally substituted with one or more independently selected R⁴, OR⁴, SR⁴, S(O)R⁴, SO₂R⁴, C(O)R⁴, CO(O)R⁴, OC(O)R⁴, OC(O)OR⁴, NH₂, NHR⁴, N(R⁴)₂, NHC(O)R⁴, NR⁴C(O)R⁴, NHS(O)₂R⁴, NR⁴S(O)₂R⁴, NHC(O)OR⁴, NR⁴C(O)OR⁴, NHC(O)NH₂, NHC(O)NHR⁴, NHC(O)N(R⁴)₂, NR⁴C(O)NHR⁴, NR⁴C(O)N(R⁴)₂, C(O)NH₂, C(O)NHR⁴, C(O)N(R⁴)₂, C(O)NHOH, C(O)NHOR⁴, C(O)NHSO₂R⁴, C(O)NR⁴SO₂R⁴, SO₂NH₂, SO₂NHR⁴, SO₂N(R⁴)₂, C(O)H, C(O)OH, OH, (O), CN, N₃, NO₂, CF₃, CF₂CF₃, OCF₃, OCF₂CF₃, F, Cl, Br or I; wherein each phenyl is optionally additionally substituted at the para position with one independently selected R⁵, OCH₂CH₂CH₂CH₂CH₂CH₃, SR⁵, S(O)R⁵, SO₂R⁵, C(O)R⁵, CO(O)R⁵, OC(O)R⁵, OC(O)OR⁵, NH₂, NHR⁵, N(R⁵)₂, NHC(O)R⁵, NR⁵C(O)R⁵, NHS(O)₂R⁵, NR⁵S(O)₂R⁵, NHC(O)OR⁵, NR⁵C(O)OR⁵, NHC(O)NH₂, NHC(O)NHR⁵, NHC(O)N(R⁵)₂, NR⁵C(O)NHR⁵, NR⁵C(O)N(R⁵)₂, C(O)NH₂, C(O)NHR⁵, C(O)N(R⁵)₂, C(O)NHOH, C(O)NHOR⁵, C(O)NHSO₂R⁵, C(O)NR⁵SO₂R⁵, SO₂NH₂, SO₂NHR⁵, SO₂N(R⁵)₂, C(O)H, C(O)OH, OH, CN, N₃, NO₂, CF₃, CF₂CF₃, OCF₃, OCF₂CF₃, Br or I; wherein each phenyl is optionally additionally substituted with one F; wherein each heterocyclyl, cycloalkyl, and cycloalkenyl is optionally substituted with one or more independently selected R⁵, OR⁵, SR⁵, S(O)R⁵, SO₂R⁵, C(O)R⁵, CO(O)R⁵, OC(O)R⁵, OC(O)OR⁵, NH₂, NHR⁵, N(R⁵)₂, NHC(O)R⁵, NR⁵C(O)R⁵, NHS(O)₂R⁵, NR⁵S(O)₂R⁵, NHC(O)OR⁵, NR⁵C(O)OR⁵, NHC(O)NH₂, NHC(O)NHR⁵, NHC(O)N(R⁵)₂, NR⁵C(O)NHR⁵, NR⁵C(O)N(R⁵)₂, C(O)NH₂, C(O)NHR⁵, C(O)N(R⁵)₂, C(O)NHOH, C(O)NHOR⁵, C(O)NHSO₂R⁵, C(O)NR⁵SO₂R⁵, SO₂NH₂, SO₂NHR⁵, SO₂N(R⁵)₂, C(O)H, C(O)OH, OH, CN, N₃, CF₃, CF₂CF₃, OCF₃, OCF₂CF₃, F, Cl, Br or I; wherein R² is not 4-methylphenyl;

R³ is alkyl, alkenyl, alkynyl, aryl, or heterocyclyl; wherein each alkyl, alkenyl, and alkynyl is optionally substituted with one or more independently selected R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, OH, (O), CN, N₃, NO₂, CF₃, CF₂CF₃, OCF₃, OCF₂CF₃, F, Cl, Br or I;

R⁴ is alkyl, alkenyl, alkynyl, aryl or heterocyclyl; wherein each alkyl, alkenyl, and alkynyl is optionally substituted with one or more independently selected R⁷, OR⁷, SR⁷, S(O)R⁷, SO₂R⁷, C(O)R⁷, CO(O)R⁷, OC(O)R⁷, OC(O)OR⁷, NH₂, NHR⁷, N(R⁷)₂, NHC(O)R⁷, NR⁷C(O)R⁷, NHS(O)₂R⁷, NR⁷S(O)₂R⁷, NHC(O)OR⁷, NR⁷C(O)OR⁷, NHC(O)NH₂, NHC(O)NHR⁷, NHC(O)N(R⁷)₂, NR⁷C(O)NHR⁷, NR⁷C(O)N(R⁷)₂, C(O)NH₂, C(O)NHR⁷, C(O)N(R⁷)₂, C(O)NHOH, C(O)NHOR⁷, C(O)NHSO₂R⁷, C(O)NR⁷SO₂R⁷, SO₂NH₂, SO₂NHR⁷, SO₂N(R⁷)₂, C(O)H, C(O)OH, OH, (O), CN, N₃, NO₂, CF₃, CF₂CF₃, OCF₃, OCF₂CF₃, F, Cl, Br or I; wherein each aryl and heterocycyl is optionally substituted with one or more independently selected R⁸, OR⁸, SR⁸, S(O)R⁸, SO₂R⁸, C(O)R⁸, CO(O)R⁸, OC(O)R⁸, OC(O)OR⁸, NH₂, NHR⁸, N(R⁸)₂, NHC(O)R⁸, NR⁸C(O)R⁸, NHS(O)₂R⁸, NR⁸S(O)₂R⁸, NHC(O)OR⁸, NR⁸C(O)OR⁸, NHC(O)NH₂, NHC(O)NHR⁸, NHC(O)N(R⁸)₂, NR⁸C(O)NHR⁸, NR⁸C(O)N(R⁸)₂, C(O)NH₂, C(O)NHR⁸, C(O)N(R⁸)₂, C(O)NHOH, C(O)NHOR⁸, C(O)NHSO₂R⁸, C(O)NR⁸SO₂R⁸, SO₂NH₂, SO₂NHR⁸, SO₂N(R⁸)₂, C(O)H, C(O)OH, OH, CN, N₃, NO₂, CF₃, CF₂CF₃, OCF₃, OCF₂CF₃, F, Cl, Br or I;

R⁵ is alkyl, alkenyl, alkynyl, aryl, heterocyclyl, cycloalkyl, or cycloalkenyl; wherein each alkyl, alkenyl, and alkynyl is optionally substituted with one or more independently selected R⁹, OR⁹, SR⁹, S(O)R⁹, SO₂R⁹, C(O)R⁹, CO(O)R⁹, OC(O)R⁹, OC(O)OR⁹, NH₂, NHR⁹, N(R⁹)₂, NHC(O)R⁹, NR⁹C(O)R⁹, NHS(O)₂R⁹, NR⁹S(O)₂R⁹, NHC(O)OR⁹, NR⁹C(O)OR⁹, NHC(O)NH₂, NHC(O)NHR⁹, NHC(O)N(R⁹)₂, NR⁹C(O)NHR⁹, NR⁹C(O)N(R⁹)₂, C(O)NH₂, C(O)NHR⁹, C(O)N(R⁹)₂, C(O)NHOH, C(O)NHOR⁹, C(O)NHSO₂R⁹, C(O)NR⁹SO₂R⁹, SO₂NH₂, SO₂NHR⁹, SO₂N(R⁹)₂, C(O)H, C(O)OH, OH, (O), CN, N₃, NO₂, CF₃, CF₂CF₃, OCF₃, OCF₂CF₃, F, Cl, Br or I;

R⁶ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁰, OR¹⁰, SR¹⁰, S(O)R¹⁰, SO₂R¹⁰, NHR¹⁰, N(R¹⁰)₂, C(O)R¹⁰, C(O)NH₂, C(O)NHR¹⁰, C(O)N(R¹⁰)₂, NHC(O)R¹⁰, NR¹⁰C(O)R¹⁰, NHSO₂R¹⁰, NHC(O)OR¹⁰, SO₂NH₂, SO₂NHR¹⁰, SO₂N(R¹⁰)₂, NHC(O)NH₂, NHC(O)NHR¹⁰, OH, (O), C(O)OH, N₃, CN, NH₂, CF₃, CF₂CF₃, F, Cl, Br or I;

R⁷ is alkyl, alkenyl, alkynyl, aryl, heterocyclyl, cycloalkyl, or cycloalkenyl; wherein each alkyl, alkenyl, and alkynyl is optionally substituted with one or more independently selected R¹¹, OR¹¹, SR¹¹, S(O)R¹¹, SO₂R¹¹, NHR¹¹, N(R¹¹)₂, C(O)R¹¹, C(O)NH₂, C(O)NHR¹¹, C(O)N(R¹¹)₂, NHC(O)R¹¹, NR¹¹C(O)R¹¹, NHSO₂R¹¹, NHC(O)OR¹¹, SO₂NH₂, SO₂NHR¹¹, SO₂N(R¹¹)₂, NHC(O)NH₂, NHC(O)NHR¹¹, OH, (O), C(O)OH, N₃, CN, NH₂, CF₃, CF₂CF₃, F, Cl, Br or I;

R⁸ is alkyl, alkenyl, alkynyl, aryl, heterocyclyl, cycloalkyl, or cycloalkenyl; wherein each alkyl, alkenyl, and alkynyl is optionally substituted with one or more independently selected R¹², OR¹², SR¹², S(O)R¹², SO₂R¹², NHR¹², N(R¹²)₂, C(O)R¹², C(O)NH₂, C(O)NHR¹², C(O)N(R¹²)₂, NHC(O)R¹², NR¹²C(O)R¹², NHSO₂R¹², NHC(O)OR¹², SO₂NH₂, SO₂NHR¹², SO₂N(R¹²)₂, NHC(O)NH₂, NHC(O)NHR¹², OH, (O), C(O)OH, N₃, CN, NH₂, CF₃, CF₂CF₃, F, Cl, Br or I;

R⁹ is alkyl, alkenyl, alkynyl, aryl, heterocyclyl, cycloalkyl, or cycloalkenyl; wherein each alkyl, alkenyl, and alkynyl is optionally substituted with one or more independently selected OCH₃, aryl, or heterocyclyl;

R¹⁰ is alkyl, alkenyl, alkynyl, aryl, heterocyclyl, cycloalkyl, or cycloalkenyl;

R¹¹ is alkyl, alkenyl, alkynyl, aryl, heterocyclyl, cycloalkyl, or cycloalkenyl;

R¹² is alkyl, alkenyl, alkynyl, aryl, heterocyclyl, cycloalkyl, or cycloalkenyl;

wherein the cyclic moieties represented by R³, R⁵, R⁶, R⁷, R⁸, R⁹, R¹⁰, R¹¹, and R¹² are optionally substituted with one or more independently selected R¹³, OR¹³, SR¹³, S(O)R¹³, SO₂R¹³, C(O)R¹³, CO(O)R¹³, OC(O)R¹³, OC(O)OR¹³, NH₂, NHR¹³, N(R¹³)₂, NHC(O)R¹³, NR¹³C(O)R¹³, NHS(O)₂R¹³, NR¹³S(O)₂R¹³, NHC(O)OR¹³, NR¹³C(O)OR¹³, NHC(O)NH₂, NHC(O)NHR¹³, NHC(O)N(R¹³)₂, NR¹³C(O)NHR¹³, NR¹³C(O)N(R¹³)₂, C(O)NH₂, C(O)NHR¹³, C(O)N(R¹³)₂, C(O)NHOH, C(O)NHOR¹³, C(O)NHSO₂R¹³, C(O)NR¹³SO₂R¹³, SO₂NH₂, SO₂NHR¹³, SO₂N(R¹³)₂, C(O)H, C(O)OH, OH, CN, N₃, NO₂, CF₃, CF₂CF₃, OCF₃, OCF₂CF₃, F, Cl, Br or I;

R¹³ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁴, OR¹⁴, SR¹⁴, S(O)R¹⁴, SO₂R¹⁴, C(O)R¹⁴, CO(O)R¹⁴, OC(O)R¹⁴, OC(O)OR¹⁴, NH₂, NHR¹⁴, N(R¹⁴)₂, NHC(O)R¹⁴, NR¹⁴C(O)R¹⁴, NHS(O)₂R¹⁴, NR¹⁴S(O)₂R¹⁴, NHC(O)OR¹⁴, NR¹⁴C(O)OR¹⁴, NHC(O)NH₂, NHC(O)NHR¹⁴, NHC(O)N(R¹⁴)₂, NR¹⁴C(O)NHR¹⁴, NR¹⁴C(O)N(R¹⁴)₂, C(O)NH₂, C(O)NHR¹⁴, C(O)N(R¹⁴)₂, C(O)NHOH, C(O)NHOR¹⁴, C(O)NHSO₂R¹⁴, C(O)NR¹⁴SO₂R¹⁴, SO₂NH₂, SO₂NHR¹⁴, SO₂N(R¹⁴)₂, C(O)H, C(O)OH, OH, (O), CN, N₃, NO₂, CF₃, CF₂CF₃, OCF₃, OCF₂CF₃, F, Cl, Br or I; wherein each aryl, heterocyclyl, cycloalkyl, and cycloalkenyl is optionally substituted with one or more independently selected R¹⁵, OR¹⁵, SR¹⁵, S(O)R¹⁵, SO₂R¹⁵, C(O)R¹⁵, CO(O)R¹⁵, OC(O)R¹⁵, OC(O)OR¹⁵, NH₂, NHR¹⁵, N(R¹⁵)₂, NHC(O)R¹⁵, NR¹⁵C(O)R¹⁵, NHS(O)₂R¹⁵, NR¹⁵S(O)₂R¹⁵, NHC(O)OR¹⁵, NR¹⁵C(O)OR¹⁵, NHC(O)NH₂, NHC(O)NHR¹⁵, NHC(O)N(R¹⁵)₂, NR¹⁵C(O)NHR¹⁵, NR¹⁵C(O)N(R¹⁵)₂, C(O)NH₂, C(O)NHR¹⁵, C(O)N(R¹⁵)₂, C(O)NHOH, C(O)NHOR¹⁵, C(O)NHSO₂R¹⁵, C(O)NR¹⁵SO₂R¹⁵, SO₂NH₂, SO₂NHR¹⁵, SO₂N(R¹⁵)₂, C(O)H, C(O)OH, OH, CN, N₃, NO₂, CF₃, CF₂CF₃, OCF₃, OCF₂CF₃, F, Cl, Br or I;

R¹⁴ is alkyl, alkenyl, alkynyl, aryl, heterocyclyl, cycloalkyl, or cycloalkenyl; wherein each alkyl, alkenyl, and alkynyl is optionally substituted with one or more independently selected NH₂, SO₂NH₂, C(O)H, C(O)OH, OH, (O), CN, N₃, NO₂, CF₃, CF₂CF₃, OCF₃, OCF₂CF₃, F, Cl, Br or I; and

R¹⁵ is alkyl.

Another embodiment of this invention pertains to compounds or pharmaceutically acceptable salts thereof; which are useful as inhibitors of NAMPT, the compounds having Formula (V)

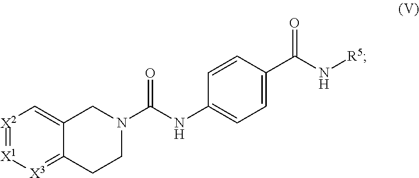

wherein

X¹ and X² and X³ are CH; or

X¹ and X³ are CH; and X² is N; or

X² and X³ are H; and X¹ is N; or

X¹ is CR¹; and X² and X³ are CH; or

X² is CR¹; and X¹ and X³ are CH; or

X³ is CR¹; and X¹ and X² are CH;

R¹ is R³, OR³, SR³, S(O)R³, SO₂R³, C(O)R³, C(O)OR³, OC(O)R³, NHR³, N(R³)₂, C(O)NH₂, C(O)NHR³, C(O)N(R³)₂, NHC(O)R³, NR³C(O)R³, NHC(O)OR³, NR³C(O)OR³, SO₂NH₂, SO₂NHR³, SO₂N(R³)₂, NHSO₂R³, NR³SO₂R³, NHSO₂NHR³, NHSO₂N(R³)₂, NR³SO₂NHR³, NR³SO₂N(R³)₂, C(O)NHSO₂R³, NHSO₂NHR³, F, Cl, Br, I, CN, NH₂, NO₂, N₃, OH, C(O)H, CF₃, C(O)OH, or C(O)NH₂;

R³ is alkyl, alkenyl, alkynyl, aryl, or heterocyclyl; wherein each alkyl, alkenyl, and alkynyl is optionally substituted with one or more independently selected R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, OH, (O), CN, N₃, NO₂, CF₃, CF₂CF₃, OCF₃, OCF₂CF₃, F, Cl, Br or I;

R⁵ is alkyl, alkenyl, alkynyl, aryl, heterocyclyl, cycloalkyl, or cycloalkenyl; wherein each alkyl, alkenyl, and alkynyl is optionally substituted with one or more independently selected R⁹, OR⁹, SR⁹, S(O)R⁹, SO₂R⁹, C(O)R⁹, CO(O)R⁹, OC(O)R⁹, OC(O)OR⁹, NH₂, NHR⁹, N(R⁹)₂, NHC(O)R⁹, NR⁹C(O)R⁹, NHS(O)₂R⁹, NR⁹S(O)₂R⁹, NHC(O)OR⁹, NR⁹C(O)OR⁹, NHC(O)NH₂, NHC(O)NHR⁹, NHC(O)N(R⁹)₂, NR⁹C(O)NHR⁹, NR⁹C(O)N(R⁹)₂, C(O)NH₂, C(O)NHR⁹, C(O)N(R⁹)₂, C(O)NHOH, C(O)NHOR⁹, C(O)NHSO₂R⁹, C(O)NR⁹SO₂R⁹, SO₂NH₂, SO₂NHR⁹, SO₂N(R⁹)₂, C(O)H, C(O)OH, OH, (O), CN, N₃, NO₂, CF₃, CF₂CF₃, OCF₃, OCF₂CF₃, F, Cl, Br or I;

R⁶ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁰, OR¹⁰, SR¹⁰, S(O)R¹⁰, SO₂R¹⁰, NHR¹⁰, N(R¹⁰)₂, C(O)R¹⁰, C(O)NH₂, C(O)NHR¹⁰, C(O)N(R¹⁰)₂, NHC(O)R¹⁰, NR¹⁰C(O)R¹⁰, NHSO₂R¹⁰, NHC(O)OR¹⁰, SO₂NH₂, SO₂NHR¹⁰, SO₂N(R¹⁰)₂, NHC(O)NH₂, NHC(O)NHR¹⁰, OH, (O), C(O)OH, N₃, CN, NH₂, CF₃, CF₂CF₃, F, Cl, Br or I;

R⁹ is alkyl, alkenyl, alkynyl, aryl, heterocyclyl, cycloalkyl, or cycloalkenyl; wherein each alkyl, alkenyl, and alkynyl is optionally substituted with one or more independently selected OCH₃, aryl, or heterocyclyl;

R¹⁰ is alkyl, alkenyl, alkynyl, aryl, heterocyclyl, cycloalkyl, or cycloalkenyl;

wherein the cyclic moieties represented by R³, R⁵, R⁶, R⁹, and R¹⁰, are optionally substituted with one or more independently selected R¹³, OR¹³, SR¹³, S(O)R¹³, SO₂R¹³, C(O)R¹³, CO(O)R¹³, OC(O)R¹³, OC(O)OR¹³, NH₂, NHR¹³, N(R¹³)₂, NHC(O)R¹³, NR¹³C(O)R¹³, NHS(O)₂R¹³, NR¹³S(O)₂R¹³, NHC(O)OR¹³, NR¹³C(O)OR¹³, NHC(O)NH₂, NHC(O)NHR¹³, NHC(O)N(R¹³)₂, NR¹³C(O)NHR¹³, NR¹³C(O)N(R¹³)₂, C(O)NH₂, C(O)NHR¹³, C(O)N(R¹³)₂, C(O)NHOH, C(O)NHOR¹³, C(O)NHSO₂R¹³, C(O)NR¹³SO₂R¹³, SO₂NH₂, SO₂NHR¹³, SO₂N(R¹³)₂, C(O)H, C(O)OH, OH, CN, N₃, NO₂, CF₃, CF₂CF₃, OCF₃, OCF₂CF₃, F, Cl, Br or I;

R¹³ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁴, OR¹⁴, SR¹⁴, S(O)R¹⁴, SO₂R¹⁴, C(O)R¹⁴, CO(O)R¹⁴, OC(O)R¹⁴, OC(O)OR¹⁴, NH₂, NHR¹⁴, N(R¹⁴)₂, NHC(O)R¹⁴, NR¹⁴C(O)R¹⁴, NHS(O)₂R¹⁴, NR¹⁴S(O)₂R¹⁴, NHC(O)OR¹⁴, NR¹⁴C(O)OR¹⁴, NHC(O)NH₂, NHC(O)NHR¹⁴, NHC(O)N(R¹⁴)₂, NR¹⁴C(O)NHR¹⁴, NR¹⁴C(O)N(R¹⁴)₂, C(O)NH₂, C(O)NHR¹⁴, C(O)N(R¹⁴)₂, C(O)NHOH, C(O)NHOR¹⁴, C(O)NHSO₂R¹⁴, C(O)NR¹⁴SO₂R¹⁴, SO₂NH₂, SO₂NHR¹⁴, SO₂N(R¹⁴)₂, C(O)H, C(O)OH, OH, (O), CN, N₃, NO₂, CF₃, CF₂CF₃, OCF₃, OCF₂CF₃, F, Cl, Br or I; wherein each aryl, heterocyclyl, cycloalkyl, and cycloalkenyl is optionally substituted with one or more independently selected R¹⁵, OR¹⁵, SR¹⁵, S(O)R¹⁵, SO₂R¹⁵, C(O)R¹⁵, CO(O)R¹⁵, OC(O)R¹⁵, OC(O)OR¹⁵, NH₂, NHR¹⁵, N(R¹⁵)₂, NHC(O)R¹⁵, NR¹⁵C(O)R¹⁵, NHS(O)₂R¹⁵, NR¹⁵S(O)₂R¹⁵, NHC(O)OR¹⁵, NR¹⁵C(O)OR¹⁵, NHC(O)NH₂, NHC(O)NHR¹⁵, NHC(O)N(R¹⁵)₂, NR¹⁵C(O)NHR¹⁵, NR¹⁵C(O)N(R¹⁵)₂, C(O)NH₂, C(O)NHR¹⁵, C(O)N(R¹⁵)₂, C(O)NHOH, C(O)NHOR¹⁵, C(O)NHSO₂R¹⁵, C(O)NR¹⁵SO₂R¹⁵, SO₂NH₂, SO₂NHR¹⁵, SO₂N(R¹⁵)₂, C(O)H, C(O)OH, OH, CN, N₃, NO₂, CF₃, CF₂CF₃, OCF₃, OCF₂CF₃, F, Cl, Br or I;

R¹⁴ is alkyl, alkenyl, alkynyl, aryl, heterocyclyl, cycloalkyl, or cycloalkenyl; wherein each alkyl, alkenyl, and alkynyl is optionally substituted with one or more independently selected NH₂, SO₂NH₂, C(O)H, C(O)OH, OH, (O), CN, N₃, NO₂, CF₃, CF₂CF₃, OCF₃, OCF₂CF₃, F, Cl, Br or I; and

R¹⁵ is alkyl.

Another embodiment of this invention pertains to compounds or pharmaceutically acceptable salts thereof; which are useful as inhibitors of NAMPT, the compounds having Formula (IV)

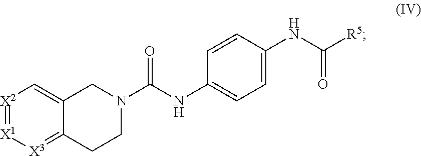

wherein

X¹ and X² and X³ are CH; or

X¹ and X³ are CH; and X² is N; or

X² and X³ are H; and X¹ is N; or

X¹ is CR¹; and X² and X³ are CH; or

X² is CR¹; and X¹ and X³ are CH; or

X³ is CR¹; and X¹ and X² are CH;

R¹ is R³, OR³, SR³, S(O)R³, SO₂R³, C(O)R³, C(O)OR³, OC(O)R³, NHR³, N(R³)₂, C(O)NH₂, C(O)NHR³, C(O)N(R³)₂, NHC(O)R³, NR³C(O)R³, NHC(O)OR³, NR³C(O)OR³, SO₂NH₂, SO₂NHR³, SO₂N(R³)₂, NHSO₂R³, NR³SO₂R³, NHSO₂NHR³, NHSO₂N(R³)₂, NR³SO₂NHR³, NR³SO₂N(R³)₂, C(O)NHSO₂R³, NHSO₂NHR³, F, Cl, Br, I, CN, NH₂, NO₂, N₃, OH, C(O)H, CF₃, C(O)OH, or C(O)NH₂;

R³ is alkyl, alkenyl, alkynyl, aryl, or heterocyclyl; wherein each alkyl, alkenyl, and alkynyl is optionally substituted with one or more independently selected R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, OH, (O), CN, N₃, NO₂, CF₃, CF₂CF₃, OCF₃, OCF₂CF₃, F, Cl, Br or I;

R⁵ is alkyl, alkenyl, alkynyl, aryl, heterocyclyl, cycloalkyl, or cycloalkenyl; wherein each alkyl, alkenyl, and alkynyl is optionally substituted with one or more independently selected R⁹, OR⁹, SR⁹, S(O)R⁹, SO₂R⁹, C(O)R⁹, CO(O)R⁹, OC(O)R⁹, OC(O)OR⁹, NH₂, NHR⁹, N(R⁹)₂, NHC(O)R⁹, NR⁹C(O)R⁹, NHS(O)₂R⁹, NR⁹S(O)₂R⁹, NHC(O)OR⁹, NR⁹C(O)OR⁹, NHC(O)NH₂, NHC(O)NHR⁹, NHC(O)N(R⁹)₂, NR⁹C(O)NHR⁹, NR⁹C(O)N(R⁹)₂, C(O)NH₂, C(O)NHR⁹, C(O)N(R⁹)₂, C(O)NHOH, C(O)NHOR⁹, C(O)NHSO₂R⁹, C(O)NR⁹SO₂R⁹, SO₂NH₂, SO₂NHR⁹, SO₂N(R⁹)₂, C(O)H, C(O)OH, OH, (O), CN, N₃, NO₂, CF₃, CF₂CF₃, OCF₃, OCF₂CF₃, F, Cl, Br or I;

R⁶ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁰, OR¹⁰, SR¹⁰, S(O)R¹⁰, SO₂R¹⁰, NHR¹⁰, N(R¹⁰)₂, C(O)R¹⁰, C(O)NH₂, C(O)NHR¹⁰, C(O)N(R¹⁰)₂, NHC(O)R¹⁰, NR¹⁰C(O)R¹⁰, NHSO₂R¹⁰, NHC(O)OR¹⁰, SO₂NH₂, SO₂NHR¹⁰, SO₂N(R¹⁰)₂, NHC(O)NH₂, NHC(O)NHR¹⁰, OH, (O), C(O)OH, N₃, CN, NH₂, CF₃, CF₂CF₃, F, Cl, Br or I;

R⁹ is alkyl, alkenyl, alkynyl, aryl, heterocyclyl, cycloalkyl, or cycloalkenyl; wherein each alkyl, alkenyl, and alkynyl is optionally substituted with one or more independently selected OCH₃, aryl, or heterocyclyl;

R¹⁰ is alkyl, alkenyl, alkynyl, aryl, heterocyclyl, cycloalkyl, or cycloalkenyl;

wherein the cyclic moieties represented by R³, R⁵, R⁶, R⁹, and R¹⁰, are optionally substituted with one or more independently selected R¹³, OR¹³, SR¹³, S(O)R¹³, SO₂R¹³, C(O)R¹³, CO(O)R¹³, OC(O)R¹³, OC(O)OR¹³, NH₂, NHR¹³, N(R¹³)₂, NHC(O)R¹³, NR¹³C(O)R¹³, NHS(O)₂R¹³, NR¹³S(O)₂R¹³, NHC(O)OR¹³, NR¹³C(O)OR¹³, NHC(O)NH₂, NHC(O)NHR¹³, NHC(O)N(R¹³)₂, NR¹³C(O)NHR¹³, NR¹³C(O)N(R¹³)₂, C(O)NH₂, C(O)NHR¹³, C(O)N(R¹³)₂, C(O)NHOH, C(O)NHOR¹³, C(O)NHSO₂R¹³, C(O)NR¹³SO₂R¹³, SO₂NH₂, SO₂NHR¹³, SO₂N(R¹³)₂, C(O)H, C(O)OH, OH, CN, N₃, NO₂, CF₃, CF₂CF₃, OCF₃, OCF₂CF₃, F, Cl, Br or I;

R¹³ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁴, OR¹⁴, SR¹⁴, S(O)R¹⁴, SO₂R¹⁴, C(O)R¹⁴, CO(O)R¹⁴, OC(O)R¹⁴, OC(O)OR¹⁴, NH₂, NHR¹⁴, N(R¹⁴)₂, NHC(O)R¹⁴, NR¹⁴C(O)R¹⁴, NHS(O)₂R¹⁴, NR¹⁴S(O)₂R¹⁴, NHC(O)OR¹⁴, NR¹⁴C(O)OR¹⁴, NHC(O)NH₂, NHC(O)NHR¹⁴, NHC(O)N(R¹⁴)₂, NR¹⁴C(O)NHR¹⁴, NR¹⁴C(O)N(R¹⁴)₂, C(O)NH₂, C(O)NHR¹⁴, C(O)N(R¹⁴)₂, C(O)NHOH, C(O)NHOR¹⁴, C(O)NHSO₂R¹⁴, C(O)NR¹⁴SO₂R¹⁴, SO₂NH₂, SO₂NHR¹⁴, SO₂N(R¹⁴)₂, C(O)H, C(O)OH, OH, (O), CN, N₃, NO₂, CF₃, CF₂CF₃, OCF₃, OCF₂CF₃, F, Cl, Br or I; wherein each aryl, heterocyclyl, cycloalkyl, and cycloalkenyl is optionally substituted with one or more independently selected R¹⁵, OR¹⁵, SR¹⁵, S(O)R¹⁵, SO₂R¹⁵, C(O)R¹⁵, CO(O)R¹⁵, OC(O)R¹⁵, OC(O)OR¹⁵, NH₂, NHR¹⁵, N(R¹⁵)₂, NHC(O)R¹⁵, NR¹⁵C(O)R¹⁵, NHS(O)₂R¹⁵, NR¹⁵S(O)₂R¹⁵, NHC(O)OR¹⁵, NR¹⁵C(O)OR¹⁵, NHC(O)NH₂, NHC(O)NHR¹⁵, NHC(O)N(R¹⁵)₂, NR¹⁵C(O)NHR¹⁵, NR¹⁵C(O)N(R¹⁵)₂, C(O)NH₂, C(O)NHR¹⁵, C(O)N(R¹⁵)₂, C(O)NHOH, C(O)NHOR¹⁵, C(O)NHSO₂R¹⁵, C(O)NR¹⁵SO₂R¹⁵, SO₂NH₂, SO₂NHR¹⁵, SO₂N(R¹⁵)₂, C(O)H, C(O)OH, OH, CN, N₃, NO₂, CF₃, CF₂CF₃, OCF₃, OCF₂CF₃, F, Cl, Br or I;

R¹⁴ is alkyl, alkenyl, alkynyl, aryl, heterocyclyl, cycloalkyl, or cycloalkenyl; wherein each alkyl, alkenyl, and alkynyl is optionally substituted with one or more independently selected NH₂, SO₂NH₂, C(O)H, C(O)OH, OH, (O), CN, N₃, NO₂, CF₃, CF₂CF₃, OCF₃, OCF₂CF₃, F, Cl, Br or I; and

R¹⁵ is alkyl.

Another embodiment of this invention pertains to compounds or pharmaceutically acceptable salts thereof; which are useful as inhibitors of NAMPT, the compounds having Formula (VI)

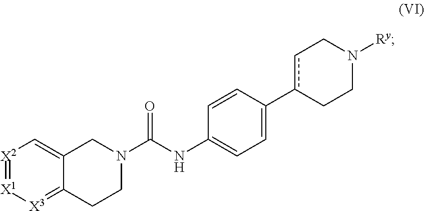

wherein

[symbol] indicates a single or a double bond;

X¹ and X² and X³ are CH; or

X¹ and X³ are CH; and X² is N; or

X² and X³ are H; and X¹ is N; or

X¹ is CR¹; and X² and X³ are CH; or

X² is CR¹; and X¹ and X³ are CH; or

X³ is CR¹; and X¹ and X² are CH;

R¹ is R³, OR³, SR³, S(O)R³, SO₂R³C(O)R³, C(O)OR³, OC(O)R³, NHR³, N(R³)₂, C(O)NH₂, C(O)NHR³, C(O)N(R³)₂, NHC(O)R³, NR³C(O)R³, NHC(O)OR³, NR³C(O)OR³, SO₂NH₂, SO₂NHR³, SO₂N(R³)₂, NHSO₂R³, NR³SO₂R³, NHSO₂NHR³, NHSO₂N(R³)₂, NR³SO₂NHR³, NR³SO₂N(R³)₂, C(O)NHSO₂R³, NHSO₂NHR³, F, Cl, Br, I, CN, NH₂, NO₂, N₃, OH, C(O)H, CF₃, C(O)OH, or C(O)NH₂;

R³ is alkyl, alkenyl, alkynyl, aryl, or heterocyclyl; wherein each alkyl, alkenyl, and alkynyl is optionally substituted with one or more independently selected R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, OH, (O), CN, N₃, NO₂, CF₃, CF₂CF₃, OCF₃, OCF₂CF₃, F, Cl, Br or I;

R⁶ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁰, OR¹⁰, SR¹⁰, S(O)R¹⁰, SO₂R¹⁰, NHR¹⁰, N(R¹⁰)₂, C(O)R¹⁰, C(O)NH₂, C(O)NHR¹⁰, C(O)N(R¹⁰)₂, NHC(O)R¹⁰, NR¹⁰C(O)R¹⁰, NHSO₂R¹⁰, NHC(O)OR¹⁰, SO₂NH₂, SO₂NHR¹⁰, SO₂N(R¹⁰)₂, NHC(O)NH₂, NHC(O)NHR¹⁰, OH, (O), C(O)OH, N₃, CN, NH₂, CF₃, CF₂CF₃, F, Cl, Br or I;

R¹⁰ is alkyl, alkenyl, alkynyl, aryl, heterocyclyl, cycloalkyl, or cycloalkenyl;

R^{y} is R¹³, OR¹³, SR¹³, S(O)R¹³, SO₂R¹³, C(O)R¹³, CO(O)R¹³, OC(O)R¹³, OC(O)OR¹³, NH₂, NHR¹³, N(R¹³)₂, NHC(O)R¹³, NR¹³C(O)R¹³, NHS(O)₂R¹³, NR¹³S(O)₂R¹³, NHC(O)OR¹³, NR¹³C(O)OR¹³, NHC(O)NH₂, NHC(O)NHR¹³, NHC(O)N(R¹³)₂, NR¹³C(O)NHR¹³, NR¹³C(O)N(R¹³)₂, C(O)NH₂, C(O)NHR¹³, C(O)N(R¹³)₂, C(O)NHOH, C(O)NHOR¹³, C(O)NHSO₂R¹³, C(O)NR¹³SO₂R¹³, SO₂NH₂, SO₂NHR¹³, SO₂N(R¹³)₂, C(O)H, C(O)OH, OH, CN, N₃, NO₂, CF₃, CF₂CF₃, OCF₃, OCF₂CF₃, F, Cl, Br or I;

R¹³ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁴, OR¹⁴, SR¹⁴, S(O)R¹⁴, SO₂R¹⁴, C(O)R¹⁴, CO(O)R¹⁴, OC(O)R¹⁴, OC(O)OR¹⁴, NH₂, NHR¹⁴, N(R¹⁴)₂, NHC(O)R¹⁴, NR¹⁴C(O)R¹⁴, NHS(O)₂R¹⁴, NR¹⁴S(O)₂R¹⁴, NHC(O)OR¹⁴, NR¹⁴C(O)OR¹⁴, NHC(O)NH₂, NHC(O)NHR¹⁴, NHC(O)N(R¹⁴)₂, NR¹⁴C(O)NHR¹⁴, NR¹⁴C(O)N(R¹⁴)₂, C(O)NH₂, C(O)NHR¹⁴, C(O)N(R¹⁴)₂, C(O)NHOH, C(O)NHOR¹⁴, C(O)NHSO₂R¹⁴, C(O)NR¹⁴SO₂R¹⁴, SO₂NH₂, SO₂NHR¹⁴, SO₂N(R¹⁴)₂, C(O)H, C(O)OH, OH, (O), CN, N₃, NO₂, CF₃, CF₂CF₃, OCF₃, OCF₂CF₃, F, Cl, Br or I; wherein each aryl, heterocyclyl, cycloalkyl, and cycloalkenyl is optionally substituted with one or more independently selected R¹⁵, OR¹⁵, SR¹⁵, S(O)R¹⁵, SO₂R¹⁵, C(O)R¹⁵, CO(O)R¹⁵, OC(O)R¹⁵, OC(O)OR¹⁵, NH₂, NHR¹⁵, N(R¹⁵)₂, NHC(O)R¹⁵, NR¹⁵C(O)R¹⁵, NHS(O)₂R¹⁵, NR¹⁵S(O)₂R¹⁵, NHC(O)OR¹⁵, NR¹⁵C(O)OR¹⁵, NHC(O)NH₂, NHC(O)NHR¹⁵, NHC(O)N(R¹⁵)₂, NR¹⁵C(O)NHR¹⁵, NR¹⁵C(O)N(R¹⁵)₂, C(O)NH₂, C(O)NHR¹⁵, C(O)N(R¹⁵)₂, C(O)NHOH, C(O)NHOR¹⁵, C(O)NHSO₂R¹⁵, C(O)NR¹⁵SO₂R¹⁵, SO₂NH₂, SO₂NHR¹⁵, SO₂N(R¹⁵)₂, C(O)H, C(O)OH, OH, CN, N₃, NO₂, CF₃, CF₂CF₃, OCF₃, OCF₂CF₃, F, Cl, Br or I;

R¹⁴ is alkyl, alkenyl, alkynyl, aryl, heterocyclyl, cycloalkyl, or cycloalkenyl; wherein each alkyl, alkenyl, and alkynyl is optionally substituted with one or more independently selected NH₂, SO₂NH₂, C(O)H, C(O)OH, OH, (O), CN, N₃, NO₂, CF₃, CF₂CF₃, OCF₃, OCF₂CF₃, F, Cl, Br or I; and

R¹⁵ is alkyl.

In one embodiment of Formula (VI), [symbol] is a double bond.

Another embodiment pertains to compounds of Formula (I), (IV), (V), (VI), and pharmaceutically acceptable salts thereof; wherein X¹, X², and X³ are CH; or X¹ is CR¹ and X² and X³ are CH; or X² is CR¹ and X¹ and X³ are CH.

Another embodiment pertains to compounds of (I), (IV), (V), (VI), and pharmaceutically acceptable salts thereof; wherein X¹ and X³ are CH; and X² is N; or X² and X³ are CH; and X¹ is N.

Still another embodiment pertains to compounds, which are
N-{4-[(3-methylbutyl)carbamoyl]phenyl}-3,4-dihydroisoquinoline-2(1H)-carboxamide;
N-(4-{[4-(pyridin-2-yl)piperazin-1-yl]carbonyl}phenyl)-3,4-dihydroisoquinoline-2(1H)-carboxamide;
6-fluoro-N-{4-[(3-phenylpropyl)carbamoyl]phenyl}-3,4-dihydroisoquinoline-2(1H)-carboxamide;
N-{4-[(3-phenylpropyl)carbamoyl]phenyl}-3,4-dihydro-2,6-naphthyridine-2(1H)-carboxamide;
N-{4-[(3-methylbutyl)carbamoyl]phenyl}-3,4-dihydro-2,6-naphthyridine-2(1H)-carboxamide;
6-fluoro-N-{4-[(3-methylbutyl)carbamoyl]phenyl}-3,4-dihydroisoquinoline-2(1H)-carboxamide;
N-{4-[(3-phenylpropyl)carbamoyl]phenyl}-3,4-dihydroisoquinoline-2(1H)-carboxamide;
N-[4-(benzylcarbamoyl)phenyl]-3,4-dihydroisoquinoline-2(1H)-carboxamide;
N-{5-[(3-phenylpropyl)carbamoyl]pyridin-2-yl}-3,4-dihydroisoquinoline-2(1H)-carboxamide;
N-{5-[(3-methylbutyl)carbamoyl]pyridin-2-yl}-3,4-dihydroisoquinoline-2(1H)-carboxamide;
N-(4-{[1-(3-methylbutyl)-1H-pyrazol-4-yl]carbamoyl}phenyl)-3,4-dihydroisoquinoline-2(1H)-carboxamide;
N-(4-{[2-(2-thienyl)ethyl]carbamoyl}phenyl)-3,4-dihydro-2,6-naphthyridine-2(1H)-carboxamide;
6-fluoro-N-(4-{[2-(2-thienyl)ethyl]carbamoyl}phenyl)-3,4-dihydroisoquinoline-2(1H)-carboxamide;
N-{4-[(1-benzyl-1H-pyrazol-4-yl)carbamoyl]phenyl}-3,4-dihydroisoquinoline-2(1H)-carboxamide;
N-{4-[(2-phenylethyl)carbamoyl]phenyl}-3,4-dihydroisoquinoline-2(1H)-carboxamide;
7-fluoro-N-{4-[(3-methylbutyl)carbamoyl]phenyl}-3,4-dihydroisoquinoline-2(1H)-carboxamide;
N-(4-{[2-(2-thienyl)ethyl]carbamoyl}phenyl)-3,4-dihydroisoquinoline-2(1H)-carboxamide;
N-{6-[(3-methylbutyl)carbamoyl]pyridin-3-yl}-3,4-dihydroisoquinoline-2(1H)-carboxamide;
N-(5-{[2-(2-thienyl)ethyl]carbamoyl}pyridin-2-yl)-3,4-dihydroisoquinoline-2(1H)-carboxamide;
7-fluoro-N-{4-[(3-phenylpropyl)carbamoyl]phenyl}-3,4-dihydroisoquinoline-2(1H)-carboxamide;
N-{6-[(3-phenylpropyl)carbamoyl]pyridin-3-yl}-3,4-dihydroisoquinoline-2(1H)-carboxamide;
N-(6-{[2-(2-thienyl)ethyl]carbamoyl}pyridin-3-yl)-3,4-dihydroisoquinoline-2(1H)-carboxamide;
7-fluoro-N-(4-{[2-(2-thienyl)ethyl]carbamoyl}phenyl)-3,4-dihydroisoquinoline-2(1H)-carboxamide;
N-{4-[(3-phenylpropyl)carbamoyl]phenyl}-3,4-dihydro-2,7-naphthyridine-2(1H)-carboxamide;
N-[4-(2-oxo-2-{[2-(2-thienyl)ethyl]amino}ethyl)phenyl]-3,4-dihydroisoquinoline-2(1H)-carboxamide;
N-(4-{2-oxo-2-[(3-phenylpropyl)amino]ethyl}phenyl)-3,4-dihydroisoquinoline-2(1H)-carboxamide;
N-{4-[(3-methylbutyl)carbamoyl]phenyl}-3,4-dihydro-2,7-naphthyridine-2(1H)-carboxamide;
N-(4-{2-[(3-methylbutyl)amino]-2-oxoethyl}phenyl)-3,4-dihydroisoquinoline-2(1H)-carboxamide;
N-(4-{[2-(2-thienyl)ethyl]carbamoyl}phenyl)-3,4-dihydro-2,7-naphthyridine-2(1H)-carboxamide;
N-{4-[(4-methylpentanoyl)amino]phenyl}-3,4-dihydroisoquinoline-2(1H)-carboxamide;
N-{4-[(4-phenylbutanoyl)amino]phenyl}-3,4-dihydroisoquinoline-2(1H)-carboxamide;
N-(4-{[3-(2-thienyl)propanoyl]amino}phenyl)-3,4-dihydroisoquinoline-2(1H)-carboxamide;
N-[4-(1-isobutyl-1H-pyrazol-4-yl)phenyl]-3,4-dihydroisoquinoline-2(1H)-carboxamide;
N-[4-(1-propyl-1H-pyrazol-4-yl)phenyl]-3,4-dihydroisoquinoline-2(1H)-carboxamide;
N-{4-[1-((2R)-2-hydroxypropyl)-1H-pyrazol-4-yl]phenyl}-3,4-dihydroisoquinoline-2(1H)-carboxamide;
N-{4-[1-(3-methylbutyl)-1H-pyrazol-4-yl]phenyl}-3,4-dihydroisoquinoline-2(1H)-carboxamide;
N-[4-(1-benzyl-1H-pyrazol-4-yl)phenyl]-3,4-dihydroisoquinoline-2(1H)-carboxamide;
N-{4-[(1E)-5-phenylpent-1-en-1-yl]phenyl}-3,4-dihydroisoquinoline-2(1H)-carboxamide;
N-[4-(1-ethyl-1H-pyrazol-4-yl)phenyl]-3,4-dihydroisoquinoline-2(1H)-carboxamide;
N-{4-[1-(2-hydroxyethyl)-1H-pyrazol-4-yl]phenyl}-3,4-dihydroisoquinoline-2(1H)-carboxamide;
N-[4-(1-methyl-1H-pyrazol-4-yl)phenyl]-3,4-dihydroisoquinoline-2(1H)-carboxamide;
N-[4-(1-benzoyl-1,2,3,6-tetrahydropyridin-4-yl)phenyl]-3,4-dihydroisoquinoline-2(1H)-carboxamide;
N-[4-(1-butyryl-1,2,3,6-tetrahydropyridin-4-yl)phenyl]-3,4-dihydroisoquinoline-2(1H)-carboxamide;
N-{4-[1-(isopropylsulfonyl)-1,2,3,6-tetrahydropyridin-4-yl]phenyl}-3,4-dihydroisoquinoline-2(1H)-carboxamide;
N-[4-(1-isobutyryl-1,2,3,6-tetrahydropyridin-4-yl)phenyl]-3,4-dihydroisoquinoline-2(1H)-carboxamide;
N-{4-[1-(3-methylbutanoyl)-1,2,3,6-tetrahydropyridin-4-yl]phenyl}-3,4-dihydroisoquinoline-2(1H)-carboxamide;
N-{4-[1-(methylcarbamoyl)-1,2,3,6-tetrahydropyridin-4-yl]phenyl}-3,4-dihydroisoquinoline-2(1H)-carboxamide;
tert-butyl 4-{4-[(3,4-dihydroisoquinolin-2(1H)-ylcarbonyl)amino]phenyl}-3,6-dihydropyridine-1(2H)-carboxylate;
N-[4-(1-acetyl-1,2,3,6-tetrahydropyridin-4-yl)phenyl]-3,4-dihydroisoquinoline-2(1H)-carboxamide;
N-{4-[1-(isobutylsulfonyl)-1,2,3,6-tetrahydropyridin-4-yl]phenyl}-3,4-dihydroisoquinoline-2(1H)-carboxamide;
N-[4-(1-benzyl-1,2,3,6-tetrahydropyridin-4-yl)phenyl]-3,4-dihydroisoquinoline-2(1H)-carboxamide;
N-{4-[1-(methylsulfonyl)-1,2,3,6-tetrahydropyridin-4-yl]phenyl}-3,4-dihydroisoquinoline-2(1H)-carboxamide;
N-[4-(1,2,3,6-tetrahydropyridin-4-yl)phenyl]-3,4-dihydroisoquinoline-2(1H)-carboxamide;
N-{4-[1-(3-methylbutyl)-1,2,3,6-tetrahydropyridin-4-yl]phenyl}-3,4-dihydroisoquinoline-2(1H)-carboxamide;
N-[4-(5-propyl-1,2,4-oxadiazol-3-yl)phenyl]-3,4-dihydroisoquinoline-2(1H)-carboxamide;
N-[4-(5-benzyl-1,2,4-oxadiazol-3-yl)phenyl]-3,4-dihydroisoquinoline-2(1H)-carboxamide;
N-{4-[5-(3-methylbutyl)-1,2,4-oxadiazol-3-yl]phenyl}-3,4-dihydroisoquinoline-2(1H)-carboxamide;
N-hexyl-3,4-dihydroisoquinoline-2(1H)-carboxamide;
ethyl 6-[(3,4-dihydroisoquinolin-2(1H)-ylcarbonyl)amino]hexanoate;
N-(4-{2-[(phenylacetyl)amino]ethyl}phenyl)-3,4-dihydroisoquinoline-2(1H)-carboxamide;
N-{4-[2-(isobutyrylamino)ethyl]phenyl}-3,4-dihydroisoquinoline-2(1H)-carboxamide;
N-(4-{[(benzyloxy)acetyl]amino}phenyl)-3,4-dihydroisoquinoline-2(1H)-carboxamide;
N-(4-{[(4-methoxycyclohexyl)carbonyl]amino}phenyl)-3,4-dihydroisoquinoline-2(1H)-carboxamide;
N-(4-{[(1-acetylpiperidin-4-yl)carbonyl]amino}phenyl)-3,4-dihydroisoquinoline-2(1H)-carboxamide;
N-(4-{[4-oxo-4-(2-thienyl)butanoyl]amino}phenyl)-3,4-dihydroisoquinoline-2(1H)-carboxamide;
N-(4-{[3-(phenylsulfonyl)propanoyl]amino}phenyl)-3,4-dihydroisoquinoline-2(1H)-carboxamide;
N-{4-[((2R)-2,3-dihydro-1-benzofuran-2-ylcarbonyl)amino]phenyl}-3,4-dihydroisoquinoline-2(1H)-carboxamide and N-{4-[((2S)-2,3-dihydro-1-benzofuran-2-ylcarbonyl)amino]phenyl}-3,4-dihydroisoquinoline-2(1H)-carboxamide;
N N-{4-[((3R)-3-methylpentanoyl)amino]phenyl}-3,4-dihydroisoquinoline-2(1H)-carboxamide and N-{4-[((3S)-3-methylpentanoyl)amino]phenyl}-3,4-dihydroisoquinoline-2(1H)-carboxamide;
N-{4-[(2,2-dimethylbutanoyl)amino]phenyl}-3,4-dihydroisoquinoline-2(1H)-carboxamide;
N-{4-[(3,3-dimethylbutanoyl)amino]phenyl}-3,4-dihydroisoquinoline-2(1H)-carboxamide;
N-[4-(heptanoylamino)phenyl]-3,4-dihydroisoquinoline-2(1H)-carboxamide;
N-{4-[(4,4,4-trifluorobutanoyl)amino]phenyl}-3,4-dihydroisoquinoline-2(1H)-carboxamide;
N-(4-{[(2-methoxyethoxy)acetyl]amino}phenyl)-3,4-dihydroisoquinoline-2(1H)-carboxamide;
N-{4-[((3R)-tetrahydrofuran-3-ylcarbonyl)amino]phenyl}-3,4-dihydroisoquinoline-2(1H)-carboxamide and N-{4-[((3S)-tetrahydrofuran-3-ylcarbonyl)amino]phenyl}-3,4-dihydroisoquinoline-2(1H)-carboxamide;
N-(4-{[3-(methylthio)propanoyl]amino}phenyl)-3,4-dihydroisoquinoline-2(1H)-carboxamide;
N-{4-[(cyclopentylacetyl)amino]phenyl}-3,4-dihydroisoquinoline-2(1H)-carboxamide;
N-{4-[(cyclohexylcarbonyl)amino]phenyl}-3,4-dihydroisoquinoline-2(1H)-carboxamide;
N-{4-[(cyclohexylacetyl)amino]phenyl}-3,4-dihydroisoquinoline-2(1H)-carboxamide;
N-[4-(benzoylamino)phenyl]-3,4-dihydroisoquinoline-2(1H)-carboxamide;
N-{4-[(phenylacetyl)amino]phenyl}-3,4-dihydroisoquinoline-2(1H)-carboxamide;
N-(4-{[3-(4-aminophenyl)propanoyl]amino}phenyl)-3,4-dihydroisoquinoline-2(1H)-carboxamide;
N-[4-(3-furoylamino)phenyl]-3,4-dihydroisoquinoline-2(1H)-carboxamide;
N-{4-[(2,5-dimethyl-3-furoyl)amino]phenyl}-3,4-dihydroisoquinoline-2(1H)-carboxamide;
N-{4-[(3-thienylcarbonyl)amino]phenyl}-3,4-dihydroisoquinoline-2(1H)-carboxamide;
N-{4-[(1H-pyrrol-2-ylcarbonyl)amino]phenyl}-3,4-dihydroisoquinoline-2(1H)-carboxamide;
N-{4-[(1,3-thiazol-5-ylcarbonyl)amino]phenyl}-3,4-dihydroisoquinoline-2(1H)-carboxamide;
N-{4-[(1H-pyrazol-5-ylcarbonyl)amino]phenyl}-3,4-dihydroisoquinoline-2(1H)-carboxamide;
N-{4-[(1H-pyrazol-4-ylcarbonyl)amino]phenyl}-3,4-dihydroisoquinoline-2(1H)-carboxamide;
N-{4-[(1,2-oxazol-5-ylcarbonyl)amino]phenyl}-3,4-dihydroisoquinoline-2(1H)-carboxamide;
N-{4-[(pyridin-2-ylacetyl)amino]phenyl}-3,4-dihydroisoquinoline-2(1H)-carboxamide;
N-{4-[(N,N-dimethyl-beta-alanyl)amino]phenyl}-3,4-dihydroisoquinoline-2(1H)-carboxamide;
N-(4-{[3-(piperidin-1-yl)propanoyl]amino}phenyl)-3,4-dihydroisoquinoline-2(1H)-carboxamide;
N-{4-[(morpholin-4-ylacetyl)amino]phenyl}-3,4-dihydroisoquinoline-2(1H)-carboxamide;
N-(4-{[3-(morpholin-4-yl)propanoyl]amino}phenyl)-3,4-dihydroisoquinoline-2(1H)-carboxamide;
N-(4-{[3-(4-methylpiperazin-1-yl)propanoyl]amino}phenyl)-3,4-dihydroisoquinoline-2(1H)-carboxamide;
N-[4-(trifluoromethyl)phenyl]-3,4-dihydroisoquinoline-2(1H)-carboxamide;
N-{4-[(cyclopentylacetyl)amino]phenyl}-5-[(methylsulfonyl)amino]-3,4-dihydroisoquinoline-2(1H)-carboxamide; and pharmaceutically acceptable salts thereof.

Still another embodiment pertains to compounds of Formula (V), which are
N-{4-[(3-methylbutyl)carbamoyl]phenyl}-3,4-dihydroisoquinoline-2(1H)-carboxamide;
N-(4-{[4-(pyridin-2-yl)piperazin-1-yl]carbonyl}phenyl)-3,4-dihydroisoquinoline-2(1H)-carboxamide;
6-fluoro-N-{4-[(3-phenylpropyl)carbamoyl]phenyl}-3,4-dihydroisoquinoline-2(1H)-carboxamide;
N-{4-[(3-phenylpropyl)carbamoyl]phenyl}-3,4-dihydro-2,6-naphthyridine-2(1H)-carboxamide;
N-{4-[(3-methylbutyl)carbamoyl]phenyl}-3,4-dihydro-2,6-naphthyridine-2(1H)-carboxamide;
6-fluoro-N-{4-[(3-methylbutyl)carbamoyl]phenyl}-3,4-dihydroisoquinoline-2(1H)-carboxamide;
N-{4-[(3-phenylpropyl)carbamoyl]phenyl}-3,4-dihydroisoquinoline-2(1H)-carboxamide;
N-[4-(benzylcarbamoyl)phenyl]-3,4-dihydroisoquinoline-2(1H)-carboxamide;
N-(4-{[1-(3-methylbutyl)-1H-pyrazol-4-yl]carbamoyl}phenyl)-3,4-dihydroisoquinoline-2(1H)-carboxamide;
N-(4-{[2-(2-thienyl)ethyl]carbamoyl}phenyl)-3,4-dihydro-2,6-naphthyridine-2(1H)-carboxamide;
6-fluoro-N-(4-{[2-(2-thienyl)ethyl]carbamoyl}phenyl)-3,4-dihydroisoquinoline-2(1H)-carboxamide;
N-{4-[(1-benzyl-1H-pyrazol-4-yl)carbamoyl]phenyl}-3,4-dihydroisoquinoline-2(1H)-carboxamide;
N-{4-[(2-phenylethyl)carbamoyl]phenyl}-3,4-dihydroisoquinoline-2(1H)-carboxamide;
7-fluoro-N-{4-[(3-methylbutyl)carbamoyl]phenyl}-3,4-dihydroisoquinoline-2(1H)-carboxamide;
N-(4-{[2-(2-thienyl)ethyl]carbamoyl}phenyl)-3,4-dihydroisoquinoline-2(1H)-carboxamide;
7-fluoro-N-{4-[(3-phenylpropyl)carbamoyl]phenyl}-3,4-dihydroisoquinoline-2(1H)-carboxamide;
7-fluoro-N-(4-{[2-(2-thienyl)ethyl]carbamoyl}phenyl)-3,4-dihydroisoquinoline-2(1H)-carboxamide;
N-{4-[(3-phenylpropyl)carbamoyl]phenyl}-3,4-dihydro-2,7-naphthyridine-2(1H)-carboxamide;
N-{4-[(3-methylbutyl)carbamoyl]phenyl}-3,4-dihydro-2,7-naphthyridine-2(1H)-carboxamide;
N-(4-{[2-(2-thienyl)ethyl]carbamoyl}phenyl)-3,4-dihydro-2,7-naphthyridine-2(1H)-carboxamide; and pharmaceutically acceptable salts thereof.

Still another embodiment pertains to compounds of Formula (IV), which are
N-{4-[(4-methylpentanoyl)amino]phenyl}-3,4-dihydroisoquinoline-2(1H)-carboxamide;
N-{4-[(4-phenylbutanoyl)amino]phenyl}-3,4-dihydroisoquinoline-2(1H)-carboxamide;
N-(4-{[3-(2-thienyl)propanoyl]amino}phenyl)-3,4-dihydroisoquinoline-2(1H)-carboxamide;
N-(4-{[(benzyloxy)acetyl]amino}phenyl)-3,4-dihydroisoquinoline-2(1H)-carboxamide;
N-(4-{[(4-methoxycyclohexyl)carbonyl]amino}phenyl)-3,4-dihydroisoquinoline-2(1H)-carboxamide;
N-(4-{[(1-acetylpiperidin-4-yl)carbonyl]amino}phenyl)-3,4-dihydroisoquinoline-2(1H)-carboxamide;
N-(4-{[4-oxo-4-(2-thienyl)butanoyl]amino}phenyl)-3,4-dihydroisoquinoline-2(1H)-carboxamide;
N-(4-{[3-(phenylsulfonyl)propanoyl]amino}phenyl)-3,4-dihydroisoquinoline-2(1H)-carboxamide;
N-{4-[((2R)-2,3-dihydro-1-benzofuran-2-ylcarbonyl)amino]phenyl}-3,4-dihydroisoquinoline-2(1H)-carboxamide and N-{4-[((2S)-2,3-dihydro-1-benzofuran-2-ylcarbonyl)amino]phenyl}-3,4-dihydroisoquinoline-2(1H)-carboxamide;
N N-{4-[((3R)-3-methylpentanoyl)amino]phenyl}-3,4-dihydroisoquinoline-2(1H)-carboxamide and N-{4-[((3S)-3-methylpentanoyl)amino]phenyl}-3,4-dihydroisoquinoline-2(1H)-carboxamide;
N-{4-[(2,2-dimethylbutanoyl)amino]phenyl}-3,4-dihydroisoquinoline-2(1H)-carboxamide;
N-{4-[(3,3-dimethylbutanoyl)amino]phenyl}-3,4-dihydroisoquinoline-2(1H)-carboxamide;
N-[4-(heptanoylamino)phenyl]-3,4-dihydroisoquinoline-2(1H)-carboxamide;
N-{4-[(4,4,4-trifluorobutanoyl)amino]phenyl}-3,4-dihydroisoquinoline-2(1H)-carboxamide;
N-(4-{[(2-methoxyethoxy)acetyl]amino}phenyl)-3,4-dihydroisoquinoline-2(1H)-carboxamide;
N-{4-[((3R)-tetrahydrofuran-3-ylcarbonyl)amino]phenyl}-3,4-dihydroisoquinoline-2(1H)-carboxamide and N-{4-[((3S)-tetrahydrofuran-3-ylcarbonyl)amino]phenyl}-3,4-dihydroisoquinoline-2(1H)-carboxamide;
N-(4-{[3-(methylthio)propanoyl]amino}phenyl)-3,4-dihydroisoquinoline-2(1H)-carboxamide;
N-{4-[(cyclopentylacetyl)amino]phenyl}-3,4-dihydroisoquinoline-2(1H)-carboxamide;
N-{4-[(cyclohexylcarbonyl)amino]phenyl}-3,4-dihydroisoquinoline-2(1H)-carboxamide;
N-{4-[(cyclohexylacetyl)amino]phenyl}-3,4-dihydroisoquinoline-2(1H)-carboxamide;
N-[4-(benzoylamino)phenyl]-3,4-dihydroisoquinoline-2(1H)-carboxamide;
N-{4-[(phenylacetyl)amino]phenyl}-3,4-dihydroisoquinoline-2(1H)-carboxamide;
N-(4-{[3-(4-aminophenyl)propanoyl]amino}phenyl)-3,4-dihydroisoquinoline-2(1H)-carboxamide;
N-[4-(3-furoylamino)phenyl]-3,4-dihydroisoquinoline-2(1H)-carboxamide;
N-{4-[(2,5-dimethyl-3-furoyl)amino]phenyl}-3,4-dihydroisoquinoline-2(1H)-carboxamide;
N-{4-[(3-thienylcarbonyl)amino]phenyl}-3,4-dihydroisoquinoline-2(1H)-carboxamide;
N-{4-[(1H-pyrrol-2-ylcarbonyl)amino]phenyl}-3,4-dihydroisoquinoline-2(1H)-carboxamide;
N-{4-[(1,3-thiazol-5-ylcarbonyl)amino]phenyl}-3,4-dihydroisoquinoline-2(1H)-carboxamide;
N-{4-[(1H-pyrazol-5-ylcarbonyl)amino]phenyl}-3,4-dihydroisoquinoline-2(1H)-carboxamide;
N-{4-[(1H-pyrazol-4-ylcarbonyl)amino]phenyl}-3,4-dihydroisoquinoline-2(1H)-carboxamide;
N-{4-[(1,2-oxazol-5-ylcarbonyl)amino]phenyl}-3,4-dihydroisoquinoline-2(1H)-carboxamide;
N-{4-[(pyridin-2-ylacetyl)amino]phenyl}-3,4-dihydroisoquinoline-2(1H)-carboxamide;
N-{4-[(N,N-dimethyl-beta-alanyl)amino]phenyl}-3,4-dihydroisoquinoline-2(1H)-carboxamide;
N-(4-{[3-(piperidin-1-yl)propanoyl]amino}phenyl)-3,4-dihydroisoquinoline-2(1H)-carboxamide;
N-{4-[(morpholin-4-ylacetyl)amino]phenyl}-3,4-dihydroisoquinoline-2(1H)-carboxamide;
N-(4-{[3-(morpholin-4-yl)propanoyl]amino}phenyl)-3,4-dihydroisoquinoline-2(1H)-carboxamide;
N-(4-{[3-(4-methylpiperazin-1-yl)propanoyl]amino}phenyl)-3,4-dihydroisoquinoline-2(1H)-carboxamide;
N-{4-[(cyclopentylacetyl)amino]phenyl}-5-[(methylsulfonyl)amino]-3,4-dihydroisoquinoline-2(1H)-carboxamide; and pharmaceutically acceptable salts thereof.

Still another embodiment pertains to compounds of Formula (VI), which are
N-[4-(1-benzoyl-1,2,3,6-tetrahydropyridin-4-yl)phenyl]-3,4-dihydroisoquinoline-2(1H)-carboxamide;
N-[4-(1-butyryl-1,2,3,6-tetrahydropyridin-4-yl)phenyl]-3,4-dihydroisoquinoline-2(1H)-carboxamide;
N-{4-[1-(isopropylsulfonyl)-1,2,3,6-tetrahydropyridin-4-yl]phenyl}-3,4-dihydroisoquinoline-2(1H)-carboxamide;
N-[4-(1-isobutyryl-1,2,3,6-tetrahydropyridin-4-yl)phenyl]-3,4-dihydroisoquinoline-2(1H)-carboxamide;
N-{4-[1-(3-methylbutanoyl)-1,2,3,6-tetrahydropyridin-4-yl]phenyl}-3,4-dihydroisoquinoline-2(1H)-carboxamide;
N-{4-[1-(methylcarbamoyl)-1,2,3,6-tetrahydropyridin-4-yl]phenyl}-3,4-dihydroisoquinoline-2(1H)-carboxamide;
tert-butyl 4-{4-[(3,4-dihydroisoquinolin-2(1H)-ylcarbonyl)amino]phenyl}-3,6-dihydropyridine-1(2H)-carboxylate;
N-[4-(1-acetyl-1,2,3,6-tetrahydropyridin-4-yl)phenyl]-3,4-dihydroisoquinoline-2(1H)-carboxamide;
N-{4-[1-(isobutylsulfonyl)-1,2,3,6-tetrahydropyridin-4-yl]phenyl}-3,4-dihydroisoquinoline-2(1H)-carboxamide;
N-[4-(1-benzyl-1,2,3,6-tetrahydropyridin-4-yl)phenyl]-3,4-dihydroisoquinoline-2(1H)-carboxamide;
N-{4-[1-(methylsulfonyl)-1,2,3,6-tetrahydropyridin-4-yl]phenyl}-3,4-dihydroisoquinoline-2(1H)-carboxamide;
N-{4-[1-(3-methylbutyl)-1,2,3,6-tetrahydropyridin-4-yl]phenyl}-3,4-dihydroisoquinoline-2(1H)-carboxamide; and pharmaceutically acceptable salts thereof.

Another embodiment pertains to a composition for treating inflammatory and tissue repair disorders; particularly rheumatoid arthritis, inflammatory bowel disease, asthma and COPD (chronic obstructive pulmonary disease), osteoarthritis, osteoporosis and fibrotic diseases; dermatosis, including psoriasis, atopic dermatitis and ultra-violet induced skin damage; autoimmune diseases including systemic upus erythematosis, multiple sclerosis, psoriatic arthritis, ankylosing spondylitis, tissue and organ rejection, Alzheimer's disease, stroke, athersclerosis, restenosis, diabetes, glomerulonephritis, cancer, particularly wherein the cancer is selected from breast, prostate, lung, colon, cervix, ovary, skin, CNS, bladder, pancreas, leukemia, lymphoma or Hodgkin's disease, cachexia, inflammation associated with infection and certain viral infections, including Acquired Immune Deficiency Syndrome (AIDS), adult respiratory distress syndrome, and ataxia telengiectasia, said composition comprising an excipient and a therapeutically effective amount of a compound of Formula (I), or pharmaceutically acceptable salts thereof.

Another embodiment pertains to a method of treating inflammatory and tissue repair disorders; particularly rheumatoid arthritis, inflammatory bowel disease, asthma and COPD (chronic obstructive pulmonary disease), osteoarthritis, osteoporosis and fibrotic diseases; dermatosis, including psoriasis, atopic dermatitis and ultra-violet induced skin damage; autoimmune diseases including systemic lupus erythematosis, multiple sclerosis, psoriatic arthritis, ankylosing spondylitis, tissue and organ rejection, Alzheimer's disease, stroke, athersclerosis, restenosis, diabetes, glomerulonephritis, cancer, particularly wherein the cancer is selected from breast, prostate, lung, colon, cervix, ovary, skin, CNS, bladder, pancreas, leukemia, lymphoma or Hodgkin's disease, cachexia, inflammation associated with infection and certain viral infections, including Acquired Immune Deficiency Syndrome (AIDS), adult respiratory distress syndrome, and ataxia telengiectasia in a patient, said method comprising administering to the patient a therapeutically effective amount of a compound of Formula (I) or Formula (V), or pharmaceutically acceptable salts thereof.

Another embodiment pertains to a method of treating inflammatory and tissue repair disorders; particularly rheumatoid arthritis, inflammatory bowel disease, asthma and COPD (chronic obstructive pulmonary disease), osteoarthritis, osteoporosis and fibrotic diseases; dermatosis, including psoriasis, atopic dermatitis and ultra-violet induced skin damage; autoimmune diseases including systemic lupus erythematosis, multiple sclerosis, psoriatic arthritis, ankylosing spondylitis, tissue and organ rejection, Alzheimer's disease, stroke, athersclerosis, restenosis, diabetes, glomerulonephritis, cancer, particularly wherein the cancer is selected from breast, prostate, lung, colon, cervix, ovary, skin, CNS, bladder, pancreas, leukemia, lymphoma or Hodgkin's disease, cachexia, inflammation associated with infection and certain viral infections, including Acquired Immune Deficiency Syndrome (AIDS), adult respiratory distress syndrome, and ataxia telengiectasia or spleen cancer in a patient, said method comprising administering to the patient therapeutically effective amount of the compound of Formula (I), or pharmaceutically acceptable salts thereof; and a therapeutically effective amount of one additional therapeutic agent or more than one additional therapeutic agent.

### DETAILED DESCRIPTION OF THE INVENTION

This detailed description is intended only to acquaint others skilled in the art with Applicants' invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

### ABBREVIATIONS AND DEFINITIONS

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. In this application, the use of “or” means “and/or” unless stated otherwise. Furthermore, the use of the term “including”, as well as other forms, such as “includes” and “included”, is not limiting. With reference to the use of the words “comprise” or “comprises” or “comprising” in this patent application (including the claims), Applicants note that unless the context requires otherwise, those words are used on the basis and clear understanding that they are to be interpreted inclusively, rather than exclusively, and that Applicants intend each of those words to be so interpreted in construing this patent application, including the claims below. For a variable that occurs more than one time in any substituent or in the compound of the invention or any other formulae herein, its definition on each occurrence is independent of its definition at every other occurrence. Combinations of substituents are permissible only if such combinations result in stable compounds. Stable compounds are compounds which can be isolated in a useful degree of purity from a reaction mixture.

It is meant to be understood that proper valences are maintained for all combinations herein, that monovalent moieties having more than one atom are attached through their left ends, and that divalent moieties are drawn from left to right.

As used in the specification and the appended claims, unless specified to the contrary, the following terms have the meaning indicated:

The term “alkyl” (alone or in combination with another term(s)) means a straight- or branched-chain saturated hydrocarbyl substituent typically containing from 1 to about 10 carbon atoms; or in another embodiment, from 1 to about 8 carbon atoms; in another embodiment, from 1 to about 6 carbon atoms; and in another embodiment, from 1 to about 4 carbon atoms. Examples of such substituents include methyl, ethyl, n-propyl, isopropyl, n-butyl, isobutyl, sec-butyl, tert-butyl, pentyl, iso-amyl, and hexyl and the like.

The term “alkenyl” (alone or in combination with another term(s)) means a straight- or branched-chain hydrocarbyl substituent containing one or more double bonds and typically from 2 to about 10 carbon atoms; or in another embodiment, from 2 to about 8 carbon atoms; in another embodiment, from 2 to about 6 carbon atoms; and in another embodiment, from 2 to about 4 carbon atoms. Examples of such substituents include ethenyl(vinyl), 2-propenyl, 3-propenyl, 1,4-pentadienyl, 1,4-butadienyl, 1-butenyl, 2-butenyl, and 3-butenyl and the like.

The term “alkynyl” (alone or in combination with another term(s)) means a straight- or branched-chain hydrocarbyl substituent containing one or more triple bonds and typically from 2 to about 10 carbon atoms; or in another embodiment, from 2 to about 8 carbon atoms; in another embodiment, from 2 to about 6 carbon atoms; and in another embodiment, from 2 to about 4 carbon atoms. Examples of such substituents include ethynyl, 2-propynyl, 3-propynyl, 2-butynyl, and 3-butynyl and the like.

The term “carbocyclyl” (alone or in combination with another term(s)) means a saturated cyclic (i.e., “cycloalkyl”), partially saturated cyclic (i.e., “cycloalkenyl”), or completely unsaturated (i.e., “aryl”) hydrocarbyl substituent containing from 3 to 14 carbon ring atoms (“ring atoms” are the atoms bound together to form the ring or rings of a cyclic substituent). A carbocyclyl may be a single-ring (monocyclic) or polycyclic ring structure.

A carbocyclyl may be a single ring structure, which typically contains from 3 to 8 ring atoms, more typically from 3 to 6 ring atoms, and even more typically 5 to 6 ring atoms. Examples of such single-ring carbocyclyls include cyclopropyl (cyclopropanyl), cyclobutyl (cyclobutanyl), cyclopentyl (cyclopentanyl), cyclopentenyl, cyclopentadienyl, cyclohexyl (cyclohexanyl), cyclohexenyl, cyclohexadienyl, and phenyl. A carbocyclyl may alternatively be polycyclic (i.e., may contain more than one ring). Examples of polycyclic carbocyclyls include bridged, fused, and spirocyclic carbocyclyls. In a spirocyclic carbocyclyl, one atom is common to two different rings. An example of a spirocyclic carbocyclyl is spiropentanyl. In a bridged carbocyclyl, the rings share at least two common non-adjacent atoms. Examples of bridged carbocyclyls include bicyclo[2.2.1]heptanyl, bicyclo[2.2.1]hept-2-enyl, and adamantanyl. In a fused-ring carbocyclyl system, two or more rings may be fused together, such that two rings share one common bond. Examples of two- or three-fused ring carbocyclyls include naphthalenyl, tetrahydronaphthalenyl (tetralinyl), indenyl, indanyl (dihydroindenyl), anthracenyl, phenanthrenyl, and decalinyl.

The term “cycloalkyl” (alone or in combination with another term(s)) means a saturated cyclic hydrocarbyl substituent containing from 3 to 14 carbon ring atoms. A cycloalkyl may be a single carbon ring, which typically contains from 3 to 8 carbon ring atoms and more typically from 3 to 6 ring atoms. Examples of single-ring cycloalkyls include cyclopropyl, cyclobutyl, cyclopentyl, and cyclohexyl. A cycloalkyl may alternatively be polycyclic or contain more than one ring. Examples of polycyclic cycloalkyls include bridged, fused, and spirocyclic carbocyclyls.

The term “aryl” (alone or in combination with another term(s)) means an aromatic carbocyclyl containing from 6 to 14 carbon ring atoms. An aryl may be monocyclic or polycyclic (i.e., may contain more than one ring). In the case of polycyclic aromatic rings, only one ring the polycyclic system is required to be unsaturated while the remaining ring(s) may be saturated, partially saturated or unsaturated. Examples of aryls include phenyl, naphthalenyl, indenyl, indanyl, and tetrahydronapthyl.

In some instances, the number of carbon atoms in a hydrocarbyl substituent (e.g., alkyl, alkenyl, alkynyl, or cycloalkyl) is indicated by the prefix “Cₓ-C_{y}-”, wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₈-cycloalkyl means a saturated hydrocarbyl ring containing from 3 to 8 carbon ring atoms.

The term “hydrogen” (alone or in combination with another term(s)) means a hydrogen radical, and may be depicted as —H.

The term “hydroxy” (alone or in combination with another term(s)) means —OH.

The term “carboxy” (alone or in combination with another term(s)) means —C(O)—OH.

The term “amino” (alone or in combination with another term(s)) means —NH₂.

The term “halogen” or “halo” (alone or in combination with another term(s)) means a fluorine radical (which may be depicted as —F), chlorine radical (which may be depicted as —Cl), bromine radical (which may be depicted as —Br), or iodine radical (which may be depicted as —I).

If a substituent is described as being “substituted”, a non-hydrogen radical is in the place of hydrogen radical on a carbon or nitrogen of the substituent. Thus, for example, a substituted alkyl substituent is an alkyl substituent in which at least one non-hydrogen radical is in the place of a hydrogen radical on the alkyl substituent. To illustrate, monofluoroalkyl is alkyl substituted with a fluoro radical, and difluoroalkyl is alkyl substituted with two fluoro radicals. It should be recognized that if there are more than one substitution on a substituent, each non-hydrogen radical may be identical or different (unless otherwise stated).

If a substituent is described as being “optionally substituted”, the substituent may be either (1) not substituted or (2) substituted. If a substituent is described as being optionally substituted with up to a particular number of non-hydrogen radicals, that substituent may be either (1) not substituted; or (2) substituted by up to that particular number of non-hydrogen radicals or by up to the maximum number of substitutable positions on the substituent, whichever is less. Thus, for example, if a substituent is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

This patent application uses the terms “substituent” and “radical” interchangeably. The prefix “halo” indicates that the substituent to which the prefix is attached is substituted with one or more independently selected halogen radicals. For example, haloalkyl means an alkyl substituent in which at least one hydrogen radical is replaced with a halogen radical. Examples of haloalkyls include chloromethyl, 1-bromoethyl, fluoromethyl, difluoromethyl, trifluoromethyl, and 1,1,1-trifluoroethyl. It should be recognized that if a substituent is substituted by more than one halogen radical, those halogen radicals may be identical or different (unless otherwise stated).

The prefix “perhalo” indicates that every hydrogen radical on the substituent to which the prefix is attached is replaced with independently selected halogen radicals, i.e., each hydrogen radical on the substituent is replaced with a halogen radical. If all the halogen radicals are identical, the prefix typically will identify the halogen radical. Thus, for example, the term “perfluoro” means that every hydrogen radical on the substituent to which the prefix is attached is substituted with a fluorine radical. To illustrate, the term “perfluoroalkyl” means an alkyl substituent wherein a fluorine radical is in the place of each hydrogen radical.

The term “carbonyl” (alone or in combination with another term(s)) means —C(O)—. The term “aminocarbonyl” (alone or in combination with another term(s)) means —C(O)—NH₂.

The term “oxo” (alone or in combination with another term(s)) means (═O).

The term “oxy” (alone or in combination with another term(s)) means an ether substituent, and may be depicted as —O—.

The term “alkylhydroxy” (alone or in combination with another term(s)) means -alkyl-OH.

The term “alkylamino” (alone or in combination with another term(s)) means -alkyl-NH₂.

The term “alkyloxy” (alone or in combination with another term(s)) means an alkylether substituent, i.e., —O-alkyl. Examples of such a substituent include methoxy (—O—CH₃), ethoxy, n-propoxy, isopropoxy, n-butoxy, iso-butoxy, sec-butoxy, and tert-butoxy.

The term “alkylcarbonyl” (alone or in combination with another term(s)) means —C(O)-alkyl.

The term “aminoalkylcarbonyl” (alone or in combination with another term(s)) means —C(O)-alkyl-NH₂.

The term “alkyloxycarbonyl” (alone or in combination with another term(s)) means —C(O)—O-alkyl.

The term “carbocyclylcarbonyl” (alone or in combination with another term(s)) means —C(O)-carbocyclyl.

Similarly, the term “heterocyclylcarbonyl” (alone or in combination with another term(s)) means —C(O)-heterocyclyl.

The term “carbocyclylalkylcarbonyl” (alone or in combination with another term(s)) means —C(O)-alkyl-carbocyclyl.

Similarly, the term “heterocyclylalkylcarbonyl” (alone or in combination with another term(s)) means —C(O)-alkyl-heterocyclyl.

The term “carbocyclyloxycarbonyl” (alone or in combination with another term(s)) means —C(O)—O-carbocyclyl.

The term “carbocyclylalkyloxycarbonyl” (alone or in combination with another term(s)) means —C(O)—O-alkyl-carbocyclyl.

The term “thio” or “thia” (alone or in combination with another term(s)) means a thiaether substituent, i.e., an ether substituent wherein a divalent sulfur atom is in the place of the ether oxygen atom. Such a substituent may be depicted as —S—. This, for example, “alkyl-thio-alkyl” means alkyl-S-alkyl (alkyl-sulfanyl-alkyl).

The term “thiol” or “sulfhydryl” (alone or in combination with another term(s)) means a sulfhydryl substituent, and may be depicted as —SH.

The term “(thiocarbonyl)” (alone or in combination with another term(s)) means a carbonyl wherein the oxygen atom has been replaced with a sulfur. Such a substituent may be depicted as —C(S)—.

The term “sulfonyl” (alone or in combination with another term(s)) means —S(O)₂—.

The term “aminosulfonyl” (alone or in combination with another term(s)) means —S(O)₂—NH₂.

The term “sulfinyl” or “sulfoxido” (alone or in combination with another term(s)) means —S(O)—.

The term “heterocyclyl” (alone or in combination with another term(s)) means a saturated (i.e., “heterocycloalkyl”), partially saturated (i.e., “heterocycloalkenyl”), or completely unsaturated (i.e., “heteroaryl”) ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur. A heterocyclyl may be a single-ring (monocyclic) or polycyclic ring structure.

A heterocyclyl may be a single ring, which typically contains from 3 to 7 ring atoms, more typically from 3 to 6 ring atoms, and even more typically 5 to 6 ring atoms. Examples of single-ring heterocyclyls include 1,2,3,6-tetrahydropyridine, thiomorpholinyl, tetrahydropyranyl, furanyl, dihydrofuranyl, tetrahydrofuranyl, thiophenyl (thiofuranyl), dihydrothiophenyl, tetrahydrothiophenyl, pyrrolyl, pyrrolinyl, pyrrolidinyl, imidazolyl, imidazolinyl, imidazolidinyl, pyrazolyl, pyrazolinyl, pyrazolidinyl, triazolyl, tetrazolyl, oxazolyl, oxazolidinyl, isoxazolidinyl, isoxazolyl, thiazolyl, isothiazolyl, thiazolinyl, isothiazolinyl, thiazolidinyl, isothiazolidinyl, thiodiazolyl, oxadiazolyl (including 1,2,3-oxadiazolyl, 1,2,4-oxadiazolyl, 1,2,5-oxadiazolyl(furazanyl), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl, dihydropyranyl, thiopyranyl, tetrahydrothiopyranyl, pyridinyl(azinyl), piperidinyl, diazinyl (including pyridazinyl (1,2-diazinyl), pyrimidinyl(1,3-diazinyl), or pyrazinyl(1,4-diazinyl)), piperazinyl, pyrrolidin-2-only, triazinyl (including 1,3,5-triazinyl, 1,2,4-triazinyl, and 1,2,3-triazinyl)), oxazinyl (including 1,2-oxazinyl, 1,3-oxazinyl, or 1,4-oxazinyl)), oxathiazinyl (including 1,2,3-oxathiazinyl, 1,2,4-oxathiazinyl, 1,2,5-oxathiazinyl, or 1,2,6-oxathiazinyl)), oxadiazinyl (including 1,2,3-oxadiazinyl, 1,2,4-oxadiazinyl, 1,4,2-oxadiazinyl, or 1,3,5-oxadiazinyl)), morpholinyl, azepinyl, oxepinyl, thiepinyl, and diazepinyl.

A heterocyclyl may alternatively be polycyclic (i.e., may contain more than one ring). Examples of polycyclic heterocyclyls include bridged, fused, and spirocyclic heterocyclyls. In a spirocyclic heterocyclyl, one atom is common to two different rings. In a bridged heterocyclyl, the rings share at least two common non-adjacent atoms. In a fused-ring heterocyclyl, two or more rings may be fused together, such that two rings share one common bond. Examples of fused-ring heterocyclyls include hexahydro-furo[3,4-c]pyrrole, hexahydro-furo[3,4-b]pyrrole, octahydro-pyrrolo[3,4-b]pyridine, octahydro-pyrrolo[3,4-c]pyridine, (3aR,6aR)-5-methyl-octahydro-pyrrolo[3,4-b]pyrrole, (3aR,6aR)-octahydro-pyrrolo[3,4-b]pyrrole, 6-methyl-2,6-diaza-bicyclo[3.2.0]heptane, (3aS,6aR)-2-methyl-octahydro-pyrrolo[3,4-c]pyrrole, decahydro-[1,5]naphthyridine, 2,3-dihydrobenzofuranyl, 2,3,4,9-tetrahydro-1H-pyrido[3,4-b]indolyl, thieno[3,2-c]pyridinyl, furo[3,2-c]pyridinyl, phthalazin-1(2H)-onyl, isoquinolinyl, isoquinolin-1(2H)-onyl, 5,6,7,8-tetrahydrophthalazin-1(2H)-onyl, fluorophthalazin-1(2H)-onyl, (Z)-3H-benzo[d][1,2]diazepin-4(5H)-onyl, (trifluoromethyl)phthalazin-1(2H)-onyl, pyrrolo[1,2-d][1,2,4]triazin-1(2H)-onyl, 1,2,3,4-tetrahydroisoquinolinyl, 2,3-dihydrobenzo[b][1,4]dioxinyl, 5,6,7,8-tetrahydrophthalazin-1(2H)-onyl, 5,6,7,8-tetrahydro-[1,2,4]triazolo[4,3-a]pyrazinyl, 5,6,7,8-tetrahydroimidazo[1,5-a]pyrazinyl, thieno[3,2-c]pyridinyl, furo[3,2-c]pyridinyl, indolizinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl. Other examples of fused-ring heterocyclyls include benzo-fused heterocyclyls, such as benzimidazolyl, benzo[d][1,3]dioxolyl, indolyl, isoindolyl (isobenzazolyl, pseudoisoindolyl), indoleninyl (pseudoindolyl), isoindazolyl (benzpyrazolyl), benzazinyl (including quinolinyl (1-benzazinyl) or isoquinolinyl (2-benzazinyl)), phthalazinyl, quinoxalinyl, quinazolinyl, benzodiazinyl (including cinnolinyl (1,2-benzodiazinyl) or quinazolinyl (1,3-benzodiazinyl)), benzopyranyl (including chromanyl or isochromanyl), benzoxazinyl (including 1,3,2-benzoxazinyl, 1,4,2-benzoxazinyl, 2,3,1-benzoxazinyl, or 3,1,4-benzoxazinyl), and benzisoxazinyl (including 1,2-benzisoxazinyl or 1,4-benzisoxazinyl). Examples of spirocyclic heterocyclyls include 1,4-dioxa-8-azaspiro[4.5]decanyl.

The term “heterocycloalkyl” (alone or in combination with another term(s)) means a saturated heterocyclyl.

The term “heteroaryl” (alone or in combination with another term(s)) means an aromatic heterocyclyl containing from 5 to 14 ring atoms. A heteroaryl may be a single ring or 2 or 3 fused rings. Examples of heteroaryl substituents include 6-membered ring substituents such as pyridyl, pyrazyl, pyrimidinyl, pyridazinyl, and 1,3,5-, 1,2,4- or 1,2,3-triazinyl; 5-membered ring substituents such as imidazyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, benzisoxazolyl, benzoxazolyl, purinyl, and anthranilyl; and 6/6-membered fused rings such as benzopyranyl, quinolinyl, isoquinolinyl, cinnolinyl, quinazolinyl, and benzoxazinyl.

A prefix attached to a multi-component substituent only applies to the first component. To illustrate, the term “alkylcycloalkyl” contains two components: alkyl and cycloalkyl. Thus, the C₁-C₆- prefix on C₁-C₆-alkylcycloalkyl means that the alkyl component of the alkylcycloalkyl contains from 1 to 6 carbon atoms; the C₁-C₆-prefix does not describe the cycloalkyl component. To illustrate further, the prefix “halo” on haloalkyloxyalkyl indicates that only the alkyloxy component of the alkyloxyalkyl substituent is substituted with one or more halogen radicals. If halogen substitution may alternatively or additionally occur on the alkyl component, the substituent would instead be described as “halogen-substituted alkyloxyalkyl” rather than “haloalkyloxyalkyl.” And finally, if the halogen substitution may only occur on the alkyl component, the substituent would instead be described as “alkyloxyhaloalkyl.”

The terms “treat”, “treating” and “treatment” refer to a method of alleviating or abrogating a disease and/or its attendant symptoms.

The terms “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

The term “therapeutically effective amount” refers to that amount of the compound being administered sufficient to prevent development of or alleviate to some extent one or more of the symptoms of the condition or disorder being treated.

The term “modulate” refers to the ability of a compound to increase or decrease the function, or activity, of a kinase. “Modulation”, as used herein in its various forms, is intended to encompass antagonism, agonism, partial antagonism and/or partial agonism of the activity associated with kinase. Kinase inhibitors are compounds that, e.g., bind to, partially or totally block stimulation, decrease, prevent, delay activation, inactivate, desensitize, or down regulate signal transduction. Kinase activators are compounds that, e.g., bind to, stimulate, increase, open, activate, facilitate, enhance activation, sensitize or up regulate signal transduction.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The “subject” is defined herein to include animals such as mammals, including, but not limited to, primates (e.g., humans), cows, sheep, goats, horses, dogs, cats, rabbits, rats, mice and the like. In preferred embodiments, the subject is a human.

### Isotope Enriched or Labeled Compounds

Compounds of the invention can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl and ¹²⁵I. Compounds that contain other isotopes of these and/or other atoms are within the scope of this invention.

In another embodiment, the isotope-labeled compounds contain deuterium (²H), tritium (³H) or ¹⁴C isotopes. Isotope-labeled compounds of this invention can be prepared by the general methods well known to persons having ordinary skill in the art. Such isotope-labeled compounds can be conveniently prepared by carrying out the procedures disclosed in the Examples disclosed herein and Schemes by substituting a readily available isotope-labeled reagent for a non-labeled reagent. In some instances, compounds may be treated with isotope-labeled reagents to exchange a normal atom with its isotope, for example, hydrogen for deuterium can be exchanged by the action of a deuteric acid such as D₂SO₄/D₂O. In addition to the above, relevant procedures and intermediates are disclosed, for instance, in Lizondo, J et al., Drugs Fut, 21(11), 1116 (1996); Brickner, S J et al., J Med Chem, 39(3), 673 (1996); Mallesham, B et al., Org Lett, 5(7), 963 (2003); PCT publications WO1997010223, WO2005099353, WO1995007271, WO2006008754; U.S. Pat. Nos. 7,538,189; 7,534,814; 7,531,685; 7,528,131; 7,521,421; 7,514,068; 7,511,013; and US Patent Application Publication Nos. 20090137457; 20090131485; 20090131363; 20090118238; 20090111840; 20090105338; 20090105307; 20090105147; 20090093422; 20090088416; and 20090082471, the methods are hereby incorporated by reference.

The isotope-labeled compounds of the invention may be used as standards to determine the effectiveness of Bcl-2 inhibitors in binding assays. Isotope containing compounds have been used in pharmaceutical research to investigate the in vivo metabolic fate of the compounds by evaluation of the mechanism of action and metabolic pathway of the nonisotope-labeled parent compound (Blake et al. J. Pharm. Sci. 64, 3, 367-391 (1975)). Such metabolic studies are important in the design of safe, effective therapeutic drugs, either because the in vivo active compound administered to the patient or because the metabolites produced from the parent compound prove to be toxic or carcinogenic (Foster et al., Advances in Drug Research Vol. 14, pp. 2-36, Academic press, London, 1985; Kato et al., J. Labelled Comp. Radiopharmaceut., 36(10):927-932 (1995); Kushner et al., Can. J. Physiol. Pharmacol., 77, 79-88 (1999).

In addition, non-radio active isotope containing drugs, such as deuterated drugs called “heavy drugs,” can be used for the treatment of diseases and conditions related to Bcl-2 activity. Increasing the amount of an isotope present in a compound above its natural abundance is called enrichment. Examples of the amount of enrichment include from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol %. Replacement of up to about 15% of normal atom with a heavy isotope has been effected and maintained for a period of days to weeks in mammals, including rodents and dogs, with minimal observed adverse effects (Czajka D M and Finkel A J, Ann. N.Y. Acad. Sci. 1960 84: 770; Thomson J F, Ann. New York Acad. Sci. 1960 84: 736; Czakja D M et al., Am. J. Physiol. 1961 201: 357). Acute replacement of as high as 15%-23% in human fluids with deuterium was found not to cause toxicity (Blagojevic N et al. in “Dosimetry & Treatment Planning for Neutron Capture Therapy”, Zamenhof R, Solares G and Harling O Eds. 1994. Advanced Medical Publishing, Madison Wis. pp. 125-134; Diabetes Metab. 23: 251 (1997)).

Stable isotope labeling of a drug can alter its physico-chemical properties such as pKa and lipid solubility. These effects and alterations can affect the pharmacodynamic response of the drug molecule if the isotopic substitution affects a region involved in a ligand-receptor interaction. While some of the physical properties of a stable isotope-labeled molecule are different from those of the unlabeled one, the chemical and biological properties are the same, with one important exception: because of the increased mass of the heavy isotope, any bond involving the heavy isotope and another atom will be stronger than the same bond between the light isotope and that atom. Accordingly, the incorporation of an isotope at a site of metabolism or enzymatic transformation will slow said reactions potentially altering the pharmacokinetic profile or efficacy relative to the non-isotopic compound.

### Compounds

Suitable groups for X¹, X², and R² in compounds of Formula (I) are independently selected. The described embodiments of the present invention may be combined. Such combination is contemplated and within the scope of the present invention. For example, it is contemplated that embodiments for any of X¹, X², and R² can be combined with embodiments defined for any other of X¹, X², and R².

### Embodiments of Formula (I)

One embodiment of this invention, therefore, pertains to compounds or pharmaceutically acceptable salts thereof, which are useful as inhibitors of NAMPT, the compounds having Formula (I)

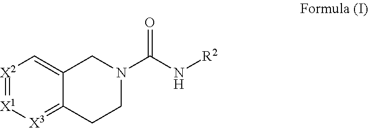

wherein

X¹ and X² and X³ are CH; or

X¹ and X³ are CH; and X² is N; or

X² and X³ are H; and X¹ is N; or

X¹ is CR¹; and X² and X³ are CH; or

X² is CR¹; and X¹ and X³ are CH; or

X³ is CR¹; and X¹ and X² are CH;

R¹ is R³, OR³, SR³, S(O)R³, SO₂R³, C(O)R³, C(O)OR³, OC(O)R³, NHR³, N(R³)₂, C(O)NH₂, C(O)NHR³, C(O)N(R³)₂, NHC(O)R³, NR³C(O)R³, NHC(O)OR³, NR³C(O)OR³, SO₂NH₂, SO₂NHR³, SO₂N(R³)₂, NHSO₂R³, NR³SO₂R³, NHSO₂NHR³, NHSO₂N(R³)₂, NR³SO₂NHR³, NR³SO₂N(R³)₂, C(O)NHSO₂R³, NHSO₂NHR³, F, Cl, Br, I, CN, NH₂, NO₂, N₃, OH, C(O)H, CF₃, C(O)OH, or C(O)NH₂;

R² is alkyl, alkenyl, alkynyl, phenyl, heterocyclyl, cycloalkyl, or cycloalkenyl; wherein each alkyl, alkenyl, and alkynyl is optionally substituted with one or more independently selected R⁴, OR⁴, SR⁴, S(O)R⁴, SO₂R⁴, C(O)R⁴, CO(O)R⁴, OC(O)R⁴, OC(O)OR⁴, NH₂, NHR⁴, N(R⁴)₂, NHC(O)R⁴, NR⁴C(O)R⁴, NHS(O)₂R⁴, NR⁴S(O)₂R⁴, NHC(O)OR⁴, NR⁴C(O)OR⁴, NHC(O)NH₂, NHC(O)NHR⁴, NHC(O)N(R⁴)₂, NR⁴C(O)NHR⁴, NR⁴C(O)N(R⁴)₂, C(O)NH₂, C(O)NHR⁴, C(O)N(R⁴)₂, C(O)NHOH, C(O)NHOR⁴, C(O)NHSO₂R⁴, C(O)NR⁴SO₂R⁴, SO₂NH₂, SO₂NHR⁴, SO₂N(R⁴)₂, C(O)H, C(O)OH, OH, (O), CN, N₃, NO₂, CF₃, CF₂CF₃, OCF₃, OCF₂CF₃, F, Cl, Br or I; wherein each phenyl is optionally additionally substituted at the para position with one independently selected R⁵, OCH₂CH₂CH₂CH₂CH₂CH₃, SR⁵, S(O)R⁵, SO₂R⁵, C(O)R⁵, CO(O)R⁵, OC(O)R⁵, OC(O)OR⁵, NH₂, NHR⁵, N(R⁵)₂, NHC(O)R⁵, NR⁵C(O)R⁵, NHS(O)₂R⁵, NR⁵S(O)₂R⁵, NHC(O)OR⁵, NR⁵C(O)OR⁵, NHC(O)NH₂, NHC(O)NHR⁵, NHC(O)N(R⁵)₂, NR⁵C(O)NHR⁵, NR⁵C(O)N(R⁵)₂, C(O)NH₂, C(O)NHR⁵, C(O)N(R⁵)₂, C(O)NHOH, C(O)NHOR⁵, C(O)NHSO₂R⁵, C(O)NR⁵SO₂R⁵, SO₂NH₂, SO₂NHR⁵, SO₂N(R⁵)₂, C(O)H, C(O)OH, OH, CN, N₃, NO₂, CF₃, CF₂CF₃, OCF₃, OCF₂CF₃, Br or I; wherein each phenyl is optionally additionally substituted with one F; wherein each heterocyclyl, cycloalkyl, and cycloalkenyl is optionally substituted with one or more independently selected R⁵, OR⁵, SR⁵, S(O)R⁵, SO₂R⁵, C(O)R⁵, CO(O)R⁵, OC(O)R⁵, OC(O)OR⁵, NH₂, NHR⁵, N(R⁵)₂, NHC(O)R⁵, NR⁵C(O)R⁵, NHS(O)₂R⁵, NR⁵S(O)₂R⁵, NHC(O)OR⁵, NR⁵C(O)OR⁵, NHC(O)NH₂, NHC(O)NHR⁵, NHC(O)N(R⁵)₂, NR⁵C(O)NHR⁵, NR⁵C(O)N(R⁵)₂, C(O)NH₂, C(O)NHR⁵, C(O)N(R⁵)₂, C(O)NHOH, C(O)NHOR⁵, C(O)NHSO₂R⁵, C(O)NR⁵SO₂R⁵, SO₂NH₂, SO₂NHR⁵, SO₂N(R⁵)₂, C(O)H, C(O)OH, OH, CN, N₃, CF₃, CF₂CF₃, OCF₃, OCF₂CF₃, F, Cl, Br or I; wherein R² is not 4-methylphenyl;

R³ is alkyl, alkenyl, alkynyl, aryl, or heterocyclyl; wherein each alkyl, alkenyl, and alkynyl is optionally substituted with one or more independently selected R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, OH, (O), CN, N₃, NO₂, CF₃, CF₂CF₃, OCF₃, OCF₂CF₃, F, Cl, Br or I;

R⁴ is alkyl, alkenyl, alkynyl, aryl or heterocyclyl; wherein each alkyl, alkenyl, and alkynyl is optionally substituted with one or more independently selected R⁷, OR⁷, SR⁷, S(O)R⁷, SO₂R⁷, C(O)R⁷, CO(O)R⁷, OC(O)R⁷, OC(O)OR⁷, NH₂, NHR⁷, N(R⁷)₂, NHC(O)R⁷, NR⁷C(O)R⁷, NHS(O)₂R⁷, NR⁷S(O)₂R⁷, NHC(O)OR⁷, NR⁷C(O)OR⁷, NHC(O)NH₂, NHC(O)NHR⁷, NHC(O)N(R⁷)₂, NR⁷C(O)NHR⁷, NR⁷C(O)N(R⁷)₂, C(O)NH₂, C(O)NHR⁷, C(O)N(R⁷)₂, C(O)NHOH, C(O)NHOR⁷, C(O)NHSO₂R⁷, C(O)NR⁷SO₂R⁷, SO₂NH₂, SO₂NHR⁷, SO₂N(R⁷)₂, C(O)H, C(O)OH, OH, (O), CN, N₃, NO₂, CF₃, CF₂CF₃, OCF₃, OCF₂CF₃, F, Cl, Br or I; wherein each aryl and heterocycyl is optionally substituted with one or more independently selected R⁸, OR⁸, SR⁸, S(O)R⁸, SO₂R⁸, C(O)R⁸, CO(O)R⁸, OC(O)R⁸, OC(O)OR⁸, NH₂, NHR⁸, N(R⁸)₂, NHC(O)R⁸, NR⁸C(O)R⁸, NHS(O)₂R⁸, NR⁸S(O)₂R⁸, NHC(O)OR⁸, NR⁸C(O)OR⁸, NHC(O)NH₂, NHC(O)NHR⁸, NHC(O)N(R⁸)₂, NR⁸C(O)NHR⁸, NR⁸C(O)N(R⁸)₂, C(O)NH₂, C(O)NHR⁸, C(O)N(R⁸)₂, C(O)NHOH, C(O)NHOR⁸, C(O)NHSO₂R⁸, C(O)NR⁸SO₂R⁸, SO₂NH₂, SO₂NHR⁸, SO₂N(R⁸)₂, C(O)H, C(O)OH, OH, CN, N₃, NO₂, CF₃, CF₂CF₃, OCF₃, OCF₂CF₃, F, Cl, Br or I;

R⁵ is alkyl, alkenyl, alkynyl, aryl, heterocyclyl, cycloalkyl, or cycloalkenyl; wherein each alkyl, alkenyl, and alkynyl is optionally substituted with one or more independently selected R⁹, OR⁹, SR⁹, S(O)R⁹, SO₂R⁹, C(O)R⁹, CO(O)R⁹, OC(O)R⁹, OC(O)OR⁹, NH₂, NHR⁹, N(R⁹)₂, NHC(O)R⁹, NR⁹C(O)R⁹, NHS(O)₂R⁹, NR⁹S(O)₂R⁹, NHC(O)OR⁹, NR⁹C(O)OR⁹, NHC(O)NH₂, NHC(O)NHR⁹, NHC(O)N(R⁹)₂, NR⁹C(O)NHR⁹, NR⁹C(O)N(R⁹)₂, C(O)NH₂, C(O)NHR⁹, C(O)N(R⁹)₂, C(O)NHOH, C(O)NHOR⁹, C(O)NHSO₂R⁹, C(O)NR⁹SO₂R⁹, SO₂NH₂, SO₂NHR⁹, SO₂N(R⁹)₂, C(O)H, C(O)OH, OH, (O), CN, N₃, NO₂, CF₃, CF₂CF₃, OCF₃, OCF₂CF₃, F, Cl, Br or I;

R⁶ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁰, OR¹⁰, SR¹⁰, S(O)R¹⁰, SO₂R¹⁰, NHR¹⁰, N(R¹⁰)₂, C(O)R¹⁰, C(O)NH₂, C(O)NHR¹⁰, C(O)N(R¹⁰)₂, NHC(O)R¹⁰, NR¹⁰C(O)R¹⁰, NHSO₂R¹⁰, NHC(O)OR¹⁰, SO₂NH₂, SO₂NHR¹⁰, SO₂N(R¹⁰)₂, NHC(O)NH₂, NHC(O)NHR¹⁰, OH, (O), C(O)OH, N₃, CN, NH₂, CF₃, CF₂CF₃, F, Cl, Br or I;

R⁷ is alkyl, alkenyl, alkynyl, aryl, heterocyclyl, cycloalkyl, or cycloalkenyl; wherein each alkyl, alkenyl, and alkynyl is optionally substituted with one or more independently selected R¹¹, OR¹¹, SR¹¹, S(O)R¹¹, SO₂R¹¹, NHR¹¹, N(R¹¹)₂, C(O)R¹¹, C(O)NH₂, C(O)NHR¹¹, C(O)N(R¹¹)₂, NHC(O)R¹¹, NR¹¹C(O)R¹¹, NHSO₂R¹¹, NHC(O)OR¹¹, SO₂NH₂, SO₂NHR¹¹, SO₂N(R¹¹)₂, NHC(O)NH₂, NHC(O)NHR¹¹, OH, (O), C(O)OH, N₃, CN, NH₂, CF₃, CF₂CF₃, F, Cl, Br or I;

R⁸ is alkyl, alkenyl, alkynyl, aryl, heterocyclyl, cycloalkyl, or cycloalkenyl; wherein each alkyl, alkenyl, and alkynyl is optionally substituted with one or more independently selected R¹², OR¹², SR¹², S(O)R¹², SO₂R¹², NHR¹², N(R¹²)₂, C(O)R¹², C(O)NH₂, C(O)NHR¹², C(O)N(R¹²)₂, NHC(O)R¹², NR¹²C(O)R¹², NHSO₂R¹², NHC(O)OR¹², SO₂NH₂, SO₂NHR¹², SO₂N(R¹²)₂, NHC(O)NH₂, NHC(O)NHR¹², OH, (O), C(O)OH, N₃, CN, NH₂, CF₃, CF₂CF₃, F, Cl, Br or I;

R⁹ is alkyl, alkenyl, alkynyl, aryl, heterocyclyl, cycloalkyl, or cycloalkenyl; wherein each alkyl, alkenyl, and alkynyl is optionally substituted with one or more independently selected OCH₃, aryl, or heterocyclyl;

R¹⁰ is alkyl, alkenyl, alkynyl, aryl, heterocyclyl, cycloalkyl, or cycloalkenyl;

R¹¹ is alkyl, alkenyl, alkynyl, aryl, heterocyclyl, cycloalkyl, or cycloalkenyl;

R¹² is alkyl, alkenyl, alkynyl, aryl, heterocyclyl, cycloalkyl, or cycloalkenyl;

wherein the cyclic moieties represented by R³, R⁵, R⁶, R⁷, R⁸, R⁹, R¹⁰, R¹¹, and R¹² are optionally substituted with one or more independently selected R¹³, OR¹³, SR¹³, S(O)R¹³, SO₂R¹³, C(O)R¹³, CO(O)R¹³, OC(O)R¹³, OC(O)OR¹³, NH₂, NHR¹³, N(R¹³)₂, NHC(O)R¹³, NR¹³C(O)R¹³, NHS(O)₂R¹³, NR¹³S(O)₂R¹³, NHC(O)OR¹³, NR¹³C(O)OR¹³, NHC(O)NH₂, NHC(O)NHR¹³, NHC(O)N(R¹³)₂, NR¹³C(O)NHR¹³, NR¹³C(O)N(R¹³)₂, C(O)NH₂, C(O)NHR¹³, C(O)N(R¹³)₂, C(O)NHOH, C(O)NHOR¹³, C(O)NHSO₂R¹³, C(O)NR¹³SO₂R¹³, SO₂NH₂, SO₂NHR¹³, SO₂N(R¹³)₂, C(O)H, C(O)OH, OH, CN, N₃, NO₂, CF₃, CF₂CF₃, OCF₃, OCF₂CF₃, F, Cl, Br or I;

R¹³ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁴, OR¹⁴, SR¹⁴, S(O)R¹⁴, SO₂R¹⁴, C(O)R¹⁴, CO(O)R¹⁴, OC(O)R¹⁴, OC(O)OR¹⁴, NH₂, NHR¹⁴, N(R¹⁴)₂, NHC(O)R¹⁴, NR¹⁴C(O)R¹⁴, NHS(O)₂R¹⁴, NR¹⁴S(O)₂R¹⁴, NHC(O)OR¹⁴, NR¹⁴C(O)OR¹⁴, NHC(O)NH₂, NHC(O)NHR¹⁴, NHC(O)N(R¹⁴)₂, NR¹⁴C(O)NHR¹⁴, NR¹⁴C(O)N(R¹⁴)₂, C(O)NH₂, C(O)NHR¹⁴, C(O)N(R¹⁴)₂, C(O)NHOH, C(O)NHOR¹⁴, C(O)NHSO₂R¹⁴, C(O)NR¹⁴SO₂R¹⁴, SO₂NH₂, SO₂NHR¹⁴, SO₂N(R¹⁴)₂, C(O)H, C(O)OH, OH, (O), CN, N₃, NO₂, CF₃, CF₂CF₃, OCF₃, OCF₂CF₃, F, Cl, Br or I; wherein each aryl, heterocyclyl, cycloalkyl, and cycloalkenyl is optionally substituted with one or more independently selected R¹⁵, OR¹⁵, SR¹⁵, S(O)R¹⁵, SO₂R¹⁵, C(O)R¹⁵, CO(O)R¹⁵, OC(O)R¹⁵, OC(O)OR¹⁵, NH₂, NHR¹⁵, N(R¹⁵)₂, NHC(O)R¹⁵, NR¹⁵C(O)R¹⁵, NHS(O)₂R¹⁵, NR¹⁵S(O)₂R¹⁵, NHC(O)OR¹⁵, NR¹⁵C(O)OR¹⁵, NHC(O)NH₂, NHC(O)NHR¹⁵, NHC(O)N(R¹⁵)₂, NR¹⁵C(O)NHR¹⁵, NR¹⁵C(O)N(R¹⁵)₂, C(O)NH₂, C(O)NHR¹⁵, C(O)N(R¹⁵)₂, C(O)NHOH, C(O)NHOR¹⁵, C(O)NHSO₂R¹⁵, C(O)NR¹⁵SO₂R¹⁵, SO₂NH₂, SO₂NHR¹⁵, SO₂N(R¹⁵)₂, C(O)H, C(O)OH, OH, CN, N₃, NO₂, CF₃, CF₂CF₃, OCF₃, OCF₂CF₃, F, Cl, Br or I;

R¹⁴ is alkyl, alkenyl, alkynyl, aryl, heterocyclyl, cycloalkyl, or cycloalkenyl; wherein each alkyl, alkenyl, and alkynyl is optionally substituted with one or more independently selected NH₂, SO₂NH₂, C(O)H, C(O)OH, OH, (O), CN, N₃, NO₂, CF₃, CF₂CF₃, OCF₃, OCF₂CF₃, F, Cl, Br or I; and

R¹⁵ is alkyl.

In one embodiment of Formula (I), X¹, X² and X³ are CH; or X¹ and X³ are CH and X² is N; or X² and X³ are CH and X¹ is N; or X¹ is CR¹ and X² and X³ are CH; or X² is CR¹ and X¹ and X³ are CH; or X³ is CR¹; and X¹ and X² are CH.

In another embodiment of Formula (I), X¹, X² and X³ are CH. In another embodiment of Formula (I), X¹ and X³ are CH and X² is N. In another embodiment of Formula (I), X² and X³ are CH and X¹ is N. In another embodiment of Formula (I), X¹ is CR¹ and X² and X³ are CH. In another embodiment of Formula (I), X² is CR¹ and X¹ and X³ are CH. In another embodiment of Formula (I), X³ is CR¹; and X¹ and X² are CH.

In one embodiment of Formula (I), R¹ is NHSO₂R³, F, Cl, Br, or I. In another embodiment of Formula (I), R¹ is F. In another embodiment of Formula (I), R¹ is NHSO₂R³; and R³ is alkyl.

In one embodiment of Formula (I), X¹ is CR¹; X² and X³ are CH; and R¹ is F. In one embodiment of Formula (I), X² is CR¹; X¹ and X³ are CH; and R¹ is F. In another embodiment of Formula (I), X³ is CR¹; and X¹ and X² are CH; and R¹ is NHSO₂R³; and R³ is alkyl.

In one embodiment of Formula (I),

X¹ and X² and X³ are CH; or

X¹ and X³ are CH; and X² is N; or

X² and X³ are H; and X¹ is N; or

X¹ is CR¹; and X² and X³ are CH; or

X² is CR¹; and X¹ and X³ are CH; or

X³ is CR¹; and X¹ and X² are CH;

R¹ is NHSO₂R³, F, Cl, Br, or I;

R² is alkyl, phenyl, or heterocyclyl; wherein each alkyl is optionally substituted with CO(O)R⁴; wherein each phenyl is optionally additionally substituted at the para position with one independently selected R⁵, C(O)R⁵, NHC(O)R⁵, or C(O)NHR⁵; wherein each heterocyclyl is optionally substituted with C(O)NHR⁵; wherein R² is not 4-methylphenyl;

R³ is alkyl;

R⁴ is alkyl;

R⁵ is alkyl, alkenyl, aryl, heterocyclyl, or cycloalkyl; wherein each alkyl and alkenyl, is optionally substituted with one or more independently selected R⁹, OR⁹, SR⁹, SO₂R⁹, C(O)R⁹, N(R⁹)₂, NHC(O)R⁹, C(O)NHR⁹, OH, or CF₃, F, Cl, Br or I;

R⁹ is alkyl, aryl, heterocyclyl, or cycloalkyl; wherein each alkyl is optionally substituted with one or more independently selected OCH₃, aryl, or heterocyclyl;

wherein the cyclic moieties represented by R⁵ and R⁹ are optionally substituted with one or more independently selected R¹³, OR¹³, SO₂R¹³, C(O)R¹³, CO(O)R¹³, NH₂, C(O)NHR¹³, F, Cl, Br or I;

R¹³ is alkyl, aryl, or heterocyclyl; wherein each alkyl is optionally substituted with one or more independently selected R¹⁴, OH, F, Cl, Br or I; and

R¹⁴ is aryl F, Cl, Br or I.

Still another embodiment pertains to compounds having Formula (I), which include
N-{4-[(3-methylbutyl)carbamoyl]phenyl}-3,4-dihydroisoquinoline-2(1H)-carboxamide;
N-(4-{[4-(pyridin-2-yl)piperazin-1-yl]carbonyl}phenyl)-3,4-dihydroisoquinoline-2(1H)-carboxamide;
6-fluoro-N-{4-[(3-phenylpropyl)carbamoyl]phenyl}-3,4-dihydroisoquinoline-2(1H)-carboxamide;
N-{4-[(3-phenylpropyl)carbamoyl]phenyl}-3,4-dihydro-2,6-naphthyridine-2(1H)-carboxamide;
N-{4-[(3-methylbutyl)carbamoyl]phenyl}-3,4-dihydro-2,6-naphthyridine-2(1H)-carboxamide;
6-fluoro-N-{4-[(3-methylbutyl)carbamoyl]phenyl}-3,4-dihydroisoquinoline-2(1H)-carboxamide;
N-{4-[(3-phenylpropyl)carbamoyl]phenyl}-3,4-dihydroisoquinoline-2(1H)-carboxamide;
N-[4-(benzylcarbamoyl)phenyl]-3,4-dihydroisoquinoline-2(1H)-carboxamide;
N-{5-[(3-phenylpropyl)carbamoyl]pyridin-2-yl}-3,4-dihydroisoquinoline-2(1H)-carboxamide;
N-{5-[(3-methylbutyl)carbamoyl]pyridin-2-yl}-3,4-dihydroisoquinoline-2(1H)-carboxamide;
N-(4-{[1-(3-methylbutyl)-1H-pyrazol-4-yl]carbamoyl}phenyl)-3,4-dihydroisoquinoline-2(1H)-carboxamide;
N-(4-{[2-(2-thienyl)ethyl]carbamoyl}phenyl)-3,4-dihydro-2,6-naphthyridine-2(1H)-carboxamide;
6-fluoro-N-(4-{[2-(2-thienyl)ethyl]carbamoyl}phenyl)-3,4-dihydroisoquinoline-2(1H)-carboxamide;
N-{4-[(1-benzyl-1H-pyrazol-4-yl)carbamoyl]phenyl}-3,4-dihydroisoquinoline-2(1H)-carboxamide;
N-{4-[(2-phenylethyl)carbamoyl]phenyl}-3,4-dihydroisoquinoline-2(1H)-carboxamide;
7-fluoro-N-{4-[(3-methylbutyl)carbamoyl]phenyl}-3,4-dihydroisoquinoline-2(1H)-carboxamide;
N-(4-{[2-(2-thienyl)ethyl]carbamoyl}phenyl)-3,4-dihydroisoquinoline-2(1H)-carboxamide;
N-{6-[(3-methylbutyl)carbamoyl]pyridin-3-yl}-3,4-dihydroisoquinoline-2(1H)-carboxamide;
N-(5-{[2-(2-thienyl)ethyl]carbamoyl}pyridin-2-yl)-3,4-dihydroisoquinoline-2(1H)-carboxamide;
7-fluoro-N-{4-[(3-phenylpropyl)carbamoyl]phenyl}-3,4-dihydroisoquinoline-2(1H)-carboxamide;
N-{6-[(3-phenylpropyl)carbamoyl]pyridin-3-yl}-3,4-dihydroisoquinoline-2(1H)-carboxamide;
N-(6-{[2-(2-thienyl)ethyl]carbamoyl}pyridin-3-yl)-3,4-dihydroisoquinoline-2(1H)-carboxamide;
7-fluoro-N-(4-{[2-(2-thienyl)ethyl]carbamoyl}phenyl)-3,4-dihydroisoquinoline-2(1H)-carboxamide;
N-{4-[(3-phenylpropyl)carbamoyl]phenyl}-3,4-dihydro-2,7-naphthyridine-2(1H)-carboxamide;
N-[4-(2-oxo-2-{[2-(2-thienyl)ethyl]amino}ethyl)phenyl]-3,4-dihydroisoquinoline-2(1H)-carboxamide;
N-(4-{2-oxo-2-[(3-phenylpropyl)amino]ethyl}phenyl)-3,4-dihydroisoquinoline-2(1H)-carboxamide;
N-{4-[(3-methylbutyl)carbamoyl]phenyl}-3,4-dihydro-2,7-naphthyridine-2(1H)-carboxamide;
N-(4-{2-[(3-methylbutyl)amino]-2-oxoethyl}phenyl)-3,4-dihydroisoquinoline-2(1H)-carboxamide;
N-(4-{[2-(2-thienyl)ethyl]carbamoyl}phenyl)-3,4-dihydro-2,7-naphthyridine-2(1H)-carboxamide;
N-{4-[(4-methylpentanoyl)amino]phenyl}-3,4-dihydroisoquinoline-2(1H)-carboxamide;
N-{4-[(4-phenylbutanoyl)amino]phenyl}-3,4-dihydroisoquinoline-2(1H)-carboxamide;
N-(4-{[3-(2-thienyl)propanoyl]amino}phenyl)-3,4-dihydroisoquinoline-2(1H)-carboxamide;
N-[4-(1-isobutyl-1H-pyrazol-4-yl)phenyl]-3,4-dihydroisoquinoline-2(1H)-carboxamide;
N-[4-(1-propyl-1H-pyrazol-4-yl)phenyl]-3,4-dihydroisoquinoline-2(1H)-carboxamide;
N-{4-[1-((2R)-2-hydroxypropyl)-1H-pyrazol-4-yl]phenyl}-3,4-dihydroisoquinoline-2(1H)-carboxamide;
N-{4-[1-(3-methylbutyl)-1H-pyrazol-4-yl]phenyl}-3,4-dihydroisoquinoline-2(1H)-carboxamide;
N-[4-(1-benzyl-1H-pyrazol-4-yl)phenyl]-3,4-dihydroisoquinoline-2(1H)-carboxamide;
N-{4-[(1E)-5-phenylpent-1-en-1-yl]phenyl}-3,4-dihydroisoquinoline-2(1H)-carboxamide;
N-[4-(1-ethyl-1H-pyrazol-4-yl)phenyl]-3,4-dihydroisoquinoline-2(1H)-carboxamide;
N-{4-[1-(2-hydroxyethyl)-1H-pyrazol-4-yl]phenyl}-3,4-dihydroisoquinoline-2(1H)-carboxamide;
N-[4-(1-methyl-1H-pyrazol-4-yl)phenyl]-3,4-dihydroisoquinoline-2(1H)-carboxamide;
N-[4-(1-benzoyl-1,2,3,6-tetrahydropyridin-4-yl)phenyl]-3,4-dihydroisoquinoline-2(1H)-carboxamide;
N-[4-(1-butyryl-1,2,3,6-tetrahydropyridin-4-yl)phenyl]-3,4-dihydroisoquinoline-2(1H)-carboxamide;
N-{4-[1-(isopropylsulfonyl)-1,2,3,6-tetrahydropyridin-4-yl]phenyl}-3,4-dihydroisoquinoline-2(1H)-carboxamide;
N-[4-(1-isobutyryl-1,2,3,6-tetrahydropyridin-4-yl)phenyl]-3,4-dihydroisoquinoline-2(1H)-carboxamide;
N-{4-[1-(3-methylbutanoyl)-1,2,3,6-tetrahydropyridin-4-yl]phenyl}-3,4-dihydroisoquinoline-2(1H)-carboxamide;
N-{4-[1-(methylcarbamoyl)-1,2,3,6-tetrahydropyridin-4-yl]phenyl}-3,4-dihydroisoquinoline-2(1H)-carboxamide;
tert-butyl 4-{4-[(3,4-dihydroisoquinolin-2(1H)-ylcarbonyl)amino]phenyl}-3,6-dihydropyridine-1(2H)-carboxylate;
N-[4-(1-acetyl-1,2,3,6-tetrahydropyridin-4-yl)phenyl]-3,4-dihydroisoquinoline-2(1H)-carboxamide;
N-{4-[1-(isobutylsulfonyl)-1,2,3,6-tetrahydropyridin-4-yl]phenyl}-3,4-dihydroisoquinoline-2(1H)-carboxamide;
N-[4-(1-benzyl-1,2,3,6-tetrahydropyridin-4-yl)phenyl]-3,4-dihydroisoquinoline-2(1H)-carboxamide;
N-{4-[1-(methylsulfonyl)-1,2,3,6-tetrahydropyridin-4-yl]phenyl}-3,4-dihydroisoquinoline-2(1H)-carboxamide;
N-[4-(1,2,3,6-tetrahydropyridin-4-yl)phenyl]-3,4-dihydroisoquinoline-2(1H)-carboxamide;
N-{4-[1-(3-methylbutyl)-1,2,3,6-tetrahydropyridin-4-yl]phenyl}-3,4-dihydroisoquinoline-2(1H)-carboxamide;
N-[4-(5-propyl-1,2,4-oxadiazol-3-yl)phenyl]-3,4-dihydroisoquinoline-2(1H)-carboxamide;
N-[4-(5-benzyl-1,2,4-oxadiazol-3-yl)phenyl]-3,4-dihydroisoquinoline-2(1H)-carboxamide;
N-{4-[5-(3-methylbutyl)-1,2,4-oxadiazol-3-yl]phenyl}-3,4-dihydroisoquinoline-2(1H)-carboxamide;
N-hexyl-3,4-dihydroisoquinoline-2(1H)-carboxamide;
ethyl 6-[(3,4-dihydroisoquinolin-2(1H)-ylcarbonyl)amino]hexanoate;
N-(4-{2-[(phenylacetyl)amino]ethyl}phenyl)-3,4-dihydroisoquinoline-2(1H)-carboxamide;
N-{4-[2-(isobutyrylamino)ethyl]phenyl}-3,4-dihydroisoquinoline-2(1H)-carboxamide;
N-(4-{[(benzyloxy)acetyl]amino}phenyl)-3,4-dihydroisoquinoline-2(1H)-carboxamide;
N-(4-{[(4-methoxycyclohexyl)carbonyl]amino}phenyl)-3,4-dihydroisoquinoline-2(1H)-carboxamide;
N-(4-{[(1-acetylpiperidin-4-yl)carbonyl]amino}phenyl)-3,4-dihydroisoquinoline-2(1H)-carboxamide;
N-(4-{[4-oxo-4-(2-thienyl)butanoyl]amino}phenyl)-3,4-dihydroisoquinoline-2(1H)-carboxamide;
N-(4-{[3-(phenylsulfonyl)propanoyl]amino}phenyl)-3,4-dihydroisoquinoline-2(1H)-carboxamide;
N-{4-[((2R)-2,3-dihydro-1-benzofuran-2-ylcarbonyl)amino]phenyl}-3,4-dihydroisoquinoline-2(1H)-carboxamide and N-{4-[((2S)-2,3-dihydro-1-benzofuran-2-ylcarbonyl)amino]phenyl}-3,4-dihydroisoquinoline-2(1H)-carboxamide;
N N-{4-[((3R)-3-methylpentanoyl)amino]phenyl}-3,4-dihydroisoquinoline-2(1H)-carboxamide and N-{4-[((3S)-3-methylpentanoyl)amino]phenyl}-3,4-dihydroisoquinoline-2(1H)-carboxamide;
N-{4-[(2,2-dimethylbutanoyl)amino]phenyl}-3,4-dihydroisoquinoline-2(1H)-carboxamide;
N-{4-[(3,3-dimethylbutanoyl)amino]phenyl}-3,4-dihydroisoquinoline-2(1H)-carboxamide;
N-[4-(heptanoylamino)phenyl]-3,4-dihydroisoquinoline-2(1H)-carboxamide;
N-{4-[(4,4,4-trifluorobutanoyl)amino]phenyl}-3,4-dihydroisoquinoline-2(1H)-carboxamide;
N-(4-{[(2-methoxyethoxy)acetyl]amino}phenyl)-3,4-dihydroisoquinoline-2(1H)-carboxamide;
N-{4-[((3R)-tetrahydrofuran-3-ylcarbonyl)amino]phenyl}-3,4-dihydroisoquinoline-2(1H)-carboxamide and N-{4-[((3S)-tetrahydrofuran-3-ylcarbonyl)amino]phenyl}-3,4-dihydroisoquinoline-2(1H)-carboxamide;
N-(4-{[3-(methylthio)propanoyl]amino}phenyl)-3,4-dihydroisoquinoline-2(1H)-carboxamide;
N-{4-[(cyclopentylacetyl)amino]phenyl}-3,4-dihydroisoquinoline-2(1H)-carboxamide;
N-{4-[(cyclohexylcarbonyl)amino]phenyl}-3,4-dihydroisoquinoline-2(1H)-carboxamide;
N-{4-[(cyclohexylacetyl)amino]phenyl}-3,4-dihydroisoquinoline-2(1H)-carboxamide;
N-[4-(benzoylamino)phenyl]-3,4-dihydroisoquinoline-2(1H)-carboxamide;
N-{4-[(phenylacetyl)amino]phenyl}-3,4-dihydroisoquinoline-2(1H)-carboxamide;
N-(4-{[3-(4-aminophenyl)propanoyl]amino}phenyl)-3,4-dihydroisoquinoline-2(1H)-carboxamide;
N-[4-(3-furoylamino)phenyl]-3,4-dihydroisoquinoline-2(1H)-carboxamide;
N-{4-[(2,5-dimethyl-3-furoyl)amino]phenyl}-3,4-dihydroisoquinoline-2(1H)-carboxamide;
N-{4-[(3-thienylcarbonyl)amino]phenyl}-3,4-dihydroisoquinoline-2(1H)-carboxamide;
N-{4-[(1H-pyrrol-2-ylcarbonyl)amino]phenyl}-3,4-dihydroisoquinoline-2(1H)-carboxamide;
N-{4-[(1,3-thiazol-5-ylcarbonyl)amino]phenyl}-3,4-dihydroisoquinoline-2(1H)-carboxamide;
N-{4-[(1H-pyrazol-5-ylcarbonyl)amino]phenyl}-3,4-dihydroisoquinoline-2(1H)-carboxamide;
N-{4-[(1H-pyrazol-4-ylcarbonyl)amino]phenyl}-3,4-dihydroisoquinoline-2(1H)-carboxamide;
N-{4-[(1,2-oxazol-5-ylcarbonyl)amino]phenyl}-3,4-dihydroisoquinoline-2(1H)-carboxamide;
N-{4-[(pyridin-2-ylacetyl)amino]phenyl}-3,4-dihydroisoquinoline-2(1H)-carboxamide;
N-{4-[(N,N-dimethyl-beta-alanyl)amino]phenyl}-3,4-dihydroisoquinoline-2(1H)-carboxamide;
N-(4-{[3-(piperidin-1-yl)propanoyl]amino}phenyl)-3,4-dihydroisoquinoline-2(1H)-carboxamide;
N-{4-[(morpholin-4-ylacetyl)amino]phenyl}-3,4-dihydroisoquinoline-2(1H)-carboxamide;
N-(4-{[3-(morpholin-4-yl)propanoyl]amino}phenyl)-3,4-dihydroisoquinoline-2(1H)-carboxamide;
N-(4-{[3-(4-methylpiperazin-1-yl)propanoyl]amino}phenyl)-3,4-dihydroisoquinoline-2(1H)-carboxamide;
N-[4-(trifluoromethyl)phenyl]-3,4-dihydroisoquinoline-2(1H)-carboxamide;
N-{4-[(cyclopentylacetyl)amino]phenyl}-5-[(methylsulfonyl)amino]-3,4-dihydroisoquinoline-2(1H)-carboxamide; and pharmaceutically acceptable salts thereof.

### Embodiments of Formula (II)

In another aspect, the present invention provides compounds of Formula (II)

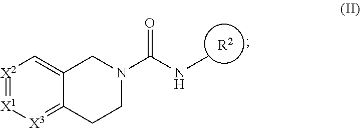

and pharmaceutically acceptable salts thereof; wherein X¹, X², and X³ are as described herein for Formula (I); and R² is phenyl, heterocyclyl, cycloalkyl, or cycloalkenyl; wherein the phenyl, heterocyclyl, cycloalkyl, and cycloalkenyl are optionally substituted as described herein for Formula (I).

In one embodiment of Formula (II), X¹, X² and X³ are CH; or X¹ and X³ are CH and X² is N; or X² and X³ are CH and X¹ is N; or X¹ is CR¹ and X² and X³ are CH; or X² is CR¹ and X¹ and X³ are CH; or X³ is CR¹; and X¹ and X² are CH.

In another embodiment of Formula (II), X¹, X² and X³ are CH. In another embodiment of Formula (II), X¹ and X³ are CH and X² is N. In another embodiment of Formula (II), X² and X³ are CH and X¹ is N. In another embodiment of Formula (II), X¹ is CR¹ and X² and X³ are CH. In another embodiment of Formula (II), X² is CR¹ and X¹ and X³ are CH. In another embodiment of Formula (II), X³ is CR¹; and X¹ and X² are CH.

In one embodiment of Formula (II), R¹ is NHSO₂R³, F, Cl, Br, or I. In another embodiment of Formula (II), R¹ is F. In another embodiment of Formula (II), R¹ is NHSO₂R³; and R³ is alkyl.

In one embodiment of Formula (II), X¹ is CR¹; X² and X³ are CH; and R¹ is F. In one embodiment of Formula (II), X² is CR¹; X¹ and X³ are CH; and R¹ is F. In another embodiment of Formula (II), X³ is CR¹; and X¹ and X² are CH; and R¹ is NHSO₂R³; and R³ is alkyl.

In one embodiment of Formula (II),

X¹ and X² and X³ are CH; or

X¹ and X³ are CH; and X² is N; or

X² and X³ are H; and X¹ is N; or

X¹ is CR¹; and X² and X³ are CH; or

X² is CR¹; and X¹ and X³ are CH; or

X³ is CR¹; and X¹ and X² are CH;

R¹ is NHSO₂R³, F, Cl, Br, or I;

R² is phenyl, or heterocyclyl; wherein each phenyl is optionally additionally substituted at the para position with one independently selected R⁵, C(O)R⁵, NHC(O)R⁵, or C(O)NHR⁵; wherein each heterocyclyl is optionally substituted with C(O)NHR⁵; wherein R² is not 4-methylphenyl;

R³ is alkyl;

R⁵ is alkyl, alkenyl, aryl, heterocyclyl, or cycloalkyl; wherein each alkyl and alkenyl, is optionally substituted with one or more independently selected R⁹, OR⁹, SR⁹, SO₂R⁹, C(O)R⁹, N(R⁹)₂, NHC(O)R⁹, C(O)NHR⁹, OH, or CF₃, F, Cl, Br or I;

R⁹ is alkyl, aryl, heterocyclyl, or cycloalkyl; wherein each alkyl is optionally substituted with one or more independently selected OCH₃, aryl, or heterocyclyl;

wherein the cyclic moieties represented by R⁵ and R⁹ are optionally substituted with one or more independently selected R¹³, OR¹³, SO₂R¹³, C(O)R¹³, CO(O)R¹³, NH₂, C(O)NHR¹³, F, Cl, Br or I;

R¹³ is alkyl, aryl, or heterocyclyl; wherein each alkyl is optionally substituted with one or more independently selected R¹⁴, OH, F, Cl, Br or I; and

R¹⁴ is aryl F, Cl, Br or I.

Still another embodiment pertains to compounds having Formula (II), which include
N-{4-[(3-methylbutyl)carbamoyl]phenyl}-3,4-dihydroisoquinoline-2(1H)-carboxamide;
N-(4-{[4-(pyridin-2-yl)piperazin-1-yl]carbonyl}phenyl)-3,4-dihydroisoquinoline-2(1H)-carboxamide;
6-fluoro-N-{4-[(3-phenylpropyl)carbamoyl]phenyl}-3,4-dihydroisoquinoline-2(1H)-carboxamide;
N-{4-[(3-phenylpropyl)carbamoyl]phenyl}-3,4-dihydro-2,6-naphthyridine-2(1H)-carboxamide;
N-{4-[(3-methylbutyl)carbamoyl]phenyl}-3,4-dihydro-2,6-naphthyridine-2(1H)-carboxamide;
6-fluoro-N-{4-[(3-methylbutyl)carbamoyl]phenyl}-3,4-dihydroisoquinoline-2(1H)-carboxamide;
N-{4-[(3-phenylpropyl)carbamoyl]phenyl}-3,4-dihydroisoquinoline-2(1H)-carboxamide;
N-[4-(benzylcarbamoyl)phenyl]-3,4-dihydroisoquinoline-2(1H)-carboxamide;
N-{5-[(3-phenylpropyl)carbamoyl]pyridin-2-yl}-3,4-dihydroisoquinoline-2(1H)-carboxamide;
N-{5-[(3-methylbutyl)carbamoyl]pyridin-2-yl}-3,4-dihydroisoquinoline-2(1H)-carboxamide;
N-(4-{[1-(3-methylbutyl)-1H-pyrazol-4-yl]carbamoyl}phenyl)-3,4-dihydroisoquinoline-2(1H)-carboxamide;
N-(4-{[2-(2-thienyl)ethyl]carbamoyl}phenyl)-3,4-dihydro-2,6-naphthyridine-2(1H)-carboxamide;
6-fluoro-N-(4-{[2-(2-thienyl)ethyl]carbamoyl}phenyl)-3,4-dihydroisoquinoline-2(1H)-carboxamide;
N-{4-[(1-benzyl-1H-pyrazol-4-yl)carbamoyl]phenyl}-3,4-dihydroisoquinoline-2(1H)-carboxamide;
N-{4-[(2-phenylethyl)carbamoyl]phenyl}-3,4-dihydroisoquinoline-2(1H)-carboxamide;
7-fluoro-N-{4-[(3-methylbutyl)carbamoyl]phenyl}-3,4-dihydroisoquinoline-2(1H)-carboxamide;
N-(4-{[2-(2-thienyl)ethyl]carbamoyl}phenyl)-3,4-dihydroisoquinoline-2(1H)-carboxamide;
N-{6-[(3-methylbutyl)carbamoyl]pyridin-3-yl}-3,4-dihydroisoquinoline-2(1H)-carboxamide;
N-(5-{[2-(2-thienyl)ethyl]carbamoyl}pyridin-2-yl)-3,4-dihydroisoquinoline-2(1H)-carboxamide;
7-fluoro-N-{4-[(3-phenylpropyl)carbamoyl]phenyl}-3,4-dihydroisoquinoline-2(1H)-carboxamide;
N-{6-[(3-phenylpropyl)carbamoyl]pyridin-3-yl}-3,4-dihydroisoquinoline-2(1H)-carboxamide;
N-(6-{[2-(2-thienyl)ethyl]carbamoyl}pyridin-3-yl)-3,4-dihydroisoquinoline-2(1H)-carboxamide;
7-fluoro-N-(4-{[2-(2-thienyl)ethyl]carbamoyl}phenyl)-3,4-dihydroisoquinoline-2(1H)-carboxamide;
N-{4-[(3-phenylpropyl)carbamoyl]phenyl}-3,4-dihydro-2,7-naphthyridine-2(1H)-carboxamide;
N-[4-(2-oxo-2-{[2-(2-thienyl)ethyl]amino}ethyl)phenyl]-3,4-dihydroisoquinoline-2(1H)-carboxamide;
N-(4-{2-oxo-2-[(3-phenylpropyl)amino]ethyl}phenyl)-3,4-dihydroisoquinoline-2(1H)-carboxamide;
N-{4-[(3-methylbutyl)carbamoyl]phenyl}-3,4-dihydro-2,7-naphthyridine-2(1H)-carboxamide;
N-(4-{2-[(3-methylbutyl)amino]-2-oxoethyl}phenyl)-3,4-dihydroisoquinoline-2(1H)-carboxamide;
N-(4-{[2-(2-thienyl)ethyl]carbamoyl}phenyl)-3,4-dihydro-2,7-naphthyridine-2(1H)-carboxamide;
N-{4-[(4-methylpentanoyl)amino]phenyl}-3,4-dihydroisoquinoline-2(1H)-carboxamide;
N-{4-[(4-phenylbutanoyl)amino]phenyl}-3,4-dihydroisoquinoline-2(1H)-carboxamide;
N-(4-{[3-(2-thienyl)propanoyl]amino}phenyl)-3,4-dihydroisoquinoline-2(1H)-carboxamide;
N-[4-(1-isobutyl-1H-pyrazol-4-yl)phenyl]-3,4-dihydroisoquinoline-2(1H)-carboxamide;
N-[4-(1-propyl-1H-pyrazol-4-yl)phenyl]-3,4-dihydroisoquinoline-2(1H)-carboxamide;
N-{4-[1-((2R)-2-hydroxypropyl)-1H-pyrazol-4-yl]phenyl}-3,4-dihydroisoquinoline-2(1H)-carboxamide;
N-{4-[1-(3-methylbutyl)-1H-pyrazol-4-yl]phenyl}-3,4-dihydroisoquinoline-2(1H)-carboxamide;
N-[4-(1-benzyl-1H-pyrazol-4-yl)phenyl]-3,4-dihydroisoquinoline-2(1H)-carboxamide;
N-{4-[(1E)-5-phenylpent-1-en-1-yl]phenyl}-3,4-dihydroisoquinoline-2(1H)-carboxamide;
N-[4-(1-ethyl-1H-pyrazol-4-yl)phenyl]-3,4-dihydroisoquinoline-2(1H)-carboxamide;
N-{4-[1-(2-hydroxyethyl)-1H-pyrazol-4-yl]phenyl}-3,4-dihydroisoquinoline-2(1H)-carboxamide;
N-[4-(1-methyl-1H-pyrazol-4-yl)phenyl]-3,4-dihydroisoquinoline-2(1H)-carboxamide;
N-[4-(1-benzoyl-1,2,3,6-tetrahydropyridin-4-yl)phenyl]-3,4-dihydroisoquinoline-2(1H)-carboxamide;
N-[4-(1-butyryl-1,2,3,6-tetrahydropyridin-4-yl)phenyl]-3,4-dihydroisoquinoline-2(1H)-carboxamide;
N-{4-[1-(isopropylsulfonyl)-1,2,3,6-tetrahydropyridin-4-yl]phenyl}-3,4-dihydroisoquinoline-2(1H)-carboxamide;
N-[4-(1-isobutyryl-1,2,3,6-tetrahydropyridin-4-yl)phenyl]-3,4-dihydroisoquinoline-2(1H)-carboxamide;
N-{4-[1-(3-methylbutanoyl)-1,2,3,6-tetrahydropyridin-4-yl]phenyl}-3,4-dihydroisoquinoline-2(1H)-carboxamide;
N-{4-[1-(methylcarbamoyl)-1,2,3,6-tetrahydropyridin-4-yl]phenyl}-3,4-dihydroisoquinoline-2(1H)-carboxamide;
tert-butyl 4-{4-[(3,4-dihydroisoquinolin-2(1H)-ylcarbonyl)amino]phenyl}-3,6-dihydropyridine-1(2H)-carboxylate;
N-[4-(1-acetyl-1,2,3,6-tetrahydropyridin-4-yl)phenyl]-3,4-dihydroisoquinoline-2(1H)-carboxamide;
N-{4-[1-(isobutylsulfonyl)-1,2,3,6-tetrahydropyridin-4-yl]phenyl}-3,4-dihydroisoquinoline-2(1H)-carboxamide;
N-[4-(1-benzyl-1,2,3,6-tetrahydropyridin-4-yl)phenyl]-3,4-dihydroisoquinoline-2(1H)-carboxamide;
N-{4-[1-(methylsulfonyl)-1,2,3,6-tetrahydropyridin-4-yl]phenyl}-3,4-dihydroisoquinoline-2(1H)-carboxamide;
N-[4-(1,2,3,6-tetrahydropyridin-4-yl)phenyl]-3,4-dihydroisoquinoline-2(1H)-carboxamide;
N-{4-[1-(3-methylbutyl)-1,2,3,6-tetrahydropyridin-4-yl]phenyl}-3,4-dihydroisoquinoline-2(1H)-carboxamide;
N-[4-(5-propyl-1,2,4-oxadiazol-3-yl)phenyl]-3,4-dihydroisoquinoline-2(1H)-carboxamide;
N-[4-(5-benzyl-1,2,4-oxadiazol-3-yl)phenyl]-3,4-dihydroisoquinoline-2(1H)-carboxamide;
ethyl 6-[(3,4-dihydroisoquinolin-2(1H)-ylcarbonyl)amino]hexanoate;
N-(4-{2-[(phenylacetyl)amino]ethyl}phenyl)-3,4-dihydroisoquinoline-2(1H)-carboxamide;
N-{4-[2-(isobutyrylamino)ethyl]phenyl}-3,4-dihydroisoquinoline-2(1H)-carboxamide;
N-(4-{[(benzyloxy)acetyl]amino}phenyl)-3,4-dihydroisoquinoline-2(1H)-carboxamide;
N-(4-{[(4-methoxycyclohexyl)carbonyl]amino}phenyl)-3,4-dihydroisoquinoline-2(1H)-carboxamide;
N-(4-{[(1-acetylpiperidin-4-yl)carbonyl]amino}phenyl)-3,4-dihydroisoquinoline-2(1H)-carboxamide;
N-(4-{[4-oxo-4-(2-thienyl)butanoyl]amino}phenyl)-3,4-dihydroisoquinoline-2(1H)-carboxamide;
N-(4-{[3-(phenylsulfonyl)propanoyl]amino}phenyl)-3,4-dihydroisoquinoline-2(1H)-carboxamide;
N-{4-[((2R)-2,3-dihydro-1-benzofuran-2-ylcarbonyl)amino]phenyl}-3,4-dihydroisoquinoline-2(1H)-carboxamide and N-{4-[((2S)-2,3-dihydro-1-benzofuran-2-ylcarbonyl)amino]phenyl}-3,4-dihydroisoquinoline-2(1H)-carboxamide;
N N-{4-[((3R)-3-methylpentanoyl)amino]phenyl}-3,4-dihydroisoquinoline-2(1H)-carboxamide and N-{4-[((3S)-3-methylpentanoyl)amino]phenyl}-3,4-dihydroisoquinoline-2(1H)-carboxamide;
N-{4-[(2,2-dimethylbutanoyl)amino]phenyl}-3,4-dihydroisoquinoline-2(1H)-carboxamide;
N-{4-[(3,3-dimethylbutanoyl)amino]phenyl}-3,4-dihydroisoquinoline-2(1H)-carboxamide;
N-[4-(heptanoylamino)phenyl]-3,4-dihydroisoquinoline-2(1H)-carboxamide;
N-{4-[(4,4,4-trifluorobutanoyl)amino]phenyl}-3,4-dihydroisoquinoline-2(1H)-carboxamide;
N-(4-{[(2-methoxyethoxy)acetyl]amino}phenyl)-3,4-dihydroisoquinoline-2(1H)-carboxamide;
N-{4-[((3R)-tetrahydrofuran-3-ylcarbonyl)amino]phenyl}-3,4-dihydroisoquinoline-2(1H)-carboxamide and N-{4-[((3S)-tetrahydrofuran-3-ylcarbonyl)amino]phenyl}-3,4-dihydroisoquinoline-2(1H)-carboxamide;
N-(4-{[3-(methylthio)propanoyl]amino}phenyl)-3,4-dihydroisoquinoline-2(1H)-carboxamide;
N-{4-[(cyclopentylacetyl)amino]phenyl}-3,4-dihydroisoquinoline-2(1H)-carboxamide;
N-{4-[(cyclohexylcarbonyl)amino]phenyl}-3,4-dihydroisoquinoline-2(1H)-carboxamide;
N-{4-[(cyclohexylacetyl)amino]phenyl}-3,4-dihydroisoquinoline-2(1H)-carboxamide;
N-[4-(benzoylamino)phenyl]-3,4-dihydroisoquinoline-2(1H)-carboxamide;
N-{4-[(phenylacetyl)amino]phenyl}-3,4-dihydroisoquinoline-2(1H)-carboxamide;
N-(4-{[3-(4-aminophenyl)propanoyl]amino}phenyl)-3,4-dihydroisoquinoline-2(1H)-carboxamide;
N-[4-(3-furoylamino)phenyl]-3,4-dihydroisoquinoline-2(1H)-carboxamide;
N-{4-[(2,5-dimethyl-3-furoyl)amino]phenyl}-3,4-dihydroisoquinoline-2(1H)-carboxamide;
N-{4-[(3-thienylcarbonyl)amino]phenyl}-3,4-dihydroisoquinoline-2(1H)-carboxamide;
N-{4-[(1H-pyrrol-2-ylcarbonyl)amino]phenyl}-3,4-dihydroisoquinoline-2(1H)-carboxamide;
N-{4-[(1,3-thiazol-5-ylcarbonyl)amino]phenyl}-3,4-dihydroisoquinoline-2(1H)-carboxamide;
N-{4-[(1H-pyrazol-5-ylcarbonyl)amino]phenyl}-3,4-dihydroisoquinoline-2(1H)-carboxamide;
N-{4-[(1H-pyrazol-4-ylcarbonyl)amino]phenyl}-3,4-dihydroisoquinoline-2(1H)-carboxamide;
N-{4-[(1,2-oxazol-5-ylcarbonyl)amino]phenyl}-3,4-dihydroisoquinoline-2(1H)-carboxamide;
N-{4-[(pyridin-2-ylacetyl)amino]phenyl}-3,4-dihydroisoquinoline-2(1H)-carboxamide;
N-{4-[(N,N-dimethyl-beta-alanyl)amino]phenyl}-3,4-dihydroisoquinoline-2(1H)-carboxamide;
N-(4-{[3-(piperidin-1-yl)propanoyl]amino}phenyl)-3,4-dihydroisoquinoline-2(1H)-carboxamide;
N-{4-[(morpholin-4-ylacetyl)amino]phenyl}-3,4-dihydroisoquinoline-2(1H)-carboxamide;
N-(4-{[3-(morpholin-4-yl)propanoyl]amino}phenyl)-3,4-dihydroisoquinoline-2(1H)-carboxamide;
N-(4-{[3-(4-methylpiperazin-1-yl)propanoyl]amino}phenyl)-3,4-dihydroisoquinoline-2(1H)-carboxamide;
N-[4-(trifluoromethyl)phenyl]-3,4-dihydroisoquinoline-2(1H)-carboxamide;
N-{4-[(cyclopentylacetyl)amino]phenyl}-5-[(methylsulfonyl)amino]-3,4-dihydroisoquinoline-2(1H)-carboxamide; and pharmaceutically acceptable salts thereof.

### Embodiments of Formula (III)

In another aspect, the present invention provides compounds of Formula (III)

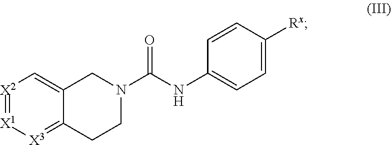

and pharmaceutically acceptable salts thereof; wherein X¹, X², and X³ are as described herein for Formula (I); and R^{X} is as described herein for substituents in Formula (I) when R² is phenyl.

In one embodiment of Formula (III), X¹, X² and X³ are CH; or X¹ and X³ are CH and X² is N; or X² and X³ are CH and X¹ is N; or X¹ is CR¹ and X² and X³ are CH; or X² is CR¹ and X¹ and X³ are CH; or X³ is CR¹; and X¹ and X² are CH.

In another embodiment of Formula (III), X¹, X² and X³ are CH. In another embodiment of Formula (III), X¹ and X³ are CH and X² is N. In another embodiment of Formula (III), X² and X³ are CH and X¹ is N. In another embodiment of Formula (III), X¹ is CR¹ and X² and X³ are CH. In another embodiment of Formula (III), X² is CR¹ and X¹ and X³ are CH. In another embodiment of Formula (III), X³ is CR¹; and X¹ and X² are CH.

In one embodiment of Formula (III), R¹ is NHSO₂R³, F, Cl, Br, or I. In another embodiment of Formula (III), R¹ is F. In another embodiment of Formula (III), R¹ is NHSO₂R³; and R³ is alkyl.

In one embodiment of Formula (III), X¹ is CR¹; X² and X³ are CH; and R¹ is F. In one embodiment of Formula (III), X² is CR¹; X¹ and X³ are CH; and R¹ is F. In another embodiment of Formula (III), X³ is CR¹; and X¹ and X² are CH; and R¹ is NHSO₂R³; and R³ is alkyl.

In one embodiment of Formula (III),

X¹ and X² and X³ are CH; or

X¹ and X³ are CH; and X² is N; or

X² and X³ are H; and X¹ is N; or

X¹ is CR¹; and X² and X³ are CH; or

X² is CR¹; and X¹ and X³ are CH; or

X³ is CR¹; and X¹ and X² are CH;

R¹ is NHSO₂R³, F, Cl, Br, or I;

R³ is alkyl;

R^{x} is R⁵, C(O)R⁵, NHC(O)R⁵, or C(O)NHR⁵; wherein R^{x} is not methyl;

R⁵ is alkyl, alkenyl, aryl, heterocyclyl, or cycloalkyl; wherein each alkyl and alkenyl, is optionally substituted with one or more independently selected R⁹, OR⁹, SR⁹, SO₂R⁹, C(O)R⁹, N(R⁹)₂, NHC(O)R⁹, C(O)NHR⁹, OH, or CF₃, F, Cl, Br or I;

R⁹ is alkyl, aryl, heterocyclyl, or cycloalkyl; wherein each alkyl is optionally substituted with one or more independently selected OCH₃, aryl, or heterocyclyl;

wherein the cyclic moieties represented by R⁵ and R⁹ are optionally substituted with one or more independently selected R¹³, OR¹³, SO₂R¹³, C(O)R¹³, CO(O)R¹³, NH₂, C(O)NHR¹³, F, Cl, Br or I;

R¹³ is alkyl, aryl, or heterocyclyl; wherein each alkyl is optionally substituted with one or more independently selected R¹⁴, OH, F, Cl, Br or I; and

R¹⁴ is aryl F, Cl, Br or I.

Still another embodiment pertains to compounds having Formula (III), which include
N-{4-[(3-methylbutyl)carbamoyl]phenyl}-3,4-dihydroisoquinoline-2(1H)-carboxamide;
N-(4-{[4-(pyridin-2-yl)piperazin-1-yl]carbonyl}phenyl)-3,4-dihydroisoquinoline-2(1H)-carboxamide;
6-fluoro-N-{4-[(3-phenylpropyl)carbamoyl]phenyl}-3,4-dihydroisoquinoline-2(1H)-carboxamide;
N-{4-[(3-phenylpropyl)carbamoyl]phenyl}-3,4-dihydro-2,6-naphthyridine-2(1H)-carboxamide;
N-{4-[(3-methylbutyl)carbamoyl]phenyl}-3,4-dihydro-2,6-naphthyridine-2(1H)-carboxamide;
6-fluoro-N-{4-[(3-methylbutyl)carbamoyl]phenyl}-3,4-dihydroisoquinoline-2(1H)-carboxamide;
N-{4-[(3-phenylpropyl)carbamoyl]phenyl}-3,4-dihydroisoquinoline-2(1H)-carboxamide;
N-[4-(benzylcarbamoyl)phenyl]-3,4-dihydroisoquinoline-2(1H)-carboxamide;
N-(4-{[1-(3-methylbutyl)-1H-pyrazol-4-yl]carbamoyl}phenyl)-3,4-dihydroisoquinoline-2(1H)-carboxamide;
N-(4-{[2-(2-thienyl)ethyl]carbamoyl}phenyl)-3,4-dihydro-2,6-naphthyridine-2(1H)-carboxamide;
6-fluoro-N-(4-{[2-(2-thienyl)ethyl]carbamoyl}phenyl)-3,4-dihydroisoquinoline-2(1H)-carboxamide;
N-{4-[(1-benzyl-1H-pyrazol-4-yl)carbamoyl]phenyl}-3,4-dihydroisoquinoline-2(1H)-carboxamide;
N-{4-[(2-phenylethyl)carbamoyl]phenyl}-3,4-dihydroisoquinoline-2(1H)-carboxamide;
7-fluoro-N-{4-[(3-methylbutyl)carbamoyl]phenyl}-3,4-dihydroisoquinoline-2(1H)-carboxamide;
N-(4-{[2-(2-thienyl)ethyl]carbamoyl}phenyl)-3,4-dihydroisoquinoline-2(1H)-carboxamide;
7-fluoro-N-{4-[(3-phenylpropyl)carbamoyl]phenyl}-3,4-dihydroisoquinoline-2(1H)-carboxamide;
7-fluoro-N-(4-{[2-(2-thienyl)ethyl]carbamoyl}phenyl)-3,4-dihydroisoquinoline-2(1H)-carboxamide;
N-{4-[(3-phenylpropyl)carbamoyl]phenyl}-3,4-dihydro-2,7-naphthyridine-2(1H)-carboxamide;
N-[4-(2-oxo-2-{[2-(2-thienyl)ethyl]amino}ethyl)phenyl]-3,4-dihydroisoquinoline-2(1H)-carboxamide;
N-(4-{2-oxo-2-[(3-phenylpropyl)amino]ethyl}phenyl)-3,4-dihydroisoquinoline-2(1H)-carboxamide;
N-{4-[(3-methylbutyl)carbamoyl]phenyl}-3,4-dihydro-2,7-naphthyridine-2(1H)-carboxamide;
N-(4-{2-[(3-methylbutyl)amino]-2-oxoethyl}phenyl)-3,4-dihydroisoquinoline-2(1H)-carboxamide;
N-(4-{[2-(2-thienyl)ethyl]carbamoyl}phenyl)-3,4-dihydro-2,7-naphthyridine-2(1H)-carboxamide;
N-{4-[(4-methylpentanoyl)amino]phenyl}-3,4-dihydroisoquinoline-2(1H)-carboxamide;
N-{4-[(4-phenylbutanoyl)amino]phenyl}-3,4-dihydroisoquinoline-2(1H)-carboxamide;
N-(4-{[3-(2-thienyl)propanoyl]amino}phenyl)-3,4-dihydroisoquinoline-2(1H)-carboxamide;
N-[4-(1-isobutyl-1H-pyrazol-4-yl)phenyl]-3,4-dihydroisoquinoline-2(1H)-carboxamide;
N-[4-(1-propyl-1H-pyrazol-4-yl)phenyl]-3,4-dihydroisoquinoline-2(1H)-carboxamide;
N-{4-[1-((2R)-2-hydroxypropyl)-1H-pyrazol-4-yl]phenyl}-3,4-dihydroisoquinoline-2(1H)-carboxamide;
N-{4-[1-(3-methylbutyl)-1H-pyrazol-4-yl]phenyl}-3,4-dihydroisoquinoline-2(1H)-carboxamide;
N-[4-(1-benzyl-1H-pyrazol-4-yl)phenyl]-3,4-dihydroisoquinoline-2(1H)-carboxamide;
N-{4-[(1E)-5-phenylpent-1-en-1-yl]phenyl}-3,4-dihydroisoquinoline-2(1H)-carboxamide;
N-[4-(1-ethyl-1H-pyrazol-4-yl)phenyl]-3,4-dihydroisoquinoline-2(1H)-carboxamide;
N-{4-[1-(2-hydroxyethyl)-1H-pyrazol-4-yl]phenyl}-3,4-dihydroisoquinoline-2(1H)-carboxamide;
N-[4-(1-methyl-1H-pyrazol-4-yl)phenyl]-3,4-dihydroisoquinoline-2(1H)-carboxamide;
N-[4-(1-benzoyl-1,2,3,6-tetrahydropyridin-4-yl)phenyl]-3,4-dihydroisoquinoline-2(1H)-carboxamide;
N-[4-(1-butyryl-1,2,3,6-tetrahydropyridin-4-yl)phenyl]-3,4-dihydroisoquinoline-2(1H)-carboxamide;
N-{4-[1-(isopropylsulfonyl)-1,2,3,6-tetrahydropyridin-4-yl]phenyl}-3,4-dihydroisoquinoline-2(1H)-carboxamide;
N-[4-(1-isobutyryl-1,2,3,6-tetrahydropyridin-4-yl)phenyl]-3,4-dihydroisoquinoline-2(1H)-carboxamide;
N-{4-[1-(3-methylbutanoyl)-1,2,3,6-tetrahydropyridin-4-yl]phenyl}-3,4-dihydroisoquinoline-2(1H)-carboxamide;
N-{4-[1-(methylcarbamoyl)-1,2,3,6-tetrahydropyridin-4-yl]phenyl}-3,4-dihydroisoquinoline-2(1H)-carboxamide;
tert-butyl 4-{4-[(3,4-dihydroisoquinolin-2(1H)-ylcarbonyl)amino]phenyl}-3,6-dihydropyridine-1(2H)-carboxylate;
N-[4-(1-acetyl-1,2,3,6-tetrahydropyridin-4-yl)phenyl]-3,4-dihydroisoquinoline-2(1H)-carboxamide;
N-{4-[1-(isobutylsulfonyl)-1,2,3,6-tetrahydropyridin-4-yl]phenyl}-3,4-dihydroisoquinoline-2(1H)-carboxamide;
N-[4-(1-benzyl-1,2,3,6-tetrahydropyridin-4-yl)phenyl]-3,4-dihydroisoquinoline-2(1H)-carboxamide;
N-{4-[1-(methylsulfonyl)-1,2,3,6-tetrahydropyridin-4-yl]phenyl}-3,4-dihydroisoquinoline-2(1H)-carboxamide;
N-[4-(1,2,3,6-tetrahydropyridin-4-yl)phenyl]-3,4-dihydroisoquinoline-2(1H)-carboxamide;
N-{4-[1-(3-methylbutyl)-1,2,3,6-tetrahydropyridin-4-yl]phenyl}-3,4-dihydroisoquinoline-2(1H)-carboxamide;
N-[4-(5-propyl-1,2,4-oxadiazol-3-yl)phenyl]-3,4-dihydroisoquinoline-2(1H)-carboxamide;
N-[4-(5-benzyl-1,2,4-oxadiazol-3-yl)phenyl]-3,4-dihydroisoquinoline-2(1H)-carboxamide;
ethyl 6-[(3,4-dihydroisoquinolin-2(1H)-ylcarbonyl)amino]hexanoate;
N-(4-{2-[(phenylacetyl)amino]ethyl}phenyl)-3,4-dihydroisoquinoline-2(1H)-carboxamide;
N-{4-[2-(isobutyrylamino)ethyl]phenyl}-3,4-dihydroisoquinoline-2(1H)-carboxamide;
N-(4-{[(benzyloxy)acetyl]amino}phenyl)-3,4-dihydroisoquinoline-2(1H)-carboxamide;
N-(4-{[(4-methoxycyclohexyl)carbonyl]amino}phenyl)-3,4-dihydroisoquinoline-2(1H)-carboxamide;
N-(4-{[(1-acetylpiperidin-4-yl)carbonyl]amino}phenyl)-3,4-dihydroisoquinoline-2(1H)-carboxamide;
N-(4-{[4-oxo-4-(2-thienyl)butanoyl]amino}phenyl)-3,4-dihydroisoquinoline-2(1H)-carboxamide;
N-(4-{[3-(phenylsulfonyl)propanoyl]amino}phenyl)-3,4-dihydroisoquinoline-2(1H)-carboxamide;
N-{4-[((2R)-2,3-dihydro-1-benzofuran-2-ylcarbonyl)amino]phenyl}-3,4-dihydroisoquinoline-2(1H)-carboxamide and N-{4-[((2S)-2,3-dihydro-1-benzofuran-2-ylcarbonyl)amino]phenyl}-3,4-dihydroisoquinoline-2(1H)-carboxamide;
N N-{4-[((3R)-3-methylpentanoyl)amino]phenyl}-3,4-dihydroisoquinoline-2(1H)-carboxamide and N-{4-[((3S)-3-methylpentanoyl)amino]phenyl}-3,4-dihydroisoquinoline-2(1H)-carboxamide;
N-{4-[(2,2-dimethylbutanoyl)amino]phenyl}-3,4-dihydroisoquinoline-2(1H)-carboxamide;
N-{4-[(3,3-dimethylbutanoyl)amino]phenyl}-3,4-dihydroisoquinoline-2(1H)-carboxamide;
N-[4-(heptanoylamino)phenyl]-3,4-dihydroisoquinoline-2(1H)-carboxamide;
N-{4-[(4,4,4-trifluorobutanoyl)amino]phenyl}-3,4-dihydroisoquinoline-2(1H)-carboxamide;
N-(4-{[(2-methoxyethoxy)acetyl]amino}phenyl)-3,4-dihydroisoquinoline-2(1H)-carboxamide;
N-{4-[((3R)-tetrahydrofuran-3-ylcarbonyl)amino]phenyl}-3,4-dihydroisoquinoline-2(1H)-carboxamide and N-{4-[((3S)-tetrahydrofuran-3-ylcarbonyl)amino]phenyl}-3,4-dihydroisoquinoline-2(1H)-carboxamide;
N-(4-{[3-(methylthio)propanoyl]amino}phenyl)-3,4-dihydroisoquinoline-2(1H)-carboxamide;
N-{4-[(cyclopentylacetyl)amino]phenyl}-3,4-dihydroisoquinoline-2(1H)-carboxamide;
N-{4-[(cyclohexylcarbonyl)amino]phenyl}-3,4-dihydroisoquinoline-2(1H)-carboxamide;
N-{4-[(cyclohexylacetyl)amino]phenyl}-3,4-dihydroisoquinoline-2(1H)-carboxamide;
N-[4-(benzoylamino)phenyl]-3,4-dihydroisoquinoline-2(1H)-carboxamide;
N-{4-[(phenylacetyl)amino]phenyl}-3,4-dihydroisoquinoline-2(1H)-carboxamide;
N-(4-{[3-(4-aminophenyl)propanoyl]amino}phenyl)-3,4-dihydroisoquinoline-2(1H)-carboxamide;
N-[4-(3-furoylamino)phenyl]-3,4-dihydroisoquinoline-2(1H)-carboxamide;
N-{4-[(2,5-dimethyl-3-furoyl)amino]phenyl}-3,4-dihydroisoquinoline-2(1H)-carboxamide;
N-{4-[(3-thienylcarbonyl)amino]phenyl}-3,4-dihydroisoquinoline-2(1H)-carboxamide;
N-{4-[(1H-pyrrol-2-ylcarbonyl)amino]phenyl}-3,4-dihydroisoquinoline-2(1H)-carboxamide;
N-{4-[(1,3-thiazol-5-ylcarbonyl)amino]phenyl}-3,4-dihydroisoquinoline-2(1H)-carboxamide;
N-{4-[(1H-pyrazol-5-ylcarbonyl)amino]phenyl}-3,4-dihydroisoquinoline-2(1H)-carboxamide;
N-{4-[(1H-pyrazol-4-ylcarbonyl)amino]phenyl}-3,4-dihydroisoquinoline-2(1H)-carboxamide;
N-{4-[(1,2-oxazol-5-ylcarbonyl)amino]phenyl}-3,4-dihydroisoquinoline-2(1H)-carboxamide;
N-{4-[(pyridin-2-ylacetyl)amino]phenyl}-3,4-dihydroisoquinoline-2(1H)-carboxamide;
N-{4-[(N,N-dimethyl-beta-alanyl)amino]phenyl}-3,4-dihydroisoquinoline-2(1H)-carboxamide;
N-(4-{[3-(piperidin-1-yl)propanoyl]amino}phenyl)-3,4-dihydroisoquinoline-2(1H)-carboxamide;
N-{4-[(morpholin-4-ylacetyl)amino]phenyl}-3,4-dihydroisoquinoline-2(1H)-carboxamide;
N-(4-{[3-(morpholin-4-yl)propanoyl]amino}phenyl)-3,4-dihydroisoquinoline-2(1H)-carboxamide;
N-(4-{[3-(4-methylpiperazin-1-yl)propanoyl]amino}phenyl)-3,4-dihydroisoquinoline-2(1H)-carboxamide;
N-[4-(trifluoromethyl)phenyl]-3,4-dihydroisoquinoline-2(1H)-carboxamide;
N-{4-[(cyclopentylacetyl)amino]phenyl}-5-[(methylsulfonyl)amino]-3,4-dihydroisoquinoline-2(1H)-carboxamide; and pharmaceutically acceptable salts thereof.

### Embodiments of Formula (IV)

In another aspect, the present invention provides compounds of Formula (IV)

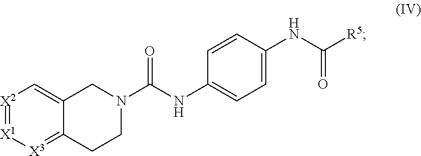

and pharmaceutically acceptable salts thereof; wherein X¹, X², X³ and R⁵ are as described herein for Formula (I).

In one embodiment of Formula (IV), X¹, X² and X³ are CH; or X¹ and X³ are CH and X² is N; or X² and X³ are CH and X¹ is N; or X¹ is CR¹ and X² and X³ are CH; or X² is CR¹ and X¹ and X³ are CH; or X³ is CR¹; and X¹ and X² are CH.

In another embodiment of Formula (IV), X¹, X² and X³ are CH. In another embodiment of Formula (IV), X¹ and X³ are CH and X² is N. In another embodiment of Formula (IV), X² and X³ are CH and X¹ is N. In another embodiment of Formula (IV), X¹ is CR¹ and X² and X³ are CH. In another embodiment of Formula (IV), X² is CR¹ and X¹ and X³ are CH. In another embodiment of Formula (IV), X³ is CR¹; and X¹ and X² are CH.

In one embodiment of Formula (IV), R¹ is NHSO₂R³, F, Cl, Br, or I. In another embodiment of Formula (IV), R¹ is F. In another embodiment of Formula (IV), R¹ is NHSO₂R³; and R³ is alkyl.

In one embodiment of Formula (IV), X¹ is CR¹; X² and X³ are CH; and R¹ is F. In one embodiment of Formula (IV), X² is CR¹; X¹ and X³ are CH; and R¹ is F. In another embodiment of Formula (IV), X³ is CR¹; and X¹ and X² are CH; and R¹ is NHSO₂R³; and R³ is alkyl.

In one embodiment of Formula (IV),

X¹ and X² and X³ are CH; or

X¹ and X³ are CH; and X² is N; or

X² and X³ are H; and X¹ is N; or

X¹ is CR¹; and X² and X³ are CH; or

X² is CR¹; and X¹ and X³ are CH; or

X³ is CR¹; and X¹ and X² are CH;

R¹ is NHSO₂R³, F, Cl, Br, or I;

R³ is alkyl;

R⁵ is alkyl, alkenyl, aryl, heterocyclyl, or cycloalkyl; wherein each alkyl and alkenyl, is optionally substituted with one or more independently selected R⁹, OR⁹, SR⁹, SO₂R⁹, C(O)R⁹, N(R⁹)₂, NHC(O)R⁹, C(O)NHR⁹, OH, or CF₃, F, Cl, Br or I;

R⁹ is alkyl, aryl, heterocyclyl, or cycloalkyl; wherein each alkyl is optionally substituted with one or more independently selected OCH₃, aryl, or heterocyclyl;

wherein the cyclic moieties represented by R⁵ and R⁹ are optionally substituted with one or more independently selected R¹³, OR¹³, SO₂R¹³, C(O)R¹³, CO(O)R¹³, NH₂, (O)NHR¹³, F, Cl, Br or I;

R¹³ is alkyl, aryl, or heterocyclyl; wherein each alkyl is optionally substituted with one or more independently selected R¹⁴, OH, F, Cl, Br or I; and

R¹⁴ is aryl F, Cl, Br or I.

Still another embodiment pertains to compounds having Formula (IV), which include
N-{4-[(4-methylpentanoyl)amino]phenyl}-3,4-dihydroisoquinoline-2(1H)-carboxamide;
N-{4-[(4-phenylbutanoyl)amino]phenyl}-3,4-dihydroisoquinoline-2(1H)-carboxamide;
N-(4-{[3-(2-thienyl)propanoyl]amino}phenyl)-3,4-dihydroisoquinoline-2(1H)-carboxamide;
N-(4-{[(benzyloxy)acetyl]amino}phenyl)-3,4-dihydroisoquinoline-2(1H)-carboxamide;
N-(4-{[(4-methoxycyclohexyl)carbonyl]amino}phenyl)-3,4-dihydroisoquinoline-2(1H)-carboxamide;
N-(4-{[(1-acetylpiperidin-4-yl)carbonyl]amino}phenyl)-3,4-dihydroisoquinoline-2(1H)-carboxamide;
N-(4-{[4-oxo-4-(2-thienyl)butanoyl]amino}phenyl)-3,4-dihydroisoquinoline-2(1H)-carboxamide;
N-(4-{[3-(phenylsulfonyl)propanoyl]amino}phenyl)-3,4-dihydroisoquinoline-2(1H)-carboxamide;
N-{4-[((2R)-2,3-dihydro-1-benzofuran-2-ylcarbonyl)amino]phenyl}-3,4-dihydroisoquinoline-2(1H)-carboxamide and N-{4-[((2S)-2,3-dihydro-1-benzofuran-2-ylcarbonyl)amino]phenyl}-3,4-dihydroisoquinoline-2(1H)-carboxamide;
N N-{4-[((3R)-3-methylpentanoyl)amino]phenyl}-3,4-dihydroisoquinoline-2(1H)-carboxamide and N-{4-[((3S)-3-methylpentanoyl)amino]phenyl}-3,4-dihydroisoquinoline-2(1H)-carboxamide;
N-{4-[(2,2-dimethylbutanoyl)amino]phenyl}-3,4-dihydroisoquinoline-2(1H)-carboxamide;
N-{4-[(3,3-dimethylbutanoyl)amino]phenyl}-3,4-dihydroisoquinoline-2(1H)-carboxamide;
N-[4-(heptanoylamino)phenyl]-3,4-dihydroisoquinoline-2(1H)-carboxamide;
N-{4-[(4,4,4-trifluorobutanoyl)amino]phenyl}-3,4-dihydroisoquinoline-2(1H)-carboxamide;
N-(4-{[(2-methoxyethoxy)acetyl]amino}phenyl)-3,4-dihydroisoquinoline-2(1H)-carboxamide;
N-{4-[((3R)-tetrahydrofuran-3-ylcarbonyl)amino]phenyl}-3,4-dihydroisoquinoline-2(1H)-carboxamide and N-{4-[((3S)-tetrahydrofuran-3-ylcarbonyl)amino]phenyl}-3,4-dihydroisoquinoline-2(1H)-carboxamide;
N-(4-{[3-(methylthio)propanoyl]amino}phenyl)-3,4-dihydroisoquinoline-2(1H)-carboxamide;
N-{4-[(cyclopentylacetyl)amino]phenyl}-3,4-dihydroisoquinoline-2(1H)-carboxamide;
N-{4-[(cyclohexylcarbonyl)amino]phenyl}-3,4-dihydroisoquinoline-2(1H)-carboxamide;
N-{4-[(cyclohexylacetyl)amino]phenyl}-3,4-dihydroisoquinoline-2(1H)-carboxamide;
N-[4-(benzoylamino)phenyl]-3,4-dihydroisoquinoline-2(1H)-carboxamide;
N-{4-[(phenylacetyl)amino]phenyl}-3,4-dihydroisoquinoline-2(1H)-carboxamide;
N-(4-{[3-(4-aminophenyl)propanoyl]amino}phenyl)-3,4-dihydroisoquinoline-2(1H)-carboxamide;
N-[4-(3-furoylamino)phenyl]-3,4-dihydroisoquinoline-2(1H)-carboxamide;
N-{4-[(2,5-dimethyl-3-furoyl)amino]phenyl}-3,4-dihydroisoquinoline-2(1H)-carboxamide;
N-{4-[(3-thienylcarbonyl)amino]phenyl}-3,4-dihydroisoquinoline-2(1H)-carboxamide;
N-{4-[(1H-pyrrol-2-ylcarbonyl)amino]phenyl}-3,4-dihydroisoquinoline-2(1H)-carboxamide;
N-{4-[(1,3-thiazol-5-ylcarbonyl)amino]phenyl}-3,4-dihydroisoquinoline-2(1H)-carboxamide;
N-{4-[(1H-pyrazol-5-ylcarbonyl)amino]phenyl}-3,4-dihydroisoquinoline-2(1H)-carboxamide;
N-{4-[(1H-pyrazol-4-ylcarbonyl)amino]phenyl}-3,4-dihydroisoquinoline-2(1H)-carboxamide;
N-{4-[(1,2-oxazol-5-ylcarbonyl)amino]phenyl}-3,4-dihydroisoquinoline-2(1H)-carboxamide;
N-{4-[(pyridin-2-ylacetyl)amino]phenyl}-3,4-dihydroisoquinoline-2(1H)-carboxamide;
N-{4-[(N,N-dimethyl-beta-alanyl)amino]phenyl}-3,4-dihydroisoquinoline-2(1H)-carboxamide;
N-(4-{[3-(piperidin-1-yl)propanoyl]amino}phenyl)-3,4-dihydroisoquinoline-2(1H)-carboxamide;
N-{4-[(morpholin-4-ylacetyl)amino]phenyl}-3,4-dihydroisoquinoline-2(1H)-carboxamide;
N-(4-{[3-(morpholin-4-yl)propanoyl]amino}phenyl)-3,4-dihydroisoquinoline-2(1H)-carboxamide;
N-(4-{[3-(4-methylpiperazin-1-yl)propanoyl]amino}phenyl)-3,4-dihydroisoquinoline-2(1H)-carboxamide;
N-{4-[(cyclopentylacetyl)amino]phenyl}-5-[(methylsulfonyl)amino]-3,4-dihydroisoquinoline-2(1H)-carboxamide; and pharmaceutically acceptable salts thereof.

### Embodiments of Formula (V)

In another aspect, the present invention provides compounds of Formula (V)

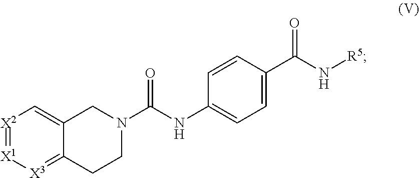

and pharmaceutically acceptable salts thereof; wherein X¹, X², X³, and R⁵ are as described herein for Formula (I).

In one embodiment of Formula (V), X¹, X² and X³ are CH; or X¹ and X³ are CH and X² is N; or X² and X³ are CH and X¹ is N; or X¹ is CR¹ and X² and X³ are CH; or X² is CR¹ and X¹ and X³ are CH; or X³ is CR¹; and X¹ and X² are CH.

In another embodiment of Formula (V), X¹, X² and X³ are CH. In another embodiment of Formula (V), X¹ and X³ are CH and X² is N. In another embodiment of Formula (V), X² and X³ are CH and X¹ is N. In another embodiment of Formula (V), X¹ is CR¹ and X² and X³ are CH. In another embodiment of Formula (V), X² is CR¹ and X¹ and X³ are CH. In another embodiment of Formula (V), X³ is CR¹; and X¹ and X² are CH.

In one embodiment of Formula (V), R¹ is NHSO₂R³, F, Cl, Br, or I. In another embodiment of Formula (V), R¹ is F. In another embodiment of Formula (V), R¹ is NHSO₂R³; and R³ is alkyl.

In one embodiment of Formula (V), X¹ is CR¹; X² and X³ are CH; and R¹ is F. In one embodiment of Formula (V), X² is CR¹; X¹ and X³ are CH; and R¹ is F. In another embodiment of Formula (V), X³ is CR¹; and X¹ and X² are CH; and R¹ is NHSO₂R³; and R³ is alkyl.

In one embodiment of Formula (V),

X¹ and X² and X³ are CH; or

X¹ and X³ are CH; and X² is N; or

X² and X³ are H; and X¹ is N; or

X¹ is CR¹; and X² and X³ are CH; or

X² is CR¹; and X¹ and X³ are CH; or

X³ is CR¹; and X¹ and X² are CH;

R¹ is NHSO₂R³, F, Cl, Br, or I;

R³ is alkyl;

R⁵ is alkyl, alkenyl, aryl, heterocyclyl, or cycloalkyl; wherein each alkyl and alkenyl, is optionally substituted with one or more independently selected R⁹, OR⁹, SR⁹, SO₂R⁹, C(O)R⁹, N(R⁹)₂, NHC(O)R⁹, C(O)NHR⁹, OH, or CF₃, F, Cl, Br or I;

R⁹ is alkyl, aryl, heterocyclyl, or cycloalkyl; wherein each alkyl is optionally substituted with one or more independently selected OCH₃, aryl, or heterocyclyl;

wherein the cyclic moieties represented by R⁵ and R⁹ are optionally substituted with one or more independently selected R¹³, OR¹³, SO₂R¹³, C(O)R¹³, CO(O)R¹³, NH₂, (O)NHR¹³, F, Cl, Br or I;

R¹³ is alkyl, aryl, or heterocyclyl; wherein each alkyl is optionally substituted with one or more independently selected R¹⁴, OH, F, Cl, Br or I; and

R¹⁴ is aryl F, Cl, Br or I.

Still another embodiment pertains to compounds having Formula (V), which include
N-{4-[(3-methylbutyl)carbamoyl]phenyl}-3,4-dihydroisoquinoline-2(1H)-carboxamide;
N-(4-{[4-(pyridin-2-yl)piperazin-1-yl]carbonyl}phenyl)-3,4-dihydroisoquinoline-2(1H)-carboxamide;
6-fluoro-N-{4-[(3-phenylpropyl)carbamoyl]phenyl}-3,4-dihydroisoquinoline-2(1H)-carboxamide;
N-{4-[(3-phenylpropyl)carbamoyl]phenyl}-3,4-dihydro-2,6-naphthyridine-2(1H)-carboxamide;
N-{4-[(3-methylbutyl)carbamoyl]phenyl}-3,4-dihydro-2,6-naphthyridine-2(1H)-carboxamide;
6-fluoro-N-{4-[(3-methylbutyl)carbamoyl]phenyl}-3,4-dihydroisoquinoline-2(1H)-carboxamide;
N-{4-[(3-phenylpropyl)carbamoyl]phenyl}-3,4-dihydroisoquinoline-2(1H)-carboxamide;
N-[4-(benzylcarbamoyl)phenyl]-3,4-dihydroisoquinoline-2(1H)-carboxamide;
N-(4-{[1-(3-methylbutyl)-1H-pyrazol-4-yl]carbamoyl}phenyl)-3,4-dihydroisoquinoline-2(1H)-carboxamide;
N-(4-{[2-(2-thienyl)ethyl]carbamoyl}phenyl)-3,4-dihydro-2,6-naphthyridine-2(1H)-carboxamide;
6-fluoro-N-(4-{[2-(2-thienyl)ethyl]carbamoyl}phenyl)-3,4-dihydroisoquinoline-2(1H)-carboxamide;
N-{4-[(1-benzyl-1H-pyrazol-4-yl)carbamoyl]phenyl}-3,4-dihydroisoquinoline-2(1H)-carboxamide;
N-{4-[(2-phenylethyl)carbamoyl]phenyl}-3,4-dihydroisoquinoline-2(1H)-carboxamide;
7-fluoro-N-{4-[(3-methylbutyl)carbamoyl]phenyl}-3,4-dihydroisoquinoline-2(1H)-carboxamide;
N-(4-{[2-(2-thienyl)ethyl]carbamoyl}phenyl)-3,4-dihydroisoquinoline-2(1H)-carboxamide;
7-fluoro-N-{4-[(3-phenylpropyl)carbamoyl]phenyl}-3,4-dihydroisoquinoline-2(1H)-carboxamide;
7-fluoro-N-(4-{[2-(2-thienyl)ethyl]carbamoyl}phenyl)-3,4-dihydroisoquinoline-2(1H)-carboxamide;
N-{4-[(3-phenylpropyl)carbamoyl]phenyl}-3,4-dihydro-2,7-naphthyridine-2(1H)-carboxamide;
N-{4-[(3-methylbutyl)carbamoyl]phenyl}-3,4-dihydro-2,7-naphthyridine-2(1H)-carboxamide;
N-(4-{[2-(2-thienyl)ethyl]carbamoyl}phenyl)-3,4-dihydro-2,7-naphthyridine-2(1H)-carboxamide; and pharmaceutically acceptable salts thereof.

### Embodiments of Formula (VI)

In another aspect, the present invention provides compounds of Formula (VI)

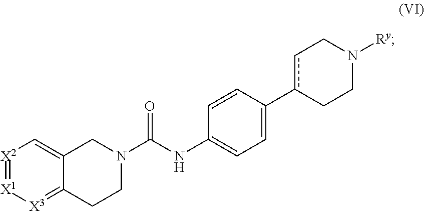

and pharmaceutically acceptable salts thereof; wherein X¹, X², and X³, are as described herein for Formula (I), R^{y} is as described for suitable substituents on R⁵, and [symbol] indicates a single or a double bond.

In one embodiment of Formula (VI), X¹, X² and X³ are CH; or X¹ and X³ are CH and X² is N; or X² and X³ are CH and X¹ is N; or X¹ is CR¹ and X² and X³ are CH; or X² is CR¹ and X¹ and X³ are CH; or X³ is CR¹; and X¹ and X² are CH.

In another embodiment of Formula (VI), X¹, X² and X³ are CH. In another embodiment of Formula (VI), X¹ and X³ are CH and X² is N. In another embodiment of Formula (VI), X² and X³ are CH and X¹ is N. In another embodiment of Formula (VI), X¹ is CR¹ and X² and X³ are CH. In another embodiment of Formula (VI), X² is CR¹ and X¹ and X³ are CH. In another embodiment of Formula (VI), X³ is CR¹; and X¹ and X² are CH.

In one embodiment of Formula (VI), R¹ is NHSO₂R³, F, Cl, Br, or I. In another embodiment of Formula (VI), R¹ is F. In another embodiment of Formula (VI), R¹ is NHSO₂R³; and R³ is alkyl.

In one embodiment of Formula (VI), X¹ is CR¹; X² and X³ are CH; and R¹ is F. In one embodiment of Formula (VI), X² is CR¹; X¹ and X³ are CH; and R¹ is F. In another embodiment of Formula (VI), X³ is CR¹; and X¹ and X² are CH; and R¹ is NHSO₂R³; and R³ is alkyl.

In one embodiment of Formula (VI), [symbol] is a single bond. In another embodiment of Formula (VI), [symbol] is a double bond.

In one embodiment of Formula (VI),

X¹ and X² and X³ are CH; or

X¹ and X³ are CH; and X² is N; or

X² and X³ are H; and X¹ is N; or

X¹ is CR¹; and X² and X³ are CH; or

X² is CR¹; and X¹ and X³ are CH; or

X³ is CR¹; and X¹ and X² are CH;

R¹ is NHSO₂R³, F, Cl, Br, or I;

R³ is alkyl;

R^{y} is R¹³, OR¹³, SO₂R¹³, C(O)R¹³, CO(O)R¹³, NH₂, or C(O)NHR¹³;

R¹³ is alkyl, aryl, or heterocyclyl; wherein each alkyl is optionally substituted with one or more independently selected R¹⁴, OH, F, Cl, Br or I; and

R¹⁴ is aryl F, Cl, Br or I.

Still another embodiment pertains to compounds having Formula (VI), which include
N-[4-(1-benzoyl-1,2,3,6-tetrahydropyridin-4-yl)phenyl]-3,4-dihydroisoquinoline-2(1H)-carboxamide;
N-[4-(1-butyryl-1,2,3,6-tetrahydropyridin-4-yl)phenyl]-3,4-dihydroisoquinoline-2(1H)-carboxamide;
N-{4-[1-(isopropylsulfonyl)-1,2,3,6-tetrahydropyridin-4-yl]phenyl}-3,4-dihydroisoquinoline-2(1H)-carboxamide;
N-[4-(1-isobutyryl-1,2,3,6-tetrahydropyridin-4-yl)phenyl]-3,4-dihydroisoquinoline-2(1H)-carboxamide;
N-{4-[1-(3-methylbutanoyl)-1,2,3,6-tetrahydropyridin-4-yl]phenyl}-3,4-dihydroisoquinoline-2(1H)-carboxamide;
N-{4-[1-(methylcarbamoyl)-1,2,3,6-tetrahydropyridin-4-yl]phenyl}-3,4-dihydroisoquinoline-2(1H)-carboxamide;
tert-butyl 4-{4-[(3,4-dihydroisoquinolin-2(1H)-ylcarbonyl)amino]phenyl}-3,6-dihydropyridine-1(2H)-carboxylate;
N-[4-(1-acetyl-1,2,3,6-tetrahydropyridin-4-yl)phenyl]-3,4-dihydroisoquinoline-2(1H)-carboxamide;
N-{4-[1-(isobutylsulfonyl)-1,2,3,6-tetrahydropyridin-4-yl]phenyl}-3,4-dihydroisoquinoline-2(1H)-carboxamide;
N-[4-(1-benzyl-1,2,3,6-tetrahydropyridin-4-yl)phenyl]-3,4-dihydroisoquinoline-2(1H)-carboxamide;
N-{4-[1-(methylsulfonyl)-1,2,3,6-tetrahydropyridin-4-yl]phenyl}-3,4-dihydroisoquinoline-2(1H)-carboxamide;
N-{4-[1-(3-methylbutyl)-1,2,3,6-tetrahydropyridin-4-yl]phenyl}-3,4-dihydroisoquinoline-2(1H)-carboxamide; and pharmaceutically acceptable salts thereof.

### Pharmaceutical Compositions, Combination Therapies, Methods of Treatment, and Administration

Another embodiment comprises pharmaceutical compositions comprising a compound having Formula (I) and an excipient.

Still another embodiment comprises methods of treating cancer in a mammal comprising administering thereto a therapeutically acceptable amount of a compound having Formula (I).

Still another embodiment pertains to compositions for treating diseases during which NAMPT is expressed, said compositions comprising an excipient and a therapeutically effective amount of the compound having Formula (I).

Still another embodiment pertains to methods of treating disease in a patient during which NAMPT is expressed, said methods comprising administering to the patient a therapeutically effective amount of a compound having Formula (I).

Still another embodiment pertains to compositions for treating inflammatory and tissue repair disorders; particularly rheumatoid arthritis, inflammatory bowel disease, asthma and COPD (chronic obstructive pulmonary disease), osteoarthritis, osteoporosis and fibrotic diseases; dermatosis, including psoriasis, atopic dermatitis and ultra-violet induced skin damage; autoimmune diseases including systemic lupus erythematosis, multiple sclerosis, psoriatic arthritis, ankylosing spondylitis, tissue and organ rejection, Alzheimer's disease, stroke, athersclerosis, restenosis, diabetes, glomerulonephritis, cancer, particularly wherein the cancer is selected from breast, prostate, lung, colon, cervix, ovary, skin, CNS, bladder, pancreas, leukemia, lymphoma or Hodgkin's disease, cachexia, inflammation associated with infection and certain viral infections, including Acquired Immune Deficiency Syndrome (AIDS), adult respiratory distress syndrome, and ataxia telengiectasia, said compositions comprising an excipient and a therapeutically effective amount of the compound having Formula (I).

Still another embodiment pertains to methods of treating inflammatory and tissue repair disorders; particularly rheumatoid arthritis, inflammatory bowel disease, asthma and COPD (chronic obstructive pulmonary disease), osteoarthritis, osteoporosis and fibrotic diseases; dermatosis, including psoriasis, atopic dermatitis and ultra-violet induced skin damage; autoimmune diseases including systemic lupus erythematosis, multiple sclerosis, psoriatic arthritis, ankylosing spondylitis, tissue and organ rejection, Alzheimer's disease, stroke, athersclerosis, restenosis, diabetes, glomerulonephritis, cancer, particularly wherein the cancer is selected from breast, prostate, lung, colon, cervix, ovary, skin, CNS, bladder, pancreas, leukemia, lymphoma or Hodgkin's disease, cachexia, inflammation associated with infection and certain viral infections, including Acquired Immune Deficiency Syndrome (AIDS), adult respiratory distress syndrome, and ataxia telengiectasia in a patient, said methods comprising administering to the patient a therapeutically effective amount of a compound having Formula (I).

Still another embodiment pertains to compositions for treating diseases during which NAMPT is expressed, said compositions comprising an excipient and a therapeutically effective amount of the compound having Formula (I) and a therapeutically effective amount of one additional therapeutic agent or more than one additional therapeutic agent.

Still another embodiment pertains to methods of treating disease in a patient during which NAMPT is expressed, said methods comprising administering to the patient a therapeutically effective amount of a compound having Formula (I) and a therapeutically effective amount of one additional therapeutic agent or more than one additional therapeutic agent.

Still another embodiment pertains to compositions for treating inflammatory and tissue repair disorders; particularly rheumatoid arthritis, inflammatory bowel disease, asthma and COPD (chronic obstructive pulmonary disease), osteoarthritis, osteoporosis and fibrotic diseases; dermatosis, including psoriasis, atopic dermatitis and ultra-violet induced skin damage; autoimmune diseases including systemic lupus erythematosis, multiple sclerosis, psoriatic arthritis, ankylosing spondylitis, tissue and organ rejection, Alzheimer's disease, stroke, athersclerosis, restenosis, diabetes, glomerulonephritis, cancer, particularly wherein the cancer is selected from breast, prostate, lung, colon, cervix, ovary, skin, CNS, bladder, pancreas, leukemia, lymphoma or Hodgkin's disease, cachexia, inflammation associated with infection and certain viral infections, including Acquired Immune Deficiency Syndrome (AIDS), adult respiratory distress syndrome, and ataxia telengiectasia, said compositions comprising an excipient and a therapeutically effective amount of the compound having Formula (I) and a therapeutically effective amount of one additional therapeutic agent or more than one additional therapeutic agent.

Still another embodiment pertains to methods of treating inflammatory and tissue repair disorders; particularly rheumatoid arthritis, inflammatory bowel disease, asthma and COPD (chronic obstructive pulmonary disease), osteoarthritis, osteoporosis and fibrotic diseases; dermatosis, including psoriasis, atopic dermatitis and ultra-violet induced skin damage; autoimmune diseases including systemic lupus erythematosis, multiple sclerosis, psoriatic arthritis, ankylosing spondylitis, tissue and organ rejection, Alzheimer's disease, stroke, athersclerosis, restenosis, diabetes, glomerulonephritis, cancer, particularly wherein the cancer is selected from breast, prostate, lung, colon, cervix, ovary, skin, CNS, bladder, pancreas, leukemia, lymphoma or Hodgkin's disease, cachexia, inflammation associated with infection and certain viral infections, including Acquired Immune Deficiency Syndrome (AIDS), adult respiratory distress syndrome, and ataxia telengiectasia in a patient, said methods comprising administering to the patient a therapeutically effective amount of the compound having Formula (I) and a therapeutically effective amount of one additional therapeutic agent or more than one additional therapeutic agent.

Metabolites of compounds having Formula (I), produced by in vitro or in vivo metabolic processes, may also have utility for treating diseases associated with NAMPT.

Certain precursor compounds which may be metabolized in vitro or in vivo to form compounds having Formula (I) may also have utility for treating diseases associated with NAMPT.

Compounds having Formula (I) may exist as acid addition salts, basic addition salts or zwitterions. Salts of the compounds are prepared during isolation or following purification of the compounds. Acid addition salts of the compounds are those derived from the reaction of the compounds with an acid. For example, the acetate, adipate, alginate, bicarbonate, citrate, aspartate, benzoate, benzenesulfonate, bisulfate, butyrate, camphorate, camphorsulfonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetic, trifluoroacetic, para-toluenesulfonate, and undecanoate salts of the compounds are contemplated as being embraced by this invention. Basic addition salts of the compounds are those derived from the reaction of the compounds with the hydroxide, carbonate or bicarbonate of cations such as lithium, sodium, potassium, calcium, and magnesium.

The compounds having Formula (I) may be administered, for example, bucally, ophthalmically, orally, osmotically, parenterally (intramuscularly, intraperitoneally intrasternally, intravenously, subcutaneously), rectally, topically, transdermally or vaginally.

Therapeutically effective amounts of compounds having Formula (I) depend on the recipient of the treatment, the disorder being treated and the severity thereof, the composition containing the compound, the time of administration, the route of administration, the duration of treatment, the compound potency, its rate of clearance and whether or not another drug is co-administered. The amount of a compound of this invention having Formula (I) used to make a composition to be administered daily to a patient in a single dose or in divided doses is from about 0.03 to about 200 mg/kg body weight. Single dose compositions contain these amounts or a combination of submultiples thereof.

Compounds having Formula (I) may be administered with or without an excipient. Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof.

Excipients for preparation of compositions comprising a compound having Formula (I) to be administered orally in solid dosage form include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof. Excipients for preparation of compositions comprising a compound of this invention having Formula (I) to be administered ophthalmically or orally in liquid dosage forms include, for example, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water and mixtures thereof. Excipients for preparation of compositions comprising a compound of this invention having Formula (I) to be administered osmotically include, for example, chlorofluorohydrocarbons, ethanol, water and mixtures thereof. Excipients for preparation of compositions comprising a compound of this invention having Formula (I) to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof. Excipients for preparation of compositions comprising a compound of this invention having Formula (I) to be administered rectally or vaginally include, for example, cocoa butter, polyethylene glycol, wax and mixtures thereof.

Compounds having Formula (I) are expected to be useful when used with alkylating agents, angiogenesis inhibitors, antibodies, antimetabolites, antimitotics, antiproliferatives, antivirals, aurora kinase inhibitors, apoptosis promoters (for example, Bcl-xL, Bcl-w and Bfl-1) inhibitors, activators of death receptor pathway, Bcr-Abl kinase inhibitors, BiTE (Bi-Specific T cell Engager) antibodies, antibody drug conjugates, biologic response modifiers, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNA's, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteosome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, and the like, and in combination with one or more of these agents.

BiTE antibodies are bi-specific antibodies that direct T-cells to attack cancer cells by simultaneously binding the two cells. The T-cell then attacks the target cancer cell. Examples of BiTE antibodies include adecatumumab (Micromet MT201), blinatumomab (Micromet MT103) and the like. Without being limited by theory, one of the mechanisms by which T-cells elicit apoptosis of the target cancer cell is by exocytosis of cytolytic granule components, which include perforin and granzyme B. In this regard, Bcl-2 has been shown to attenuate the induction of apoptosis by both perforin and granzyme B. These data suggest that inhibition of Bcl-2 could enhance the cytotoxic effects elicited by T-cells when targeted to cancer cells (V. R. Sutton, D. L. Vaux and J. A. Trapani, J. of Immunology 1997, 158 (12), 5783).

SiRNAs are molecules having endogenous RNA bases or chemically modified nucleotides. The modifications do not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides, combinations thereof and the like. The siRNA can have varying lengths (e.g., 10-200 bps) and structures (e.g., hairpins, single/double strands, bulges, nicks/gaps, mismatches) and are processed in cells to provide active gene silencing. A double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand.

Multivalent binding proteins are binding proteins comprising two or more antigen binding sites. Multivalent binding proteins are engineered to have the three or more antigen binding sites and are generally not naturally occurring antibodies. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific (i.e., capable of binding one antigen) or multispecific (i.e., capable of binding two or more antigens). DVD binding proteins comprising two heavy chain DVD polypeptides and two light chain DVD polypeptides are referred to as DVD Ig's. Each half of a DVD Ig comprises a heavy chain DVD polypeptide, a light chain DVD polypeptide, and two antigen binding sites. Each binding site comprises a heavy chain variable domain and a light chain variable domain with a total of 6 CDRs involved in antigen binding per antigen binding site.

Alkylating agents include altretamine, AMD-473, AP-5280, apaziquone, bendamustine, brostallicin, busulfan, carboquone, carmustine (BCNU), chlorambucil, CLORETAZINE® (laromustine, VNP 40101M), cyclophosphamide, decarbazine, estramustine, fotemustine, glufosfamide, ifosfamide, KW-2170, lomustine (CCNU), mafosfamide, melphalan, mitobronitol, mitolactol, nimustine, nitrogen mustard N-oxide, ranimustine, temozolomide, thiotepa, TREANDA® (bendamustine), treosulfan, trofosfamide and the like.

Angiogenesis inhibitors include endothelial-specific receptor tyrosine kinase (Tie-2) inhibitors, epidermal growth factor receptor (EGFR) inhibitors, insulin growth factor-2 receptor (IGFR-2) inhibitors, matrix metalloproteinase-2 (MMP-2) inhibitors, matrix metalloproteinase-9 (MMP-9) inhibitors, platelet-derived growth factor receptor (PDGFR) inhibitors, thrombospondin analogs, vascular endothelial growth factor receptor tyrosine kinase (VEGFR) inhibitors and the like.

Antimetabolites include ALIMTA® (pemetrexed disodium, LY231514, MTA), 5-azacitidine, XELODA® (capecitabine), carmofur, LEUSTAT® (cladribine), clofarabine, cytarabine, cytarabine ocfosfate, cytosine arabinoside, decitabine, deferoxamine, doxifluridine, eflornithine, EICAR (5-ethynyl-1-β-D-ribofuranosylimidazole-4-carboxamide), enocitabine, ethnylcytidine, fludarabine, 5-fluorouracil alone or in combination with leucovorin, GEMZAR® (gemcitabine), hydroxyurea, ALKERAN® (melphalan), mercaptopurine, 6-mercaptopurine riboside, methotrexate, mycophenolic acid, nelarabine, nolatrexed, ocfosfate, pelitrexol, pentostatin, raltitrexed, Ribavirin, triapine, trimetrexate, S-1, tiazofurin, tegafur, TS-1, vidarabine, UFT and the like.

Antivirals include ritonavir, hydroxychloroquine and the like.

Aurora kinase inhibitors include ABT-348, AZD-1152, MLN-8054, VX-680, Aurora A-specific kinase inhibitors, Aurora B-specific kinase inhibitors and pan-Aurora kinase inhibitors and the like.

Bcl-2 protein inhibitors include AT-101 ((−)gossypol), GENASENSE® (G3139 or oblimersen (Bcl-2-targeting antisense oligonucleotide)), IPI-194, IPI-565, N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide) (ABT-737), N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide (ABT-263), GX-070 (obatoclax) and the like.

Bcr-Abl kinase inhibitors include DASATINIB® (BMS-354825), GLEEVEC® (imatinib) and the like.

CDK inhibitors include AZD-5438, BMI-1040, BMS-032, BMS-387, CVT-2584, flavopyridol, GPC-286199, MCS-5A, PD0332991, PHA-690509, seliciclib (CYC-202, R-roscovitine), ZK-304709 and the like.

COX-2 inhibitors include ABT-963, ARCOXIA® (etoricoxib), BEXTRA® (valdecoxib), BMS347070, CELEBREX® (celecoxib), COX-189 (lumiracoxib), CT-3, DERAMAXX® (deracoxib), JTE-522, 4-methyl-2-(3,4-dimethylphenyl)-1-(4-sulfamoylphenyl-1H-pyrrole), MK-663 (etoricoxib), NS-398, parecoxib, RS-57067, SC-58125, SD-8381, SVT-2016, S-2474, T-614, VIOXX® (rofecoxib) and the like.

EGFR inhibitors include ABX-EGF, anti-EGFR immunoliposomes, EGF-vaccine, EMD-7200, ERBITUX® (cetuximab), HR3, IgA antibodies, IRESSA® (gefitinib), TARCEVA® (erlotinib or OSI-774), TP-38, EGFR fusion protein, TYKERB® (lapatinib) and the like.

ErbB2 receptor inhibitors include CP-724-714, CI-1033 (canertinib), HERCEPTIN® (trastuzumab), TYKERB® (lapatinib), OMNITARG® (2C4, petuzumab), TAK-165, GW-572016 (ionafarnib), GW-282974, EKB-569, PI-166, dHER2 (HER2 vaccine), APC-8024 (HER-2 vaccine), anti-HER/2neu bispecific antibody, B7.her2IgG3, AS HER2 trifunctional bispecfic antibodies, mAB AR-209, mAB 2B-1 and the like.

Histone deacetylase inhibitors include depsipeptide, LAQ-824, MS-275, trapoxin, suberoylanilide hydroxamic acid (SAHA), TSA, valproic acid and the like.

HSP-90 inhibitors include 17-AAG-nab, 17-AAG, CNF-101, CNF-1010, CNF-2024, 17-DMAG, geldanamycin, IPI-504, KOS-953, MYCOGRAB® (human recombinant antibody to HSP-90), NCS-683664, PU24FCl, PU-3, radicicol, SNX-2112, STA-9090 VER49009 and the like.

Inhibitors of inhibitors of apoptosis proteins include HGS1029, GDC-0145, GDC-0152, LCL-161, LBW-242 and the like.

Antibody drug conjugates include anti-CD22-MC-MMAF, anti-CD22-MC-MMAE, anti-CD22-MCC-DM1, CR-011-vcMMAE, PSMA-ADC, MEDI-547, SGN-19 Am SGN-35, SGN-75 and the like

Activators of death receptor pathway include TRAIL, antibodies or other agents that target TRAIL or death receptors (e.g., DR4 and DR5) such as Apomab, conatumumab, ETR2-ST01, GDC0145 (lexatumumab), HGS-1029, LBY-135, PRO-1762 and trastuzumab.

Kinesin inhibitors include Eg5 inhibitors such as AZD4877, ARRY-520; CENPE inhibitors such as GSK923295A and the like.

JAK-2 inhibitors include CEP-701 (lesaurtinib), XL019 and INCB018424 and the like.

MEK inhibitors include ARRY-142886, ARRY-438162 PD-325901, PD-98059 and the like.

mTOR inhibitors include AP-23573, CCI-779, everolimus, RAD-001, rapamycin, temsirolimus, ATP-competitive TORC1/TORC2 inhibitors, including PI-103, PP242, PP30, Torin 1 and the like.

Non-steroidal anti-inflammatory drugs include AMIGESIC® (salsalate), DOLOBID® (diflunisal), MOTRIN® (ibuprofen), ORUDIS® (ketoprofen), RELAFEN® (nabumetone), FELDENE® (piroxicam), ibuprofen cream, ALEVE® (naproxen) and NAPROSYN® (naproxen), VOLTAREN® (diclofenac), INDOCIN® (indomethacin), CLINORIL® (sulindac), TOLECTIN® (tolmetin), LODINE® (etodolac), TORADOL® (ketorolac), DAYPRO® (oxaprozin) and the like.

PDGFR inhibitors include C-451, CP-673, CP-868596 and the like.

Platinum chemotherapeutics include cisplatin, ELOXATIN® (oxaliplatin) eptaplatin, lobaplatin, nedaplatin, PARAPLATIN® (carboplatin), satraplatin, picoplatin and the like.

Polo-like kinase inhibitors include BI-2536 and the like.

Phosphoinositide-3 kinase (PI3K) inhibitors include wortmannin, LY294002, XL-147, CAL-120, ONC-21, AEZS-127, ETP-45658, PX-866, GDC-0941, BGT226, BEZ235, XL765 and the like.

Thrombospondin analogs include ABT-510, ABT-567, ABT-898, TSP-1 and the like.

VEGFR inhibitors include AVASTIN® (bevacizumab), ABT-869, AEE-788, ANGIOZYME™ (a ribozyme that inhibits angiogenesis (Ribozyme Pharmaceuticals (Boulder, Colo.) and Chiron, (Emeryville, Calif.)), axitinib (AG-13736), AZD-2171, CP-547,632, IM-862, MACUGEN (pegaptamib), NEXAVAR® (sorafenib, BAY43-9006), pazopanib (GW-786034), vatalanib (PTK-787, ZK-222584), SUTENT® (sunitinib, SU-11248), VEGF trap, ZACTIMA™ (vandetanib, ZD-6474) and the like.

Antibiotics include intercalating antibiotics aclarubicin, actinomycin D, amrubicin, annamycin, adriamycin, BLENOXANE® (bleomycin), daunorubicin, CAELYX® or MYOCET® (liposomal doxorubicin), elsamitrucin, epirbucin, glarbuicin, ZAVEDOS® (idarubicin), mitomycin C, nemorubicin, neocarzinostatin, peplomycin, pirarubicin, rebeccamycin, stimalamer, streptozocin, VALSTAR® (valrubicin), zinostatin and the like.

Topoisomerase inhibitors include aclarubicin, 9-aminocamptothecin, amonafide, amsacrine, becatecarin, belotecan, BN-80915, CAMPTOSAR® (irinotecan hydrochloride), camptothecin, CARDIOXANE® (dexrazoxine), diflomotecan, edotecarin, ELLENCE® or PHARMORUBICIN® (epirubicin), etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

Antibodies include AVASTIN® (bevacizumab), CD40-specific antibodies, chTNT-1/B, denosumab, ERBITUX® (cetuximab), HUMAX-CD4® (zanolimumab), IGF1R-specific antibodies, lintuzumab, PANOREX® (edrecolomab), RENCAREX® (WX G250), RITUXAN® (rituximab), ticilimumab, trastuzimab, CD20 antibodies types I and II and the like.

Hormonal therapies include ARIMIDEX® (anastrozole), AROMASIN® (exemestane), arzoxifene, CASODEX® (bicalutamide), CETROTIDE® (cetrorelix), degarelix, deslorelin, DESOPAN® (trilostane), dexamethasone, DROGENIL® (flutamide), EVISTA® (raloxifene), AFEMA™ (fadrozole), FARESTON® (toremifene), FASLODEX® (fulvestrant), FEMARA® (letrozole), formestane, glucocorticoids, HECTOROL® (doxercalciferol), RENAGEL® (sevelamer carbonate), lasofoxifene, leuprolide acetate, MEGACE® (megesterol), MIFEPREX® (mifepristone), NILANDRON™ (nilutamide), NOLVADEX® (tamoxifen citrate), PLENAXIS™ (abarelix), prednisone, PROPECIA® (finasteride), rilostane, SUPREFACT® (buserelin), TRELSTAR® (luteinizing hormone releasing hormone (LHRH)), VANTAS® (Histrelin implant), VETORYL® (trilostane or modrastane), ZOLADEX® (fosrelin, goserelin) and the like.

Deltoids and retinoids include seocalcitol (EB1089, CB1093), lexacalcitrol (KH1060), fenretinide, PANRETIN® (aliretinoin), ATRAGEN® (liposomal tretinoin), TARGRETIN® (bexarotene), LGD-1550 and the like.

PARP inhibitors include ABT-888 (veliparib), olaparib, KU-59436, AZD-2281, AG-014699, BSI-201, BGP-15, INO-1001, ONO-2231 and the like.

Plant alkaloids include, but are not limited to, vincristine, vinblastine, vindesine, vinorelbine and the like.

Proteasome inhibitors include VELCADE® (bortezomib), MG132, NPI-0052, PR-171 and the like.

Examples of immunologicals include interferons and other immune-enhancing agents. Interferons include interferon alpha, interferon alpha-2a, interferon alpha-2b, interferon beta, interferon gamma-1a, ACTIMMUNE® (interferon gamma-1b) or interferon gamma-n1, combinations thereof and the like. Other agents include ALFAFERONE®, (IFN-α), BAM-002 (oxidized glutathione), BEROMUN® (tasonermin), BEXXAR® (tositumomab), CAMPATH® (alemtuzumab), CTLA4 (cytotoxic lymphocyte antigen 4), decarbazine, denileukin, epratuzumab, GRANOCYTE® (lenograstim), lentinan, leukocyte alpha interferon, imiquimod, MDX-010 (anti-CTLA-4), melanoma vaccine, mitumomab, molgramostim, MYLOTARG™ (gemtuzumab ozogamicin), NEUPOGEN® (filgrastim), OncoVAC-CL, OVAREX® (oregovomab), pemtumomab (Y-muHMFG1), PROVENGE® (sipuleucel-T), sargaramostim, sizofilan, teceleukin, THERACYS® (Bacillus Calmette-Guerin), ubenimex, VIRULIZIN® (immunotherapeutic, Lorus Pharmaceuticals), Z-100 (Specific Substance of Maruyama (SSM)), WF-10 (Tetrachlorodecaoxide (TCDO)), PROLEUKIN® (aldesleukin), ZADAXIN® (thymalfasin), ZENAPAX® (daclizumab), ZEVALIN® (90Y-Ibritumomab tiuxetan) and the like.

Biological response modifiers are agents that modify defense mechanisms of living organisms or biological responses, such as survival, growth or differentiation of tissue cells to direct them to have anti-tumor activity and include krestin, lentinan, sizofuran, picibanil PF-3512676 (CpG-8954), ubenimex and the like.

Pyrimidine analogs include cytarabine (ara C or Arabinoside C), cytosine arabinoside, doxifluridine, FLUDARA® (fludarabine), 5-FU (5-fluorouracil), floxuridine, GEMZAR® (gemcitabine), TOMUDEX® (ratitrexed), TROXATYL™ (triacetyluridine troxacitabine) and the like.

Purine analogs include LANVIS® (thioguanine) and PURI-NETHOL® (mercaptopurine).

Antimitotic agents include batabulin, epothilone D (KOS-862), N-(2-((4-hydroxyphenyl)amino)pyridin-3-yl)-4-methoxybenzenesulfonamide, ixabepilone (BMS 247550), paclitaxel, TAXOTERE® (docetaxel), PNU100940 (109881), patupilone, XRP-9881 (larotaxel), vinflunine, ZK-EPO (synthetic epothilone) and the like.

Ubiquitin ligase inhibitors include MDM2 inhibitors, such as nutlins, NEDD8 inhibitors such as MLN4924 and the like.

Compounds of this invention can also be used as radiosensitizers that enhance the efficacy of radiotherapy. Examples of radiotherapy include external beam radiotherapy, teletherapy, brachytherapy and sealed, unsealed source radiotherapy and the like.

Additionally, compounds having Formula (I) may be combined with other chemotherapeutic agents such as ABRAXANE™ (ABI-007), ABT-100 (farnesyl transferase inhibitor), ADVEXIN® (Ad5CMV-p53 vaccine), ALTOCOR® or MEVACOR® (lovastatin), AMPLIGEN® (poly I:poly C12U, a synthetic RNA), APTOSYN® (exisulind), AREDIA® (pamidronic acid), arglabin, L-asparaginase, atamestane (1-methyl-3,17-dione-androsta-1,4-diene), AVAGE® (tazarotene), AVE-8062 (combreastatin derivative) BEC2 (mitumomab), cachectin or cachexin (tumor necrosis factor), canvaxin (vaccine), CEAVAC® (cancer vaccine), CELEUK® (celmoleukin), CEPLENE® (histamine dihydrochloride), CERVARIX® (human papillomavirus vaccine), CHOP® (C: CYTOXAN® (cyclophosphamide); H: ADRIAMYCIN® (hydroxydoxorubicin); O: Vincristine (ONCOVIN®); P: prednisone), CYPAT™ (cyproterone acetate), combrestatin A4P, DAB(389)EGF (catalytic and translocation domains of diphtheria toxin fused via a His-Ala linker to human epidermal growth factor) or TransMID-107R™ (diphtheria toxins), dacarbazine, dactinomycin, 5,6-dimethylxanthenone-4-acetic acid (DMXAA), eniluracil, EVIZON™ (squalamine lactate), DIMERICINE® (T4N5 liposome lotion), discodermolide, DX-8951f (exatecan mesylate), enzastaurin, EPO906 (epithilone B), GARDASIL® (quadrivalent human papillomavirus (Types 6, 11, 16, 18) recombinant vaccine), GASTRIMMUNE®, GENASENSE®, GMK (ganglioside conjugate vaccine), GVAX® (prostate cancer vaccine), halofuginone, histerelin, hydroxycarbamide, ibandronic acid, IGN-101, IL-13-PE38, IL-13-PE38QQR (cintredekin besudotox), IL-13-pseudomonas exotoxin, interferon-α, interferon-γ, JUNOVAN™ or MEPACT™ (mifamurtide), lonafarnib, 5,10-methylenetetrahydrofolate, miltefosine (hexadecylphosphocholine), NEOVASTAT® (AE-941), NEUTREXIN® (trimetrexate glucuronate), NIPENT® (pentostatin), ONCONASE® (a ribonuclease enzyme), ONCOPHAGE® (melanoma vaccine treatment), ONCOVAX® (IL-2 Vaccine), ORATHECIN™ (rubitecan), OSIDEM® (antibody-based cell drug), OVAREX® MAb (murine monoclonal antibody), paclitaxel, PANDIMEX™ (aglycone saponins from ginseng comprising 20(S)protopanaxadiol (aPPD) and 20(S)protopanaxatriol (aPPT)), panitumumab, PANVAC®-VF (investigational cancer vaccine), pegaspargase, PEG Interferon A, phenoxodiol, procarbazine, rebimastat, REMOVAB® (catumaxomab), REVLIMID® (lenalidomide), RSR13 (efaproxiral), SOMATULINE® LA (lanreotide), SORIATANE® (acitretin), staurosporine (Streptomyces staurospores), talabostat (PT100), TARGRETIN® (bexarotene), TAXOPREXIN® (DHA-paclitaxel), TELCYTA® (canfosfamide, TLK286), temilifene, TEMODAR® (temozolomide), tesmilifene, thalidomide, THERATOPE® (STn-KLH), thymitaq (2-amino-3,4-dihydro-6-methyl-4-oxo-5-(4-pyridylthio)quinazoline dihydrochloride), TNFERADE™ (adenovector: DNA carrier containing the gene for tumor necrosis factor-α), TRACLEER® or ZAVESCA® (bosentan), tretinoin (Retin-A), tetrandrine, TRISENOX® (arsenic trioxide), VIRULIZIN®, ukrain (derivative of alkaloids from the greater celandine plant), vitaxin (anti-alphavbeta3 antibody), XCYTRIN® (motexafin gadolinium), XINLAY™ (atrasentan), XYOTAX™ (paclitaxel poliglumex), YONDELIS® (trabectedin), ZD-6126, ZINECARD® (dexrazoxane), ZOMETA® (zolendronic acid), zorubicin and the like.

### Data

Determination of the utility of compounds having Formula (I) as binders to and inhibitors of NAMPT was performed using a Time-Resolved Fluorescence Resonance Energy Transfer (TR-FRET) binding assay.

### Time-Resolved Fluorescence Resonance Energy Transfer (TR-FRET) Binding Assay of NAMPT

The assay was carried out in 18 uL low volume plates (Owens Corning) in reaction buffer (50 mM HEPES (NaOH), pH 7.5, 100 mM NaCl, 10 mM MgCl₂, 1 mM DTT, 1% Glycerol) using 6.8 nM recombinant, human, C-terminally-His tagged NAMPT, 1 nM Tb-anti-His antibody (Invitrogen, Cat # PV5895), and 200 nM probe (Oregon Green 488-conjugated APO866; A-1251667.0). Plates were covered, and reactions were carried out for 2-3 hours. Plates were read with Envision (Laser Lantha low volume protocol) after 2 to 3 hours. Excitation was carried out at 337 nm, and the ratio of emission of Oregon Green (520 nm) to terbium (492 nm) was determined and used to calculate IC₅₀ values of test compounds.

TABLE 1 shows the utility of compounds having Formula Ito functionally inhibit NAMPT.

TABLE 1

| | TR-FRET |
|---|---|
| | Binding - IC50 |
| Example | (μM) |
| 1 | 0.0167 |
| 2 | 0.0132 |
| 3 | 0.0138 |
| 4 | 0.015 |
| 5 | 0.0157 |
| 6 | 0.0103 |
| 7 | 0.0207 |
| 8 | 0.0237 |
| 9 | 0.0291 |
| 10 | 0.0294 |
| 11 | 0.0433 |
| 12 | 0.0452 |
| 13 | 0.0458 |
| 14 | 0.0471 |
| 15 | 0.0976 |
| 16 | 0.171 |
| 17 | 0.178 |
| 18 | 0.178 |
| 19 | 0.195 |
| 20 | 0.248 |
| 21 | 0.286 |
| 22 | 0.309 |
| 23 | 0.366 |
| 25 | 0.597 |
| 26 | 0.80 |
| 27 | 1.03 |
| 28 | 1.25 |
| 29 | 1.5 |
| 30 | 2.11 |
| 31 | 0.00616 |
| 32 | 0.012 |
| 33 | 0.0404 |
| 34 | 0.0388 |
| 35 | 0.103 |
| 36 | 0.185 |
| 37 | 0.298 |
| 38 | 0.391 |
| 39 | 0.611 |
| 40 | 0.645 |
| 41 | 0.819 |
| 42 | 1.29 |
| 43 | 0.0101 |
| 44 | 0.0136 |
| 45 | 0.0138 |
| 46 | 0.0153 |
| 47 | 0.0202 |
| 48 | 0.0326 |
| 49 | 0.0487 |
| 50 | 0.0586 |
| 51 | 0.0749 |
| 52 | 0.0805 |
| 53 | 0.112 |
| 54 | 0.289 |
| 55 | 0.435 |
| 56 | 0.189 |
| 57 | 0.887 |
| 58 | 1.26 |
| 59 | 0.864 |
| 60 | 1.37 |
| 61 | 1.25 |
| 62 | 1.47 |
| 63 | 0.0107 |
| 64 | 0.055 |
| 65 | 0.116 |
| 66 | 0.0153 |
| 67 | 0.0167 |
| 68 | 0.00445 |
| 69 | 0.00649 |
| 70 | 0.109 |
| 71 | 0.00275 |
| 72 | 0.0168 |
| 73 | 0.0332 |
| 74 | 0.0862 |
| 75 | 0.196 |
| 76 | 0.0148 |
| 77 | 0.00516 |
| 78 | 0.0663 |
| 79 | 0.0053 |
| 80 | 0.191 |
| 81 | Nd |
| 82 | 0.049 |
| 83 | 0.269 |
| 84 | 0.0858 |
| 85 | 0.242 |
| 86 | 0.377 |
| 87 | 0.116 |
| 88 | 0.134 |
| 89 | 0.203 |
| 90 | 0.314 |
| 91 | 0.016 |
| 92 | 5.84 |
| 93 | 0.53 |
| 94 | 0.0692 |
| 95 | 0.158 |
| 96 | 0.128 |
| 97 | 1.49 |
| 98 | 0.003 |

nd = no data

Compounds which inhibit NAMPT are useful for treating diseases in which activation of NF-KB is implicated. Such methods are useful in the treatment of a variety of diseases including inflammatory and tissue repair disorders; particularly rheumatoid arthritis, inflammatory bowel disease, asthma and COPD (chronic obstructive pulmonary disease), osteoarthritis, osteoporosis and fibrotic diseases; dermatosis, including psoriasis, atopic dermatitis and ultra-violet induced skin damage; autoimmune diseases including systemic lupus erythematosis, multiple sclerosis, psoriatic arthritis, ankylosing spondylitis, tissue and organ rejection, Alzheimer's disease, stroke, athersclerosis, restenosis, diabetes, glomerulonephritis, cancer, particularly wherein the cancer is selected from breast, prostate, lung, colon, cervix, ovary, skin, CNS, bladder, pancreas, leukaemia, lymphoma or Hodgkin's disease, cachexia, inflammation associated with infection and certain viral infections, including Acquired Immune Deficiency Syndrome (AIDS), adult respiratory distress syndrome, and ataxia telengiectasia.

Involvement of NAMPT in the treatment of cancer is described in WO 97/48696. Involvement of NAMPT in immuno-supression is described in WO 97/48397. Involvement of NAMPT for the treatment of diseases involving angiogenesis is described in WO 2003/80054. Involvement of NAMPT for the treatment of rheumatoid arthritis and septic shock is described in WO 2008/025857. Involvement of NAMPT for the prophlaxis and treatment of ischaemia is described in WO 2009/109610.

Cancers include, but are not limited to, hematologic and solid tumor types such as acoustic neuroma, acute leukemia, acute lymphoblastic leukemia, acute myelogenous leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer (including estrogen-receptor positive breast cancer), bronchogenic carcinoma, Burkitt's lymphoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, gastric carcinoma, germ cell testicular cancer, gestational trophobalstic disease, glioblastoma, head and neck cancer, heavy chain disease, hemangioblastoma, hepatoma, hepatocellular cancer, hormone insensitive prostate cancer, leiomyosarcoma, liposarcoma, lung cancer (including small cell lung cancer and non-small cell lung cancer), lymphangioendothelio-sarcoma, lymphangiosarcoma, lymphoblastic leukemia, lymphoma (lymphoma, including diffuse large B-cell lymphoma, follicular lymphoma, Hodgkin's lymphoma and non-Hodgkin's lymphoma), malignancies and hyperproliferative disorders of the bladder, breast, colon, lung, ovaries, pancreas, prostate, skin and uterus, lymphoid malignancies of T-cell or B-cell origin, leukemia, medullary carcinoma, medulloblastoma, melanoma, meningioma, mesothelioma, multiple myeloma, myelogenous leukemia, myeloma, myxosarcoma, neuroblastoma, oligodendroglioma, oral cancer, osteogenic sarcoma, ovarian cancer, pancreatic cancer, papillary adenocarcinomas, papillary carcinoma, peripheral T-cell lymphoma, pinealoma, polycythemia vera, prostate cancer (including hormone-insensitive (refractory) prostate cancer), rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), stomach cancer, squamous cell carcinoma, synovioma, sweat gland carcinoma, testicular cancer (including germ cell testicular cancer), thyroid cancer, Waldenström's macroglobulinemia, testicular tumors, uterine cancer, Wilms' tumor and the like.

### Schemes and Experimentals

The following abbreviations have the meanings indicated. ADDP means 1,1′-(azodicarbonyl)dipiperidine; AD-mix-β means a mixture of (DHQD)₂PHAL, K₃Fe(CN)₆, K₂CO₃, and K₂SO₄; 9-BBN means 9-borabicyclo(3.3.1)nonane; Boc means tert-butoxycarbonyl; (DHQD)₂PHAL means hydroquinidine 1,4-phthalazinediyl diethyl ether; DBU means 1,8-diazabicyclo[5.4.0]undec-7-ene; DIBAL means diisobutylaluminum hydride; DIEA means diisopropylethylamine; DMAP means N,N-dimethylaminopyridine; DMF means N,N-dimethylformamide; dmpe means 1,2-bis(dimethylphosphino)ethane; DMSO means dimethylsulfoxide; dppb means 1,4-bis(diphenylphosphino)-butane; dppe means 1,2-bis(diphenylphosphino)ethane; dppf means 1,1′-bis(diphenylphosphino)ferrocene; dppm means 1,1-bis(diphenylphosphino)methane; EDAC.HCl means 1-(3-dimethylaminopropyl)-3-ethylcarbodiimide hydrochloride; Fmoc means fluorenylmethoxycarbonyl; HATU means O-(7-azabenzotriazol-1-yl)-N,N′N′N′-tetramethyluronium hexafluorophosphate; HMPA means hexamethylphosphoramide; IPA means isopropyl alcohol; MP-BH₃ means macroporous triethylammonium methylpolystyrene cyanoborohydride; TEA means triethylamine; TFA means trifluoroacetic acid; THF means tetrahydrofuran; NCS means N-chlorosuccinimide; NMM means N-methylmorpholine; NMP means N-methylpyrrolidine; PPh₃ means triphenylphosphine.

The following schemes are presented to provide what is believed to be the most useful and readily understood description of procedures and conceptual aspects of this invention. Compounds of this invention may be made by synthetic chemical processes, examples of which are shown herein. It is meant to be understood that the order of the steps in the processes may be varied, that reagents, solvents and reaction conditions may be substituted for those specifically mentioned, and that vulnerable moieties may be protected and deprotected, as necessary.

### Schemes

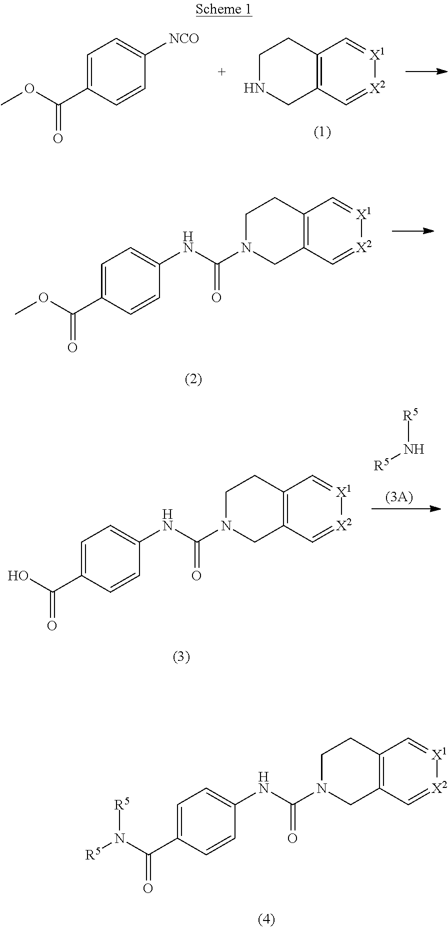

As shown in Scheme 1, compounds of formula (1), wherein X¹ and X² are as described herein, can be reacted with methyl 4-isocyanatobenzoate to provide compounds of formula (2). The reaction is typically performed in a solvent such as but not limited to tetrahydrofuran. The methyl 4-isocyanatobenzoate is typically added at low temperature followed by stirring at room temperature. Compounds of formula (3) can be prepared by reacting compounds of formula (2) with aqueous lithium hydroxide. The reaction is typically performed in a solvent such as but not limited to tetrahydrofuran, methanol, or mixtures thereof. Compounds of formula (3) can be reacted with amine of formula (3A), wherein each R⁵ is hydrogen or is as described herein, using coupling conditions known to those skilled in the art and readily available in the literature to provide compounds of formula (4), which are representative of the compounds of this invention.

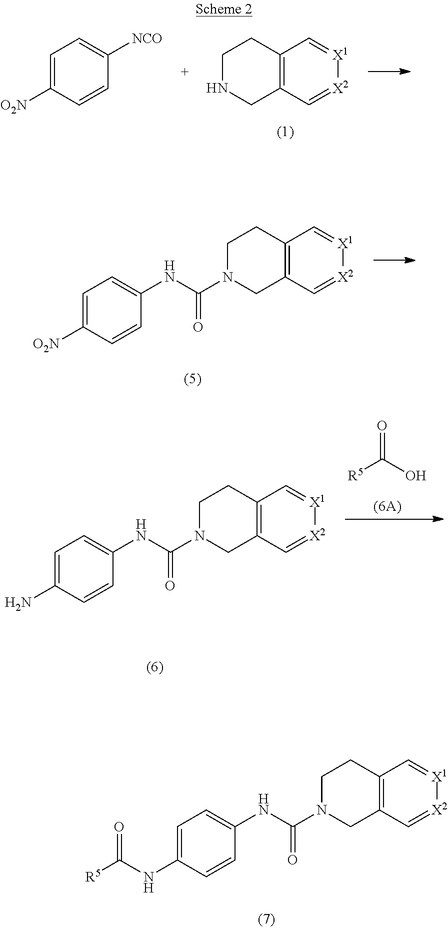

As shown in Scheme 2, compounds of formula (1), wherein X¹ and X² are as described herein, can be reacted with 4-nitrophenylisocyanate to provide compounds of formula (5). The reaction is typically performed in a solvent such as but not limited to tetrahydrofuran. Compounds of formula (5) can be treated with a hydrogen balloon in the presence of 10% Pd on carbon to provide compounds of formula (6). The reaction is typically performed at ambient temperature in a solvent such as but not limited to dimethylformamide. Compounds of formula (6) can be reacted with acid of formula (6A), wherein R⁵ is as described herein, using coupling conditions known to those skilled in the art and readily available in the literature to provide compounds of formula (7), which are representative of the compounds of this invention.

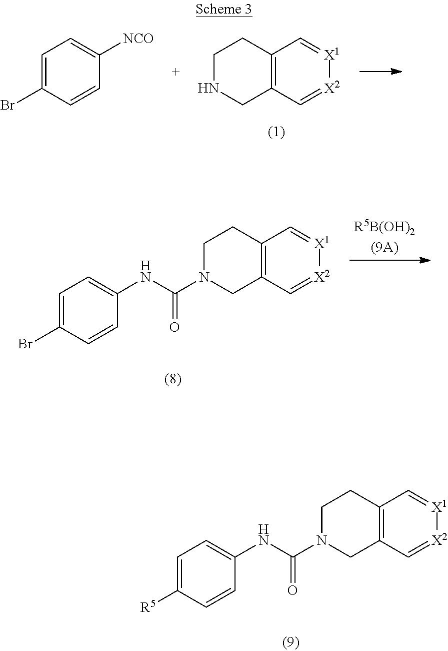

As shown in Scheme 3, compounds of formula (1), wherein X¹ and X² are as described herein, can be reacted with 4-bromophenyl isocyanate to provide compounds of formula (8). The reaction is typically performed in a solvent such as but not limited to tetrahydrofuran. Compounds of formula (8) can be reacted with a boronic acid of formula (9A) (or an appropriate boronate ester), wherein R⁵ is as described herein, using Suzuki Coupling conditions known to those skilled in the art and readily available in the literature to provide compounds of formula (9), which are representative of the compounds of this invention.

The following examples are presented to provide what is believed to be the most useful and readily understood description of procedures and conceptual aspects of this invention. The exemplified compounds were named using ACD/ChemSketch Version 12.01 (13 May 2009), Advanced Chemistry Development Inc., Toronto, Ontario), or ChemDraw® Ver. 9.0.5 (CambridgeSoft, Cambridge, Mass.). Intermediates were named using ChemDraw® Ver. 9.0.5 (CambridgeSoft, Cambridge, Mass.).

### EXPERIMENTALS

### Example 1

### N-{4-[(3-methylbutyl)carbamoyl]phenyl}-3,4-dihydroisoquinoline-2(1H)-carboxamide

### Example 1A

### methyl 4-(1,2,3,4-tetrahydroisoquinoline-2-carboxamido)benzoate

In a 250 mL round bottom flask was mixed 1,2,3,4-tetrahydroisoquinoline (1.90 ml, 15.02 mmol) in anhydrous dichloromethane (70 ml) at 0° C. Methyl 4-isocyanatobenzoate (2.93 g, 16.52 mmol) was added in a single portion and the reaction was stirred at 0° C. for 2 hours and over the weekend at room temperature. The solvent was evaporated and the resulting foam was triturated with ether and filtered to give the title compound.

### Example 1B

### 4-(1,2,3,4-tetrahydroisoquinoline-2-carboxamido)benzoic acid

In a 250 mL round bottom flask was mixed methyl 4-(1,2,3,4-tetrahydroisoquinoline-2-carboxamido)benzoate (3.43 g, 11.05 mmol) in tetrahydrofuran (40 ml) and methanol (40 ml) at room temperature. To this solution was added 4N aqueous sodium hydroxide (13.82 ml, 55.3 mmol) and the solution was stirred at room temperature for 6 hours. The organic solvents were evaporated and the aqueous solution was acidified to pH 3 using 3N aqueous HCl. The resulting suspension was filtered with water washes to give the title compound after vacuum drying.

### Example 1C

### N-{4-[(3-methylbutyl)carbamoyl]phenyl}-3,4-dihydroisoquinoline-2(1H)-carboxamide

4-(1,2,3,4-Tetrahydroisoquinoline-2-carboxamido)benzoic acid (25 mg, 0.084 mmol), 1-hydroxybenzotriazole (19.38 mg, 0.127 mmol) and diisopropylethylamine (0.044 ml, 0.253 mmol) were dissolved in dimethylformamide (1 ml) and treated with 3-methylbutan-1-amine (0.015 ml, 0.127 mmol) and N-(3-dimethylaminopropyl)-N′-ethylcarbodiimide hydrochloride (24.26 mg, 0.127 mmol). The homogeneous solution was stirred at ambient temperature overnight. The mixture was diluted with water, extracted with ethyl acetate, washed with brine, dried over MgSO₄, filtered and concentrated. The residue was triturated with methylene chloride and filtered to provide the title compound. ¹H NMR (300 MHz, DMSO-d₆) δ ppm 8.79 (s, 1H), 8.20 (t, J=5.6 Hz, 1H), 7.70-7.75 (m, 2H), 7.54-7.58 (m, 2H), 7.16-7.22 (m, 4H), 4.64-4.65 (bs, 2H), 3.71 (t, J=5.9 Hz, 2H), 3.25 (q, J=6.7 Hz, 2H), 2.86 (t, J=5.8 Hz, 2H), 1.61 (dp, J=13.3, 6.6 Hz, 1H), 1.37-1.44 (m, 2H), 0.90 (d, J=6.6 Hz, 6H); (ESI(+)) m/e 366 (M+H)⁺.

TABLE 2
The following examples were prepared essentially as described in Example 1,
substituting the appropriate amine for 1,2,3,4-tetrahydroisoquinoline in Example
1A and the appropriate amine for 3-methylbutan-1-amine in Example 1C.

| Ex | Name | ¹H NMR | MS |
|---|---|---|---|
| 2 | N-(4-{[4-(pyridin-2- | ¹H NMR (400 MHz, DMSO-d₆) δ ppm 8.79 | (ESI(+)) |
| | yl)piperazin-1- | (s, 1H), 8.13 (ddd, J = 4.8, 2.0, 0.8 Hz, 1H), | m/e 442 |
| | yl]carbonyl}phenyl)-3,4- | 7.56-7.60 (m, 2H), 7.55 (ddd, J = 8.5, 7.2, | (M + H)⁺ |
| | dihydroisoquinoline- | 2.0 Hz, 1H), 7.34-7.37 (m, 2H), 7.19-7.20 | |
| | 2(1H)-carboxamide | (m, 4H), 6.84 (dt, J = 8.6, 0.9 Hz, 1H), 6.67 | |
| | | (ddd, J = 7.0, 4.9, 0.8 Hz, 1H), 4.65-4.66 | |
| | | (bs, 2H), 3.72 (t, J = 5.9 Hz, 2H), 3.49-3.68 | |
| | | (m, 8H), 2.86 (t, J = 5.8 Hz, 2H) | |
| 3 | 6-fluoro-N-{4-[(3- | ¹H NMR (400 MHz, DMSO-d₆) δ ppm 8.81 | (ESI(−)) |
| | phenylpropyl)carbamoyl] | (s, 1H), 8.29 (t, J = 5.5 Hz, 1H), 7.73-7.76 | m/e 430 |
| | phenyl}-3,4- | (m, 2H), 7.55-7.57 (m, 2H), 7.25-7.32 (m, | (M − H)⁻ |
| | dihydroisoquinoline- | 2H), 7.20-7.25 (m, 3H), 7.15-7.20 (m, 1H), | |
| | 2(1H)-carboxamide | 7.02-7.07 (m, 2H), 4.62-4.63 (bs, 2H), 3.70 | |
| | | (t, J = 5.9 Hz, 2H), 3.18-3.27 (m, 2H), 2.87 | |
| | | (t, J = 5.9 Hz, 2H), 2.62 (t, J = 7.6 Hz, 2H), | |
| | | 1.82 (p, J = 7.4 Hz, 2H) | |
| 4 | N-{4-[(3- | ¹H NMR (400 MHz, DMSO-d₆) δ ppm 8.88 | (ESI(+)) |
| | phenylpropyl)carbamoyl] | (s, 1H), 8.41 (s, 1H), 8.35 (d, J = 5.0 Hz, | m/e 415 |
| | phenyl}-3,4-dihydro-2,6- | 1H), 8.29 (t, J = 5.6 Hz, 1H), 7.74-7.77 (m, | (M + H)⁺ |
| | naphthyridine-2(1H)- | 2H), 7.54-7.58 (m, 2H), 7.26-7.31 (m, 2H), | |
| | carboxamide | 7.20-7.25 (m, 3H), 7.15-7.20 (m, 1H), 4.67- | |
| | | 4.68 (bs, 2H), 3.76 (t, J = 5.8 Hz, 2H), 3.22- | |
| | | 3.29 (m, 2H), 2.87 (t, J = 5.7 Hz, 2H), 2.62 | |
| | | (t, J = 7.6 Hz, 2H), 1.82 (p, J = 7.4 Hz, 2H) | |
| 5 | N-{4-[(3- | ¹H NMR (400 MHz, DMSO-d₆) δ ppm 8.87 | (ESI(+)) |
| | methylbutyl)carbamoyl] | (s, 1H), 8.40 (s, 1H), 8.35 (d, J = 5.0 Hz, | m/e 367 |
| | phenyl}-3,4-dihydro-2,6- | 1H), 8.21 (t, J = 5.6 Hz, 1H), 7.72-7.76 (m, | (M + H)⁺ |
| | naphthyridine-2(1H)- | 2H), 7.54-7.57 (m, 2H), 7.21 (d, J = 5.1 Hz, | |
| | carboxamide | 1H), 4.67-4.68 (bs, 2H), 3.75 (t, J = 5.7 Hz, | |
| | | 2H), 3.22-3.27 (m, 2H), 2.85-2.89 (m, 2H), | |
| | | 1.54-1.66 (m, 1H), 1.41 (q, J = 7.1 Hz, 2H), | |
| | | 0.87-0.95 (m, 6H) | |
| 6 | 6-fluoro-N-{4-[(3- | ¹H NMR (400 MHz, DMSO-d₆) δ ppm 8.82 | (ESI(+)) |
| | methylbutyl)carbamoyl] | (s, 1H), 8.22 (t, J = 5.6 Hz, 1H), 7.72-7.75 | m/e 384 |
| | phenyl}-3,4- | (m, 2H), 7.54-7.57 (m, 2H), 7.19-7.25 (m, | (M + H)⁺ |
| | dihydroisoquinoline- | 1H), 7.02-7.07 (m, 2H), 4.62-4.63 (bs, 2H), | |
| | 2(1H)-carboxamide | 3.70 (t, J = 5.8 Hz, 2H), 3.22-3.28 (m, 2H), | |
| | | 2.85-2.89 (m, 2H), 1.54-1.66 (m, 1H), 1.41 | |
| | | (q, J = 7.1 Hz, 2H), 0.90 (d, J = 6.6 Hz, 6H) | |
| 7 | N-{4-[(3- | ¹H NMR (300 MHz, DMSO-d₆) δ ppm 8.80 | (ESI(+)) |
| | phenylpropyl)carbamoyl] | (s, 1H), 8.28 (t, J = 5.5 Hz, 1H), 7.72-7.77 | m/e 414 |
| | phenyl}-3,4- | (m, 2H), 7.55-7.59 (m, 2H), 7.18-7.29 (m, | (M + H)⁺ |
| | dihydroisoquinoline- | 9H), 4.65-4.66 (bs, 2H), 3.71 (t, J = 5.8 Hz, | |
| | 2(1H)-carboxamide | 2H), 3.21-3.30 (m, 2H), 2.86 (t, J = 5.8 Hz, | |
| | | 2H), 2.62 (t, J = 7.6 Hz, 2H), 1.76-1.87 (m, | |
| | | 2H) | |
| 8 | N-[4- | ¹H NMR (400 MHz, DMSO-d₆) δ ppm 8.85 | (ESI(+)) |
| | (benzylcarbamoyl)phenyl]- | (t, J = 6.0 Hz, 1H), 8.81-8.83 (bs, 1H), 7.79- | m/e 386 |
| | 3,4-dihydroisoquinoline- | 7.82 (m, 2H), 7.57-7.60 (m, 2H), 7.29-7.35 | (M + H)⁺ |
| | 2(1H)-carboxamide | (m, 4H), 7.20-7.25 (m, 1H), 7.17-7.21 (m, | |
| | | 4H), 4.65-4.66 (bs, 2H), 4.46 (d, J = 5.9 Hz, | |
| | | 2H), 3.71 (t, J = 5.9 Hz, 2H), 2.86 (t, J = 5.8 | |
| | | Hz, 2H) | |
| 11 | N-(4-{[1-(3- | ¹H NMR (500 MHz, DMSO-d₆) δ ppm | (ESI(+)) |
| | methylbutyl)-1H-pyrazol- | 10.21 (s, 1H), 8.86 (s, 1H), 8.02 (s, 1H), | m/e 432 |
| | 4-yl]carbamoyl}phenyl)- | 7.84-7.87 (m, 2H), 7.62-7.64 (m, 2H), 7.56 | (M + H)⁺ |
| | 3,4-dihydroisoquinoline- | (s, 1H), 7.20 (s, 3H), 7.20 (s, 1H), 4.66-4.67 | |
| | 2(1H)-carboxamide | (bs, 2H), 4.09 (t, J = 7.2 Hz, 2H), 3.73 (t, | |
| | | J = 5.9 Hz, 2H), 2.87 (t, J = 5.8 Hz, 2H), 1.65 | |
| | | (q, J = 7.0 Hz, 2H), 1.40-1.55 (m, 1H), 0.90 | |
| | | (d, J = 6.6 Hz, 6H) | |
| 12 | N-(4-{[2-(2- | ¹H NMR (400 MHz, DMSO-d₆) δ ppm 8.90 | (ESI(+)) |
| | thienyl)ethyl]carbamoyl} | (s, 1H), 8.45 (t, J = 5.3 Hz, 1H), 8.39-8.43 | m/e 407 |
| | phenyl)-3,4-dihydro-2,6- | (bs, 1H), 8.36 (d, J = 4.9 Hz, 1H), 7.74-7.77 | (M + H)⁺ |
| | naphthyridine-2(1H)- | (m, 2H), 7.55-7.58 (m, 2H), 7.33 (d, J = 5.0 | |
| | carboxamide | Hz, 1H), 7.22 (d, J = 5.0 Hz, 1H), 6.90-6.97 | |
| | | (m, 2H), 4.67-4.69 (bs, 2H), 3.74-3.78 (m, | |
| | | 2H), 3.42-3.51 (m, 2H), 3.00-3.10 (m, 2H), | |
| | | 2.85-2.89 (m, 2H) | |
| 13 | 6-fluoro-N-(4-{[2-(2- | ¹H NMR (400 MHz, DMSO-d₆) δ ppm 8.83 | (ESI(+)) |
| | thienyl)ethyl]carbamoyl} | (s, 1H), 8.43-8.47 (m, 1H), 7.73-7.76 (m, | m/e 424 |
| | phenyl)-3,4- | 2H), 7.55-7.58 (m, 2H), 7.33 (d, J = 5.1 Hz, | (M + H)⁺ |
| | dihydroisoquinoline- | 1H), 7.19-7.27 (m, 1H), 7.01-7.07 (m, 2H), | |
| | 2(1H)-carboxamide | 6.95 (dd, J = 5.0, 3.5 Hz, 1H), 6.91 (d, J = | |
| | | 3.4 Hz, 1H), 4.62-4.63 (bs, 2H), 3.70 (t, J = | |
| | | 5.8 Hz, 2H), 3.42-3.53 (m, 2H), 3.05 (t, J = | |
| | | 7.2 Hz, 2H), 2.85-2.89 (m, 2H) | |
| 14 | N-{4-[(1-benzyl-1H- | ¹H NMR (500 MHz, DMSO-d₆) δ ppm | (ESI(+)) |
| | pyrazol-4- | 10.25 (s, 1 H) 8.86 (s, 1 H) 8.11 (s, 1 H) | m/e 452 |
| | yl)carbamoyl]phenyl}- | 7.84 (d, J = 8.9 Hz, 2 H) 7.57-7.67 (m, 3 H) | (M + H)⁺ |
| | 3,4-dihydroisoquinoline- | 7.16-7.38 (m, 9 H) 5.31 (s, 2 H) 4.66 (s, 2 H) | |
| | 2(1H)-carboxamide | 3.72 (t, J = 6.0 Hz, 2 H) 2.81-2.93 (m, 2 H) | |
| 15 | N-{4-[(2- | ¹H NMR (500 MHz, DMSO-d₆) δ ppm 8.80 | (ESI(+)) |
| | phenylethyl)carbamoyl] | (s, 1H), 8.38 (t, J = 5.6 Hz, 1H), 7.72-7.75 | m/e 400 |
| | phenyl}-3,4- | (m, 2H), 7.55-7.58 (m, 2H), 7.27-7.33 (m, | (M + H)⁺ |
| | dihydroisoquinoline- | 2H), 7.22-7.26 (m, 2H), 7.15-7.22 (m, 5H), | |
| | 2(1H)-carboxamide | 4.65-4.66 (bs, 2H), 3.71 (t, J = 5.9 Hz, 2H), | |
| | | 3.44-3.48 (m, 2H), 2.82-2.87 (m, 4H) | |
| 16 | 7-fluoro-N-{4-[(3- | ¹H NMR (400 MHz, DMSO-d₆) δ ppm 8.82 | (ESI(+)) |
| | methylbutyl)carbamoyl] | (s, 1H), 8.21 (t, J = 5.6 Hz, 1H), 7.72-7.75 | m/e 384 |
| | phenyl}-3,4- | (m, 2H), 7.53-7.57 (m, 2H), 7.23 (dd, J = | (M + H)⁺ |
| | dihydroisoquinoline- | 8.3, 5.8 Hz, 1H), 6.98-7.07 (m, 2H), 4.64- | |
| | 2(1H)-carboxamide | 4.65 (bs, 2H), 3.71 (t, J = 5.8 Hz, 2H), 3.22- | |
| | | 3.27 (m, 2H), 2.81-2.90 (m, 2H), 1.61 (dq, | |
| | | J = 13.3, 6.6 Hz, 1H), 1.41 (q, J = 7.1 Hz, | |
| | | 2H), 0.90 (d, J = 6.6 Hz, 6H) | |
| 17 | N-(4-{[2-(2- | ¹H NMR (400 MHz, DMSO-d₆) δ ppm | (ESI(+)) |
| | thienyl)ethyl]carbamoyl} | 8.92-8.93 (bs, 1H), 8.48-8.52 (m, 1H), 7.74- | m/e 406 |
| | phenyl)-3,4- | 7.77 (m, 2H), 7.59-7.62 (m, 2H), 7.33 (dd, | (M + H)⁺ |
| | dihydroisoquinoline- | J = 5.0, 1.3 Hz, 1H), 7.19 (s, 4H), 6.95 (dd, | |
| | 2(1H)-carboxamide | J = 5.1, 3.3 Hz, 1H), 6.91 (d, J = 3.3 Hz, 1H), | |
| | | 4.66-4.67 (bs, 2H), 3.73 (t, J = 5.8 Hz, 2H), | |
| | | 3.45-3.51 (m, 2H), 3.05 (t, J = 7.2 Hz, 2H), | |
| | | 2.86 (t, J = 5.8 Hz, 2H) | |
| 20 | 7-fluoro-N-{4-[(3- | ¹H NMR (400 MHz, DMSO-d₆) δ ppm 8.83 | (ESI(+)) |
| | phenylpropyl)carbamoyl] | (s, 1H), 8.29 (t, J = 5.5 Hz, 1H), 7.73-7.76 | m/e 432 |
| | phenyl}-3,4- | (m, 2H), 7.54-7.57 (m, 2H), 7.26-7.31 (m, | (M + H)⁺ |
| | dihydroisoquinoline- | 2H), 7.20-7.25 (m, 3H), 7.15-7.20 (m, 1H), | |
| | 2(1H)-carboxamide | 6.98-7.07 (m, 2H), 4.64-4.65 (bs, 2H), 3.71 | |
| | | (t, J = 5.8 Hz, 2H), 3.21-3.29 (m, 2H), 2.81- | |
| | | 2.85 (m, 2H), 2.62 (t, J = 7.6 Hz, 2H), 1.82 | |
| | | (p, J = 7.3 Hz, 2H) | |
| 23 | 7-fluoro-N-(4-{[2-(2- | ¹H NMR (400 MHz, DMSO-d₆) δ ppm 8.84 | (ESI(+)) |
| | thienyl)ethyl]carbamoyl} | (s, 1H), 8.45 (t, J = 5.6 Hz, 1H), 7.73-7.76 | m/e 424 |
| | phenyl)-3,4- | (m, 2H), 7.55-7.58 (m, 2H), 7.33 (dd, J = | (M + H)⁺ |
| | dihydroisoquinoline- | 5.0, 1.2 Hz, 1H), 7.23 (dd, J = 8.3, 5.8 Hz, | |
| | 2(1H)-carboxamide | 1H), 7.00-7.08 (m, 2H), 6.95 (dd, J = 5.1, | |
| | | 3.4 Hz, 1H), 6.90-6.92 (m, 1H), 4.64-4.65 | |
| | | (bs, 2H), 3.71 (t, J = 5.8 Hz, 2H), 3.45-3.51 | |
| | | (m, 2H), 3.05 (t, J = 7.2 Hz, 2H), 2.82-2.89 | |
| | | (m, 2H) | |
| 25 | N-{4-[(3- | ¹H NMR (500 MHz, DMSO-d₆) δ ppm 8.88 | (ESI(+)) |
| | phenylpropyl)carbamoyl] | (s, 1H), 8.41 (s, 1H), 8.34 (d, J = 5.0 Hz, | m/e 415 |
| | phenyl}-3,4-dihydro-2,7- | 1H), 8.29 (t, J = 5.6 Hz, 1H), 7.74-7.76 (m, | (M + H)⁺ |
| | naphthyridine-2(1H)- | 2H), 7.55-7.57 (m, 2H), 7.27-7.30 (m, 2H), | |
| | carboxamide | 7.21-7.24 (m, 3H), 7.16-7.19 (m, 1H), 4.69- | |
| | | 4.70 (bs, 2H), 3.74 (t, J = 5.9 Hz, 2H), 3.26 | |
| | | (q, J = 6.5 Hz, 2H), 2.87 (t, J = 5.9 Hz, 2H), | |
| | | 2.62 (t, J = 7.6 Hz, 2H), 1.82 (p, J = 7.4 Hz, | |
| | | 2H) | |
| 28 | N-{4-[(3- | ¹H NMR (500 MHz, DMSO-d₆) δ ppm 8.88 | (ESI(+)) |
| | methylbutyl)carbamoyl] | (s, 1H), 8.41 (s, 1H), 8.34 (d, J = 5.0 Hz, | m/e 367 |
| | phenyl}-3,4-dihydro-2,7- | 1H), 8.23 (t, J = 5.6 Hz, 1H), 7.73-7.75 (m, | (M + H)⁺ |
| | naphthyridine-2(1H)- | 2H), 7.54-7.56 (m, 2H), 7.22 (d, J = 5.0 Hz, | |
| | carboxamide | 1H), 4.69-4.70 (bs, 2H), 3.73 (t, J = 5.9 Hz, | |
| | | 2H), 3.23-3.28 (m, 2H), 2.88 (t, J = 5.9 Hz, | |
| | | 2H), 1.61 (dq, J = 13.3, 6.7 Hz, 1H), 1.41 | |
| | | (q, J = 7.1 Hz, 2H), 0.90 (d, J = 6.6 Hz, 6H) | |
| 30 | N-(4-{[2-(2- | ¹H NMR (400 MHz, DMSO-d₆) δ ppm 8.90 | (ESI(+)) |
| | thienyl)ethyl]carbamoyl} | (s, 1H), 8.46 (t, J = 5.6 Hz, 1H), 8.41 (s, | m/e 407 |
| | phenyl)-3,4-dihydro-2,7- | 1H), 8.34 (d, J = 5.0 Hz, 1H), 7.74-7.77 (m, | (M + H)⁺ |
| | naphthyridine-2(1H)- | 2H), 7.55-7.58 (m, 2H), 7.33 (dd, J = 5.0, | |
| | carboxamide | 1.2 Hz, 1H), 7.22 (d, J = 5.0 Hz, 1H), 6.96 | |
| | | (dd, J = 5.1, 3.4 Hz, 1H), 6.91 (d, J = 3.9 | |
| | | Hz, 1H), 4.69-4.70 (bs, 2H), 3.74 (t, J = 5.8 | |
| | | Hz, 2H), 3.44-3.52 (m, 2H), 3.05 (t, J = 7.2 | |
| | | Hz, 2H), 2.88 (t, J = 5.8 Hz, 2H) | |

### Example 9

### N-{5-[(3-phenylpropyl)carbamoyl]pyridin-2-yl}-3,4-dihydroisoquinoline-2(1H)-carboxamide

### Example 9A

### methyl 6-(1,2,3,4-tetrahydroisoquinoline-2-carboxamido)nicotinate

In a 500 mL round bottom flask was mixed at room temperature triphosgene (0.722 g, 2.432 mmol) in anhydrous dichloromethane (50 mL). To this solution was added dropwise over 15 minutes a slurry of methyl 6-aminonicotinate (1.00 g, 6.57 mmol) and diisopropylethylamine (1.263 ml, 7.23 mmol) in anhydrous dichloromethane (40 mL). The mixture was stirred overnight at room temperature and a solution of 1,2,3,4-tetrahydroisoquinoline (0.834 ml, 6.57 mmol) and diisopropylethylamine (1.263 ml, 7.23 mmol) in anhydrous dichloromethane (40 mL) was added in one portion. The reaction mixture was stirred at room temperature overnight, the solvents were evaporated and the title compound was isolated by normal-phase flash chromatography.

### Example 9B

### 6-(1,2,3,4-tetrahydroisoquinoline-2-carboxamido)nicotinic acid

The title compound was prepared as described in Example 1B, substituting methyl 6-(1,2,3,4-tetrahydroisoquinoline-2-carboxamido)nicotinate for methyl 4-(1,2,3,4-tetrahydroisoquinoline-2-carboxamido)benzoate.

### Example 9C

### N-{5-[(3-phenylpropyl)carbamoyl]pyridin-2-yl}-3,4-dihydroisoquinoline-2(1H)-carboxamide

The title compound was prepared as described in Example 1C, substituting 3-phenylpropan-1-amine for 3-methylbutan-1-amine and 6-(1,2,3,4-tetrahydroisoquinoline-2-carboxamido)nicotinic acid for 4-(1,2,3,4-tetrahydroisoquinoline-2-carboxamido)benzoic acid. ¹H NMR (400 MHz, DMSO-d₆) δ ppm 9.54 (s, 1H), 8.71 (d, J=2.4 Hz, 1H), 8.47 (t, J=5.5 Hz, 1H), 8.11 (dd, J=8.8, 2.4 Hz, 1H), 7.87 (d, J=8.8 Hz, 1H), 7.18-7.29 (m, 9H), 4.67-4.68 (bs, 2H), 3.73 (t, J=5.9 Hz, 2H), 3.24-3.33 (m, 2H), 2.86 (t, J=5.8 Hz, 2H), 2.64 (t, J=7.6 Hz, 2H), 1.83 (p, J=7.3 Hz, 2H); (ESI(−)) m/e 413 (M−H)⁻.

### Example 10

### N-{5-[(3-methylbutyl)carbamoyl]pyridin-2-yl}-3,4-dihydroisoquinoline-2(1H)-carboxamide

The title compound was prepared as described in Example 1C, substituting 6-(1,2,3,4-tetrahydroisoquinoline-2-carboxamido)nicotinic acid for 4-(1,2,3,4-tetrahydroisoquinoline-2-carboxamido)benzoic acid. ¹H NMR (400 MHz, DMSO-d₆) δ ppm 9.54 (s, 1H), 8.71 (d, J=2.4 Hz, 1H), 8.40 (t, J=5.5 Hz, 1H), 8.10 (dd, J=8.8, 2.4 Hz, 1H), 7.87 (d, J=8.8 Hz, 1H), 7.18 (s, 4H), 4.67-4.68 (bs, 2H), 3.73 (t, J=5.9 Hz, 2H), 3.23-3.31 (m, 2H), 2.85 (t, J=5.8 Hz, 2H), 1.57-1.67 (m, 1H), 1.42 (q, J=7.1 Hz, 2H), 0.90 (d, J=6.6 Hz, 6H); (ESI(−)) m/e 365 (M−H)⁻.

### Example 18

### N-{6-[(3-methylbutyl)carbamoyl]pyridin-3-yl}-3,4-dihydroisoquinoline-2(1H)-carboxamide

### Example 18A

### methyl 5-(1,2,3,4-tetrahydroisoquinoline-2-carboxamido)picolinate

The title compound was prepared as described in Example 9A, substituting methyl 5-aminopicolinate for methyl 6-aminonicotinate.

### Example 18B

### 5-(1,2,3,4-tetrahydroisoquinoline-2-carboxamido)picolinic acid

The title compound was prepared as described in Example 1B, substituting methyl 5-(1,2,3,4-tetrahydroisoquinoline-2-carboxamido)picolinate for methyl 4-(1,2,3,4-tetrahydroisoquinoline-2-carboxamido)benzoate.

### Example 18C

### N-{6-[(3-methylbutyl)carbamoyl]pyridin-3-yl}-3,4-dihydroisoquinoline-2(1H)-carboxamide

The title compound was prepared as described in Example 1C, substituting 5-(1,2,3,4-tetrahydroisoquinoline-2-carboxamido)picolinic acid for 4-(1,2,3,4-tetrahydroisoquinoline-2-carboxamido)benzoic acid. ¹H NMR (400 MHz, DMSO-d₆) δ ppm 9.05 (s, 1H), 8.76 (d, J=2.5 Hz, 1H), 8.55 (t, J=6.0 Hz, 1H), 8.07 (dd, J=8.5, 2.5 Hz, 1H), 7.92 (d, J=8.5 Hz, 1H), 7.19-7.21 (m, 4H), 4.67-4.68 (bs, 2H), 3.73 (t, J=5.8 Hz, 2H), 3.26-3.33 (m, 2H), 2.88 (t, J=5.8 Hz, 2H), 1.53-1.63 (m, 1H), 1.42 (q, J=7.1 Hz, 2H), 0.89 (d, J=6.6 Hz, 6H); (ESI(−)) m/e 365 (M−H)⁻.

### Example 19

### N-(5-{[2-(2-thienyl)ethyl]carbamoyl}pyridin-2-yl)-3,4-dihydroisoquinoline-2(1H)-carboxamide

The title compound was prepared as described in Example 1C, substituting 2-(thiophen-2-yl)ethanamine for 3-methylbutan-1-amine and 6-(1,2,3,4-tetrahydroisoquinoline-2-carboxamido)nicotinic acid for 4-(1,2,3,4-tetrahydroisoquinoline-2-carboxamido)benzoic acid. ¹H NMR (400 MHz, DMSO-d₆) δ ppm 9.56 (s, 1H), 8.72 (d, J=2.4 Hz, 1H), 8.64 (t, J=5.5 Hz, 1H), 8.10 (dd, J=8.8, 2.4 Hz, 1H), 7.88 (d, J=8.8 Hz, 1H), 7.34 (dd, J=5.0, 1.2 Hz, 1H), 7.18 (s, 4H), 6.96 (dd, J=5.1, 3.4 Hz, 1H), 6.92 (d, J=3.4 Hz, 1H), 4.67-4.68 (bs, 2H), 3.74 (t, J=5.8 Hz, 2H), 3.47-3.52 (m, 2H), 3.07 (t, J=7.1 Hz, 2H), 2.86 (t, J=5.8 Hz, 2H); (ESI(−)) m/e 405 (M−H)⁻.

### Example 21

### N-{6-[(3-phenylpropyl)carbamoyl]pyridin-3-yl}-3,4-dihydroisoquinoline-2(1H)-carboxamide

The title compound was prepared as described in Example 1C, substituting 3-phenylpropan-1-amine for 3-methylbutan-1-amine and 5-(1,2,3,4-tetrahydroisoquinoline-2-carboxamido)picolinic acid for 4-(1,2,3,4-tetrahydroisoquinoline-2-carboxamido)benzoic acid. ¹H NMR (400 MHz, DMSO-d₆) δ ppm 9.06 (s, 1H), 8.77 (d, J=2.5 Hz, 1H), 8.66 (t, J=6.0 Hz, 1H), 8.07 (dd, J=8.6, 2.5 Hz, 1H), 7.93 (d, J=8.5 Hz, 1H), 7.25-7.30 (m, 2H), 7.14-7.24 (m, 7H), 4.67-4.68 (bs, 2H), 3.74 (t, J=5.8 Hz, 2H), 3.26-3.34 (m, 2H), 2.88 (t, J=5.8 Hz, 2H), 2.60 (t, J=7.6 Hz, 2H), 1.83 (p, J=7.3 Hz, 2H); (ESI(+)) m/e 415 (M+H)⁺.

### Example 22

### N-(6-{[2-(2-thienyl)ethyl]carbamoyl}pyridin-3-yl)-3,4-dihydroisoquinoline-2(1H)-carboxamide

The title compound was prepared as described in Example 1C, substituting 2-(thiophen-2-yl)ethanamine for 3-methylbutan-1-amine and 5-(1,2,3,4-tetrahydroisoquinoline-2-carboxamido)picolinic acid for 4-(1,2,3,4-tetrahydroisoquinoline-2-carboxamido)benzoic acid. ¹H NMR (400 MHz, DMSO-d₆) δ ppm 9.07 (s, 1H), 8.77 (d, J=2.0 Hz, 1H), 8.76 (t, J=6.3 Hz, 1H), 8.08 (dd, J=8.5, 2.5 Hz, 1H), 7.94 (d, J=8.5 Hz, 1H), 7.33 (dd, J=5.0, 1.2 Hz, 1H), 7.20 (s, 4H), 6.95 (dd, J=5.0, 3.4 Hz, 1H), 6.90-6.92 (m, 1H), 4.67-4.68 (bs, 2H), 3.74 (t, J=5.8 Hz, 2H), 3.54 (q, J=6.8 Hz, 2H), 3.07 (t, J=7.2 Hz, 2H), 2.88 (t, J=5.8 Hz, 2H); (ESI(+)) m/e 407 (M+H)⁺.

### Example 26

### N-[4-(2-oxo-2-{[2-(2-thienyl)ethyl]amino}ethyl)phenyl]-3,4-dihydroisoquinoline-2(1H)-carboxamide

### Example 26A

### ethyl 2-(4-(1,2,3,4-tetrahydroisoquinoline-2-carboxamido)phenyl)acetate

The title compound was prepared as described in Example 1A, substituting ethyl 2-(4-isocyanatophenyl)acetate for methyl 4-isocyanatobenzoate.

### Example 26B

### 2-(4-(1,2,3,4-tetrahydroisoquinoline-2-carboxamido)phenyl)acetic acid

The title compound was prepared as described in Example 1B, substituting ethyl 2-(4-(1,2,3,4-tetrahydroisoquinoline-2-carboxamido)phenyl)acetate for methyl 4-(1,2,3,4-tetrahydroisoquinoline-2-carboxamido)benzoate.

### Example 26C

### N-[4-(2-oxo-2-{[2-(2-thienyl)ethyl]amino}ethyl)phenyl]-3,4-dihydroisoquinoline-2(1H)-carboxamide

The title compound was prepared as described in Example 1C, substituting 2-(thiophen-2-yl)ethanamine for 3-methylbutan-1-amine and 2-(4-(1,2,3,4-tetrahydroisoquinoline-2-carboxamido)phenyl)acetic acid for 4-(1,2,3,4-tetrahydroisoquinoline-2-carboxamido)benzoic acid. ¹H NMR (400 MHz, DMSO-d₆) δ ppm 8.51 (s, 1H), 8.08-8.12 (m, 1H), 7.36-7.39 (m, 2H), 7.32 (dd, J=5.1, 1.2 Hz, 1H), 7.18 (s, 4H), 7.07-7.11 (m, 2H), 6.94 (dd, J=5.1, 3.4 Hz, 1H), 6.83-6.85 (m, 1H), 4.62-4.63 (bs, 2H), 3.69 (t, J=5.8 Hz, 2H), 3.31-3.33 (bs, 2H), 3.25-3.31 (m, 2H), 2.91 (t, J=7.1 Hz, 2H), 2.84 (t, J=5.8 Hz, 2H); (ESI(+)) m/e 420 (M+H)⁺.

### Example 27

### N-(4-{2-oxo-2-[(3-phenylpropyl)amino]ethyl}phenyl)-3,4-dihydroisoquinoline-2(1H)-carboxamide

The title compound was prepared as described in Example 1C, substituting phenylpropylamine for 3-methylbutan-1-amine and 2-(4-(1,2,3,4-tetrahydroisoquinoline-2-carboxamido)phenyl)acetic acid for 4-(1,2,3,4-tetrahydroisoquinoline-2-carboxamido)benzoic acid. ¹H NMR (400 MHz, DMSO-d₆) δ ppm 8.51 (s, 1H), 7.97-8.01 (m, 1H), 7.37-7.40 (m, 2H), 7.24-7.28 (m, 2H), 7.15-7.18 (m, 7H), 7.11-7.15 (m, 2H), 4.62-4.63 (bs, 2H), 3.69 (t, J=5.9 Hz, 2H), 3.31-3.32 (bs, 2H), 3.02-3.07 (m, 2H), 2.84 (t, J=5.8 Hz, 2H), 2.51-2.58 (m, 2H), 1.68 (p, J=7.3 Hz, 2H); (ESI(+)) m/e 428 (M+H)⁺.

### Example 29

### N-(4-{2-[(3-methylbutyl)amino]-2-oxoethyl}phenyl)-3,4-dihydroisoquinoline-2(1H)-carboxamide

The title compound was prepared as described in Example 1C, substituting 2-(4-(1,2,3,4-tetrahydroisoquinoline-2-carboxamido)phenyl)acetic acid for 4-(1,2,3,4-tetrahydroisoquinoline-2-carboxamido)benzoic acid. ¹H NMR (400 MHz, DMSO-d₆) δ ppm 8.51 (s, 1H), 7.88-7.91 (m, 1H), 7.36-7.39 (m, 2H), 7.18 (s, 4H), 7.09-7.12 (m, 2H), 4.62-4.63 (bs, 2H), 3.69 (t, J=5.9 Hz, 2H), 3.29 (s, 2H), 3.02-3.07 (m, 2H), 2.84 (t, J=5.8 Hz, 2H), 1.50-1.61 (m, 1H), 1.28 (q, J=7.1 Hz, 2H), 0.85 (d, J=6.6 Hz, 6H); (ESI(+)) m/e 380 (M+H)⁺.

### Example 31

### N-{4-[(4-methylpentanoyl)amino]phenyl}-3,4-dihydroisoquinoline-2(1H)-carboxamide

### Example 31A

### N-(4-nitrophenyl)-3,4-dihydroisoquinoline-2(1H)-carboxamide

The title compound was prepared as described in Example 1A, substituting 1-isocyanato-4-nitrobenzene for methyl 4-isocyanatobenzoate.

### Example 31B

### N-(4-aminophenyl)-3,4-dihydroisoquinoline-2(1H)-carboxamide

A 100 mL round bottom flask charged with 10% palladium on carbon (100 mg, 0.940 mmol) catalyst was degassed and back filled with nitrogen. To this flask was added a mixture of N-(4-nitrophenyl)-3,4-dihydroisoquinoline-2(1H)-carboxamide (1.07 g, 3.60 mmol) in methanol (30 ml). The reaction was degassed and back filled with hydrogen. The suspension was stirred overnight at room temperature and then filtered through diatomaceous earth with methanol washes. Concentration of the filtrate and normal-phase flash chromatography provided the title compound.

### Example 31C

### N-{4-[(4-methylpentanoyl)amino]phenyl}-3,4-dihydroisoquinoline-2(1H)-carboxamide

The title compound was prepared as described in Example 1C, substituting N-(4-aminophenyl)-3,4-dihydroisoquinoline-2(1H)-carboxamide for 3-methylbutan-1-amine and 4-methylpentanoic acid for 4-(1,2,3,4-tetrahydroisoquinoline-2-carboxamido)benzoic acid. ¹H NMR (400 MHz, DMSO-d₆) δ ppm 9.72 (s, 1H), 8.48 (s, 1H), 7.41-7.46 (m, 2H), 7.34-7.38 (m, 2H), 7.17-7.18 (m, 4H), 4.62-4.62 (bs, 2H), 3.68 (t, J=5.8 Hz, 2H), 2.84 (t, J=5.8 Hz, 2H), 2.27 (t, J=7.6 Hz, 2H), 1.49-1.61 (m, 1H), 1.44-1.51 (m, 2H), 0.89 (d, J=6.4 Hz, 6H); (ESI(+)) m/e 366 (M+H)⁺.

TABLE 3
The following examples were essentially prepared as described in Example 31,
substituting the appropriate acid for 4-methylpentanoic acid in Example 31C.

| Ex | Name | ¹H NMR | MS |
|---|---|---|---|
| 32 | N-{4-[(4- | ¹H NMR (400 MHz, DMSO-d₆) δ | (ESI(+)) |
| | phenylbutanoyl)amino] | ppm 9.73 (s, 1H), 8.48 (s, 1H), 7.43- | m/e 414 |
| | phenyl}-3,4-dihydroisoquinoline- | 7.49 (m, 2H), 7.35-7.38 (m, 2H), | (M + H)⁺ |
| | 2(1H)-carboxamide | 7.27-7.31 (m, 2H), 7.20-7.23 (m, 2H), | |
| | | 7.14-7.20 (m, 5H), 4.61-4.62 (bs, 2H), | |
| | | 3.68 (t, J = 5.8 Hz, 2H), 2.84 (t, J = | |
| | | 5.8 Hz, 2H), 2.62 (t, J = 7.5 Hz, 2H), | |
| | | 2.29 (t, J = 7.4 Hz, 2H), 1.81-1.93 (m, | |
| | | 2H) | |
| 33 | N-(4-{[3-(2- | ¹H NMR (500 MHz, DMSO-d₆) δ | (ESI(+)) |
| | thienyl)propanoyl]amino} | ppm 9.82 (s, 1H), 8.49 (s, 1H), 7.43- | m/e 406 |
| | phenyl)-3,4-dihydroisoquinoline- | 7.45 (m, 2H), 7.36-7.39 (m, 2H), 7.30 | (M + H)⁺ |
| | 2(1H)-carboxamide | (dd, J = 5.0, 1.2 Hz, 1H), 7.18-7.18 | |
| | | (bs, 4H), 6.93 (dd, J = 5.2, 3.4 Hz, | |
| | | 1H), 6.89 (d, J = 3.8 Hz, 1H), 4.62- | |
| | | 4.62 (bs, 2H), 3.68 (t, J = 5.8 Hz, 2H), | |
| | | 3.11 (t, J = 7.4 Hz, 2H), 2.84 (t, J = | |
| | | 5.9 Hz, 2H), 2.64 (t, J = 7.4 Hz, 2H) | |
| 63 | N-(4- | ¹H NMR (400 MHz, DMSO-d₆/ | (ESI(+)) |
| | {[(benzyloxy)acetyl]amino} | Deuterium Oxide, Temp = 90° C.) δ | m/e 416 |
| | phenyl)-3,4-dihydroisoquinoline- | ppm 7.43-7.48 (m, 2H), 7.34-7.42 (m, | (M + H)⁺ |
| | 2(1H)-carboxamide | 6H), 7.28-7.33 (m, 1H), 7.15-7.20 (m, | |
| | | 4H), 4.61-4.64 (m, 4H), 4.06 (s, 2H), | |
| | | 3.68 (t, J = 5.9 Hz, 2H), 2.86 (t, J = | |
| | | 6.0 Hz, 2H) | |
| 64 | N-(4-{[(4- | ¹H NMR (400 MHz, DMSO-d₆/ | (ESI(−)) |
| | methoxycyclohexyl)carbonyl] | Deuterium Oxide, Temp = 90° C.) δ | m/e 406 |
| | amino}phenyl)-3,4- | ppm 7.40-7.48 (m, 2H), 7.34-7.36 (m, | (M − H)⁻ |
| | dihydroisoquinoline-2(1H)- | 2H), 7.11-7.25 (m, 4H), 4.61 (s, 2H), | |
| | carboxamide | 3.68 (t, J = 6.0 Hz, 2H), 3.39 (p, J = | |
| | | 3.4 Hz, 1H), 2.86 (t, J = 6.0 Hz, 2H), | |
| | | 2.23-2.39 (m, 1H), 1.87 (dd, J = 13.6, | |
| | | 4.2 Hz, 2H) | |
| 65 | N-(4-{[(1-acetylpiperidin-4- | ¹H NMR (400 MHz, DMSO-d₆/ | (ESI(−)) |
| | yl)carbonyl]amino}phenyl)- | Deuterium Oxide, Temp = 90° C.) δ | m/e 419 |
| | 3,4-dihydroisoquinoline- | ppm 7.41-7.44 (m, 2H), 7.35-7.38 (m, | (M − H)⁻ |
| | 2(1H)-carboxamide | 2H), 7.18 (s, 3H), 7.18 (s, 1H), 4.61 | |
| | | (s, 2H), 3.81-4.37 (m, 2H), 3.66-3.70 | |
| | | (m, 2H), 2.93-3.18 (m, 1H), 2.86 (t, | |
| | | J = 6.0 Hz, 2H), 2.61-2.84 (m, 1H), | |
| | | 2.50-2.63 (m, 1H), 2.01 (s, 3H), 1.79- | |
| | | 1.85 (m, 2H), 1.49-1.64 (m, 2H) | |
| 66 | N-(4-{[4-oxo-4-(2- | ¹H NMR (400 MHz, DMSO-d₆/ | (ESI(+)) |
| | thienyl)butanoyl]amino} | Deuterium Oxide, Temp = 90° C.) δ | m/e 434 |
| | phenyl)-3,4-dihydroisoquinoline- | ppm 7.92 (dd, J = 3.7, 1.1 Hz, 1H), | (M + H)⁺ |
| | 2(1H)-carboxamide | 7.90 (dd, J = 4.9, 1.1 Hz, 1H), 7.39- | |
| | | 7.43 (m, 2H), 7.35-7.37 (m, 2H), 7.23 | |
| | | (dd, J = 4.9, 3.7 Hz, 1H), 7.13-7.19 | |
| | | (m, 4H), 4.61 (s, 2H), 3.68 (t, J = 6.0 | |
| | | Hz, 2H), 3.25 (t, J = 6.7 Hz, 2H), 2.86 | |
| | | (t, J = 6.0 Hz, 2H), 2.70 (t, J = 6.7 Hz, | |
| | | 2H) | |
| 67 | N-(4-{[3- | ¹H NMR (400 MHz, DMSO-d₆/ | (ESI(+)) |
| | (phenylsulfonyl)propanoyl] | Deuterium Oxide, Temp = 90° C.) δ | m/e 464 |
| | amino}phenyl)-3,4- | ppm 7.88-7.94 (m, 2H), 7.71-7.76 (m, | (M + H)⁺ |
| | dihydroisoquinoline-2(1H)- | 1H), 7.63-7.68 (m, 2H), 7.31-7.37 (m, | |
| | carboxamide | 4H), 7.14-7.21 (m, 4H), 4.61 (s, 2H), | |
| | | 3.68 (t, J = 5.9 Hz, 2H), 3.57 (t, J = | |
| | | 7.3 Hz, 2H), 2.85 (t, J = 6.0 Hz, 2H), | |
| | | 2.66-2.70 (m, 2H) | |
| 68 | N-{4-[((2R)-2,3-dihydro-1- | ¹H NMR (400 MHz, DMSO-d₆/ | (ESI(+)) |
| | benzofuran-2- | Deuterium Oxide, Temp = 90° C.) δ | m/e 414 |
| | ylcarbonyl)amino]phenyl}- | ppm 7.47-7.49 (m, 2H), 7.39-7.41 (m, | (M + H)⁺ |
| | 3,4-dihydroisoquinoline- | 2H), 7.22-7.25 (m, 1H), 7.15-7.20 (m, | |
| | 2(1H)-carboxamide and N-{4- | 4H), 7.11-7.16 (m, 1H), 6.86-6.90 (m, | |
| | [((2S)-2,3-dihydro-1- | 2H), 5.26 (dd, J = 10.1, 6.8 Hz, 1H), | |
| | benzofuran-2- | 4.61 (s, 2H), 3.68 (t, J = 5.9 Hz, 2H), | |
| | ylcarbonyl)amino]phenyl}- | 3.52 (dd, J = 16.0, 10.1 Hz, 1H), 3.38 | |
| | 3,4-dihydroisoquinoline- | (dd, J = 15.9, 6.9 Hz, 1H), 2.86 (t, J = | |
| | 2(1H)-carboxamide | 6.0 Hz, 2H) | |
| 69 | N-{4-[((3R)-3- | ¹H NMR (400 MHz, DMSO-d₆/ | (ESI(+)) |
| | methylpentanoyl)amino] | Deuterium Oxide, Temp = 90° C.) δ | m/e 366 |
| | phenyl}-3,4-dihydroisoquinoline- | ppm 7.40-7.43 (m, 2H), 7.34-7.37 (m, | (M + H)⁺ |
| | 2(1H)-carboxamide and N-{4- | 2H), 7.14-7.21 (m, 4H), 4.61 (s, 2H), | |
| | [((3S)-3- | 3.68 (t, J = 6.0 Hz, 2H), 2.86 (t, J = | |
| | methylpentanoyl)amino] | 6.0 Hz, 2H), 2.26 (dd, J = 13.8, 6.2 | |
| | phenyl}-3,4-dihydroisoquinoline- | Hz, 1H), 2.10 (dd, J = 13.8, 7.8 Hz, | |
| | 2(1H)-carboxamide | 1H), 1.82-1.95 (m, 1H), 1.33-1.44 (m, | |
| | | 1H), 1.18-1.29 (m, 1H), 0.92 (d, J = | |
| | | 6.7 Hz, 3H), 0.88 (t, J = 7.4 Hz, 3H) | |
| 70 | N-{4-[(2,2- | ¹H NMR (400 MHz, DMSO-d₆/ | (ESI(−)) |
| | dimethylbutanoyl)amino] | Deuterium Oxide, Temp = 90° C.) δ | m/e 364 |
| | phenyl}-3,4-dihydroisoquinoline- | ppm 7.40-7.44 (m, 2H), 7.34-7.38 (m, | (M − H)⁻ |
| | 2(1H)-carboxamide | 2H), 7.18 (s, 4H), 4.62 (s, 2H), 3.68 | |
| | | (t, J = 6.0 Hz, 2H), 2.86 (t, J = 6.0 Hz, | |
| | | 2H), 1.61 (q, J = 7.4 Hz, 2H), 1.18 (s, | |
| | | 6H), 0.83 (t, J = 7.4 Hz, 3H) | |
| 71 | N-{4-[(3,3- | ¹H NMR (400 MHz, DMSO-d₆/ | (ESI(+)) |
| | dimethylbutanoyl)amino] | Deuterium Oxide, Temp = 90° C.) δ | m/e 366 |
| | phenyl}-3,4-dihydroisoquinoline- | ppm 7.40-7.43 (m, 2H), 7.34-7.37 (m, | (M + H)⁺ |
| | 2(1H)-carboxamide | 2H), 7.13-7.22 (m, 4H), 4.61 (s, 2H), | |
| | | 3.68 (t, J = 6.0 Hz, 2H), 2.86 (t, J = | |
| | | 6.0 Hz, 2H), 2.17 (s, 2H), 1.03 (s, 9H) | |
| 72 | N-[4- | ¹H NMR (400 MHz, DMSO-d₆/ | (ESI(−)) |
| | (heptanoylamino)phenyl]-3,4- | Deuterium Oxide, Temp = 90° C.) δ | m/e 378 |
| | dihydroisoquinoline-2(1H)- | ppm 7.39-7.44 (m, 2H), 7.33-7.38 (m, | (M − H)⁻ |
| | carboxamide | 2H), 7.14-7.22 (m, 4H), 4.61 (s, 2H), | |
| | | 3.68 (t, J = 5.9 Hz, 2H), 2.86 (t, J = | |
| | | 6.0 Hz, 2H), 2.27 (t, J = 7.4 Hz, 2H), | |
| | | 1.60 (p, J = 7.1 Hz, 2H), 1.27-1.37 | |
| | | (m, 6H), 0.84-0.90 (m, 3H) | |
| 73 | N-{4-[(4,4,4- | ¹H NMR (400 MHz, DMSO-d₆/ | (ESI(−)) |
| | trifluorobutanoyl)amino] | Deuterium Oxide, Temp = 90° C.) δ | m/e 390 |
| | phenyl}-3,4-dihydroisoquinoline- | ppm 7.37-7.41 (m, 4H), 6.95-7.22 (m, | (M − H)⁻ |
| | 2(1H)-carboxamide | 4H), 4.62 (s, 2H), 3.68 (t, J = 6.0 Hz, | |
| | | 2H), 2.86 (t, J = 6.0 Hz, 2H), 2.53- | |
| | | 2.65 (m, 4H) | |
| 74 | N-(4-{[(2- | ¹H NMR (400 MHz, DMSO-d₆/ | (ESI(+)) |
| | methoxyethoxy)acetyl]amino} | Deuterium Oxide, Temp = 90° C.) δ | m/e 384 |
| | phenyl)-3,4- | ppm 7.44-7.47 (m, 2H), 7.38-7.41 (m, | (M + H)⁺ |
| | dihydroisoquinoline-2(1H)- | 2H), 7.11-7.22 (m, 4H), 4.62 (s, 2H), | |
| | carboxamide | 4.04 (s, 2H), 3.66-3.71 (m, 4H), 3.54- | |
| | | 3.57 (m, 2H), 3.32 (s, 3H), 2.86 (t, J = | |
| | | 6.0 Hz, 2H) | |
| 75 | N-{4-[((3R)-tetrahydrofuran- | ¹H NMR (400 MHz, DMSO-d₆/ | (ESI(+)) |
| | 3-ylcarbonyl)amino]phenyl}- | Deuterium Oxide, Temp = 90° C.) δ | m/e 366 |
| | 3,4-dihydroisoquinoline- | ppm 7.41-7.44 (m, 2H), 7.36-7.39 (m, | (M + H)⁺ |
| | 2(1H)-carboxamide and N-{4- | 2H), 7.17 (s, 4H), 4.61 (s, 2H), 3.93 | |
| | [((3S)-tetrahydrofuran-3- | (t, J = 8.1 Hz, 1H), 3.65-3.83 (m, 5H), | |
| | ylcarbonyl)amino]phenyl}- | 3.09-3.17 (m, 1H), 2.86 (t, J = 6.0 Hz, | |
| | 3,4-dihydroisoquinoline- | 2H), 2.00-2.12 (m, 2H) | |
| | 2(1H)-carboxamide | | |
| 76 | N-(4-{[3- | ¹H NMR (400 MHz, DMSO-d₆/ | (ESI(+)) |
| | (methylthio)propanoyl]amino} | Deuterium Oxide, Temp = 90° C.) δ | m/e 370 |
| | phenyl)-3,4- | ppm 7.32-7.50 (m, 4H), 7.11-7.26 (m, | (M + H)⁺ |
| | dihydroisoquinoline-2(1H)- | 4H), 4.61 (s, 2H), 3.68 (t, J = 6.0 Hz, | |
| | carboxamide | 2H), 2.86 (t, J = 6.0 Hz, 2H), 2.74- | |
| | | 2.78 (m, 2H), 2.59 (t, J = 7.0 Hz, 2H), | |
| | | 2.09 (s, 3H) | |
| 77 | N-{4- | ¹H NMR (400 MHz, DMSO-d₆/ | (ESI(+)) |
| | [(cyclopentylacetyl)amino] | Deuterium Oxide, Temp = 90° C.) δ | m/e 378 |
| | phenyl}-3,4-dihydroisoquinoline- | ppm 7.40-7.43 (m, 2H), 7.34-7.37 (m, | (M + H)⁺ |
| | 2(1H)-carboxamide | 2H), 7.14-7.21 (m, 4H), 4.61 (s, 2H), | |
| | | 3.68 (t, J = 5.9 Hz, 2H), 2.86 (t, J = | |
| | | 6.0 Hz, 2H), 2.24-2.29 (m, 3H), 1.72- | |
| | | 1.80 (m, 2H), 1.48-1.64 (m, 4H), | |
| | | 1.17-1.26 (m, 2H) | |
| 78 | N-{4- | ¹H NMR (400 MHz, DMSO-d₆/ | (ESI(+)) |
| | [(cyclohexylcarbonyl)amino] | Deuterium Oxide, Temp = 90° C.) δ | m/e XXX |
| | phenyl}-3,4- | ppm 7.41-7.44 (m, 2H), 7.33-7.36 (m, | (M + H)⁺ |
| | dihydroisoquinoline-2(1H)- | 2H), 7.13-7.22 (m, 4H), 4.61 (s, 2H), | |
| | carboxamide | 3.68 (t, J = 5.9 Hz, 2H), 2.86 (t, J = | |
| | | 6.0 Hz, 2H), 2.27-2.35 (m, 1H), 1.70- | |
| | | 1.82 (m, 4H), 1.62-1.67 (m, 1H), | |
| | | 1.38-1.49 (m, 2H), 1.14-1.34 (m, 3H) | |
| 79 | N-{4- | ¹H NMR (400 MHz, DMSO-d₆/ | (ESI(−)) |
| | [(cyclohexylacetyl)amino] | Deuterium Oxide, Temp = 90° C.) δ | m/e 390 |
| | phenyl}-3,4-dihydroisoquinoline- | ppm 7.40-7.43 (m, 2H), 7.34-7.36 (m, | (M − H)⁻ |
| | 2(1H)-carboxamide | 2H), 7.14-7.21 (m, 4H), 4.61 (s, 2H), | |
| | | 3.68 (t, J = 5.9 Hz, 2H), 2.86 (t, J = | |
| | | 6.0 Hz, 2H), 2.16 (d, J = 6.9 Hz, 2H), | |
| | | 1.58-1.80 (m, 6H), 1.11-1.30 (m, 3H), | |
| | | 0.95-1.06 (m, 2H) | |
| 80 | N-[4-(benzoylamino)phenyl]- | ¹H NMR (400 MHz, DMSO-d₆/ | (ESI(+)) |
| | 3,4-dihydroisoquinoline- | Deuterium Oxide, Temp = 90° C.) δ | m/e 372 |
| | 2(1H)-carboxamide | ppm 7.92-7.94 (m, 2H), 7.58-7.63 (m, | (M + H)⁺ |
| | | 2H), 7.54-7.58 (m, 1H), 7.48-7.53 (m, | |
| | | 2H), 7.42-7.45 (m, 2H), 7.18 (s, 4H), | |
| | | 4.63 (s, 2H), 3.70 (t, J = 5.9 Hz, 2H), | |
| | | 2.87 (t, J = 6.0 Hz, 2H) | |
| 81 | N-{4- | ¹H NMR (400 MHz, DMSO-d₆/ | (ESI(+)) |
| | [(phenylacetyl)amino] | Deuterium Oxide, Temp = 90° C.) δ | m/e 386 |
| | phenyl}-3,4-dihydroisoquinoline- | ppm 7.25-7.43 (m, 8H), 7.20-7.28 (m, | (M + H)⁺ |
| | 2(1H)-carboxamide | 1H), 7.17 (s, 4H), 4.61 (s, 2H), 3.68 | |
| | | (t, J = 5.9 Hz, 2H), 3.61 (s, 2H), 2.84- | |
| | | 2.87 (m, 2H) | |
| 82 | N-(4-{[3-(4- | ¹H NMR (400 MHz, DMSO-d₆/ | (ESI(+)) |
| | aminophenyl)propanoyl]amino} | Deuterium Oxide, Temp = 90° C.) δ | m/e 415 |
| | phenyl)-3,4- | ppm 7.33-7.41 (m, 4H), 7.16-7.21 (m, | (M + H)⁺ |
| | dihydroisoquinoline-2(1H)- | 2H), 7.15-7.20 (m, 4H), 6.95-6.99 (m, | |
| | carboxamide | 2H), 4.61 (s, 2H), 3.68 (t, J = 5.9 Hz, | |
| | | 2H), 2.87-2.91 (m, 2H), 2.84-2.87 (m, | |
| | | 2H), 2.55-2.60 (m, 2H) | |
| 83 | N-[4-(3-furoylamino)phenyl]- | ¹H NMR (400 MHz, DMSO-d₆/ | (ESI(−)) |
| | 3,4-dihydroisoquinoline- | Deuterium Oxide, Temp = 90° C.) δ | m/e 360 |
| | 2(1H)-carboxamide | ppm 8.25 (dd, J = 1.6, 0.8 Hz, 1H), | (M − H)⁻ |
| | | 7.68 (t, J = 1.7 Hz, 1H), 7.52-7.55 (m, | |
| | | 2H), 7.41-7.43 (m, 2H), 7.18 (s, 4H), | |
| | | 6.94 (dd, J = 1.9, 0.9 Hz, 1H), 4.63 (s, | |
| | | 2H), 3.69 (t, J = 5.9 Hz, 2H), 2.87 (t, | |
| | | J = 6.0 Hz, 2H) | |
| 84 | N-{4-[(2,5-dimethyl-3- | ¹H NMR (400 MHz, DMSO-d₆/ | (ESI(−)) |
| | furoyl)amino]phenyl}-3,4- | Deuterium Oxide, Temp = 90° C.) δ | m/e 388 |
| | dihydroisoquinoline-2(1H)- | ppm 7.51-7.54 (m, 2H), 7.38-7.41 (m, | (M − H)⁻ |
| | carboxamide | 2H), 7.14-7.21 (m, 4H), 6.53-6.54 (m, | |
| | | 1H), 4.62 (s, 2H), 3.69 (t, J = 6.0 Hz, | |
| | | 2H), 2.86 (t, J = 6.0 Hz, 2H), 2.48 (s, | |
| | | 3H), 2.25 (s, 3H) | |
| 85 | N-{4-[(3- | ¹H NMR (400 MHz, DMSO-d₆/ | (ESI(+)) |
| | thienylcarbonyl)amino] | Deuterium Oxide, Temp = 90° C.) δ | m/e 378 |
| | phenyl}-3,4-dihydroisoquinoline- | ppm 8.24 (dd, J = 2.9, 1.4 Hz, 1H), | (M + H)⁺ |
| | 2(1H)-carboxamide | 7.61 (dd, J = 5.0, 1.4 Hz, 1H), 7.54- | |
| | | 7.59 (m, 3H), 7.41-7.44 (m, 2H), 7.18 | |
| | | (s, 4H), 4.63 (s, 2H), 3.70 (t, J = 6.0 | |
| | | Hz, 2H), 2.87 (t, J = 6.0 Hz, 2H) | |
| 86 | N-{4-[(1H-pyrrol-2- | ¹H NMR (400 MHz, DMSO-d₆/ | (ESI(−)) |
| | ylcarbonyl)amino]phenyl}- | Deuterium Oxide, Temp = 90° C.) δ | m/e 359 |
| | 3,4-dihydroisoquinoline- | ppm 7.54-7.56 (m, 2H), 7.39-7.42 (m, | (M − H)⁻ |
| | 2(1H)-carboxamide | 2H), 7.16-7.20 (m, 4H), 6.94-6.98 (m, | |
| | | 2H), 6.16 (dd, J = 3.7, 2.5 Hz, 1H), | |
| | | 4.63 (s, 2H), 3.69 (t, J = 5.9 Hz, 2H), | |
| | | 2.87 (t, J = 6.0 Hz, 2H) | |
| 87 | N-{4-[(1,3-miazol-5- | ¹H NMR (400 MHz, DMSO-d₆/ | (ESI(+)) |
| | ylcarbonyl)amino]phenyl}- | Deuterium Oxide, Temp = 90° C.) δ | m/e 379 |
| | 3,4-dihydroisoquinoline- | ppm 9.19-9.20 (m, 1H), 8.59-8.60 (m, | (M + H)⁺ |
| | 2(1H)-carboxamide | 1H), 7.53-7.56 (m, 2H), 7.44-7.47 (m, | |
| | | 2H), 7.13-7.23 (m, 4H), 4.63 (s, 2H), | |
| | | 3.70 (t, J = 5.9 Hz, 2H), 2.87 (t, J = | |
| | | 5.9 Hz, 2H) | |
| 88 | N-{4-[(1H-pyrazol-5- | ¹H NMR (400 MHz, DMSO-d₆/ | (ESI(+)) |
| | ylcarbonyl)amino]phenyl}- | Deuterium Oxide, Temp = 90° C.) δ | m/e 362 |
| | 3,4-dihydroisoquinoline- | ppm 7.71-7.83 (m, 1H), 7.58-7.61 (m, | (M + H)⁺ |
| | 2(1H)-carboxamide | 2H), 7.41-7.44 (m, 2H), 7.18 (s, 4H), | |
| | | 6.70-6.85 (m, 1H), 4.63 (s, 2H), 3.70 | |
| | | (t, J = 5.9 Hz, 2H), 2.87 (t, J = 6.0 Hz, | |
| | | 2H) | |
| 89 | N-{4-[(1H-pyrazol-4- | ¹H NMR (400 MHz, DMSO-d₆/ | (ESI(−)) |
| | ylcarbonyl)amino]phenyl}- | Deuterium Oxide, Temp = 90° C.) δ | m/e 360 |
| | 3,4-dihydroisoquinoline- | ppm 7.87-8.40 (m, 2H), 7.53-7.55 (m, | (M − H)⁻ |
| | 2(1H)-carboxamide | 2H), 7.39-7.42 (m, 2H), 7.18 (s, 4H), | |
| | | 4.63 (s, 2H), 3.69 (t, J = 5.9 Hz, 2H), | |
| | | 2.87 (t, J = 5.9 Hz, 2H) | |
| 90 | N-{4-[(1,2-oxazol-5- | ¹H NMR (400 MHz, DMSO-d₆/ | (ESI(−)) |
| | ylcarbonyl)amino]phenyl}- | Deuterium Oxide, Temp = 90° C.) δ | m/e 361 |
| | 3,4-dihydroisoquinoline- | ppm 8.67 (d, J = 1.8 Hz, 1H), 7.57- | (M − H)⁻ |
| | 2(1H)-carboxamide | 7.60 (m, 2H), 7.45-7.48 (m, 2H), 7.18 | |
| | | (s, 4H), 7.13 (d, J = 2.0 Hz, 1H), 4.63 | |
| | | (s, 2H), 3.70 (t, J = 6.0 Hz, 2H), 2.87 | |
| | | (t, J = 6.0 Hz, 2H) | |
| 91 | N-{4-[(pyridin-2- | ¹H NMR (400 MHz, DMSO-d₆/ | (ESI(+)) |
| | ylacetyl)amino]phenyl}-3,4- | Deuterium Oxide, Temp = 90° C.) δ | m/e 387 |
| | dihydroisoquinoline-2(1H)- | ppm 8.62-8.65 (m, 1H), 8.06-8.12 (m, | (M + H)⁺ |
| | carboxamide | 1H), 7.64-7.69 (m, 1H), 7.54-7.59 (m, | |
| | | 1H), 7.42-7.46 (m, 2H), 7.37-7.41 (m, | |
| | | 2H), 7.14-7.21 (m, 4H), 4.61 (s, 2H), | |
| | | 3.68 (t, J = 6.0 Hz, 2H), 2.91 (s, 2H), | |
| | | 2.86 (t, J = 6.0 Hz, 2H) | |
| 92 | N-{4-[(N,N-dimethyl-beta- | ¹H NMR (400 MHz, DMSO-d₆) δ | (ESI(+)) |
| | alanyl)amino]phenyl}-3,4- | ppm 9.89 (s, 1H), 8.49 (s, 1H), 7.44 | m/e 367 |
| | dihydroisoquinoline-2(1H)- | (m, 2H), 7.37 (m, 2H), 7.17 (m, 4H), | (M + H)⁺ |
| | carboxamide | 4.62 (bs, 2H), 3.68 (t, J = 5.9 Hz, 2H), | |
| | | 2.84 (t, J = 5.8 Hz, 2H), 2.57 (t, J = | |
| | | 7.0 Hz, 2H), 2.42 (t, J = 7.0 Hz, 2H), | |
| | | 2.19(s, 6H) | |
| 93 | N-(4-{[3-(piperidin-1- | ¹H NMR (400 MHz, DMSO-d₆/ | (ESI(+)) |
| | yl)propanoyl] amino}phenyl)- | Deuterium Oxide, Temp = 90° C.) δ | m/e 407 |
| | 3,4-dihydroisoquinoline- | ppm 7.41-7.44 (m, 4H), 7.14-7.19 (m, | (M + H)⁺ |
| | 2(1H)-carboxamide | 4H), 4.62 (s, 2H), 3.68 (t, J = 6.0 Hz, | |
| | | 2H), 3.38 (t, J = 7.0 Hz, 2H), 2.92- | |
| | | 3.59 (m, 4H), 2.86 (t, J = 6.0 Hz, 2H), | |
| | | 2.81 (t, J = 7.1 Hz, 2H), 1.29-2.09 (m, | |
| | | 6H) | |
| 94 | N-{4-[(morpholin-4- | ¹H NMR (400 MHz, DMSO-d₆/ | (ESI(+)) |
| | ylacetyl)amino]phenyl}-3,4- | Deuterium Oxide, Temp = 90° C.) δ | m/e 395 |
| | dihydroisoquinoline-2(1H)- | ppm 7.25-7.50 (m, 4H), 7.11-7.24 (m, | (M + H)⁺ |
| | carboxamide | 4H), 4.62 (s, 2H), 3.96-3.97 (m, 2H), | |
| | | 3.86-3.89 (m, 4H), 3.69 (t, J = 6.0 Hz, | |
| | | 2H), 3.26-3.29 (m, 4H), 2.86 (t, J = | |
| | | 6.0 Hz, 2H) | |
| 95 | N-(4-{[3-(morpholin-4- | ¹H NMR (400 MHz, DMSO-d₆/ | (ESI(−)) |
| | yl)propanoyl] amino}phenyl)- | Deuterium Oxide, Temp = 90° C.) δ | m/e 407 |
| | 3,4-dihydroisoquinoline- | ppm 7.39-7.46 (m, 4H), 7.13-7.24 (m, | (M − H)⁻ |
| | 2(1H)-carboxamide | 4H), 4.61-4.63 (m, 2H), 3.82-3.89 (m, | |
| | | 4H), 3.69 (t, J = 5.8 Hz, 2H), 3.44 (t, | |
| | | J = 7.0 Hz, 2H), 3.25-3.30 (m, 4H), | |
| | | 2.80-2.89 (m, 4H) | |
| 96 | N-(4-{[3-(4-methylpiperazin- | ¹H NMR (400 MHz, DMSO-d₆/ | (ESI(+)) |
| | 1-yl)propanoyl]amino}phenyl)- | Deuterium Oxide, Temp = 90° C.) δ | m/e 422 |
| | 3,4-dihydroisoquinoline- | ppm 7.37-7.43 (m, 4H), 7.14-7.22 (m, | (M + H)⁺ |
| | 2(1H)-carboxamide | 4H), 4.61 (s, 2H), 3.68 (t, J = 6.0 Hz, | |
| | | 2H), 3.19-3.26 (m, 4H), 2.96-3.09 (m, | |
| | | 6H), 2.86 (t, J = 6.0 Hz, 2H), 2.77 (s, | |
| | | 3H), 2.61 (t, J = 6.9 Hz, 2H) | |

Some products were purified by flash chromatography while others were purified by reverse-phase HPLC; accordingly, some examples were isolated as trifluoroacetic acid salts.

### Example 34

### N-[4-(1-isobutyl-1H-pyrazol-4-yl)phenyl]-3,4-dihydroisoquinoline-2(1H)-carboxamide

### Example 34A

### N-(4-bromophenyl)-3,4-dihydroisoquinoline-2(1H)-carboxamide

The title compound was prepared as described in Example 1A, substituting 1-bromo-4-isocyanatobenzene for methyl 4-isocyanatobenzoate.

### Example 34B

### N-[4-(1-isobutyl-1H-pyrazol-4-yl)phenyl]-3,4-dihydroisoquinoline-2(1H)-carboxamide

In a 5 mL microwave vial were mixed N-(4-bromophenyl)-3,4-dihydroisoquinoline-2(1H)-carboxamide (75 mg, 0.226 mmol), 1-isobutyl-4-(4,4,5,5-tetramethyl-1,3,2-dioxaborolan-2-yl)-1H-pyrazole (61 mg, 0.25 mmol), sodium carbonate (50.4 mg, 0.476 mmol), and [1,1′-bis(diphenylphosphino)ferrocene]dichloropalladium(II) (5.55 mg, 6.79 μmol) in tetrahydrofuran (2 ml)/water (1 ml)/methanol (0.333 ml). The solution was put through three vacuum/nitrogen purge cycles; and the reaction vial was sealed and heated overnight at 80° C. The reaction mixture was diluted with ethyl acetate, and washed with water and saturated sodium chloride solution. Concentration of the combined organic layers gave a residue which was purified by normal-phase flash chromatography to provide the title compound. ¹H NMR (400 MHz, DMSO-d₆) δ ppm 8.56 (s, 1H), 8.04 (d, J=0.8 Hz, 1H), 7.79 (d, J=0.8 Hz, 1H), 7.43-7.49 (m, 4H), 7.15-7.22 (m, 4H), 4.64-4.64 (bs, 2H), 3.90 (d, J=7.2 Hz, 2H), 3.70 (t, J=5.9 Hz, 2H), 2.85 (t, J=5.8 Hz, 2H), 2.07-2.18 (m, 1H), 0.86 (d, J=6.7 Hz, 6H); (ESI(+)) m/e 375 (M+H)⁺.

### Example 35

### N-[4-(1-propyl-1H-pyrazol-4-yl)phenyl]-3,4-dihydroisoquinoline-2(1H)-carboxamide

The title compound was prepared as described in Example 34B, substituting 1-propyl-4-(4,4,5,5-tetramethyl-1,3,2-dioxaborolan-2-yl)-1H-pyrazole for 1-isobutyl-4-(4,4,5,5-tetramethyl-1,3,2-dioxaborolan-2-yl)-1H-pyrazole. ¹H NMR (400 MHz, DMSO-d₆) δ ppm 8.56 (s, 1H), 8.06 (d, J=0.8 Hz, 1H), 7.78 (d, J=0.8 Hz, 1H), 7.42-7.49 (m, 4H), 7.17-7.22 (m, 4H), 4.64-4.64 (bs, 2H), 4.02-4.07 (m, 2H), 3.70 (t, J=5.9 Hz, 2H), 2.85 (t, J=5.8 Hz, 2H), 1.76-1.85 (m, 2H), 0.85 (t, J=7.4 Hz, 3H); (ESI(+)) m/e 361 (M+H)⁺.

### Example 36

### N-{4-[1-((2R)-2-hydroxypropyl)-1H-pyrazol-4-yl]phenyl}-3,4-dihydroisoquinoline-2(1H)-carboxamide

The title compound was prepared as described in Example 34B, substituting 1-((2R)-2-hydroxypropyl)-4-(4,4,5,5-tetramethyl-1,3,2-dioxaborolan-2-yl)-1H-pyrazole for 1-isobutyl-4-(4,4,5,5-tetramethyl-1,3,2-dioxaborolan-2-yl)-1H-pyrazole. ¹H NMR (400 MHz, DMSO-d₆) δ ppm 8.55 (s, 1H), 8.01 (d, J=0.8 Hz, 1H), 7.79 (d, J=0.8 Hz, 1H), 7.42-7.49 (m, 4H), 7.17-7.23 (m, 4H), 4.92-4.94 (bs, 1H), 4.64-4.64 (bs, 2H), 3.99-4.00 (m, 3H), 3.70 (t, J=5.9 Hz, 2H), 2.85 (t, J=5.8 Hz, 2H), 1.04-1.06 (m, 3H); (ESI(−)) m/e 375 (M−H)⁻.

### Example 37

### N-{4-[1-(3-methylbutyl)-1H-pyrazol-4-yl]phenyl}-3,4-dihydroisoquinoline-2(1H)-carboxamide

The title compound was prepared as described in Example 34B, substituting 1-isopentyl-4-(4,4,5,5-tetramethyl-1,3,2-dioxaborolan-2-yl)-1H-pyrazole for 1-isobutyl-4-(4,4,5,5-tetramethyl-1,3,2-dioxaborolan-2-yl)-1H-pyrazole. ¹H NMR (400 MHz, DMSO-d₆) δ ppm 8.55 (s, 1H), 8.08 (s, 1H), 7.77 (s, 1H), 7.42-7.49 (m, 4H), 7.19 (s, 4H), 4.64-4.64 (bs, 2H), 4.11 (t, J=7.2 Hz, 2H), 3.70 (t, J=5.9 Hz, 2H), 2.85 (t, J=5.8 Hz, 2H), 1.69 (q, J=7.1 Hz, 2H), 1.43-1.55 (m, 1H), 0.91 (d, J=6.6 Hz, 6H); (ESI(+)) m/e 389 (M+H)⁺.

### Example 38

### N-[4-(1-benzyl-1H-pyrazol-4-yl)phenyl]-3,4-dihydroisoquinoline-2(1H)-carboxamide

The title compound was prepared as described in Example 34B, substituting 1-benzyl-4-(4,4,5,5-tetramethyl-1,3,2-dioxaborolan-2-yl)-1H-pyrazole for 1-isobutyl-4-(4,4,5,5-tetramethyl-1,3,2-dioxaborolan-2-yl)-1H-pyrazole. ¹H NMR (400 MHz, DMSO-d₆) δ ppm 8.56 (s, 1H), 8.17 (d, J=0.8 Hz, 1H), 7.84 (d, J=0.8 Hz, 1H), 7.43-7.49 (m, 4H), 7.32-7.38 (m, 2H), 7.24-7.31 (m, 3H), 7.19 (s, 2H), 7.19 (s, 2H), 5.32 (s, 2H), 4.63-4.64 (bs, 2H), 3.70 (t, J=5.9 Hz, 2H), 2.85 (t, J=5.8 Hz, 2H); (ESI(+)) m/e 409 (M+H)⁺.

### Example 39

### N-{4-[(1E)-5-phenylpent-1-en-1-yl]phenyl}-3,4-dihydroisoquinoline-2(1H)-carboxamide

The title compound was prepared as described in Example 34B, substituting 1(E)-4,4,5,5-tetramethyl-2-(5-phenylpent-1-enyl)-1,3,2-dioxaborolane for 1-isobutyl-4-(4,4,5,5-tetramethyl-1,3,2-dioxaborolan-2-yl)-1H-pyrazole. ¹H NMR (400 MHz, DMSO-d₆) δ ppm 8.57 (s, 1H), 7.42-7.45 (m, 2H), 7.25-7.31 (m, 4H), 7.15-7.23 (m, 7H), 6.31 (d, J=15.8 Hz, 1H), 6.18 (dt, J=15.8, 6.7 Hz, 1H), 4.63-4.63 (bs, 2H), 3.69 (t, J=5.9 Hz, 2H), 2.84 (t, J=5.8 Hz, 2H), 2.62 (t, J=7.6 Hz, 2H), 2.18 (q, J=7.1 Hz, 2H), 1.73 (p, J=7.5 Hz, 2H); (ESI(+)) m/e 397 (M+H)⁺.

### Example 40

### N-[4-(1-ethyl-1H-pyrazol-4-yl)phenyl]-3,4-dihydroisoquinoline-2(1H)-carboxamide

The title compound was prepared as described in Example 34B, substituting 1-ethyl-4-(4,4,5,5-tetramethyl-1,3,2-dioxaborolan-2-yl)-1H-pyrazole for 1-isobutyl-4-(4,4,5,5-tetramethyl-1,3,2-dioxaborolan-2-yl)-1H-pyrazole. ¹H NMR (400 MHz, DMSO-d₆) δ ppm 8.55 (s, 1H), 8.07 (s, 1H), 7.78 (d, J=0.8 Hz, 1H), 7.41-7.49 (m, 4H), 7.16-7.21 (m, 4H), 4.64-4.64 (bs, 2H), 4.13 (q, J=7.2 Hz, 2H), 3.70 (t, J=5.9 Hz, 2H), 2.85 (t, J=5.8 Hz, 2H), 1.39 (t, J=7.3 Hz, 3H); (ESI(−)) m/e 345 (M−H)⁻.

### Example 41

### N-{4-[1-(2-hydroxyethyl)-1H-pyrazol-4-yl]phenyl}-3,4-dihydroisoquinoline-2(1H)-carboxamide

The title compound was prepared as described in Example 34B, substituting 2-(4-(4,4,5,5-tetramethyl-1,3,2-dioxaborolan-2-yl)-1H-pyrazol-1-yl)ethanol for 1-isobutyl-4-(4,4,5,5-tetramethyl-1,3,2-dioxaborolan-2-yl)-1H-pyrazole. ¹H NMR (400 MHz, DMSO-d₆) δ ppm 8.55 (s, 1H), 8.03 (d, J=0.8 Hz, 1H), 7.79 (d, J=0.8 Hz, 1H), 7.42-7.49 (m, 4H), 7.14-7.23 (m, 4H), 4.91 (t, J=5.3 Hz, 1H), 4.64-4.64 (bs, 2H), 4.13 (t, J=5.6 Hz, 2H), 3.75 (q, J=5.5 Hz, 2H), 3.70 (t, J=6.0 Hz, 2H), 2.85 (t, J=5.8 Hz, 2H); (ESI(+)) m/e 363 (M+H)⁺.

### Example 42

### N-[4-(1-methyl-1H-pyrazol-4-yl)phenyl]-3,4-dihydroisoquinoline-2(1H)-carboxamide

The title compound was prepared as described in Example 34B, substituting 1-methyl-4-(4,4,5,5-tetramethyl-1,3,2-dioxaborolan-2-yl)-1H-pyrazole for 1-isobutyl-4-(4,4,5,5-tetramethyl-1,3,2-dioxaborolan-2-yl)-1H-pyrazole. ¹H NMR (400 MHz, DMSO-d₆) δ ppm 8.55 (s, 1H), 8.02 (s, 1H), 7.77 (s, 1H), 7.46-7.48 (m, 2H), 7.41-7.44 (m, 2H), 7.15-7.23 (m, 4H), 4.63-4.64 (bs, 2H), 3.84 (s, 3H), 3.70 (t, J=5.8 Hz, 2H), 2.85 (t, J=5.8 Hz, 2H); (ESI(−)) m/e 331 (M−H)⁻.

### Example 43

### N-[4-(1-benzoyl-1,2,3,6-tetrahydropyridin-4-yl)phenyl]-3,4-dihydroisoquinoline-2(1H)-carboxamide

In a 4 mL vial was mixed N-(4-(1,2,3,6-tetrahydropyridin-4-yl)phenyl)-3,4-dihydroisoquinoline-2(1H)-carboxamide (30 mg, 0.090 mmol) in anhydrous tetrahydrofuran (1 ml). Triethylamine (0.025 ml, 0.180 mmol) and benzoyl chloride (13 mg, 0.090 mmol) were added and the reaction mixture was stirred overnight at room temperature. The mixture was diluted with ethyl acetate and washed with water; the separated organic layer was concentrated and the title compound was isolated by normal-phase flash chromatography. ¹H NMR (400 MHz, DMSO-d₆) δ ppm 8.61 (s, 1H), 7.26-7.56 (m, 10H), 7.09-7.25 (m, 4H), 5.95-6.25 (m, 1H), 4.63-4.64 (bs, 2H), 3.95-4.35 (m, 2H), 3.80-3.87 (m, 1H), 3.70 (t, J=5.8 Hz, 2H), 3.44-3.57 (m, 2H), 2.85 (t, J=5.8 Hz, 2H); (ESI(+)) m/e 438 (M+H)⁺.

### Example 44

### N-[4-(1-butyryl-1,2,3,6-tetrahydropyridin-4-yl)phenyl]-3,4-dihydroisoquinoline-2(1H)-carboxamide

The title compound was prepared as described in Example 43, substituting butyryl chloride for benzoyl chloride. ¹H NMR (400 MHz, DMSO-d₆) δ ppm 8.60 (s, 1H), 7.46-7.49 (m, 2H), 7.30-7.36 (m, 2H), 7.14-7.24 (m, 4H), 6.07-6.10 (m, 1H), 4.63-4.64 (bs, 2H), 4.07-4.13 (m, 2H), 3.70 (t, J=5.9 Hz, 2H), 3.64 (dd, J=12.3, 6.2 Hz, 2H), 2.85 (t, J=5.8 Hz, 2H), 2.39-2.53 (m, 2H), 2.28-2.38 (m, 2H), 1.45-1.62 (m, 2H), 0.87-0.93 (m, 3H); (ESI(+)) m/e 404 (M+H)⁺.

### Example 45

### N-{4-[1-(isopropylsulfonyl)-1,2,3,6-tetrahydropyridin-4-yl]phenyl}-3,4-dihydroisoquinoline-2(1H)-carboxamide

The title compound was prepared as described in Example 43, substituting propane-2-sulfonyl chloride for benzoyl chloride. ¹H NMR (400 MHz, DMSO-d₆) δ ppm 8.61 (s, 1H), 7.47-7.50 (m, 2H), 7.32-7.35 (m, 2H), 7.15-7.23 (m, 4H), 6.10-6.12 (m, 1H), 4.63-4.64 (bs, 2H), 3.94-3.96 (m, 2H), 3.70 (t, J=5.8 Hz, 2H), 3.49 (t, J=5.6 Hz, 2H), 3.34-3.44 (m, 1H), 2.85 (t, J=5.8 Hz, 2H), 2.48-2.55 (m, 2H), 1.24 (d, J=6.8 Hz, 6H); (ESI(−)) m/e 438 (M−H)⁻.

### Example 46

### N-[4-(1-isobutyryl-1,2,3,6-tetrahydropyridin-4-yl)phenyl]-3,4-dihydroisoquinoline-2(1H)-carboxamide

The title compound was prepared as described in Example 43, substituting isobutyryl chloride for benzoyl chloride. ¹H NMR (400 MHz, DMSO-d₆) δ ppm 8.60 (s, 1H), 7.46-7.49 (m, 2H), 7.32-7.35 (m, 2H), 7.10-7.26 (m, 4H), 6.09-6.11 (bs, 1H), 4.63-4.64 (bs, 2H), 4.01-4.24 (m, 2H), 3.66-3.71 (m, 4H), 2.91-3.06 (m, 1H), 2.85 (t, J=5.8 Hz, 2H), 2.35-2.55 (m, 2H), 0.99-1.04 (m, 6H); (ESI(+)) m/e 404 (M+H)⁺.

### Example 47

### N-{4-[1-(3-methylbutanoyl)-1,2,3,6-tetrahydropyridin-4-yl]phenyl}-3,4-dihydroisoquinoline-2(1H)-carboxamide

The title compound was prepared as described in Example 43, substituting isopentyl chloride for benzoyl chloride. ¹H NMR (400 MHz, DMSO-d₆) δ ppm 8.60 (s, 1H), 7.44-7.51 (m, 2H), 7.30-7.36 (m, 2H), 7.18 (s, 4H), 6.07-6.11 (m, 1H), 4.63-4.64 (bs, 2H), 4.08-4.14 (m, 2H), 3.70 (t, J=6.0 Hz, 2H), 3.62-3.71 (m, 2H), 2.85 (t, J=5.8 Hz, 2H), 2.38-2.53 (m, 2H), 2.24 (dd, J=18.6, 6.9 Hz, 2H), 1.96-2.09 (m, 1H), 0.91 (t, J=6.2 Hz, 6H); (ESI(+)) m/e 418 (M+H)⁺.

### Example 48

### N-{4-[1-(methylcarbamoyl)-1,2,3,6-tetrahydropyridin-4-yl]phenyl}-3,4-dihydroisoquinoline-2(1H)-carboxamide

The title compound was prepared as described in Example 1A, substituting methyl isocyanate for methyl 4-isocyanatobenzoate and N-(4-(1,2,3,6-tetrahydropyridin-4-yl)phenyl)-3,4-dihydroisoquinoline-2(1H)-carboxamide for 1,2,3,4-tetrahydroisoquinoline. ¹H NMR (400 MHz, DMSO-d₆) δ ppm 8.59 (s, 1H), 7.46-7.48 (m, 2H), 7.32-7.34 (m, 2H), 7.18 (s, 4H), 6.42 (q, J=4.3 Hz, 1H), 6.07-6.09 (m, 1H), 4.63-4.64 (bs, 2H), 3.92-3.94 (m, 2H), 3.70 (t, J=5.8 Hz, 2H), 3.49 (t, J=5.6 Hz, 2H), 2.85 (t, J=5.8 Hz, 2H), 2.57-2.61 (m, 3H), 2.37-2.43 (m, 2H); (ESI(+)) m/e 391 (M+H)⁺.

### Example 49

### tert-butyl 4-{4-[(3,4-dihydroisoquinolin-2(1H)-ylcarbonyl)amino]phenyl}-3,6-dihydropyridine-1(2H)-carboxylate

The title compound was prepared as described in Example 34B, substituting tert-butyl 4-(4,4,5,5-tetramethyl-1,3,2-dioxaborolan-2-yl)-5,6-dihydropyridine-1(2H)-carboxylate for 1-isobutyl-4-(4,4,5,5-tetramethyl-1,3,2-dioxaborolan-2-yl)-1H-pyrazole. ¹H NMR (400 MHz, DMSO-d₆) δ ppm 8.60 (s, 1H), 7.46-7.49 (m, 2H), 7.31-7.34 (m, 2H), 7.17-7.20 (m, 1H), 7.18 (s, 3H), 6.05-6.08 (bs, 1H), 4.63-4.64 (bs, 2H), 3.93-3.98 (m, 2H), 3.70 (t, J=5.8 Hz, 2H), 3.50-3.54 (m, 2H), 2.85 (t, J=5.8 Hz, 2H), 2.40-2.45 (m, 2H), 1.42 (s, 9H); (ESI(−)) m/e 432 (M−H)⁻.

### Example 50

### N-[4-(1-acetyl-1,2,3,6-tetrahydropyridin-4-yl)phenyl]-3,4-dihydroisoquinoline-2(1H)-carboxamide

The title compound was prepared as described in Example 43, substituting acetyl chloride for benzoyl chloride. ¹H NMR (500 MHz, DMSO-d₆) δ ppm 8.60 (s, 1H), 7.46-7.49 (m, 2H), 7.31-7.35 (m, 2H), 7.19 (s, 4H), 6.07-6.10 (m, 1H), 4.63-4.64 (bs, 2H), 4.04-4.15 (m, 2H), 3.70 (t, J=5.9 Hz, 2H), 3.60-3.66 (m, 2H), 2.85 (t, J=5.8 Hz, 2H), 2.37-2.54 (m, 2H), 2.02-2.07 (m, 3H); (ESI(+)) m/e 376 (M+H)⁺.

### Example 51

### N-{4-[1-(isobutylsulfonyl)-1,2,3,6-tetrahydropyridin-4-yl]phenyl}-3,4-dihydroisoquinoline-2(1H)-carboxamide

The title compound was prepared as described in Example 43, substituting 2-methylpropane-1-sulfonyl chloride for benzoyl chloride. ¹H NMR (400 MHz, DMSO-d₆) δ ppm 8.62 (s, 1H), 7.47-7.50 (m, 2H), 7.33-7.36 (m, 2H), 7.19 (s, 4H), 6.10-6.13 (m, 1H), 4.63-4.64 (bs, 2H), 3.86-3.88 (m, 2H), 3.70 (t, J=5.8 Hz, 2H), 3.40 (t, J=5.7 Hz, 2H), 2.96 (d, J=6.5 Hz, 2H), 2.85 (t, J=5.8 Hz, 2H), 2.47-2.58 (m, 2H), 2.07-2.20 (m, 1H), 1.04 (d, J=6.7 Hz, 6H); (ESI(−)) m/e 452 (M−H)⁻.

### Example 52

### N-[4-(1-benzyl-1,2,3,6-tetrahydropyridin-4-yl)phenyl]-3,4-dihydroisoquinoline-2(1H)-carboxamide

In a 4 mL vial were mixed N-(4-(1,2,3,6-tetrahydropyridin-4-yl)phenyl)-3,4-dihydroisoquinoline-2(1H)-carboxamide (30 mg, 0.090 mmol) and benzaldehyde (9.12 μl, 0.090 mmol) in anhydrous dichloroethane (1 ml) at room temperature. Sodium triacetoxyborohydride (26.7 mg, 0.126 mmol) was added and the mixture was stirred overnight at room temperature. The reaction mixture was diluted with ethyl acetate and washed with saturated aqueous sodium bicarbonate and aqueous sodium chloride solutions. The organic layer was dried with sodium sulfate, filtered and concentrated to give the title compound after normal-phase flash chromatography. ¹H NMR (400 MHz, DMSO-d₆) δ ppm 8.58 (s, 1H), 7.44-7.46 (m, 2H), 7.22-7.38 (m, 7H), 7.18 (s, 4H), 6.02-6.08 (m, 1H), 4.62-4.64 (bs, 2H), 3.69 (t, J=6.0 Hz, 2H), 3.57 (s, 2H), 3.02-3.06 (m, 2H), 2.84 (t, J=5.9 Hz, 2H), 2.62 (t, J=5.6 Hz, 2H), 2.41-2.47 (m, 2H); (ESI(−)) m/e 422 (M−H)⁻.

### Example 53

### N-{4-[1-(methylsulfonyl)-1,2,3,6-tetrahydropyridin-4-yl]phenyl}-3,4-dihydroisoquinoline-2(1H)-carboxamide

The title compound was prepared as described in Example 43, substituting methanesulfonyl chloride for benzoyl chloride. ¹H NMR (400 MHz, DMSO-d₆) δ ppm 8.61 (s, 1H), 7.47-7.50 (m, 2H), 7.33-7.36 (m, 2H), 7.19 (s, 4H), 6.10-6.13 (m, 1H), 4.63-4.64 (bs, 2H), 3.83-3.85 (m, 2H), 3.70 (t, J=5.9 Hz, 2H), 3.36 (t, J=5.7 Hz, 2H), 2.92 (s, 3H), 2.85 (t, J=5.8 Hz, 2H), 2.54-2.60 (m, 2H); (ESI(+)) m/e 412 (M+H)⁺.

### Example 54

### N-[4-(1,2,3,6-tetrahydropyridin-4-yl)phenyl]-3,4-dihydroisoquinoline-2(1H)-carboxamide

In a 5 mL round bottom flask was mixed tert-butyl 4-{4-[(3,4-dihydroisoquinolin-2(1H)-ylcarbonyl)amino]phenyl}-3,6-dihydropyridine-1(2H)-carboxylate (461 mg, 1.063 mmol) in dichloromethane (5 ml). Trifluoroacetic acid (1 ml, 12.98 mmol) was added at room temperature and the mixture was stirred for 3 hours. The solution was concentrated, the residue was quenched with saturated sodium bicarbonate and the product was extracted with dichloromethane. The organic layer was dried with sodium sulfate, filtered and concentrated to give the title compound. ¹H NMR (400 MHz, DMSO-d₆) δ ppm 8.58 (s, 1H), 7.43-7.46 (m, 2H), 7.28-7.31 (m, 2H), 7.16-7.21 (m, 4H), 6.05-6.12 (m, 1H), 4.63 (s, 2H), 3.86-4.14 (m, 1H), 3.69 (t, J=5.9 Hz, 2H), 3.28 (d, J=339.1 Hz, 3H), 2.89 (t, J=5.6 Hz, 1H), 2.85 (t, J=5.9 Hz, 2H), 2.25-2.34 (m, 2H); (ESI(+)) m/e 334 (M+H)⁺.

### Example 55

### N-{4-[1-(3-methylbutyl)-1,2,3,6-tetrahydropyridin-4-yl]phenyl}-3,4-dihydroisoquinoline-2(1H)-carboxamide

The title compound was prepared as described in Example 52, substituting 4-methylpentanal for benzaldehyde. ¹H NMR (400 MHz, DMSO-d₆) δ ppm 8.57 (s, 1H), 7.44-7.46 (m, 2H), 7.29-7.32 (m, 2H), 7.18-7.20 (m, 4H), 6.05-6.07 (m, 1H), 4.63-4.64 (bs, 2H), 3.69 (t, J=5.9 Hz, 2H), 3.02-3.04 (m, 2H), 2.85 (t, J=5.8 Hz, 2H), 2.58 (t, J=5.6 Hz, 2H), 2.40-2.45 (m, 2H), 2.35-2.40 (m, 2H), 1.55-1.66 (m, 1H), 1.33-1.41 (m, 2H), 0.89 (d, J=6.6 Hz, 6H); (ESI(−)) m/e 402 (M−H)⁻.

### Example 56

### N-[4-(5-propyl-1,2,4-oxadiazol-3-yl)phenyl]-3,4-dihydroisoquinoline-2(1H)-carboxamide

### Example 56A

### N-(4-cyanophenyl)-3,4-dihydroisoquinoline-2(1H)-carboxamide

The title compound was prepared as described in Example 1A, substituting 4-isocyanatobenzonitrile for methyl 4-isocyanatobenzoate.

### Example 56B

### (Z)—N-(4-(N′-hydroxycarbamimidoyl)phenyl)-3,4-dihydroisoquinoline-2(1H)-carboxamide

In a 25 mL pressure tube were mixed N-(4-cyanophenyl)-3,4-dihydroisoquinoline-2(1H)-carboxamide (500 mg, 1.803 mmol), hydroxylamine hydrochloride (251 mg, 3.61 mmol), and triethylamine (1.26 ml, 9.01 mmol) in ethanol (6 ml)/water (0.5 ml). The reaction vessel was sealed and heated at 80° C. for about 3 hours. The solution was diluted with water and dichloromethane and the separated organic layer was dried with sodium sulfate, filtered and concentrated. Normal-phase flash chromatography of the residue provided the title compound.

### Example 56C

### N-[4-(5-propyl-1,2,4-oxadiazol-3-yl)phenyl]-3,4-dihydroisoquinoline-2(1H)-carboxamide

In a 4 mL vial were mixed (Z)—N-(4-(N′-hydroxycarbamimidoyl)phenyl)-3,4-dihydroisoquinoline-2(1H)-carboxamide (75 mg, 0.242 mmol), butyric acid (23 mg, 0.266 mmol), 1-hydroxybenzotriazole (18.50 mg, 0.121 mmol) and N-methylmorpholine (0.093 ml, 0.846 mmol) in anhydrous N,N-dimethylformamide (2 ml). To this solution was added N-(3-dimethylaminopropyl)-N′-ethylcarbodiimide hydrochloride (69.5 mg, 0.362 mmol) and the reaction mixture was stirred overnight at room temperature. The resulting solution was diluted with ethyl acetate and then washed with water, saturated aqueous sodium bicarbonate and aqueous sodium chloride solutions. The organic layer was dried with sodium sulfate, filtered and concentrated. The residue was dissolved in anhydrous toluene (2 ml) and heated at 110° C. for 5 hours; concentration and normal-phase flash chromatography gave the title compound. ¹H NMR (500 MHz, DMSO-d₆) δ ppm 8.90 (s, 1H), 7.87-7.89 (m, 2H), 7.68-7.70 (m, 2H), 7.17-7.25 (m, 4H), 4.66-4.67 (bs, 2H), 3.73 (t, J=5.9 Hz, 2H), 2.95 (t, J=7.4 Hz, 2H), 2.87 (t, J=5.9 Hz, 2H), 1.77-1.85 (m, 2H), 0.98 (t, J=7.4 Hz, 3H); (ESI(−)) m/e 361 (M−H)⁻.

### Example 57

### N-[4-(5-benzyl-1,2,4-oxadiazol-3-yl)phenyl]-3,4-dihydroisoquinoline-2(1H)-carboxamide

The title compound was prepared as described in Example 56C, substituting phenylacetic acid for butyric acid in Example 56C. ¹H NMR (500 MHz, DMSO-d₆) δ ppm 8.90 (s, 1H), 7.85-7.87 (m, 2H), 7.68-7.70 (m, 2H), 7.36-7.39 (m, 4H), 7.29-7.35 (m, 1H), 7.16-7.22 (m, 4H), 4.66-4.66 (bs, 2H), 4.41 (s, 2H), 3.72 (t, J=5.9 Hz, 2H), 2.86 (t, J=5.9 Hz, 2H); (ESI(−)) m/e 409 (M−H)⁻.

### Example 58

### N-{4-[5-(3-methylbutyl)-1,2,4-oxadiazol-3-yl]phenyl}-3,4-dihydroisoquinoline-2(1H)-carboxamide

The title compound was prepared as described in Example 56C, substituting 4-methylpentanoic acid for butyric acid. ¹H NMR (500 MHz, DMSO-d₆) δ ppm 8.90 (s, 1H), 7.87-7.89 (m, 2H), 7.68-7.71 (m, 2H), 7.19 (s, 3H), 7.19 (s, 1H), 4.66-4.67 (bs, 2H), 3.73 (t, J=5.9 Hz, 2H), 2.97 (t, J=7.7 Hz, 2H), 2.87 (t, J=5.9 Hz, 2H), 1.66-1.71 (m, 2H), 1.59-1.67 (m, 1H), 0.93 (d, J=6.4 Hz, 6H); (ESI(−)) m/e 389 (M−H)⁻.

### Example 59

### N-hexyl-3,4-dihydroisoquinoline-2(1H)-carboxamide

The title compound was prepared as described in Example 1A, substituting 1-isocyanatohexane for methyl 4-isocyanatobenzoate. ¹H NMR (400 MHz, DMSO-d₆) δ ppm 7.10-7.16 (m, 4H), 6.49 (t, J=5.4 Hz, 1H), 4.46-4.46 (bs, 2H), 3.52 (t, J=5.9 Hz, 2H), 3.00-3.05 (m, 2H), 2.75 (t, J=5.9 Hz, 2H), 1.36-1.43 (m, 2H), 1.24-1.28 (m, 6H), 0.83-0.88 (m, 3H); (ESI(+)) m/e 334 (M+H)⁺.

### Example 60

### ethyl 6-[(3,4-dihydroisoquinolin-2(1H)-ylcarbonyl)amino]hexanoate

The title compound was prepared as described in Example 1A, substituting ethyl 6-isocyanatohexanoate for methyl 4-isocyanatobenzoate. ¹H NMR (400 MHz, DMSO-d₆) δ ppm 7.10-7.17 (m, 4H), 6.51 (t, J=5.5 Hz, 1H), 4.46-4.46 (bs, 2H), 4.03 (q, J=7.1 Hz, 2H), 3.52 (t, J=5.9 Hz, 2H), 3.00-3.05 (m, 2H), 2.75 (t, J=5.9 Hz, 2H), 2.26 (t, J=7.4 Hz, 2H), 1.52 (p, J=7.5 Hz, 2H), 1.41 (p, J=7.3 Hz, 2H), 1.20-1.29 (m, 2H), 1.16 (t, J=7.1 Hz, 3H); (ESI(+)) m/e 319 (M+H)⁺.

### Example 61

### N-(4-{2-[(phenylacetyl)amino]ethyl}phenyl)-3,4-dihydroisoquinoline-2(1H)-carboxamide

### Example 61A

### N-(4-(cyanomethyl)phenyl)-3,4-dihydroisoquinoline-2(1H)-carboxamide

The title compound was prepared as described in Example 1A, substituting 2-(4-isocyanatophenyl)acetonitrile for methyl 4-isocyanatobenzoate.

### Example 61B

### N-(4-(2-aminoethyl)phenyl)-3,4-dihydroisoquinoline-2(1H)-carboxamide

N-(4-(cyanomethyl)phenyl)-3,4-dihydroisoquinoline-2(1H)-carboxamide (400 mg, 1.373 mmol) and methanol/7M ammonia-methanol (5 ml) were added to Ra—Ni 2800, water slurry (800 mg, 13.63 mmol) in a 50 ml pressure bottle and stirred for 18 hours at 30 psi and room temperature. The mixture was filtered through a nylon membrane and concentrated. The residue was purified by normal-phase flash chromatography to give the title compound.

### Example 61C

### N-(4-{2-[(phenylacetyl)amino]ethyl}phenyl)-3,4-dihydroisoquinoline-2(1H)-carboxamide

The title compound was prepared as described in Example 1C, substituting N-(4-(2-aminoethyl)phenyl)-3,4-dihydroisoquinoline-2(1H)-carboxamide for 3-methylbutan-1-amine and phenylacetic acid for 4-(1,2,3,4-tetrahydroisoquinoline-2-carboxamido)benzoic acid. ¹H NMR (400 MHz, DMSO-d₆) δ ppm 8.49 (s, 1H), 8.06 (t, J=5.5 Hz, 1H), 7.36-7.39 (m, 2H), 7.26-7.30 (m, 2H), 7.18-7.23 (m, 7H), 7.01-7.04 (m, 2H), 4.62-4.63 (bs, 2H), 3.69 (t, J=5.8 Hz, 2H), 3.35-3.38 (m, 2H), 3.17-3.27 (m, 2H), 2.85 (t, J=5.8 Hz, 2H), 2.63 (t, J=7.2 Hz, 2H); (ESI(+)) m/e 414 (M+H)⁺.

### Example 62

### N-{4-[2-(isobutyrylamino)ethyl]phenyl}-3,4-dihydroisoquinoline-2(1H)-carboxamide

The title compound was prepared as described in Example 1C, substituting N-(4-(2-aminoethyl)phenyl)-3,4-dihydroisoquinoline-2(1H)-carboxamide for 3-methylbutan-1-amine and isobutyric acid for 4-(1,2,3,4-tetrahydroisoquinoline-2-carboxamido)benzoic acid. ¹H NMR (400 MHz, DMSO-d₆) δ ppm 8.48 (s, 1H), 7.72-7.76 (m, 1H), 7.36-7.40 (m, 2H), 7.18 (s, 4H), 7.04-7.07 (m, 2H), 4.62-4.63 (bs, 2H), 3.68 (t, J=5.8 Hz, 2H), 3.18-3.24 (m, 2H), 2.84 (t, J=5.8 Hz, 2H), 2.62 (t, J=7.3 Hz, 2H), 2.31 (p, J=6.8 Hz, 1H), 0.97 (d, J=6.8 Hz, 6H); ESI(+)) m/e 366 (M+H)⁺.

### Example 97

### N-[4-(trifluoromethyl)phenyl]-3,4-dihydroisoquinoline-2(1H)-carboxamide

The title compound was prepared as described in Example 1A, substituting 1-isocyanato-4-(trifluoromethyl)benzene for methyl 4-isocyanatobenzoate. ¹H NMR (400 MHz, DMSO-d₆) δ ppm 8.96 (s, 1H) 7.72 (d, J=8.5 Hz, 2H) 7.59 (d, J=8.5 Hz, 2H) 7.17-7.22 (m, 4H) 4.66 (s, 2H) 3.72 (t, J=6.0 Hz, 2H) 2.86 (t, J=5.8 Hz, 2H); ESI(+)) m/e 321 (M+H)⁺.

### Example 98

### N-{4-[(cyclopentylacetyl)amino]phenyl}-5-[(methylsulfonyl)amino]-3,4-dihydroisoquinoline-2(1H)-carboxamide

### Example 98A

### 2-cyclopentyl-N-(4-nitrophenyl)acetamide

The title compound was prepared as described in Example 43, substituting 4-nitroaniline for N-(4-(1,2,3,6-tetrahydropyridin-4-yl)phenyl)-3,4-dihydroisoquinoline-2(1H)-carboxamide and 2-cyclopentylacetyl chloride for benzoyl chloride.

### Example 98B

### N-(4-aminophenyl)-2-cyclopentylacetamide

The title compound was prepared as described in Example 31B, substituting 2-cyclopentyl-N-(4-nitrophenyl)acetamide for N-(4-nitrophenyl)-3,4-dihydroisoquinoline-2(1H)-carboxamide.

### Example 98C

### N-{4-[(cyclopentylacetyl)amino]phenyl}-5-[(methylsulfonyl)amino]-3,4-dihydroisoquinoline-2(1H)-carboxamide

In a 25 mL round bottom flask were mixed N-(4-aminophenyl)-2-cyclopentylacetamide (51 mg, 0.234 mmol), bis(2,5-dioxopyrrolidin-1-yl) carbonate (74.8 mg, 0.292 mmol), and anhydrous pyridine (0.019 ml, 0.234 mmol) in anhydrous acetonitrile (1 ml). The mixture stirred for 1 hour at room temperature when diisopropylethylamine (0.122 ml, 0.701 mmol) was added followed by dropwise addition of a mixture of N-(1,2,3,4-tetrahydroisoquinolin-5-yl)methanesulfonamide (60.8 mg, 0.269 mmol) in anhydrous N-methylpyrollidine (3 mL). The reaction mixture was stirred overnight at room temperature and then diluted with water. The resulting suspension was filtered with water washes to give the title compound after vacuum drying. ¹H NMR (400 MHz, DMSO-d₆) δ ppm 9.70 (d, J=9.9 Hz, 1H), 9.09 (s, 1H), 8.49 (d, J=7.8 Hz, 1H), 7.46 (m, 2H), 7.35 (m, 2H), 7.21 (m, 2H), 7.11 (m, 1H), 4.62 (s, 2H), 3.66 (t, J=5.9 Hz, 2H), 2.99 (s, 3H), 2.87 (m, 2H), 2.23 (m, 3H), 1.74 (m, 2H), 1.60 (m, 2H), 1.51 (m, 2H), 1.17 (m, 2H); (ESI(+)) m/e 471 (M+H)⁺.

What is claimed is:

1. A compound of Formula (I), or pharmaceutically acceptable salts thereof 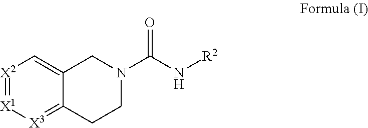
wherein
X¹ and X² and X³ are CH; or
X¹ and X³ are CH; and X² is N; or
X² and X³ are H; and X¹ is N; or
X¹ is CR¹; and X² and X³ are CH; or
X² is CR¹; and X¹ and X³ are CH; or
X³ is CR¹; and X¹ and X² are CH;
R¹ is R³, OR³, SR³, S(O)R³, SO₂R³, C(O)R³, C(O)OR³, OC(O)R³, NHR³, N(R³)₂, C(O)NH₂, C(O)NHR³, C(O)N(R³)₂, NHC(O)R³, NR³C(O)R³, NHC(O)OR³, NR³C(O)OR³, SO₂NH₂, SO₂NHR³, SO₂N(R³)₂, NHSO₂R³, NR³SO₂R³, NHSO₂NHR³, NHSO₂N(R³)₂, NR³SO₂NHR³, NR³SO₂N(R³)₂, C(O)NHSO₂R³, NHSO₂NHR³, F, Cl, Br, I, CN, NH₂, NO₂, N₃, OH, C(O)H, CF₃, C(O)OH, or C(O)NH₂;
R² is alkyl, alkenyl, alkynyl, phenyl, heterocyclyl, cycloalkyl, or cycloalkenyl; wherein each alkyl, alkenyl, and alkynyl is optionally substituted with one or more independently selected R⁴, OR⁴, SR⁴, S(O)R⁴, SO₂R⁴, C(O)R⁴, CO(O)R⁴, OC(O)R⁴, OC(O)OR⁴, NH₂, NHR⁴, N(R⁴)₂, NHC(O)R⁴, NR⁴C(O)R⁴, NHS(O)₂R⁴, NR⁴S(O)₂R⁴, NHC(O)OR⁴, NR⁴C(O)OR⁴, NHC(O)NH₂, NHC(O)NHR⁴, NHC(O)N(R⁴)₂, NR⁴C(O)NHR⁴, NR⁴C(O)N(R⁴)₂, C(O)NH₂, C(O)NHR⁴, C(O)N(R⁴)₂, C(O)NHOH, C(O)NHOR⁴, C(O)NHSO₂R⁴, C(O)NR⁴SO₂R⁴, SO₂NH₂, SO₂NHR⁴, SO₂N(R⁴)₂, C(O)H, C(O)OH, OH, (O), CN, N₃, NO₂, CF₃, CF₂CF₃, OCF₃, OCF₂CF₃, F, Cl, Br or I; wherein each phenyl is optionally additionally substituted at the para position with one independently selected R⁵, OCH₂CH₂CH₂CH₂CH₂CH₃, SR⁵, S(O)R⁵, SO₂R⁵, C(O)R⁵, CO(O)R⁵, OC(O)R⁵, OC(O)OR⁵, NH₂, NHR⁵, N(R⁵)₂, NHC(O)R⁵, NR⁵C(O)R⁵, NHS(O)₂R⁵, NR⁵S(O)₂R⁵, NHC(O)OR⁵, NR⁵C(O)OR⁵, NHC(O)NH₂, NHC(O)NHR⁵, NHC(O)N(R⁵)₂, NR⁵C(O)NHR⁵, NR⁵C(O)N(R⁵)₂, C(O)NH₂, C(O)NHR⁵, C(O)N(R⁵)₂, C(O)NHOH, C(O)NHOR⁵, C(O)NHSO₂R⁵, C(O)NR⁵SO₂R⁵, SO₂NH₂, SO₂NHR⁵, SO₂N(R⁵)₂, C(O)H, C(O)OH, OH, CN, N₃, NO₂, CF₃, CF₂CF₃, OCF₃, OCF₂CF₃, Br or I; wherein each phenyl is optionally additionally substituted with one F; wherein each heterocyclyl, cycloalkyl, and cycloalkenyl is optionally substituted with one or more independently selected R⁵, OR⁵, SR⁵, S(O)R⁵, SO₂R⁵, C(O)R⁵, CO(O)R⁵, OC(O)R⁵, OC(O)OR⁵, NH₂, NHR⁵, N(R⁵)₂, NHC(O)R⁵, NR⁵C(O)R⁵, NHS(O)₂R⁵, NR⁵S(O)₂R⁵, NHC(O)OR⁵, NR⁵C(O)OR⁵, NHC(O)NH₂, NHC(O)NHR⁵, NHC(O)N(R⁵)₂, NR⁵C(O)NHR⁵, NR⁵C(O)N(R⁵)₂, C(O)NH₂, C(O)NHR⁵, C(O)N(R⁵)₂, C(O)NHOH, C(O)NHOR⁵, C(O)NHSO₂R⁵, C(O)NR⁵SO₂R⁵, SO₂NH₂, SO₂NHR⁵, SO₂N(R⁵)₂, C(O)H, C(O)OH, OH, CN, N₃, CF₃, CF₂CF₃, OCF₃, OCF₂CF₃, F, Cl, Br or I; wherein R² is not 4-methylphenyl;
R³ is alkyl, alkenyl, alkynyl, aryl, or heterocyclyl; wherein each alkyl, alkenyl, and alkynyl is optionally substituted with one or more independently selected R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, OH, (O), CN, N₃, NO₂, CF₃, CF₂CF₃, OCF₃, OCF₂CF₃, F, Cl, Br or I;
R⁴ is alkyl, alkenyl, alkynyl, aryl or heterocyclyl; wherein each alkyl, alkenyl, and alkynyl is optionally substituted with one or more independently selected R⁷, OR⁷, SR⁷, S(O)R⁷, SO₂R⁷, C(O)R⁷, CO(O)R⁷, OC(O)R⁷, OC(O)OR⁷, NH₂, NHR⁷, N(R⁷)₂, NHC(O)R⁷, NR⁷C(O)R⁷, NHS(O)₂R⁷, NR⁷S(O)₂R⁷, NHC(O)OR⁷, NR⁷C(O)OR⁷, NHC(O)NH₂, NHC(O)NHR⁷, NHC(O)N(R⁷)₂, NR⁷C(O)NHR⁷, NR⁷C(O)N(R⁷)₂, C(O)NH₂, C(O)NHR⁷, C(O)N(R⁷)₂, C(O)NHOH, C(O)NHOR⁷, C(O)NHSO₂R⁷, C(O)NR⁷SO₂R⁷, SO₂NH₂, SO₂NHR⁷, SO₂N(R⁷)₂, C(O)H, C(O)OH, OH, (O), CN, N₃, NO₂, CF₃, CF₂CF₃, OCF₃, OCF₂CF₃, F, Cl, Br or I; wherein each aryl and heterocycyl is optionally substituted with one or more independently selected R⁸, OR⁸, SR⁸, S(O)R⁸, SO₂R⁸, C(O)R⁸, CO(O)R⁸, OC(O)R⁸, OC(O)OR⁸, NH₂, NHR⁸, N(R⁸)₂, NHC(O)R⁸, NR⁸C(O)R⁸, NHS(O)₂R⁸, NR⁸S(O)₂R⁸, NHC(O)OR⁸, NR⁸C(O)OR⁸, NHC(O)NH₂, NHC(O)NHR⁸, NHC(O)N(R⁸)₂, NR⁸C(O)NHR⁸, NR⁸C(O)N(R⁸)₂, C(O)NH₂, C(O)NHR⁸, C(O)N(R⁸)₂, C(O)NHOH, C(O)NHOR⁸, C(O)NHSO₂R⁸, C(O)NR⁸SO₂R⁸, SO₂NH₂, SO₂NHR⁸, SO₂N(R⁸)₂, C(O)H, C(O)OH, OH, CN, N₃, NO₂, CF₃, CF₂CF₃, OCF₃, OCF₂CF₃, F, Cl, Br or I;
R⁵ is alkyl, alkenyl, alkynyl, aryl, heterocyclyl, cycloalkyl, or cycloalkenyl; wherein each alkyl, alkenyl, and alkynyl is optionally substituted with one or more independently selected R⁹, OR⁹, SR⁹, S(O)R⁹, SO₂R⁹, C(O)R⁹, CO(O)R⁹, OC(O)R⁹, OC(O)OR⁹, NH₂, NHR⁹, N(R⁹)₂, NHC(O)R⁹, NR⁹C(O)R⁹, NHS(O)₂R⁹, NR⁹S(O)₂R⁹, NHC(O)OR⁹, NR⁹C(O)OR⁹, NHC(O)NH₂, NHC(O)NHR⁹, NHC(O)N(R⁹)₂, NR⁹C(O)NHR⁹, NR⁹C(O)N(R⁹)₂, C(O)NH₂, C(O)NHR⁹, C(O)N(R⁹)₂, C(O)NHOH, C(O)NHOR⁹, C(O)NHSO₂R⁹, C(O)NR⁹SO₂R⁹, SO₂NH₂, SO₂NHR⁹, SO₂N(R⁹)₂, C(O)H, C(O)OH, OH, (O), CN, N₃, NO₂, CF₃, CF₂CF₃, OCF₃, OCF₂CF₃, F, Cl, Br or I;
R⁶ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁰, OR¹⁰, SR¹⁰, S(O)R¹⁰, SO₂R¹⁰, NHR¹⁰, N(R¹⁰)₂, C(O)R¹⁰, C(O)NH₂, C(O)NHR¹⁰, C(O)N(R¹⁰)₂, NHC(O)R¹⁰, NR¹⁰C(O)R¹⁰, NHSO₂R¹⁰, NHC(O)OR¹⁰, SO₂NH₂, SO₂NHR¹⁰, SO₂N(R¹⁰)₂, NHC(O)NH₂, NHC(O)NHR¹⁰, OH, (O), C(O)OH, N₃, CN, NH₂, CF₃, CF₂CF₃, F, Cl, Br or I;
R⁷ is alkyl, alkenyl, alkynyl, aryl, heterocyclyl, cycloalkyl, or cycloalkenyl; wherein each alkyl, alkenyl, and alkynyl is optionally substituted with one or more independently selected R¹¹, OR¹¹, SR¹¹, S(O)R¹¹, SO₂R¹¹, NHR¹¹, N(R¹¹)₂, C(O)R¹¹, C(O)NH₂, C(O)NHR¹¹, C(O)N(R¹¹)₂, NHC(O)R¹¹, NR¹¹C(O)R¹¹, NHSO₂R¹¹, NHC(O)OR¹¹, SO₂NH₂, SO₂NHR¹¹, SO₂N(R¹¹)₂, NHC(O)NH₂, NHC(O)NHR¹¹, OH, (O), C(O)OH, N₃, CN, NH₂, CF₃, CF₂CF₃, F, Cl, Br or I;
R⁸ is alkyl, alkenyl, alkynyl, aryl, heterocyclyl, cycloalkyl, or cycloalkenyl; wherein each alkyl, alkenyl, and alkynyl is optionally substituted with one or more independently selected R¹², OR¹², SR¹², S(O)R¹², SO₂R¹², NHR¹², N(R¹²)₂, C(O)R¹², C(O)NH₂, C(O)NHR¹², C(O)N(R¹²)₂, NHC(O)R¹², NR¹²C(O)R¹², NHSO₂R¹², NHC(O)OR¹², SO₂NH₂, SO₂NHR¹², SO₂N(R¹²)₂, NHC(O)NH₂, NHC(O)NHR¹², OH, (O), C(O)OH, N₃, CN, NH₂, CF₃, CF₂CF₃, F, Cl, Br or I;
R⁹ is alkyl, alkenyl, alkynyl, aryl, heterocyclyl, cycloalkyl, or cycloalkenyl; wherein each alkyl, alkenyl, and alkynyl is optionally substituted with one or more independently selected OCH₃, aryl, or heterocyclyl;
R¹⁰ is alkyl, alkenyl, alkynyl, aryl, heterocyclyl, cycloalkyl, or cycloalkenyl;
R¹¹ is alkyl, alkenyl, alkynyl, aryl, heterocyclyl, cycloalkyl, or cycloalkenyl;
R¹² is alkyl, alkenyl, alkynyl, aryl, heterocyclyl, cycloalkyl, or cycloalkenyl;
wherein the cyclic moieties represented by R³, R⁵, R⁶, R⁷, R⁸, R⁹, R¹⁰, R¹¹, and R¹² are optionally substituted with one or more independently selected R¹³, OR¹³, SR¹³, S(O)R¹³, SO₂R¹³, C(O)R¹³, CO(O)R¹³, OC(O)R¹³, OC(O)OR¹³, NH₂, NHR¹³, N(R¹³)₂, NHC(O)R¹³, NR¹³C(O)R¹³, NHS(O)₂R¹³, NR¹³S(O)₂R¹³, NHC(O)OR¹³, NR¹³C(O)OR¹³, NHC(O)NH₂, NHC(O)NHR¹³, NHC(O)N(R¹³)₂, NR¹³C(O)NHR¹³, NR¹³C(O)N(R¹³)₂, C(O)NH₂, C(O)NHR¹³, C(O)N(R¹³)₂, C(O)NHOH, C(O)NHOR¹³, C(O)NHSO₂R¹³, C(O)NR¹³SO₂R¹³, SO₂NH₂, SO₂NHR¹³, SO₂N(R¹³)₂, C(O)H, C(O)OH, OH, CN, N₃, NO₂, CF₃, CF₂CF₃, OCF₃, OCF₂CF₃, F, Cl, Br or I;
R¹³ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁴, OR¹⁴, SR¹⁴, S(O)R¹⁴, SO₂R¹⁴, C(O)R¹⁴, CO(O)R¹⁴, OC(O)R¹⁴, OC(O)OR¹⁴, NH₂, NHR¹⁴, N(R¹⁴)₂, NHC(O)R¹⁴, NR¹⁴C(O)R¹⁴, NHS(O)₂R¹⁴, NR¹⁴S(O)₂R¹⁴, NHC(O)OR¹⁴, NR¹⁴C(O)OR¹⁴, NHC(O)NH₂, NHC(O)NHR¹⁴, NHC(O)N(R¹⁴)₂, NR¹⁴C(O)NHR¹⁴, NR¹⁴C(O)N(R¹⁴)₂, C(O)NH₂, C(O)NHR¹⁴, C(O)N(R¹⁴)₂, C(O)NHOH, C(O)NHOR¹⁴, C(O)NHSO₂R¹⁴, C(O)NR¹⁴SO₂R¹⁴, SO₂NH₂, SO₂NHR¹⁴, SO₂N(R¹⁴)₂, C(O)H, C(O)OH, OH, (O), CN, N₃, NO₂, CF₃, CF₂CF₃, OCF₃, OCF₂CF₃, F, Cl, Br or I; wherein each aryl, heterocyclyl, cycloalkyl, and cycloalkenyl is optionally substituted with one or more independently selected R¹⁵, OR¹⁵, SR¹⁵, S(O)R¹⁵, SO₂R¹⁵, C(O)R¹⁵, CO(O)R¹⁵, OC(O)R¹⁵, OC(O)OR¹⁵, NH₂, NHR¹⁵, N(R¹⁵)₂, NHC(O)R¹⁵, NR¹⁵C(O)R¹⁵, NHS(O)₂R¹⁵, NR¹⁵S(O)₂R¹⁵, NHC(O)OR¹⁵, NR¹⁵C(O)OR¹⁵, NHC(O)NH₂, NHC(O)NHR¹⁵, NHC(O)N(R¹⁵)₂, NR¹⁵C(O)NHR¹⁵, NR¹⁵C(O)N(R¹⁵)₂, C(O)NH₂, C(O)NHR¹⁵, C(O)N(R¹⁵)₂, C(O)NHOH, C(O)NHOR¹⁵, C(O)NHSO₂R¹⁵, C(O)NR¹⁵SO₂R¹⁵, SO₂NH₂, SO₂NHR¹⁵, SO₂N(R¹⁵)₂, C(O)H, C(O)OH, OH, CN, N₃, NO₂, CF₃, CF₂CF₃, OCF₃, OCF₂CF₃, F, Cl, Br or I;
R¹⁴ is alkyl, alkenyl, alkynyl, aryl, heterocyclyl, cycloalkyl, or cycloalkenyl; wherein each alkyl, alkenyl, and alkynyl is optionally substituted with one or more independently selected NH₂, SO₂NH₂, C(O)H, C(O)OH, OH, (O), CN, N₃, NO₂, CF₃, CF₂CF₃, OCF₃, OCF₂CF₃, F, Cl, Br or I; and
R¹⁵ is alkyl.

2. A compound having Formula (V), or pharmaceutically acceptable salts thereof 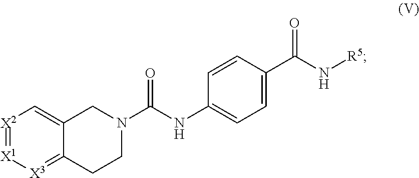
wherein
X¹ and X² and X³ are CH; or
X¹ and X³ are CH; and X² is N; or
X² and X³ are H; and X¹ is N; or
X¹ is CR¹; and X² and X³ are CH; or
X² is CR¹; and X¹ and X³ are CH; or
X³ is CR¹; and X¹ and X² are CH;
R¹ is R³, OR³, SR³, S(O)R³, SO₂R³, C(O)R³, C(O)OR³, OC(O)R³, NHR³, N(R³)₂, C(O)NH₂, C(O)NHR³, C(O)N(R³)₂, NHC(O)R³, NR³C(O)R³, NHC(O)OR³, NR³C(O)OR³, SO₂NH₂, SO₂NHR³, SO₂N(R³)₂, NHSO₂R³, NR³SO₂R³, NHSO₂NHR³, NHSO₂N(R³)₂, NR³SO₂NHR³, NR³SO₂N(R³)₂, C(O)NHSO₂R³, NHSO₂NHR³, F, Cl, Br, I, CN, NH₂, NO₂, N₃, OH, C(O)H, CF₃, C(O)OH, or C(O)NH₂;
R³ is alkyl, alkenyl, alkynyl, aryl, or heterocyclyl; wherein each alkyl, alkenyl, and alkynyl is optionally substituted with one or more independently selected R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, OH, (O), CN, N₃, NO₂, CF₃, CF₂CF₃, OCF₃, OCF₂CF₃, F, Cl, Br or I;
R⁵ is alkyl, alkenyl, alkynyl, aryl, heterocyclyl, cycloalkyl, or cycloalkenyl; wherein each alkyl, alkenyl, and alkynyl is optionally substituted with one or more independently selected R⁹, OR⁹, SR⁹, S(O)R⁹, SO₂R⁹, C(O)R⁹, CO(O)R⁹, OC(O)R⁹, OC(O)OR⁹, NH₂, NHR⁹, N(R⁹)₂, NHC(O)R⁹, NR⁹C(O)R⁹, NHS(O)₂R⁹, NR⁹S(O)₂R⁹, NHC(O)OR⁹, NR⁹C(O)OR⁹, NHC(O)NH₂, NHC(O)NHR⁹, NHC(O)N(R⁹)₂, NR⁹C(O)NHR⁹, NR⁹C(O)N(R⁹)₂, C(O)NH₂, C(O)NHR⁹, C(O)N(R⁹)₂, C(O)NHOH, C(O)NHOR⁹, C(O)NHSO₂R⁹, C(O)NR⁹SO₂R⁹, SO₂NH₂, SO₂NHR⁹, SO₂N(R⁹)₂, C(O)H, C(O)OH, OH, (O), CN, N₃, NO₂, CF₃, CF₂CF₃, OCF₃, OCF₂CF₃, F, Cl, Br or I;
R⁶ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁰, OR¹⁰, SR¹⁰, S(O)R¹⁰, SO₂R¹⁰, NHR¹⁰, N(R¹⁰)₂, C(O)R¹⁰, C(O)NH₂, C(O)NHR¹⁰, C(O)N(R¹⁰)₂, NHC(O)R¹⁰, NR¹⁰C(O)R¹⁰, NHSO₂R¹⁰, NHC(O)OR¹⁰, SO₂NH₂, SO₂NHR¹⁰, SO₂N(R¹⁰)₂, NHC(O)NH₂, NHC(O)NHR¹⁰, OH, (O), C(O)OH, N₃, CN, NH₂, CF₃, CF₂CF₃, F, Cl, Br or I;
R⁹ is alkyl, alkenyl, alkynyl, aryl, heterocyclyl, cycloalkyl, or cycloalkenyl; wherein each alkyl, alkenyl, and alkynyl is optionally substituted with one or more independently selected OCH₃, aryl, or heterocyclyl;
R¹⁰ is alkyl, alkenyl, alkynyl, aryl, heterocyclyl, cycloalkyl, or cycloalkenyl;
wherein the cyclic moieties represented by R³, R⁵, R⁶, R⁹, and R¹⁰, are optionally substituted with one or more independently selected R¹³, OR¹³, SR¹³, S(O)R¹³, SO₂R¹³, C(O)R¹³, CO(O)R¹³, OC(O)R¹³, OC(O)OR¹³, NH₂, NHR³, N(R¹³)₂, NHC(O)R¹³, NR¹³C(O)R¹³, NHS(O)₂R¹³, NR¹³S(O)₂R¹³, NHC(O)OR¹³, NR¹³C(O)OR¹³, NHC(O)NH₂, NHC(O)NHR¹³, NHC(O)N(R¹³)₂, NR¹³C(O)NHR¹³, NR¹³C(O)N(R¹³)₂, C(O)NH₂, C(O)NHR¹³, C(O)N(R¹³)₂, C(O)NHOH, C(O)NHOR¹³, C(O)NHSO₂R¹³, C(O)NR¹³SO₂R¹³, SO₂NH₂, SO₂NHR¹³, SO₂N(R¹³)₂, C(O)H, C(O)OH, OH, CN, N₃, NO₂, CF₃, CF₂CF₃, OCF₃, OCF₂CF₃, F, Cl, Br or I;
R¹³ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁴, OR¹⁴, SR¹⁴, S(O)R¹⁴, SO₂R¹⁴C(O)R¹⁴, CO(O)R¹⁴, OC(O)R¹⁴, OC(O)OR¹⁴, NH₂, NHR¹⁴, N(R¹⁴)₂, NHC(O)R¹⁴, NR¹⁴C(O)R¹⁴, NHS(O)₂R¹⁴, NR¹⁴S(O)₂R¹⁴, NHC(O)OR¹⁴, NR¹⁴C(O)OR¹⁴, NHC(O)NH₂, NHC(O)NHR¹⁴, NHC(O)N(R¹⁴)₂, NR¹⁴C(O)NHR¹⁴, NR¹⁴C(O)N(R¹⁴)₂, C(O)NH₂, C(O)NHR¹⁴, C(O)N(R¹⁴)₂, C(O)NHOH, C(O)NHOR¹⁴, C(O)NHSO₂R¹⁴, C(O)NR¹⁴SO₂R¹⁴, SO₂NH₂, SO₂NHR¹⁴, SO₂N(R¹⁴)₂, C(O)H, C(O)OH, OH, (O), CN, N₃, NO₂, CF₃, CF₂CF₃, OCF₃, OCF₂CF₃, F, Cl, Br or I; wherein each aryl, heterocyclyl, cycloalkyl, and cycloalkenyl is optionally substituted with one or more independently selected R¹⁵, OR¹⁵, SR¹⁵, S(O)R¹⁵, SO₂R¹⁵, C(O)R¹⁵, CO(O)R¹⁵, OC(O)R¹⁵, OC(O)OR¹⁵, NH₂, NHR¹⁵, N(R¹⁵)₂, NHC(O)R¹⁵, NR¹⁵C(O)R¹⁵, NHS(O)₂R¹⁵, NR¹⁵S(O)₂R¹⁵, NHC(O)OR¹⁵, NR¹⁵C(O)OR¹⁵, NHC(O)NH₂, NHC(O)NHR¹⁵, NHC(O)N(R¹⁵)₂, NR¹⁵C(O)NHR¹⁵, NR¹⁵C(O)N(R¹⁵)₂, C(O)NH₂, C(O)NHR¹⁵, C(O)N(R¹⁵)₂, C(O)NHOH, C(O)NHOR¹⁵, C(O)NHSO₂R¹⁵, C(O)NR¹⁵SO₂R¹⁵, SO₂NH₂, SO₂NHR¹⁵, SO₂N(R¹⁵)₂, C(O)H, C(O)OH, OH, CN, N₃, NO₂, CF₃, CF₂CF₃, OCF₃, OCF₂CF₃, F, Cl, Br or I;
R¹⁴ is alkyl, alkenyl, alkynyl, aryl, heterocyclyl, cycloalkyl, or cycloalkenyl; wherein each alkyl, alkenyl, and alkynyl is optionally substituted with one or more independently selected NH₂, SO₂NH₂, C(O)H, C(O)OH, OH, (O), CN, N₃, NO₂, CF₃, CF₂CF₃, OCF₃, OCF₂CF₃, F, Cl, Br or I; and
R¹⁵ is alkyl.

3. A compound having Formula (IV), or pharmaceutically acceptable salts thereof 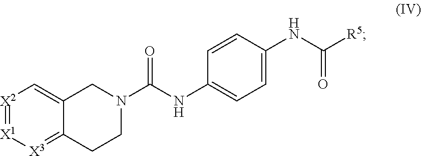
wherein
X¹ and X² and X³ are CH; or
X¹ and X³ are CH; and X² is N; or
X² and X³ are H; and X¹ is N; or
X¹ is CR¹; and X² and X³ are CH; or
X² is CR¹; and X¹ and X³ are CH; or
X³ is CR¹; and X¹ and X² are CH;
R¹ is R³, OR³, SR³, S(O)R³, SO₂R³, C(O)R³, C(O)OR³, OC(O)R³, NHR³, N(R³)₂, C(O)NH₂, C(O)NHR³, C(O)N(R³)₂, NHC(O)R³, NR³C(O)R³, NHC(O)OR³, NR³C(O)OR³, SO₂NH₂, SO₂NHR³, SO₂N(R³)₂, NHSO₂R³, NR³SO₂R³, NHSO₂NHR³, NHSO₂N(R³)₂, NR³SO₂NHR³, NR³SO₂N(R³)₂, C(O)NHSO₂R³, NHSO₂NHR³, F, Cl, Br, I, CN, NH₂, NO₂, N₃, OH, C(O)H, CF₃, C(O)OH, or C(O)NH₂;
R³ is alkyl, alkenyl, alkynyl, aryl, or heterocyclyl; wherein each alkyl, alkenyl, and alkynyl is optionally substituted with one or more independently selected R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, OH, (O), CN, N₃, NO₂, CF₃, CF₂CF₃, OCF₃, OCF₂CF₃, F, Cl, Br or I;
R⁵ is alkyl, alkenyl, alkynyl, aryl, heterocyclyl, cycloalkyl, or cycloalkenyl; wherein each alkyl, alkenyl, and alkynyl is optionally substituted with one or more independently selected R⁹, OR⁹, SR⁹, S(O)R⁹, SO₂R⁹, C(O)R⁹, CO(O)R⁹, OC(O)R⁹, OC(O)OR⁹, NH₂, NHR⁹, N(R⁹)₂, NHC(O)R⁹, NR⁹C(O)R⁹, NHS(O)₂R⁹, NR⁹S(O)₂R⁹, NHC(O)OR⁹, NR⁹C(O)OR⁹, NHC(O)NH₂, NHC(O)NHR⁹, NHC(O)N(R⁹)₂, NR⁹C(O)NHR⁹, NR⁹C(O)N(R⁹)₂, C(O)NH₂, C(O)NHR⁹, C(O)N(R⁹)₂, C(O)NHOH, C(O)NHOR⁹, C(O)NHSO₂R⁹, C(O)NR⁹SO₂R⁹, SO₂NH₂, SO₂NHR⁹, SO₂N(R⁹)₂, C(O)H, C(O)OH, OH, (O), CN, N₃, NO₂, CF₃, CF₂CF₃, OCF₃, OCF₂CF₃, F, Cl, Br or I;
R⁶ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁰, OR¹⁰, SR¹⁰, S(O)R¹⁰, SO₂R¹⁰, NHR¹⁰, N(R¹⁰)₂, C(O)R¹⁰, C(O)NH₂, C(O)NHR¹⁰, C(O)N(R¹⁰)₂, NHC(O)R¹⁰, NR¹⁰C(O)R¹⁰, NHSO₂R¹⁰, NHC(O)OR¹⁰, SO₂NH₂, SO₂NHR¹⁰, SO₂N(R¹⁰)₂, NHC(O)NH₂, NHC(O)NHR¹⁰, OH, (O), C(O)OH, N₃, CN, NH₂, CF₃, CF₂CF₃, F, Cl, Br or I;
R⁹ is alkyl, alkenyl, alkynyl, aryl, heterocyclyl, cycloalkyl, or cycloalkenyl; wherein each alkyl, alkenyl, and alkynyl is optionally substituted with one or more independently selected OCH₃, aryl, or heterocyclyl;
R¹⁰ is alkyl, alkenyl, alkynyl, aryl, heterocyclyl, cycloalkyl, or cycloalkenyl;
wherein the cyclic moieties represented by R³, R⁵, R⁶, R⁹, and R¹⁰, are optionally substituted with one or more independently selected R¹³, OR¹³, SR¹³, S(O)R¹³, SO₂R¹³, C(O)R¹³, CO(O)R¹³, OC(O)R¹³, OC(O)OR¹³, NH₂, NHR¹³, N(R¹³)₂, NHC(O)R¹³, NR¹³C(O)R¹³, NHS(O)₂R¹³, NR¹³S(O)₂R¹³, NHC(O)OR¹³, NR¹³C(O)OR¹³, NHC(O)NH₂, NHC(O)NHR¹³, NHC(O)N(R¹³)₂, NR¹³C(O)NHR¹³, NR¹³C(O)N(R¹³)₂, C(O)NH₂, C(O)NHR¹³, C(O)N(R¹³)₂, C(O)NHOH, C(O)NHOR¹³, C(O)NHSO₂R¹³, C(O)NR¹³SO₂R¹³, SO₂NH₂, SO₂NHR¹³, SO₂N(R¹³)₂, C(O)H, C(O)OH, OH, CN, N₃, NO₂, CF₃, CF₂CF₃, OCF₃, OCF₂CF₃, F, Cl, Br or I;
R¹³ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁴, OR¹⁴, SR¹⁴, S(O)R¹⁴, SO₂R¹⁴, C(O)R¹⁴, CO(O)R¹⁴, OC(O)R¹⁴, OC(O)OR¹⁴, NH₂, NHR¹⁴, N(R¹⁴)₂, NHC(O)R¹⁴, NR¹⁴C(O)R¹⁴, NHS(O)₂R¹⁴, NR¹⁴S(O)₂R¹⁴, NHC(O)OR¹⁴, NR¹⁴C(O)OR¹⁴, NHC(O)NH₂, NHC(O)NHR¹⁴, NHC(O)N(R¹⁴)₂, NR¹⁴C(O)NHR¹⁴, NR¹⁴C(O)N(R¹⁴)₂, C(O)NH₂, C(O)NHR¹⁴, C(O)N(R¹⁴)₂, C(O)NHOH, C(O)NHOR¹⁴, C(O)NHSO₂R¹⁴, C(O)NR¹⁴SO₂R¹⁴, SO₂NH₂, SO₂NHR¹⁴, SO₂N(R¹⁴)₂, C(O)H, C(O)OH, OH, (O), CN, N₃, NO₂, CF₃, CF₂CF₃, OCF₃, OCF₂CF₃, F, Cl, Br or I; wherein each aryl, heterocyclyl, cycloalkyl, and cycloalkenyl is optionally substituted with one or more independently selected R¹⁵, OR¹⁵, SR¹⁵, S(O)R¹⁵, SO₂R¹⁵, C(O)R¹⁵, CO(O)R¹⁵, OC(O)R¹⁵, OC(O)OR¹⁵, NH₂, NHR¹⁵, N(R¹⁵)₂, NHC(O)R¹⁵, NR¹⁵C(O)R¹⁵, NHS(O)₂R¹⁵, NR¹⁵S(O)₂R¹⁵, NHC(O)OR¹⁵, NR¹⁵C(O)OR¹⁵, NHC(O)NH₂, NHC(O)NHR¹⁵, NHC(O)N(R¹⁵)₂, NR¹⁵C(O)NHR¹⁵, NR¹⁵C(O)N(R¹⁵)₂, C(O)NH₂, C(O)NHR¹⁵, C(O)N(R¹⁵)₂, C(O)NHOH, C(O)NHOR¹⁵, C(O)NHSO₂R¹⁵, C(O)NR¹⁵SO₂R¹⁵, SO₂NH₂, SO₂NHR¹⁵, SO₂N(R¹⁵)₂, C(O)H, C(O)OH, OH, CN, N₃, NO₂, CF₃, CF₂CF₃, OCF₃, OCF₂CF₃, F, Cl, Br or I;
R¹⁴ is alkyl, alkenyl, alkynyl, aryl, heterocyclyl, cycloalkyl, or cycloalkenyl; wherein each alkyl, alkenyl, and alkynyl is optionally substituted with one or more independently selected NH₂, SO₂NH₂, C(O)H, C(O)OH, OH, (O), CN, N₃, NO₂, CF₃, CF₂CF₃, OCF₃, OCF₂CF₃, F, Cl, Br or I; and
R¹⁵ is alkyl.

4. A compound having Formula (VI), or pharmaceutically acceptable salts thereof 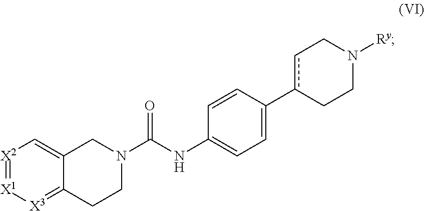
wherein [symbol]
indicates a single or a double bond;
X¹ and X² and X³ are CH; or
X¹ and X³ are CH; and X² is N; or
X² and X³ are H; and X¹ is N; or
X¹ is CR¹; and X² and X³ are CH; or
X² is CR¹; and X¹ and X³ are CH; or
X³ is CR¹; and X¹ and X² are CH;
R¹ is R³, OR³, SR³, S(O)R³, SO₂R³, C(O)R³, C(O)OR³, OC(O)R³, NHR³, N(R³)₂, C(O)NH₂, C(O)NHR³, C(O)N(R³)₂, NHC(O)R³, NR³C(O)R³, NHC(O)OR³, NR³C(O)OR³, SO₂NH₂, SO₂NHR³, SO₂N(R³)₂, NHSO₂R³, NR³SO₂R³, NHSO₂NHR³, NHSO₂N(R³)₂, NR³SO₂NHR³, NR³SO₂N(R³)₂, C(O)NHSO₂R³, NHSO₂NHR³, F, Cl, Br, I, CN, NH₂, NO₂, N₃, OH, C(O)H, CF₃, C(O)OH, or C(O)NH₂;
R³ is alkyl, alkenyl, alkynyl, aryl, or heterocyclyl; wherein each alkyl, alkenyl, and alkynyl is optionally substituted with one or more independently selected R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, OH, (O), CN, N₃, NO₂, CF₃, CF₂CF₃, OCF₃, OCF₂CF₃, F, Cl, Br or I;
R⁶ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁰, OR¹⁰, SR¹⁰, S(O)R¹⁰, SO₂R¹⁰, NHR¹⁰, N(R¹⁰)₂, C(O)R¹⁰, C(O)NH₂, C(O)NHR¹⁰, C(O)N(R¹⁰)₂, NHC(O)R¹⁰, NR¹⁰C(O)R¹⁰, NHSO₂R¹⁰, NHC(O)OR¹⁰, SO₂NH₂, SO₂NHR¹⁰, SO₂N(R¹⁰)₂, NHC(O)NH₂, NHC(O)NHR¹⁰, OH, (O), C(O)OH, N₃, CN, NH₂, CF₃, CF₂CF₃, F, Cl, Br or I;
R¹⁰ is alkyl, alkenyl, alkynyl, aryl, heterocyclyl, cycloalkyl, or cycloalkenyl;
R^{y} is R¹³, OR¹³, SR¹³, S(O)R¹³, SO₂R¹³, C(O)R¹³, CO(O)R¹³, OC(O)R¹³, OC(O)OR¹³, NH₂, NHR¹³, N(R¹³)₂, NHC(O)R¹³, NR¹³C(O)R¹³, NHS(O)₂R¹³, NR¹³S(O)₂R¹³, NHC(O)OR¹³, NR¹³C(O)OR¹³, NHC(O)NH₂, NHC(O)NHR¹³, NHC(O)N(R¹³)₂, NR¹³C(O)NHR¹³, NR¹³C(O)N(R¹³)₂, C(O)NH₂, C(O)NHR¹³, C(O)N(R¹³)₂, C(O)NHOH, C(O)NHOR¹³, C(O)NHSO₂R¹³, C(O)NR¹³SO₂R¹³, SO₂NH₂, SO₂NHR¹³, SO₂N(R¹³)₂, C(O)H, C(O)OH, OH, CN, N₃, NO₂, CF₃, CF₂CF₃, OCF₃, OCF₂CF₃, F, Cl, Br or I;
R¹³ is alkyl, alkenyl, alkynyl, aryl, heterocyclyl, cycloalkyl, or cycloalkenyl; wherein each alkyl, alkenyl, and alkynyl is optionally substituted with one or more independently selected R¹⁴, OR¹⁴, SR¹⁴, S(O)R¹⁴, SO₂R¹⁴, C(O)R¹⁴, CO(O)R¹⁴, OC(O)R¹⁴, OC(O)OR¹⁴, NH₂, NHR¹⁴, N(R¹⁴)₂, NHC(O)R¹⁴, NR¹⁴C(O)R¹⁴, NHS(O)₂R¹⁴, NR¹⁴S(O)₂R¹⁴, NHC(O)OR¹⁴, NR¹⁴C(O)OR¹⁴, NHC(O)NH₂, NHC(O)NHR¹⁴, NHC(O)N(R¹⁴)₂, NR¹⁴C(O)NHR¹⁴, NR¹⁴C(O)N(R¹⁴)₂, C(O)NH₂, C(O)NHR¹⁴, C(O)N(R¹⁴)₂, C(O)NHOH, C(O)NHOR¹⁴, C(O)NHSO₂R¹⁴, C(O)NR¹⁴SO₂R¹⁴, SO₂NH₂, SO₂NHR¹⁴, SO₂N(R¹⁴)₂, C(O)H, C(O)OH, OH, (O), CN, N₃, NO₂, CF₃, CF₂CF₃, OCF₃, OCF₂CF₃, F, Cl, Br or I; wherein each aryl, heterocyclyl, cycloalkyl, and cycloalkenyl is optionally substituted with one or more independently selected R¹⁵, OR¹⁵, SR¹⁵, S(O)R¹⁵, SO₂R¹⁵, C(O)R¹⁵, CO(O)R¹⁵, OC(O)R¹⁵, OC(O)OR¹⁵, NH₂, NHR¹⁵, N(R¹⁵)₂, NHC(O)R¹⁵, NR¹⁵C(O)R¹⁵, NHS(O)₂R¹⁵, NR¹⁵S(O)₂R¹⁵, NHC(O)OR¹⁵, NR¹⁵C(O)OR¹⁵, NHC(O)NH₂, NHC(O)NHR¹⁵, NHC(O)N(R¹⁵)₂, NR¹⁵C(O)NHR¹⁵, NR¹⁵C(O)N(R¹⁵)₂, C(O)NH₂, C(O)NHR¹⁵, C(O)N(R¹⁵)₂, C(O)NHOH, C(O)NHOR¹⁵, C(O)NHSO₂R¹⁵, C(O)NR¹⁵SO₂R¹⁵, SO₂NH₂, SO₂NHR¹⁵, SO₂N(R¹⁵)₂, C(O)H, C(O)OH, OH, CN, N₃, NO₂, CF₃, CF₂CF₃, OCF₃, OCF₂CF₃, F, Cl, Br or I;
R¹⁴ is alkyl, alkenyl, alkynyl, aryl, heterocyclyl, cycloalkyl, or cycloalkenyl; wherein each alkyl, alkenyl, and alkynyl is optionally substituted with one or more independently selected NH₂, SO₂NH₂, C(O)H, C(O)OH, OH, (O), CN, N₃, NO₂, CF₃, CF₂CF₃, OCF₃, OCF₂CF₃, F, Cl, Br or I; and
R¹⁵ is alkyl.

5. The compound of any one of claims 1-4, or pharmaceutically acceptable salts thereof; wherein
X¹, X², and X³ are CH; or
X¹ is CR¹ and X² and X³ are CH; or
X² is CR¹ and X¹ and X³ are CH.

6. The compound of any one of claims 1-4, or pharmaceutically acceptable salts thereof; wherein
X¹ and X³ are CH; and X² is N; or
X² and X³ are CH; and X¹ is N.

7. The compound of claim 4, or pharmaceutically acceptable salts thereof; wherein [symbol]
is a double bond.

8. A compound selected from the group consisting of
N-{4-[(3-methylbutyl)carbamoyl]phenyl}-3,4-dihydroisoquinoline-2(1H)-carboxamide;
N-(4-{[4-(pyridin-2-yl)piperazin-1-yl]carbonyl}phenyl)-3,4-dihydroisoquinoline-2(1H)-carboxamide;
6-fluoro-N-{4-[(3-phenylpropyl)carbamoyl]phenyl}-3,4-dihydroisoquinoline-2(1H)-carboxamide;
N-{4-[(3-phenylpropyl)carbamoyl]phenyl}-3,4-dihydro-2,6-naphthyridine-2(1H)-carboxamide;
N-{4-[(3-methylbutyl)carbamoyl]phenyl}-3,4-dihydro-2,6-naphthyridine-2(1H)-carboxamide;
6-fluoro-N-{4-[(3-methylbutyl)carbamoyl]phenyl}-3,4-dihydroisoquinoline-2(1H)-carboxamide;
N-{4-[(3-phenylpropyl)carbamoyl]phenyl}-3,4-dihydroisoquinoline-2(1H)-carboxamide;
N-[4-(benzylcarbamoyl)phenyl]-3,4-dihydroisoquinoline-2(1H)-carboxamide;
N-{5-[(3-phenylpropyl)carbamoyl]pyridin-2-yl}-3,4-dihydroisoquinoline-2(1H)-carboxamide;
N-{5-[(3-methylbutyl)carbamoyl]pyridin-2-yl}-3,4-dihydroisoquinoline-2(1H)-carboxamide;
N-(4-{[1-(3-methylbutyl)-1H-pyrazol-4-yl]carbamoyl}phenyl)-3,4-dihydroisoquinoline-2(1H)-carboxamide;
N-(4-{[2-(2-thienyl)ethyl]carbamoyl}phenyl)-3,4-dihydro-2,6-naphthyridine-2(1H)-carboxamide;
6-fluoro-N-(4-{[2-(2-thienyl)ethyl]carbamoyl}phenyl)-3,4-dihydroisoquinoline-2(1H)-carboxamide;
N-{4-[(1-benzyl-1H-pyrazol-4-yl)carbamoyl]phenyl}-3,4-dihydroisoquinoline-2(1H)-carboxamide;
N-{4-[(2-phenylethyl)carbamoyl]phenyl}-3,4-dihydroisoquinoline-2(1H)-carboxamide;
7-fluoro-N-{4-[(3-methylbutyl)carbamoyl]phenyl}-3,4-dihydroisoquinoline-2(1H)-carboxamide;
N-(4-{[2-(2-thienyl)ethyl]carbamoyl}phenyl)-3,4-dihydroisoquinoline-2(1H)-carboxamide;
N-{6-[(3-methylbutyl)carbamoyl]pyridin-3-yl}-3,4-dihydroisoquinoline-2(1H)-carboxamide;
N-(5-{[2-(2-thienyl)ethyl]carbamoyl}pyridin-2-yl)-3,4-dihydroisoquinoline-2(1H)-carboxamide;
7-fluoro-N-{4-[(3-phenylpropyl)carbamoyl]phenyl}-3,4-dihydroisoquinoline-2(1H)-carboxamide;
N-{6-[(3-phenylpropyl)carbamoyl]pyridin-3-yl}-3,4-dihydroisoquinoline-2(1H)-carboxamide;
N-(6-{[2-(2-thienyl)ethyl]carbamoyl}pyridin-3-yl)-3,4-dihydroisoquinoline-2(1H)-carboxamide;
7-fluoro-N-(4-{[2-(2-thienyl)ethyl]carbamoyl}phenyl)-3,4-dihydroisoquinoline-2(1H)-carboxamide;
N-{4-[(3-phenylpropyl)carbamoyl]phenyl}-3,4-dihydro-2,7-naphthyridine-2(1H)-carboxamide;
N-[4-(2-oxo-2-{[2-(2-thienyl)ethyl]amino}ethyl)phenyl]-3,4-dihydroisoquinoline-2(1H)-carboxamide;
N-(4-{2-oxo-2-[(3-phenylpropyl)amino]ethyl}phenyl)-3,4-dihydroisoquinoline-2(1H)-carboxamide;
N-{4-[(3-methylbutyl)carbamoyl]phenyl}-3,4-dihydro-2,7-naphthyridine-2(1H)-carboxamide;
N-(4-{2-[(3-methylbutyl)amino]-2-oxoethyl}phenyl)-3,4-dihydroisoquinoline-2(1H)-carboxamide;
N-(4-{[2-(2-thienyl)ethyl]carbamoyl}phenyl)-3,4-dihydro-2,7-naphthyridine-2(1H)-carboxamide;
N-{4-[(4-methylpentanoyl)amino]phenyl}-3,4-dihydroisoquinoline-2(1H)-carboxamide;
N-{4-[(4-phenylbutanoyl)amino]phenyl}-3,4-dihydroisoquinoline-2(1H)-carboxamide;
N-(4-{[3-(2-thienyl)propanoyl]amino}phenyl)-3,4-dihydroisoquinoline-2(1H)-carboxamide;
N-[4-(1-isobutyl-1H-pyrazol-4-yl)phenyl]-3,4-dihydroisoquinoline-2(1H)-carboxamide;
N-[4-(1-propyl-1H-pyrazol-4-yl)phenyl]-3,4-dihydroisoquinoline-2(1H)-carboxamide;
N-{4-[1-((2R)-2-hydroxypropyl)-1H-pyrazol-4-yl]phenyl}-3,4-dihydroisoquinoline-2(1H)-carboxamide;
N-{4-[1-(3-methylbutyl)-1H-pyrazol-4-yl]phenyl}-3,4-dihydroisoquinoline-2(1H)-carboxamide;
N-[4-(1-benzyl-1H-pyrazol-4-yl)phenyl]-3,4-dihydroisoquinoline-2(1H)-carboxamide;
N-{4-[(1E)-5-phenylpent-1-en-1-yl]phenyl}-3,4-dihydroisoquinoline-2(1H)-carboxamide;
N-[4-(1-ethyl-1H-pyrazol-4-yl)phenyl]-3,4-dihydroisoquinoline-2(1H)-carboxamide;
N-{4-[1-(2-hydroxyethyl)-1H-pyrazol-4-yl]phenyl}-3,4-dihydroisoquinoline-2(1H)-carboxamide;
N-[4-(1-methyl-1H-pyrazol-4-yl)phenyl]-3,4-dihydroisoquinoline-2(1H)-carboxamide;
N-[4-(1-benzoyl-1,2,3,6-tetrahydropyridin-4-yl)phenyl]-3,4-dihydroisoquinoline-2(1H)-carboxamide;
N-[4-(1-butyryl-1,2,3,6-tetrahydropyridin-4-yl)phenyl]-3,4-dihydroisoquinoline-2(1H)-carboxamide;
N-{4-[1-(isopropylsulfonyl)-1,2,3,6-tetrahydropyridin-4-yl]phenyl}-3,4-dihydroisoquinoline-2(1H)-carboxamide;
N-[4-(1-isobutyryl-1,2,3,6-tetrahydropyridin-4-yl)phenyl]-3,4-dihydroisoquinoline-2(1H)-carboxamide;
N-{4-[1-(3-methylbutanoyl)-1,2,3,6-tetrahydropyridin-4-yl]phenyl}-3,4-dihydroisoquinoline-2(1H)-carboxamide;
N-{4-[1-(methylcarbamoyl)-1,2,3,6-tetrahydropyridin-4-yl]phenyl}-3,4-dihydroisoquinoline-2(1H)-carboxamide;
tert-butyl 4-{4-[(3,4-dihydroisoquinolin-2(1H)-ylcarbonyl)amino]phenyl}-3,6-dihydropyridine-1(2H)-carboxylate;
N-[4-(1-acetyl-1,2,3,6-tetrahydropyridin-4-yl)phenyl]-3,4-dihydroisoquinoline-2(1H)-carboxamide;
N-{4-[1-(isobutylsulfonyl)-1,2,3,6-tetrahydropyridin-4-yl]phenyl}-3,4-dihydroisoquinoline-2(1H)-carboxamide;
N-[4-(1-benzyl-1,2,3,6-tetrahydropyridin-4-yl)phenyl]-3,4-dihydroisoquinoline-2(1H)-carboxamide;
N-{4-[1-(methylsulfonyl)-1,2,3,6-tetrahydropyridin-4-yl]phenyl}-3,4-dihydroisoquinoline-2(1H)-carboxamide;
N-[4-(1,2,3,6-tetrahydropyridin-4-yl)phenyl]-3,4-dihydroisoquinoline-2(1H)-carboxamide;
N-{4-[1-(3-methylbutyl)-1,2,3,6-tetrahydropyridin-4-yl]phenyl}-3,4-dihydroisoquinoline-2(1H)-carboxamide;
N-[4-(5-propyl-1,2,4-oxadiazol-3-yl)phenyl]-3,4-dihydroisoquinoline-2(1H)-carboxamide;
N-[4-(5-benzyl-1,2,4-oxadiazol-3-yl)phenyl]-3,4-dihydroisoquinoline-2(1H)-carboxamide;
N-{4-[5-(3-methylbutyl)-1,2,4-oxadiazol-3-yl]phenyl}-3,4-dihydroisoquinoline-2(1H)-carboxamide;
N-hexyl-3,4-dihydroisoquinoline-2(1H)-carboxamide;
ethyl 6-[(3,4-dihydroisoquinolin-2(1H)-ylcarbonyl)amino]hexanoate;
N-(4-{2-[(phenylacetyl)amino]ethyl}phenyl)-3,4-dihydroisoquinoline-2(1H)-carboxamide;
N-{4-[2-(isobutyrylamino)ethyl]phenyl}-3,4-dihydroisoquinoline-2(1H)-carboxamide;
N-(4-{[(benzyloxy)acetyl]amino}phenyl)-3,4-dihydroisoquinoline-2(1H)-carboxamide;
N-(4-{[(4-methoxycyclohexyl)carbonyl]amino}phenyl)-3,4-dihydroisoquinoline-2(1H)-carboxamide;
N-(4-{[(1-acetylpiperidin-4-yl)carbonyl]amino}phenyl)-3,4-dihydroisoquinoline-2(1H)-carboxamide;
N-(4-{[4-oxo-4-(2-thienyl)butanoyl]amino}phenyl)-3,4-dihydroisoquinoline-2(1H)-carboxamide;
N-(4-{[3-(phenylsulfonyl)propanoyl]amino}phenyl)-3,4-dihydroisoquinoline-2(1H)-carboxamide;
N-{4-[((2R)-2,3-dihydro-1-benzofuran-2-ylcarbonyl)amino]phenyl}-3,4-dihydroisoquinoline-2(1H)-carboxamide and N-{4-[((2S)-2,3-dihydro-1-benzofuran-2-ylcarbonyl)amino]phenyl}-3,4-dihydroisoquinoline-2(1H)-carboxamide;
N N-{4-[((3R)-3-methylpentanoyl)amino]phenyl}-3,4-dihydroisoquinoline-2(1H)-carboxamide and N-{4-[((3S)-3-methylpentanoyl)amino]phenyl}-3,4-dihydroisoquinoline-2(1H)-carboxamide;
N-{4-[(2,2-dimethylbutanoyl)amino]phenyl}-3,4-dihydroisoquinoline-2(1H)-carboxamide;
N-{4-[(3,3-dimethylbutanoyl)amino]phenyl}-3,4-dihydroisoquinoline-2(1H)-carboxamide;
N-[4-(heptanoylamino)phenyl]-3,4-dihydroisoquinoline-2(1H)-carboxamide;
N-{4-[(4,4,4-trifluorobutanoyl)amino]phenyl}-3,4-dihydroisoquinoline-2(1H)-carboxamide;
N-(4-{[(2-methoxyethoxy)acetyl]amino}phenyl)-3,4-dihydroisoquinoline-2(1H)-carboxamide;
N-{4-[((3R)-tetrahydrofuran-3-ylcarbonyl)amino]phenyl}-3,4-dihydroisoquinoline-2(1H)-carboxamide and N-{4-[((3S)-tetrahydrofuran-3-ylcarbonyl)amino]phenyl}-3,4-dihydroisoquinoline-2(1H)-carboxamide;
N-(4-{[3-(methylthio)propanoyl]amino}phenyl)-3,4-dihydroisoquinoline-2(1H)-carboxamide;
N-{4-[(cyclopentylacetyl)amino]phenyl}-3,4-dihydroisoquinoline-2(1H)-carboxamide;
N-{4-[(cyclohexylcarbonyl)amino]phenyl}-3,4-dihydroisoquinoline-2(1H)-carboxamide;
N-{4-[(cyclohexylacetyl)amino]phenyl}-3,4-dihydroisoquinoline-2(1H)-carboxamide;
N-[4-(benzoylamino)phenyl]-3,4-dihydroisoquinoline-2(1H)-carboxamide;
N-{4-[(phenylacetyl)amino]phenyl}-3,4-dihydroisoquinoline-2(1H)-carboxamide;
N-(4-{[3-(4-aminophenyl)propanoyl]amino}phenyl)-3,4-dihydroisoquinoline-2(1H)-carboxamide;
N-[4-(3-furoylamino)phenyl]-3,4-dihydroisoquinoline-2(1H)-carboxamide;
N-{4-[(2,5-dimethyl-3-furoyl)amino]phenyl}-3,4-dihydroisoquinoline-2(1H)-carboxamide;
N-{4-[(3-thienylcarbonyl)amino]phenyl}-3,4-dihydroisoquinoline-2(1H)-carboxamide;
N-{4-[(1H-pyrrol-2-ylcarbonyl)amino]phenyl}-3,4-dihydroisoquinoline-2(1H)-carboxamide;
N-{4-[(1,3-thiazol-5-ylcarbonyl)amino]phenyl}-3,4-dihydroisoquinoline-2(1H)-carboxamide;
N-{4-[(1H-pyrazol-5-ylcarbonyl)amino]phenyl}-3,4-dihydroisoquinoline-2(1H)-carboxamide;
N-{4-[(1H-pyrazol-4-ylcarbonyl)amino]phenyl}-3,4-dihydroisoquinoline-2(1H)-carboxamide;
N-{4-[(1,2-oxazol-5-ylcarbonyl)amino]phenyl}-3,4-dihydroisoquinoline-2(1H)-carboxamide;
N-{4-[(pyridin-2-ylacetyl)amino]phenyl}-3,4-dihydroisoquinoline-2(1H)-carboxamide;
N-{4-[(N,N-dimethyl-beta-alanyl)amino]phenyl}-3,4-dihydroisoquinoline-2(1H)-carboxamide;
N-(4-{[3-(piperidin-1-yl)propanoyl]amino}phenyl)-3,4-dihydroisoquinoline-2(1H)-carboxamide;
N-{4-[(morpholin-4-ylacetyl)amino]phenyl}-3,4-dihydroisoquinoline-2(1H)-carboxamide;
N-(4-{[3-(morpholin-4-yl)propanoyl]amino}phenyl)-3,4-dihydroisoquinoline-2(1H)-carboxamide;
N-(4-{[3-(4-methylpiperazin-1-yl)propanoyl]amino}phenyl)-3,4-dihydroisoquinoline-2(1H)-carboxamide;
N-[4-(trifluoromethyl)phenyl]-3,4-dihydroisoquinoline-2(1H)-carboxamide;
N-{4-[(cyclopentylacetyl)amino]phenyl}-5-[(methylsulfonyl)amino]-3,4-dihydroisoquinoline-2(1H)-carboxamide;
and pharmaceutically acceptable salts thereof.

9. The compound of claim 2 selected from the group consisting of
N-{4-[(3-methylbutyl)carbamoyl]phenyl}-3,4-dihydroisoquinoline-2(1H)-carboxamide;
N-(4-{[4-(pyridin-2-yl)piperazin-1-yl]carbonyl}phenyl)-3,4-dihydroisoquinoline-2(1H)-carboxamide;
6-fluoro-N-{4-[(3-phenylpropyl)carbamoyl]phenyl}-3,4-dihydroisoquinoline-2(1H)-carboxamide;
N-{4-[(3-phenylpropyl)carbamoyl]phenyl}-3,4-dihydro-2,6-naphthyridine-2(1H)-carboxamide;
N-{4-[(3-methylbutyl)carbamoyl]phenyl}-3,4-dihydro-2,6-naphthyridine-2(1H)-carboxamide;
6-fluoro-N-{4-[(3-methylbutyl)carbamoyl]phenyl}-3,4-dihydroisoquinoline-2(1H)-carboxamide;
N-{4-[(3-phenylpropyl)carbamoyl]phenyl}-3,4-dihydroisoquinoline-2(1H)-carboxamide;
N-[4-(benzylcarbamoyl)phenyl]-3,4-dihydroisoquinoline-2(1H)-carboxamide;
N-(4-{[1-(3-methylbutyl)-1H-pyrazol-4-yl]carbamoyl}phenyl)-3,4-dihydroisoquinoline-2(1H)-carboxamide;
N-(4-{[2-(2-thienyl)ethyl]carbamoyl}phenyl)-3,4-dihydro-2,6-naphthyridine-2(1H)-carboxamide;
6-fluoro-N-(4-{[2-(2-thienyl)ethyl]carbamoyl}phenyl)-3,4-dihydroisoquinoline-2(1H)-carboxamide;
N-{4-[(1-benzyl-1H-pyrazol-4-yl)carbamoyl]phenyl}-3,4-dihydroisoquinoline-2(1H)-carboxamide;
N-{4-[(2-phenylethyl)carbamoyl]phenyl}-3,4-dihydroisoquinoline-2(1H)-carboxamide;
7-fluoro-N-{4-[(3-methylbutyl)carbamoyl]phenyl}-3,4-dihydroisoquinoline-2(1H)-carboxamide;
N-(4-{[2-(2-thienyl)ethyl]carbamoyl}phenyl)-3,4-dihydroisoquinoline-2(1H)-carboxamide;
7-fluoro-N-{4-[(3-phenylpropyl)carbamoyl]phenyl}-3,4-dihydroisoquinoline-2(1H)-carboxamide;
7-fluoro-N-(4-{[2-(2-thienyl)ethyl]carbamoyl}phenyl)-3,4-dihydroisoquinoline-2(1H)-carboxamide;
N-{4-[(3-phenylpropyl)carbamoyl]phenyl}-3,4-dihydro-2,7-naphthyridine-2(1H)-carboxamide;
N-{4-[(3-methylbutyl)carbamoyl]phenyl}-3,4-dihydro-2,7-naphthyridine-2(1H)-carboxamide;
N-(4-{[2-(2-thienyl)ethyl]carbamoyl}phenyl)-3,4-dihydro-2,7-naphthyridine-2(1H)-carboxamide;
and pharmaceutically acceptable salts thereof.

10. The compound of claim 3 selected from the group consisting of
N-{4-[(4-methylpentanoyl)amino]phenyl}-3,4-dihydroisoquinoline-2(1H)-carboxamide;
N-{4-[(4-phenylbutanoyl)amino]phenyl}-3,4-dihydroisoquinoline-2(1H)-carboxamide;
N-(4-{[3-(2-thienyl)propanoyl]amino}phenyl)-3,4-dihydroisoquinoline-2(1H)-carboxamide;
N-(4-{[(benzyloxy)acetyl]amino}phenyl)-3,4-dihydroisoquinoline-2(1H)-carboxamide;
N-(4-{[(4-methoxycyclohexyl)carbonyl]amino}phenyl)-3,4-dihydroisoquinoline-2(1H)-carboxamide;
N-(4-{[(1-acetylpiperidin-4-yl)carbonyl]amino}phenyl)-3,4-dihydroisoquinoline-2(1H)-carboxamide;
N-(4-{[4-oxo-4-(2-thienyl)butanoyl]amino}phenyl)-3,4-dihydroisoquinoline-2(1H)-carboxamide;
N-(4-{[3-(phenylsulfonyl)propanoyl]amino}phenyl)-3,4-dihydroisoquinoline-2(1H)-carboxamide;
N-{4-[((2R)-2,3-dihydro-1-benzofuran-2-ylcarbonyl)amino]phenyl}-3,4-dihydroisoquinoline-2(1H)-carboxamide and N-{4-[((2S)-2,3-dihydro-1-benzofuran-2-ylcarbonyl)amino]phenyl}-3,4-dihydroisoquinoline-2(1H)-carboxamide;
N N-{4-[((3R)-3-methylpentanoyl)amino]phenyl}-3,4-dihydroisoquinoline-2(1H)-carboxamide and N-{4-[((3S)-3-methylpentanoyl)amino]phenyl}-3,4-dihydroisoquinoline-2(1H)-carboxamide;
N-{4-[(2,2-dimethylbutanoyl)amino]phenyl}-3,4-dihydroisoquinoline-2(1H)-carboxamide;
N-{4-[(3,3-dimethylbutanoyl)amino]phenyl}-3,4-dihydroisoquinoline-2(1H)-carboxamide;
N-[4-(heptanoylamino)phenyl]-3,4-dihydroisoquinoline-2(1H)-carboxamide;
N-{4-[(4,4,4-trifluorobutanoyl)amino]phenyl}-3,4-dihydroisoquinoline-2(1H)-carboxamide;
N-(4-{[(2-methoxyethoxy)acetyl]amino}phenyl)-3,4-dihydroisoquinoline-2(1H)-carboxamide;
N-{4-[((3R)-tetrahydrofuran-3-ylcarbonyl)amino]phenyl}-3,4-dihydroisoquinoline-2(1H)-carboxamide and N-{4-[((3S)-tetrahydrofuran-3-ylcarbonyl)amino]phenyl}-3,4-dihydroisoquinoline-2(1H)-carboxamide;
N-(4-{[3-(methylthio)propanoyl]amino}phenyl)-3,4-dihydroisoquinoline-2(1H)-carboxamide;
N-{4-[(cyclopentylacetyl)amino]phenyl}-3,4-dihydroisoquinoline-2(1H)-carboxamide;
N-{4-[(cyclohexylcarbonyl)amino]phenyl}-3,4-dihydroisoquinoline-2(1H)-carboxamide;
N-{4-[(cyclohexylacetyl)amino]phenyl}-3,4-dihydroisoquinoline-2(1H)-carboxamide;
N-[4-(benzoylamino)phenyl]-3,4-dihydroisoquinoline-2(1H)-carboxamide;
N-{4-[(phenylacetyl)amino]phenyl}-3,4-dihydroisoquinoline-2(1H)-carboxamide;
N-(4-{[3-(4-aminophenyl)propanoyl]amino}phenyl)-3,4-dihydroisoquinoline-2(1H)-carboxamide;
N-[4-(3-furoylamino)phenyl]-3,4-dihydroisoquinoline-2(1H)-carboxamide;
N-{4-[(2,5-dimethyl-3-furoyl)amino]phenyl}-3,4-dihydroisoquinoline-2(1H)-carboxamide;
N-{4-[(3-thienylcarbonyl)amino]phenyl}-3,4-dihydroisoquinoline-2(1H)-carboxamide;
N-{4-[(1H-pyrrol-2-ylcarbonyl)amino]phenyl}-3,4-dihydroisoquinoline-2(1H)-carboxamide;
N-{4-[(1,3-thiazol-5-ylcarbonyl)amino]phenyl}-3,4-dihydroisoquinoline-2(1H)-carboxamide;
N-{4-[(1H-pyrazol-5-ylcarbonyl)amino]phenyl}-3,4-dihydroisoquinoline-2(1H)-carboxamide;
N-{4-[(1H-pyrazol-4-ylcarbonyl)amino]phenyl}-3,4-dihydroisoquinoline-2(1H)-carboxamide;
N-{4-[(1,2-oxazol-5-ylcarbonyl)amino]phenyl}-3,4-dihydroisoquinoline-2(1H)-carboxamide;
N-{4-[(pyridin-2-ylacetyl)amino]phenyl}-3,4-dihydroisoquinoline-2(1H)-carboxamide;
N-{4-[(N,N-dimethyl-beta-alanyl)amino]phenyl}-3,4-dihydroisoquinoline-2(1H)-carboxamide;
N-(4-{[3-(piperidin-1-yl)propanoyl]amino}phenyl)-3,4-dihydroisoquinoline-2(1H)-carboxamide;
N-{4-[(morpholin-4-ylacetyl)amino]phenyl}-3,4-dihydroisoquinoline-2(1H)-carboxamide;
N-(4-{[3-(morpholin-4-yl)propanoyl]amino}phenyl)-3,4-dihydroisoquinoline-2(1H)-carboxamide;
N-(4-{[3-(4-methylpiperazin-1-yl)propanoyl]amino}phenyl)-3,4-dihydroisoquinoline-2(1H)-carboxamide;
N-{4-[(cyclopentylacetyl)amino]phenyl}-5-[(methylsulfonyl)amino]-3,4-dihydroisoquinoline-2(1H)-carboxamide;
and pharmaceutically acceptable salts thereof.

11. The compound of claim 4 selected from the group consisting of
N-[4-(1-benzoyl-1,2,3,6-tetrahydropyridin-4-yl)phenyl]-3,4-dihydroisoquinoline-2(1H)-carboxamide;
N-[4-(1-butyryl-1,2,3,6-tetrahydropyridin-4-yl)phenyl]-3,4-dihydroisoquinoline-2(1H)-carboxamide;
N-{4-[1-(isopropylsulfonyl)-1,2,3,6-tetrahydropyridin-4-yl]phenyl}-3,4-dihydroisoquinoline-2(1H)-carboxamide;
N-[4-(1-isobutyryl-1,2,3,6-tetrahydropyridin-4-yl)phenyl]-3,4-dihydroisoquinoline-2(1H)-carboxamide;
N-{4-[1-(3-methylbutanoyl)-1,2,3,6-tetrahydropyridin-4-yl]phenyl}-3,4-dihydroisoquinoline-2(1H)-carboxamide;
N-{4-[1-(methylcarbamoyl)-1,2,3,6-tetrahydropyridin-4-yl]phenyl}-3,4-dihydroisoquinoline-2(1H)-carboxamide;
tert-butyl 4-{4-[(3,4-dihydroisoquinolin-2(1H)-ylcarbonyl)amino]phenyl}-3,6-dihydropyridine-1(2H)-carboxylate;
N-[4-(1-acetyl-1,2,3,6-tetrahydropyridin-4-yl)phenyl]-3,4-dihydroisoquinoline-2(1H)-carboxamide;
N-{4-[1-(isobutylsulfonyl)-1,2,3,6-tetrahydropyridin-4-yl]phenyl}-3,4-dihydroisoquinoline-2(1H)-carboxamide;
N-[4-(1-benzyl-1,2,3,6-tetrahydropyridin-4-yl)phenyl]-3,4-dihydroisoquinoline-2(1H)-carboxamide;
N-{4-[1-(methylsulfonyl)-1,2,3,6-tetrahydropyridin-4-yl]phenyl}-3,4-dihydroisoquinoline-2(1H)-carboxamide;
N-{4-[1-(3-methylbutyl)-1,2,3,6-tetrahydropyridin-4-yl]phenyl}-3,4-dihydroisoquinoline-2(1H)-carboxamide;
and pharmaceutically acceptable salts thereof.

12. A composition for treating inflammatory and tissue repair disorders; particularly rheumatoid arthritis, inflammatory bowel disease, asthma and COPD (chronic obstructive pulmonary disease), osteoarthritis, osteoporosis and fibrotic diseases; dermatosis, including psoriasis, atopic dermatitis and ultra-violet induced skin damage; autoimmune diseases including systemic lupus erythematosis, multiple sclerosis, psoriatic arthritis, ankylosing spondylitis, tissue and organ rejection, Alzheimer's disease, stroke, athersclerosis, restenosis, diabetes, glomerulonephritis, cancer, particularly wherein the cancer is selected from breast, prostate, lung, colon, cervix, ovary, skin, CNS, bladder, pancreas, leukemia, lymphoma or Hodgkin's disease, cachexia, inflammation associated with infection and certain viral infections, including Acquired Immune Deficiency Syndrome (AIDS), adult respiratory distress syndrome, and ataxia telengiectasia, said composition comprising an excipient and a therapeutically effective amount of a compound of claim 1, or pharmaceutically acceptable salts thereof.

13. A method of treating inflammatory and tissue repair disorders; particularly rheumatoid arthritis, inflammatory bowel disease, asthma and COPD (chronic obstructive pulmonary disease), osteoarthritis, osteoporosis and fibrotic diseases; dermatosis, including psoriasis, atopic dermatitis and ultra-violet induced skin damage; autoimmune diseases including systemic lupus erythematosis, multiple sclerosis, psoriatic arthritis, ankylosing spondylitis, tissue and organ rejection, Alzheimer's disease, stroke, athersclerosis, restenosis, diabetes, glomerulonephritis, cancer, particularly wherein the cancer is selected from breast, prostate, lung, colon, cervix, ovary, skin, CNS, bladder, pancreas, leukemia, lymphoma or Hodgkin's disease, cachexia, inflammation associated with infection and certain viral infections, including Acquired Immune Deficiency Syndrome (AIDS), adult respiratory distress syndrome, and ataxia telengiectasia in a patient, said method comprising administering to the patient a therapeutically effective amount of a compound of claim 1, or pharmaceutically acceptable salts thereof.

14. A method of treating inflammatory and tissue repair disorders; particularly rheumatoid arthritis, inflammatory bowel disease, asthma and COPD (chronic obstructive pulmonary disease), osteoarthritis, osteoporosis and fibrotic diseases; dermatosis, including psoriasis, atopic dermatitis and ultra-violet induced skin damage; autoimmune diseases including systemic lupus erythematosis, multiple sclerosis, psoriatic arthritis, ankylosing spondylitis, tissue and organ rejection, Alzheimer's disease, stroke, athersclerosis, restenosis, diabetes, glomerulonephritis, cancer, particularly wherein the cancer is selected from breast, prostate, lung, colon, cervix, ovary, skin, CNS, bladder, pancreas, leukemia, lymphoma or Hodgkin's disease, cachexia, inflammation associated with infection and certain viral infections, including Acquired Immune Deficiency Syndrome (AIDS), adult respiratory distress syndrome, and ataxia telengiectasia or spleen cancer in a patient, said method comprising administering to the patient therapeutically effective amount of the compound of claim 1, or pharmaceutically acceptable salts thereof; and a therapeutically effective amount of one additional therapeutic agent or more than one additional therapeutic agent.
